(19) United States
(12) Patent Application Publication
(10) Pub. No.: US 2025/0099420 A1
(43) Pub. Date: Mar. 27, 2025

(54) SMALL MOLECULE MODULATORS OF GPR139 COMPLEX

(71) Applicant: University of Florida Research Foundation, Incorporated, Gainesville, FL (US)

(72) Inventors: Kirill Martemyanov, Jupiter, FL (US); Hannah Stoveken, Hamilton Township, NJ (US); Timothy Patrick Spicer, Stuart, FL (US); Louis Scampavia, West Palm Beach, FL (US)

(21) Appl. No.: 18/726,503

(22) PCT Filed: Jan. 25, 2023
(86) PCT No.: PCT/US2023/061229

§ 371 Date: Jul. 3, 2024

Related U.S. Application Data
(60) Provisional application No. 63/303,198, filed on Jan. 26, 2022.

(51) Int. Cl. A61K 31/353; A61K 31/167; A61K 31/18; A61K 31/235; A61K 31/277; A61K 31/341; A61K 31/357; A61K 31/366; A61K 31/381; A61K 31/4015; A61K 31/403; A61K 31/4035; A61K 31/415; A61K 31/4164; A61K 31/4166; A61K 31/4168; A61K 31/4178; A61K 31/4184; A61K 31/42; A61K 31/4245; A61K 31/4355; A61K 31/44; A61K 31/445; A61K 31/47; A61K 31/4745; A61K 31/495; A61K 31/50; A61K 31/501; A61K 31/5025; A61K 31/513; A61K 31/517; A61K 31/519; A61K 31/53; A61K 31/5415; A61K 31/55; A61K 31/551; A61K 45/06

(52) U.S. Cl. CPC A61K 31/353; A61K 31/167; A61K 31/18; A61K 31/235; A61K 31/277; A61K 31/341; A61K 31/357; A61K 31/366; A61K 31/381; A61K 31/4015; A61K 31/403; A61K 31/4035; A61K 31/415; A61K 31/4164; A61K 31/4166; A61K 31/4168; A61K 31/4178; A61K 31/4184; A61K 31/42; A61K 31/4245; A61K 31/4355; A61K 31/44; A61K 31/445; A61K 31/47; A61K 31/4745; A61K 31/495; A61K 31/50; A61K 31/501; A61K 31/5025; A61K 31/513; A61K 31/517; A61K 31/519; A61K 31/53; A61K 31/5415; A61K 31/55; A61K 31/551; A61K 45/06

(57) ABSTRACT

Described are GPR139 modulators, agonists and antagonists of GPR139 activity and/or GPR139 signaling. Pharmaceutical compositions comprising the GPR139 modulators are also described. The GPR139 modulators and pharmaceutical compositions comprising the GPR139 modulators can be used to enhance opioid analgesic efficacy, suppress symptoms associated with opioid withdrawal, decrease opioid addiction or dependence, or treat neuropsychiatric disorders.

### CROSS-REFERENCE TO RELATED APPLICATIONS

This application claims the benefit of U.S. Provisional Application No. 63/303,198, filed Jan. 26, 2022, which is incorporated herein by reference

### STATEMENT REGARDING FEDERALLY SPONSORED RESEARCH OR DEVELOPMENT

This invention was made with government support under grant numbers R01 DA042746 and F32 DA047771, awarded by the National Institutes of Health/National Institute on Drug Abuse. The government has certain rights in the invention.

### BACKGROUND

G protein coupled receptors (GPCRs) constitute the largest class of cell surface receptors and are responsible for sensory perception and cellular communication via hormones and neurotransmitters. GPCRs are also heavily involved in disease and are the most prominent drug targets. Insufficient understanding of GPCR signaling significantly hampers their targeting by drugs in a safe and effective manner. This is well illustrated by opioid analgesics that act on the μ-opioid receptor (MOR): they offer unsurpassed efficacy for pain management. However, virtually all FDA approved opioid drugs come with substantial liabilities including: dependence, loss of efficacy over time, and somatic side effects. Extensive investigation of MOR pharmacology led to the concept that activated MOR triggers distinct signaling events that can be differentially dissociated to control various physiological reactions. Nonetheless, very little is known about the identity of biasing factors that route MOR signals and determine their efficacy in vivo.

We have recently discovered orphan receptor GPR139 for its powerful “anti-opioid” activity. GPR139 forms heteromers with μ-opioid receptor (MOR) in distinct neural circuits involved in analgesia, reward and withdrawal, including the medial habenula (mHb) and the locus coeruleus (LC). Studies with GPR139 knockout mice show an augmentation of opioid induced analgesia with substantially reduced dependence, as evidenced by lack of somatic withdrawal upon cessation of chronic morphine administration. These data provide strong rationale for pharmacological inhibition of GPR139 to alter the processing of MOR signals to combat addiction.

There are unmet needs in the art for means to better understand MOR signaling in endogenous neural circuits, and for novel agents that can increase efficacy and safety of opioid analgesics by modulating MOR signaling in vivo. There is also a need in the art for more effective treatment for opioid dependence and withdrawal.

In addition, we have recently discovered that mice lacking GPR139 display prominent neuropsychiatric phenotypes that include hyperactivity, anxiety, deficits on movement initiation, cognitive issues, social deficits and particularly strongly featuring traits that correlate with psychosis in humans. This includes deficits in social interactions, pre-pulse inhibition, and spontaneous head twitching (Dao et al. Neuropsychopharmacology January 2021). Opioid and dopaminergic antagonism was effective in reversing these abnormal behaviors in adult mice suggesting that GPR139 agonists could show efficacy as novel anti-psychotics. The present invention addresses these and other needs.

### SUMMARY

Described are modulators, agonists and antagonists, of GPR139 and/or GPR139 signaling. Antagonists of GPR139 and/or GPR139 signaling are shown in FIG. 1(A-H). Agonists of GPR139 and/or GPR139 signaling are shown in FIG. 2 (A-B2). Pharmaceutical compositions comprising the GPR139 antagonists and GPR139 agonists are also described.

Described are methods for antagonizing, or reducing, GPR139 activity and/or GPR139 signaling activity. The methods comprise administering to a subject a therapeutically effective amount of any one or more of the compounds of Table 1 (GPR139 antagonists). Administering a therapeutically effective amount of the GPR139 antagonist includes, but is not limited to, administering to the subject a pharmaceutical composition that contains the therapeutically effective amount of the GPR139 antagonist. A GPR139 antagonist can be used to enhance an analgesic effect mediated by an μ-opioid receptor (MOR) in a subject. Reducing GPR139 activity and/or GPR139 signaling can lead to stimulation or enhancement of MOR mediated analgesic effect in the subject. Reducing GPR139 activity is also expected to ameliorate withdrawal and thus be beneficial for combating dependence by facilitating opioid abstinence. In some methods, the administration of a GPR139 antagonist promotes or enhances MOR signaling mediated by endogenous ligands in the subject.

In some embodiments, a subject is administered with an opioid drug for pain relief and one or more GPR139 antagonists, wherein the one or more GPR 139 antagonists enhances the effect of the opioid drug. The opioid drug can be administered to the subject prior to, simultaneously with, or subsequent to administration of the one or more GPR139 antagonists. In some embodiments, the opioid drug and the GPR139 antagonist are formulated together. In some embodiments, the opioid drug and the GPR139 antagonist are formulated for separate administration. The opioid drug can be, but is not limited to, oxycodone, hydrocodone, morphine, codeine, dihydrocodeine, fentanyl, buprenorphine, or methadone. The subject can be, but is not limited to, a human.

In some embodiments, methods are described for suppressing or ameliorating withdrawal symptoms in subjects suffering from chronic use of an opioid drug (e.g., addiction), to assist a subject in reducing dependence on an opioid drug, to assist a subject in reducing opioid use, or to treat an opioid use disorder. These methods comprise administering to the subject an effective amount of one or more GPR139 antagonists, thereby suppressing or ameliorating one or more withdrawal symptoms in the subject. The subject can be suffering from one or more opioid withdrawal symptoms or at risk of suffering from one or more opioid withdrawal symptoms. In some methods, a GPR139 antagonist is administered to a subject after discontinuing or reducing use of the opioid drug. In some methods, a GPR139 antagonist is administered to a subject prior to discontinuing or reducing use of the opioid drug. Reducing use of the opioid drug includes, but is not limited to, reducing dosage or frequency of administration of the opioid drug. The opioid drug can be, but is not limited to, oxycodone, hydrocodone, morphine, codeine, dihydrocodeine, heroin, opium, or fentanyl. The subject can be, but is not limited to, a human.

In some embodiments, a described GPR139 antagonist can be used to treat an anxiety disorder. Described are methods of treating an anxiety disorder in a subject comprising administering to the subject an effective amount of one or more GPR139 antagonists of Table 1.

Described are methods for activating, or increasing, GPR139 activity and/or GPR139 signaling activity. The methods comprise administering to a subject a therapeutically effective amount of any one or more of the compounds of Table 2 (GPR139 agonists). Administering a therapeutically effective amount of the GPR139 agonist includes, but is not limited to, administering to the subject a pharmaceutical composition that contains the therapeutically effective amount of the GPR139 agonist.

Described are methods for antagonizing, or reducing, GPR139 activity and/or GPR139 signaling activity in a cell by contacting the cell with one or more of GPR139 antagonists of FIG. 1 (A-H). In some embodiments, the cell is in a mammal, such as, but not limited to, a human.

In some embodiments, a GPR139 agonist is administered to a subject to reduce the reward (e.g., euphoric effects) associated with opioid use or to diminish the reinforcing effects of opioid use. Reducing the reward associated with opioid use or diminishing the reinforcing effects of opioid use can be used to treat or prevent addiction in a subject. Methods of treating or preventing opioid addiction comprise administering to the subject an effective amount of one or more GPR139 agonists of Table 2. Administering the GPR agonist to the subject can increase expression or cellular activity of GPR139 or an ortholog thereof and/or increase GPR139 signaling activity. The subject can be, but is not limited to, a human.

In some embodiments, the GPR139 agonists can be used to treat neuropsychiatric disorders or conditions or psychoses. Described are methods of treating neuropsychiatric disorders or conditions or psychoses in a subject comprising administering to the subject an effective amount of one or more GPR139 agonists of Table 2. Also described are methods of treating one or more symptoms associated with a neuropsychiatric disorder or condition or psychosis in a subject comprising administering to the subject an effective amount of one or more GPR139 agonists of Table 2. Treating one or more symptoms associated with a neuropsychiatric disorder or condition or psychosis includes, but is not limited to, decreasing or suppressing the one or more symptoms, decreasing the frequency of the one or more symptoms, decreasing the severity of the one or more symptoms, or decreasing progression of the one or more symptoms. Treating the neuropsychiatric disorders or conditions or psychoses can also include improving the quality of life of the person suffering from the neuropsychiatric disorder or condition or psychosis or decreasing the dosage one or more other therapeutics taken by the subject to treat the neuropsychiatric disorder or condition or psychosis. The neuropsychiatric disorder or condition or psychosis can be, but is not limited to, deficits in social interactions, pre-pulse inhibition, and spontaneous head twitching. The GPR139 agonists as be used as stand alone antipsychotics or the may be combined with one or more additional antipsychotics. The subject can be, but is not limited to, a human.

Described are methods for increasing GPR139 activity and/or GPR139 signaling activity in a cell comprising contacting the cell with one or more of GPR139 agonists of FIG. 2 (A-B2). In some embodiments, the cell is in a mammal, such as, but not limited to, a human.

A further understanding of the nature and advantages of the present invention may be realized by reference to the remaining portions of the specification and claims.

### BRIEF DESCRIPTION OF THE DRAWINGS

FIG. 1A. Graphs illustrating dose response curves for the GPR139 antagonists. For each of FIG. 1A-1H, the x axis is the concentration (molarity) of the compound and they axis is the percent activation (GPR139 response).

FIG. 1B. Graphs illustrating dose response curves for the GPR139 antagonists.

FIG. 1C. Graphs illustrating dose response curves for the GPR139 antagonists.

FIG. 1D. Graphs illustrating dose response curves for the GPR139 antagonists.

FIG. 1E Graphs illustrating dose response curves for the GPR 139 antagonists.

FIG. 1F. Graphs illustrating dose response curves for the GPR139 antagonists.

FIG. 1G. Graphs illustrating dose response curves for the GPR139 antagonists.

FIG. 1H. Graphs illustrating dose response curves for the GPR139 antagonists.

FIG. 2A. Graphs illustrating dose response curves for the GPR139 agonists. For each of FIG. 2A-2B2, the x axis is the concentration (molarity) of the compound and the y axis is the percent activation (GPR139 response).

FIG. 2B. Graphs illustrating dose response curves for the GPR139 agonists.

FIG. 2C. Graphs illustrating dose response curves for the GPR139 agonists.

FIG. 2D. Graphs illustrating dose response curves for the GPR139 agonists.

FIG. 2E. Graphs illustrating dose response curves for the GPR139 agonists.

FIG. 2F. Graphs illustrating dose response curves for the GPR139 agonists.

FIG. 2G. Graphs illustrating dose response curves for the GPR139 agonists.

FIG. 2H. Graphs illustrating dose response curves for the GPR139 agonists.

FIG. 2I. Graphs illustrating dose response curves for the GPR139 agonists.

FIG. 2J. Graphs illustrating dose response curves for the GPR139 agonists.

FIG. 2K. Graphs illustrating dose response curves for the GPR139 agonists.

FIG. 2L. Graphs illustrating dose response curves for the GPR139 agonists.

FIG. 2M. Graphs illustrating dose response curves for the GPR139 agonists.

FIG. 2N. Graphs illustrating dose response curves for the GPR139 agonists.

FIG. 2O. Graphs illustrating dose response curves for the GPR139 agonists.

FIG. 2P. Graphs illustrating dose response curves for the GPR139 agonists.

FIG. 2Q. Graphs illustrating dose response curves for the GPR139 agonists.

FIG. 2R. Graphs illustrating dose response curves for the GPR139 agonists.

FIG. 2S. Graphs illustrating dose response curves for the GPR139 agonists.

FIG. 2T. Graphs illustrating dose response curves for the GPR139 agonists.

FIG. 2U. Graphs illustrating dose response curves for the GPR139 agonists.

FIG. 2V. Graphs illustrating dose response curves for the GPR139 agonists.

FIG. 2W. Graphs illustrating dose response curves for the GPR139 agonists.

FIG. 2X. Graphs illustrating dose response curves for the GPR139 agonists.

FIG. 2Y. Graphs illustrating dose response curves for the GPR 139 agonists.

FIG. 2Z. Graphs illustrating dose response curves for the GPR139 agonists.

FIG. 2A1. Graphs illustrating dose response curves for the GPR139 agonists.

FIG. 2B1. Graphs illustrating dose response curves for the GPR139 agonists.

FIG. 2C1. Graphs illustrating dose response curves for the GPR139 agonists.

FIG. 2D1. Graphs illustrating dose response curves for the GPR139 agonists.

FIG. 2E1. Graphs illustrating dose response curves for the GPR139 agonists.

FIG. 2F1. Graphs illustrating dose response curves for the GPR139 agonists.

FIG. 2G1. Graphs illustrating dose response curves for the GPR139 agonists.

FIG. 2H1. Graphs illustrating dose response curves for the GPR139 agonists.

FIG. 2I1. Graphs illustrating dose response curves for the GPR139 agonists.

FIG. 2J1. Graphs illustrating dose response curves for the GPR139 agonists.

FIG. 2K1. Graphs illustrating dose response curves for the GPR139 agonists.

FIG. 2L1. Graphs illustrating dose response curves for the GPR139 agonists.

FIG. 2M1. Graphs illustrating dose response curves for the GPR139 agonists.

FIG. 2N1. Graphs illustrating dose response curves for the GPR139 agonists.

FIG. 2O1. Graphs illustrating dose response curves for the GPR139 agonists.

FIG. 2P1. Graphs illustrating dose response curves for the GPR139 agonists.

FIG. 2Q1. Graphs illustrating dose response curves for the GPR139 agonists.

FIG. 2R1. Graphs illustrating dose response curves for the GPR139 agonists.

FIG. 2S1. Graphs illustrating dose response curves for the GPR139 agonists.

FIG. 2T1. Graphs illustrating dose response curves for the GPR139 agonists.

FIG. 2U1. Graphs illustrating dose response curves for the GPR139 agonists.

FIG. 2V1. Graphs illustrating dose response curves for the GPR139 agonists.

FIG. 2W1. Graphs illustrating dose response curves for the GPR139 agonists.

FIG. 2X1. Graphs illustrating dose response curves for the GPR139 agonists.

FIG. 2Y1. Graphs illustrating dose response curves for the GPR139 agonists.

FIG. 2Z1. Graphs illustrating dose response curves for the GPR139 agonists.

FIG. 2A2. Graphs illustrating dose response curves for the GPR139 agonists.

FIG. 2B2. Graphs illustrating dose response curves for the GPR139 agonists.

FIG. 3A. Graph illustrating radioligand binding of [³H]JNJ-63533054 on membranes from HEK293T/17 cells transfected with human GPR139.

FIG. 3B-C. Graphs illustrating competition of [³H]JNJ-63533054 binding by SR-01000506165, SR-01000253143 and SR-01000110577 on membranes from HEK293T/17 cells transfected with human GPR139. Results are presented as means±SEM (n=3).

### DETAILED DESCRIPTION

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s), or method step(s) that do not materially change the basic and novel characteristics of the compositions and methods described herein may be included in those compositions or methods.

All ranges are to be interpreted as encompassing the endpoints in the absence of express exclusions, such as “not including the endpoints”; thus, for example. “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When values are expressed as approximations, by use of the antecedent “about,” it will be understood that the particular value forms a further aspect. For example, if the value “about 10” is disclosed, then “10” is also disclosed.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, which is the drug substance itself, and excipients, which are the ingredients of the tablet, or the liquid in which the active agent is suspended, or other material that is pharmaceutically inert. During formulation development, the excipients can be selected so that the active ingredient can reach the target site in the body at the desired rate and extent.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An “effective amount” can also refer to the amount of, for example an excipient, in a pharmaceutical composition that is sufficient to achieve the desired property of the composition. An effective amount can be administered in one or more administrations, applications, or dosages.

As used herein, “dose.” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions. Treating can refer to both therapeutic treatment alone, prophylactic treatment alone, or both therapeutic and prophylactic treatment. Those in need of treatment (subjects in need thereof) can include those already with disease or condition or those in which disease or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom.

“Opioid” includes opioid drugs and opioid-related drugs or compounds that are members of a class of drugs either derived from, or chemically similar to, compounds found in opium poppies. Examples of opioids include legal prescription painkillers like oxycodone (OxyContin®), hydrocodone (Vicodin®), morphine, codeine, dihydrocodeine, fentanyl, and the like, and illegal drugs such as opium and heroin. Opioids also include antagonist drugs such as naloxone, and endogenous peptides such as endorphins. In some embodiments, opioid compounds can also include partial agonists of MOR, e.g. buprenorphine and methadone.

An “opioid use disorder” is a substance use disorder (persistent use of a drug despite harm and adverse consequences) relating to the use of an opioid. Signs of the disorder include a strong desire to use opioids, impaired control over its use, increased tolerance to opioids, persistent use despite harmful consequences, trouble reducing use, and withdrawal symptoms with discontinuation. Opioid withdrawal symptoms include, but are not limited to, nausea, muscle aches, diarrhea, trouble sleeping, agitation, and a low mood. Addiction and dependence are components of a substance use disorder.

Withdrawal, withdrawal symptoms, or withdrawal syndrome refers to a collection of symptoms and the degree of severity which the symptoms occur on cessation or abrupt reduction of use of a psychoactive substance (e.g., an opioid drug) that has been taken repeatedly, usually for a prolonged period and/or in high doses. The syndrome may be accompanied by signs of physiological and/or emotional disturbance. A withdrawal symptom is one of the indicators of a dependence syndrome.

The μ-opioid receptors (MOR) are a class of opioid receptors with a high affinity for endogenous opioid peptides enkephalins and beta-endorphin, but a low affinity for dynorphins. They are also referred to as μ-opioid peptide (MOP) receptors. The prototypical exogenous μ-opioid receptor agonist is morphine, the primary psychoactive alkaloid in opium. It is an inhibitory G-protein coupled receptor that activates several inhibitory G protein subunits, including Gᵢα, Gₒα, G_{z}α, and G_{βγ} (G beta-gamma), inhibiting activity of adenylate cyclase to lower cAMP levels and several ion channels to reduce neuronal excitability and synaptic transmission. Activation of the μ-opioid receptor by an agonist, such as morphine, causes analgesia, sedation, slightly reduced blood pressure, itching, nausea, euphoria, decreased respiration, miosis (constricted pupils), and decreased bowel motility often leading to constipation. Some of these effects, such as analgesia, sedation, euphoria, itching, and decreased respiration, tend to lessen with continued use as tolerance develops. As with other G protein-coupled receptors, signaling by the μ-opioid receptor is terminated through several different mechanisms, which are upregulated with chronic use, leading to rapid tachyphylaxis.

GPR139 (G (Gq)-protein coupled receptor 139) is a protein encoded by the GPR139 gene (in human). GPR139 is an orphan receptor identified from bioinformatics analysis of the human genome. It has been shown to have a high mRNA expression in the brain, particularly in the striatum and hypothalamus, and pituitary. L-Tryptophan, L-phenylalanine, and a-MSH derived peptides have been identified as putative endogenous ligands of GPR139. But the main signal transduction pathway of GPR139 has not been established. GPR139 is believed to be involved in movement control and/or the regulation of food intake/metabolism, and could play a role in the control of locomotor activity. GPR139 has been suggested as a potential target for the treatment of Parkinson's disease, obesity, eating disorders, and/or diabetes.

GPR139 has been shown to be extensively coexpressed with MOR in a number of neuronal populations in these areas including medial habenula, striatum, locus coeruleus, and periaqueductal grey matter (US20210338680). Deletion of GPR139 in mice (Gpr139−/−) resulted in no overt effects on animal health and body composition. Gpr139−/− mice were indistinguishable from their wild-type littermates in all baseline behaviors tested, including baseline nociception, learning, locomotor activity, habituation to novel environment, and motor coordination. Gpr139−/− mice exhibited significantly increased morphine analgesia, including maximal response and duration of effect across multiple drug doses. Gpr139−/− mice also showed substantially augmented responses to the rewarding effects of morphine in a conditioned place preference paradigm. These results show that deletion of GPR139 increases sensitivity to the acute effects of morphine. Gpr139−/− mice also exhibited lower dependence associated with chronic morphine administration and exhibited diminished withdrawal symptoms across a spectrum of behaviors. We have recently discovered that mice lacking GPR139 display neuropsychiatric phenotypes, including featuring traits that correlate with psychosis in humans. Such neuropsychiatric phenotypes includes deficits in social interactions, pre-pulse inhibition, and spontaneous head twitching. Opioid and dopaminergic antagonism was effective in reversing these abnormal behaviors in adult mice suggesting that GPR139 agonists can be useful as anti-psychotics, either as stand alone therapeutics or in combination with other anti-psychotics.

“Orthologs” are genes and products thereof in different species that evolved from a common ancestral gene by speciation and retain the same or similar function. An ortholog is a gene that is related by vertical descent and is responsible for substantially the same or identical functions in different organisms. For example, mouse GPR139 and human GPR139 can be considered orthologs. Genes may share sequence similarity of sufficient amount to indicate they are orthologs. Protein may share three-dimensional structure of sufficient amount to indicate the proteins and the genes encoding them are orthologs. Methods of identifying orthologs are known in the art.

A GPR Antagonist is a compound that down-regulates or inhibits expression, levels, or cellular activity of GPR139 or a GPR139 ortholog, down-regulates or inhibits GPR139 signaling activity, or down-regulates or inhibits GPCR signaling activity of GPR139 or an ortholog thereof.

A GPR agonist is a compound that up-regulates or increase expression, levels, or cellular activity of GPR139 or a GPR139 ortholog, up-regulates or increase GPR139 signaling activity, or up-regulates or increase GPCR signaling activity of GPR139 or an ortholog thereof.

An “analog” refers to a molecule that structurally resembles a reference molecule (e.g., a GPR139 antagonist) but which has been modified in a targeted and controlled manner, by replacing a specific substituent of the reference molecule with an alternate substituent. Compared to the reference molecule, an analog would be expected, by one skilled in the art, to exhibit the same or similar utility. Synthesis and screening of analogs to identify variants of known compounds having improved characteristics (such as higher binding affinity for a target molecule) is an approach that is well known in pharmaceutical chemistry.

A “derivative” of a first compound is a compound that has a three dimensional structure that is similar to at least a part of the first compound. In some embodiments, a derivative is a compound that is derived from, or imagined to derive from, another compound such as by substitution of one atom or group with another atom or group. In some embodiments, derivatives are compounds that at least theoretically can be formed from a common precursor compound.

Opioid analgesics offer unrivaled pain management but have severe abuse liability. Opioids produce clinically significant effects via the μ-opioid receptor (MOR), a member of the G protein Coupled Receptor (GPCR) family. Saturation of MOR as a drug target presents a pressing need to discover new modifiers that alter MOR signaling outcomes. Cell-based assays have revealed that GPR139 inhibits MOR. GPR139 is coexpressed with MOR in select brain circuits underlying opioid action. Elimination of GPR139 in mice augments morphine-induced analgesia and reward, but diminishes dependence. Thus, GPR139 is a viable target for increasing safety of opioid pharmacotherapies.

Described are GPR139 agonists and antagonists, pharmaceutical compositions containing the GPR139 agonists and antagonists, and methods of using the GPR139 agonists and antagonists.

In some embodiments, methods for modulating analgesic response in subjects who are taking opioid related drugs are provided. Administering a GPR139 antagonist to a subject can be used to increase efficacy of an opioid drug and/or diminishing the dependence-causing liability of the opioid drug. In some embodiments, GPR139 agonists are administered to a subject to diminish reinforcing effects (reward) of opioids. Such use can be used to treat or prevent addiction. The subject can be, but is not limited to, a subject that is an acute user or a chronic user of opioids. In some embodiments, a GPR antagonist enhancing endogenous opioid signaling. Enhancing endogenous opioid signaling can be used to provide or enhance analgesic response, such as by an opioid. Increased efficacy of an opioid includes, but is not limited to, enhancing maximal response to the opioid, increased response to a given dose of opioid, decreasing the amount of the opioid required to provide an effective analgesic response, increasing duration of analgesic response to the opioid, and decreasing risk of addiction or dependence on the opioid.

In some embodiments, methods are provided for modulating cellular activities mediated by the μ-opioid receptor (MOR) signaling, comprising administering to a subject a GPR139 antagonist or GPR139 agonist. Down-regulation of GPR139, using a described GPR139 antagonist, enhances MOR signaling and sensitivity to opioid compounds. Up-regulation of GPR139, using a described GPR139 agonist, inhibits MOR signaling and opioid efficacy.

In some embodiments, a subject in need of treatment, such as opioid treatment, is administered a described GPR139 antagonist in addition to the opioid, to promote MOR mediated signaling activity and enhance opioid efficacy. The GPR139 antagonist can be administered prior to administration of the opioid, concurrent with opioid administration, or subsequent to opioid administration. The GPR139 antagonist may be administered to the subject to improve pain relief associated with opioid treatment.

In some embodiments, a described GPR139 antagonist is administered to a subject affected by chronic use of an opioid to diminish dependence and/or ameliorate withdrawal symptoms. The GPR139 antagonist can be administered prior to administration of the opioid, concurrent with opioid administration, subsequent to opioid administration (after the subject has stopped taking the opioid), or subsequent to a reduction in opioid use by the subject.

In some embodiments, a described GPR139 antagonist is administered to a subject to reduce dependence on an opioid, control relapse, or suppress or ameliorate withdrawal symptoms, wherein the subject has been chronically taking one or more opioids or suffers from an opioid use disorder. The GPR139 antagonist can be administered to the subject prior to the subject discontinuing or reducing opioid drug use, concurrent with discontinuing or reducing opioid drug use, or after discontinuing or reducing opioid drug use. In some embodiments, the GPR139 antagonist is administered to a subject that has discontinued or reduced opioid use and is suffering from one or more withdrawal symptoms. The GPR139 antagonist can be administered to the subject for as long as the one or more withdrawal symptoms persist.

Subjects taking any opioid related drugs are amenable to treatment with the described GPR139 modulators. The opioid drugs include, but are not limited to, morphine, synthetic opioid related compounds, methadone, oxycodone, hydrocodone, codeine, dihydrocodeine, pethidine, hydromorphone, heroin, opium, and fentanyl.

In some embodiments, the GPR139 antagonists can be used to treat an anxiety disorder. Described are methods of treating an anxiety disorder in a subject comprising administering to the subject an effective amount of one or more GPR139 antagonists of Table 1. Also described are methods of treating one or more symptoms associated with an anxiety disorder in a subject comprising administering to the subject an effective amount of one or more GPR139 antagonists of Table 1. Treating one or more symptoms associated with an anxiety disorder, but is not limited to, decreasing or suppressing the one or more symptoms, decreasing the frequency of the one or more symptoms, decreasing the severity of the one or more symptoms, or decreasing progression of the one or more symptoms. Treating the anxiety disorder can also include improving the quality of life of the person suffering from the anxiety disorder decreasing the dosage of one or more other therapeutics taken by the subject to treat the anxiety disorder.

TABLE 1
GBP139 Antagonists.
COMPOUNDS/STRUCTURES
SR-01000438114 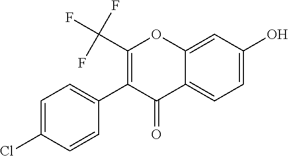 [SMILES: O=c1c(-c2ccc(Cl)cc2)c(C(F)(F)F)oc2cc(O)ccc12]
SR-01000555431 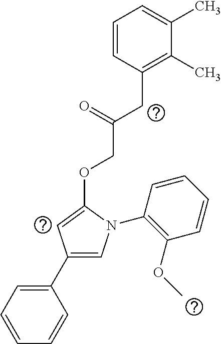 [SMILES: COc1ccccc1-n1cc(-c2ccccc2)cc1OCC(=O)Cc1cccc(C)c1C]
SR-01000471218 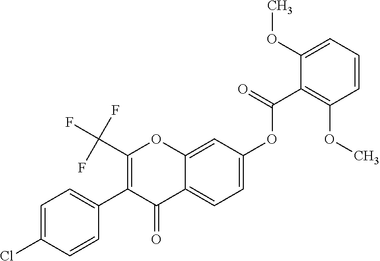 [SMILES: COc1cccc(OC)c1C(=O)Oc1ccc2c(=O)c(-c3ccc(Cl)cc3)c(C(F)(F)F)oc2c1]
SR-01000555475 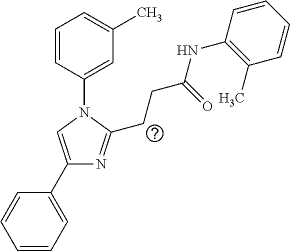 [SMILES: Cc1cccc(-n2cc(-c3ccccc3)nc2CCC(=O)Nc2ccccc2C)c1]
SR-07000003837 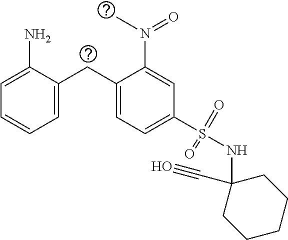
SR-07000003837-2-B 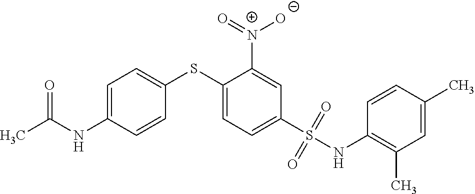 [SMILES: CC(=O)Nc1ccc(Sc2ccc(S(=O)(=O)Nc3ccc(C)cc3C)cc2[N+](=O)[O-])cc1]
SR-01000220077 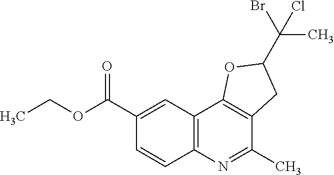 [SMILES: CCOC(=O)c1ccc2nc(C)c3c(c2c1)OC(C(C)(Cl)Br)C3]
SR-01000555409 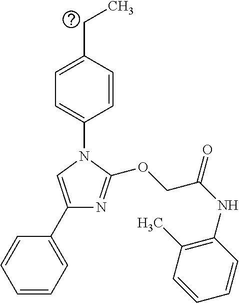 [SMILES: CCc1ccc(-n2cc(-c3ccccc3)nc2OCC(=O)Nc2ccccc2C)cc1]
SR-01000110742 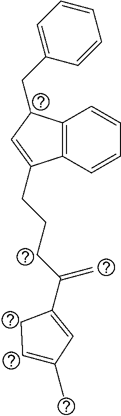 [SMILES: C=C(CCCC1=CC(Cc2ccccc2)c2ccccc21)C1=CC(C)=CC1]
SR-01000555413 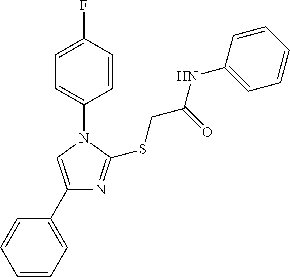 [SMILES: O=C(CSc1nc(-c2ccccc2)cn1-c1ccc(F)cc1)Nc1ccccc1]
SR-01000555462 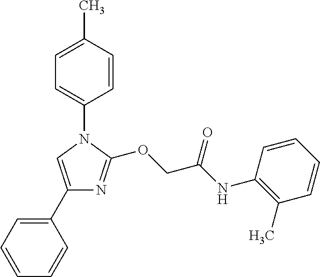 [SMILES: Cc1ccc(-n2cc(-c3ccccc3)nc2OCC(=O)Nc2ccccc2C)cc1]
SR-01000308363 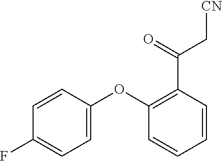 [SMILES: N#CCC(=O)c1ccccc1Oc1ccc(F)cc1]
SR-07000003837-2-C 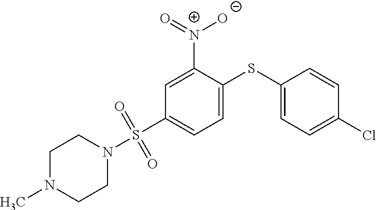 [SMILES: CN1CCN(S(=O)(=O)c2ccc(Sc3ccc(Cl)cc3)c([N+](=O)[O-])c2)CC1]
SR-01000907648 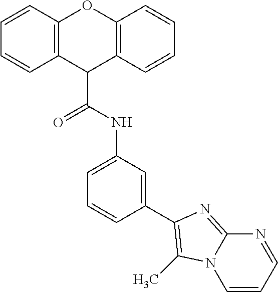 [SMILES: Cc1c(-c2cccc(NC(=O)C3c4ccccc4Oc4ccccc43)c2)nc2ncccn12]
SR-01000907648-1-B 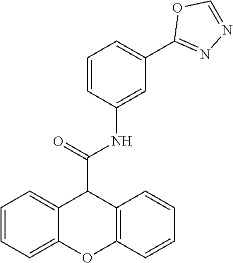 [SMILES: O=C(Nc1cccc(-c2nnco2)c1)C1c2ccccc2Oc2ccccc21]
SR-01000907209 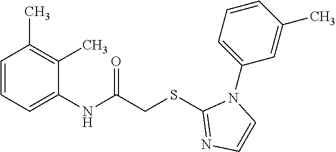 [SMILES: Cc1cccc(-n2ccnc2SCC(=O)Nc2cccc(C)c2C)c1]
SR-01000564946 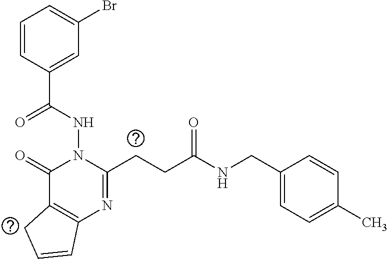 [SMILES: Cc1ccc(CNC(=O)CCc2nc3c(c(=O)n2NC(=O)c2cccc(Br)c2)CC=C3)cc1]
SR-01000263867 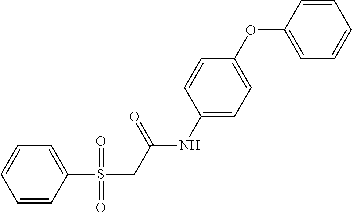 [SMILES: O=C(CS(=O)(=O)c1ccccc1)Nc1ccc(Oc2ccccc2)cc1]
SR-01000914310 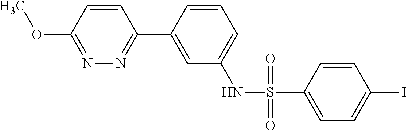 [SMILES: COc1ccc(-c2cccc(NS(=O)(=O)c3ccc(I)cc3)c2)nn1]
SR-01000581959 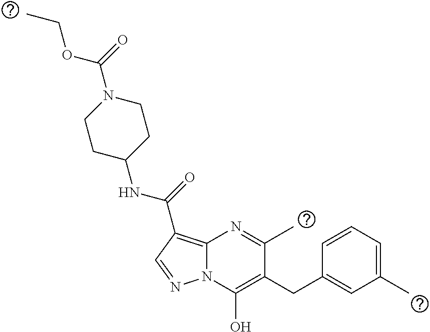 [SMILES: CCOC(=O)N1CCC(NC(=O)c2cnn3c(O)c(Cc4cccc(C)c4)c(C)nc23)CC1]
SR-01000479575 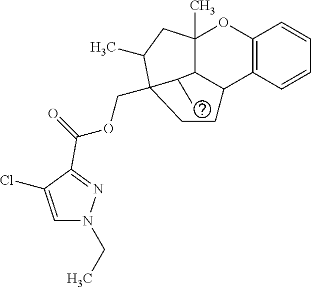 [SMILES: CCn1cc(Cl)c(C(=O)OCC23CCC4c5ccccc5OC(C)(CC2C)C4C3C)n1]
SR-01000195221 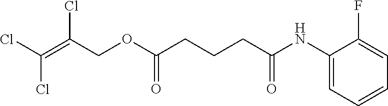 [SMILES: O=C(CCCC(=O)OCC(Cl)=C(Cl)Cl)Nc1ccccc1F]
SR-01000555444 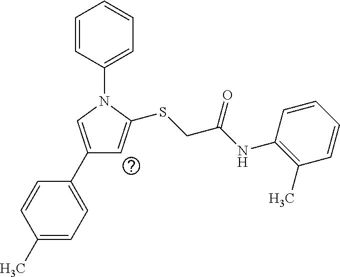 [SMILES: Cc1ccc(-c2cc(SCC(=O)Nc3ccccc3C)n(-c3ccccc3)c2)cc1]
SR-01000110577 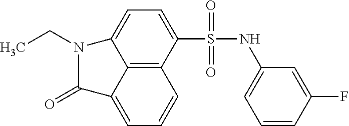 [SMILES: CCN1C(=O)c2cccc3c(S(=O)(=O)Nc4cccc(F)c4)ccc1c23]
SR-01000419723 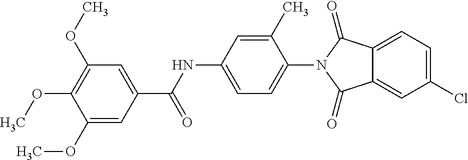 [SMILES: COc1cc(C(=O)Nc2ccc(N3C(=O)c4ccc(Cl)cc4C3=O)c(C)c2)cc(OC)c1OC]
SR-01000512383 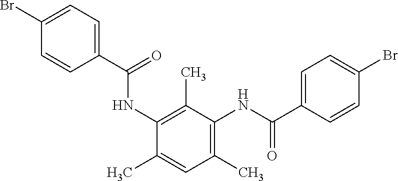 [SMILES: Cc1cc(C)c(NC(=O)c2ccc(Br)cc2)c(C)c1NC(=O)c1ccc(Br)cc1]
SR-01000258561 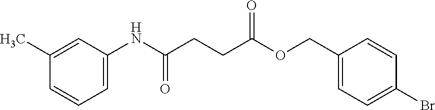 [SMILES: Cc1cccc(NC(=O)CCC(=O)OCc2ccc(Br)cc2)c1]
SR-01000508699 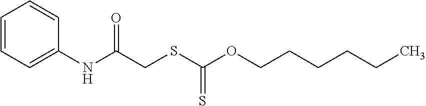 [SMILES: CCCCCCOC(=S)SCC(=O)Nc1ccccc1]
SR-01000227608 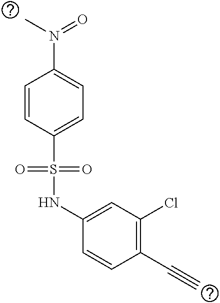
SR-05000022540 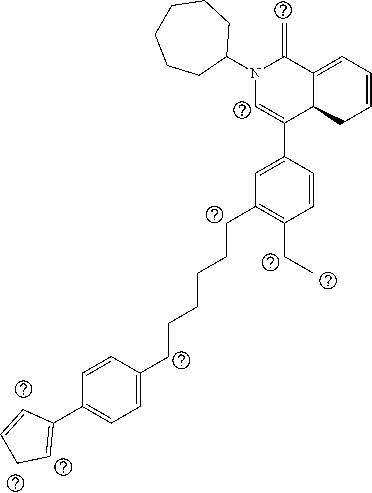 [SMILES: C=C1C2=CC=CC[C@H]2C(c2ccc(CC)c(CCCCCCc3ccc(C4=CCC=C4)cc3)c2)=CN1C1CCCCCC1]
SR-01000244770 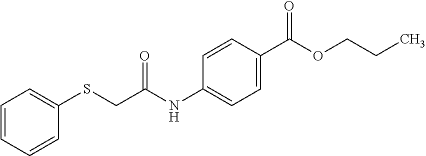 [SMILES: CCCOC(=O)c1ccc(NC(=O)CSc2ccccc2)cc1]
SR-01000433654 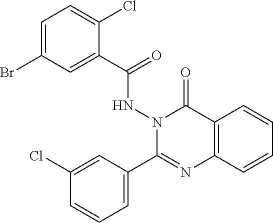 [SMILES: O=C(Nn1c(-c2cccc(Cl)c2)nc2ccccc2c1=O)c1cc(Br)ccc1Cl]
SR-01000555394 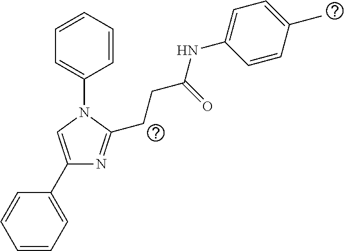 [SMILES: Cc1ccc(NC(=O)CCc2nc(-c3ccccc3)cn2-c2ccccc2)cc1]
SR-01000581835 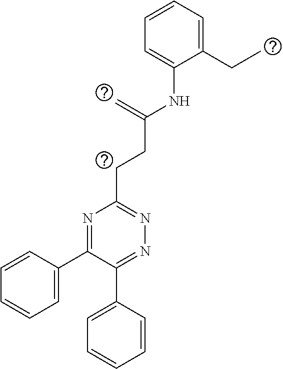 [SMILES: C=C(CCc1nnc(-c2ccccc2)c(-c2ccccc2)n1)Nc1ccccc1CC]
SR-01000421299 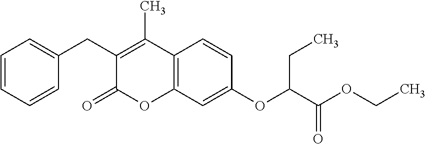 [SMILES: CCOC(=O)C(CC)Oc1ccc2c(C)c(Cc3ccccc3)c(=O)oc2c1]
SR-01000575726 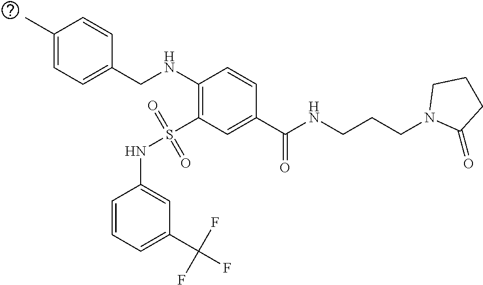 [SMILES: Cc1ccc(CNc2ccc(C(=O)NCCCN3CCCC3=O)cc2S(=O)(=O)Nc2cccc(C(F)(F)F)c2)cc1]
SR-01000506165 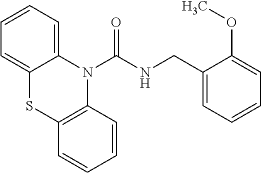 [SMILES: COc1ccccc1CNC(=O)N1c2ccccc2Sc2ccccc21]
SR-01000228964 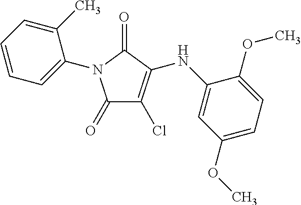 [SMILES: COc1ccc(OC)c(NC2=C(Cl)C(=O)N(c3ccccc3C)C2=O)c1]
SR-01000579741 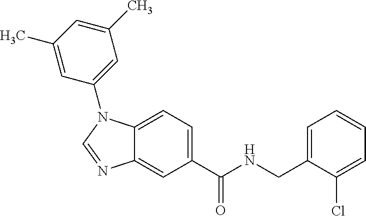 [SMILES: Cc1cc(C)cc(-n2cnc3cc(C(=O)NCc4ccccc4Cl)ccc32)c1]
SR-01000201144 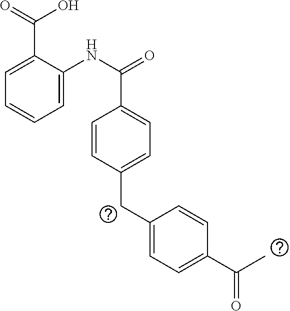 [SMILES: CC(=O)c1ccc(Cc2ccc(C(=O)Nc3ccccc3C(=O)O)cc2)cc1]
SR-01000390845 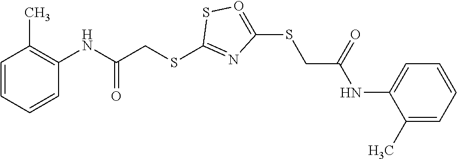
SR-01000452455 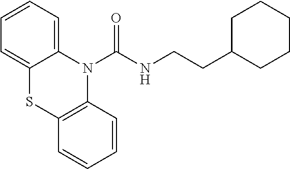 [SMILES: O=C(NCCC1CCCCC1)N1c2ccccc2Sc2ccccc21]
SR-01000594343 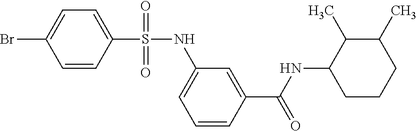 [SMILES: CC1CCCC(NC(=O)c2cccc(NS(=O)(=O)c3ccc(Br)cc3)c2)C1C]
SR-01000419112 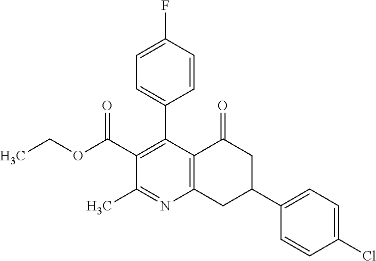 [SMILES: CCOC(=O)c1c(C)nc2c(c1-c1ccc(F)cc1)C(=O)CC(c1ccc(Cl)cc1)C2]
SR-01000529482 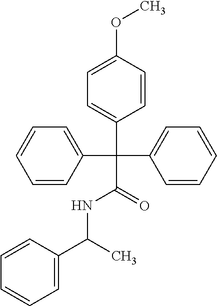 [SMILES: COc1ccc(C(C(=O)NC(C)c2ccccc2)(c2ccccc2)c2ccccc2)cc1]
SR-01000169333 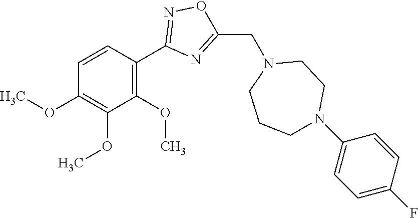 [SMILES: COc1ccc(-c2noc(CN3CCCN(c4ccc(F)cc4)CC3)n2)c(OC)c1OC]
SR-01000514922 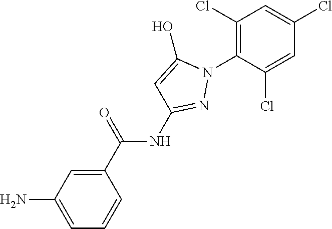 [SMILES: Nc1cccc(C(=O)Nc2cc(O)n(-c3c(Cl)cc(Cl)cc3Cl)n2)c1]
SR-01000000597 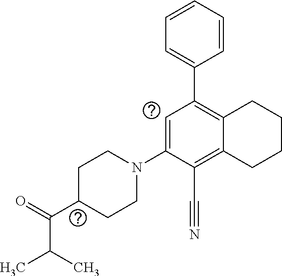 [SMILES: CC(C)C(=O)C1CCN(c2cc(-c3ccccc3)c3c(c2C#N)CCCC3)CC1]
SR-01000452455-1-B 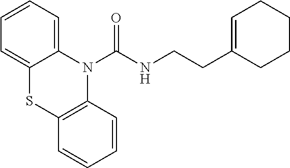 [SMILES: O=C(NCCC1=CCCCC1)N1c2ccccc2Sc2ccccc21]
SR-01000259336 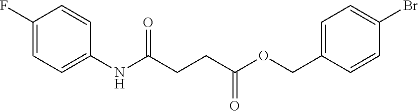 [SMILES: O=C(CCC(=O)OCc1ccc(Br)cc1)Nc1ccc(F)cc1]
SR-01000043246 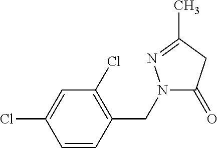 [SMILES: CC1=NN(Cc2ccc(Cl)cc2Cl)C(=O)C1]
SR-01000555461 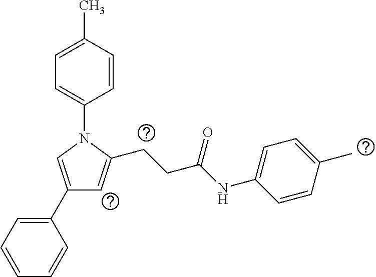 [SMILES: Cc1ccc(NC(=O)CCc2cc(-c3ccccc3)cn2-c2ccc(C)cc2)cc1]
SR-01000253143 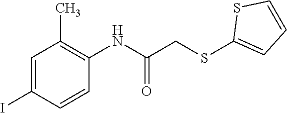 [SMILES: Cc1cc(I)ccc1NC(=O)CSc1cccs1]
SR-01000445554 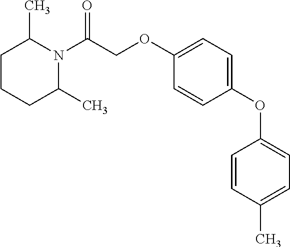 [SMILES: Cc1ccc(Oc2ccc(OCC(=O)N3C(C)CCCC3C)cc2)cc1]
SR-01000462122 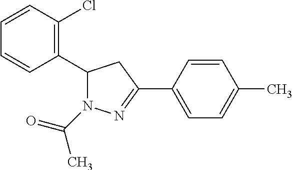 [SMILES: CC(=O)N1N=C(c2ccc(C)cc2)CC1c1ccccc1Cl]
SR-01000451510 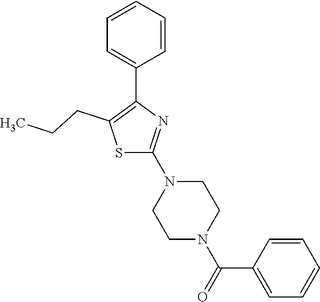 [SMILES: CCCc1sc(N2CCN(C(=O)c3ccccc3)CC2)nc1-c1ccccc1]
SR-01000524226 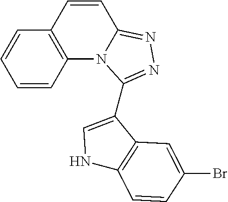 [SMILES: Brc1ccc2[nH]cc(-c3nnc4ccc5ccccc5n34)c2c1]
SR-01000555455 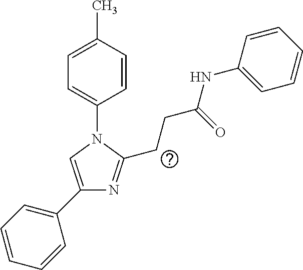 [SMILES: Cc1ccc(-n2cc(-c3ccccc3)nc2CCC(=O)Nc2ccccc2)cc1]
SR-01000503729 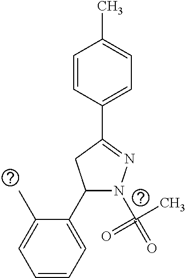
SR-01000507831 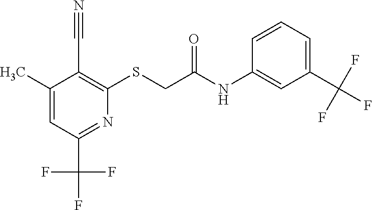 [SMILES: Cc1cc(C(F)(F)F)nc(SCC(=O)Nc2cccc(C(F)(F)F)c2)c1C#N]
SR-01000555460 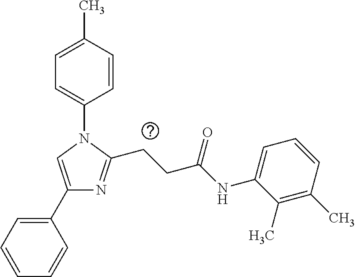 [SMILES: Cc1ccc(-n2cc(-c3ccccc3)nc2CCC(=O)Nc2cccc(C)c2C)cc1]
SR-01000429850

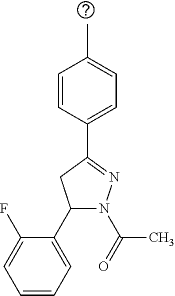 [SMILES: CC(=O)N1N=C(c2ccc(C)cc2)CC1c1ccccc1F]

In some embodiments, a described GP139 antagonist or a pharmaceutical composition containing the GP139 antagonist is administered with an opioid drug. In some embodiments, a pharmaceutical composition containing a GP139 antagonist further comprises with an opioid drug.

The compounds and pharmaceutical compositions disclosed herein can be administered to a subject once per day, more than once a day, for example, 2, 3, 4, 5, or 6 times a day, or as needed.

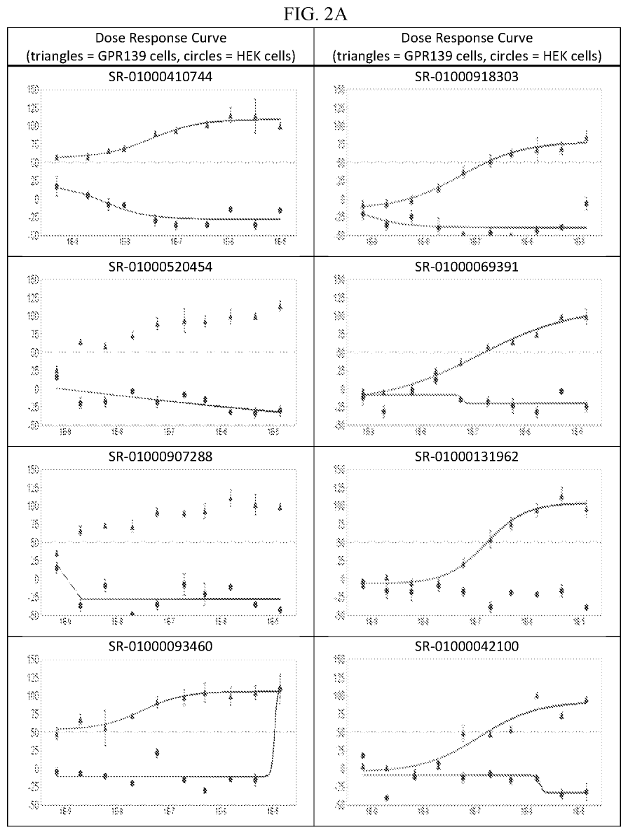

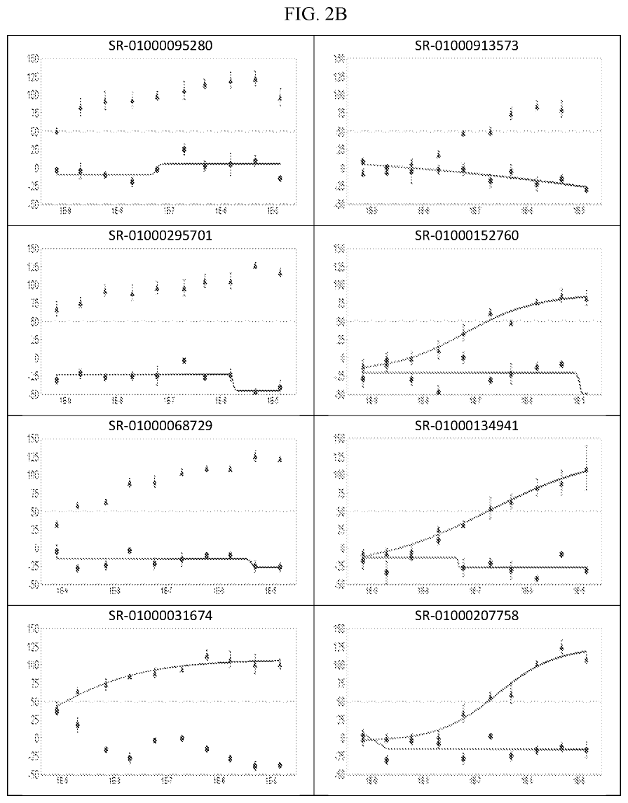

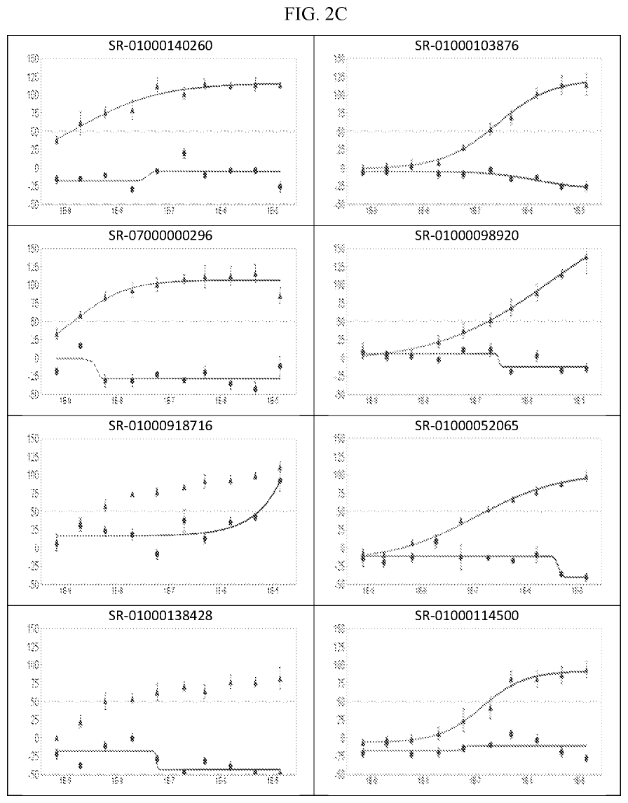

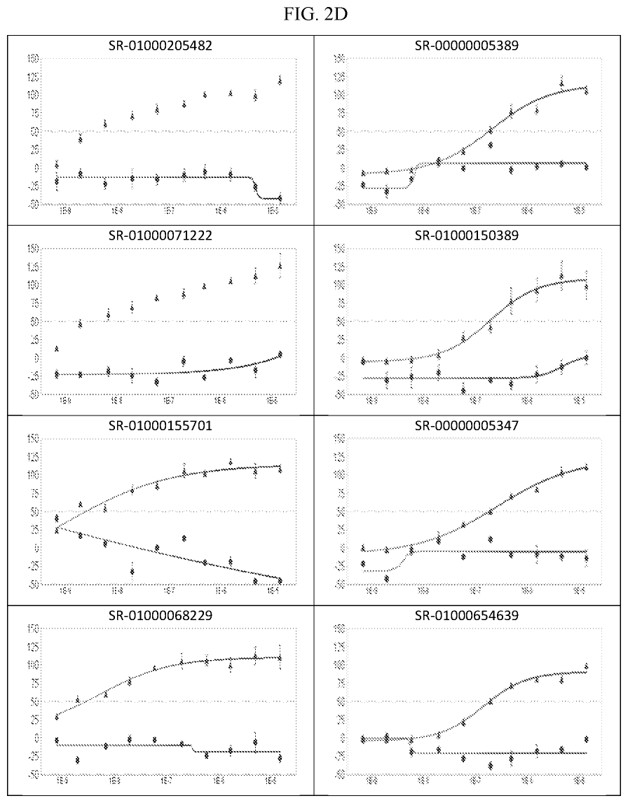

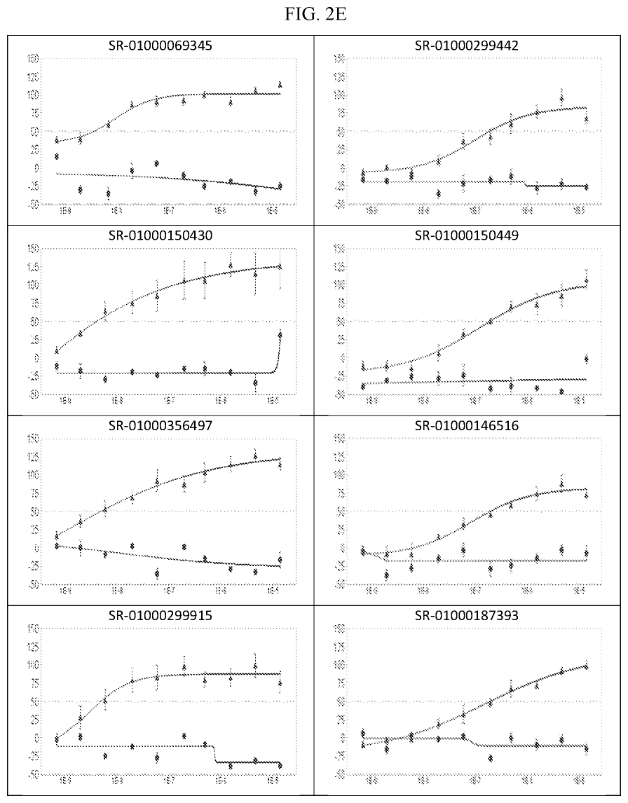

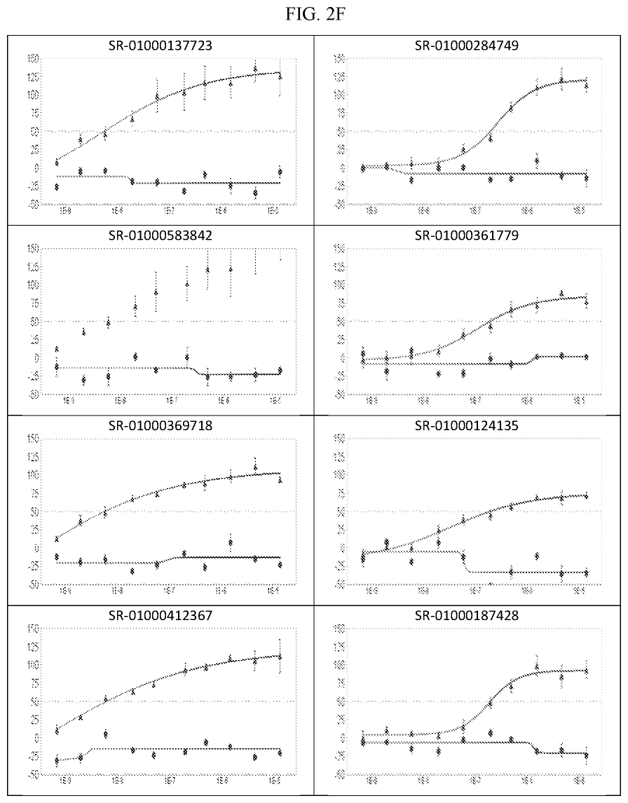

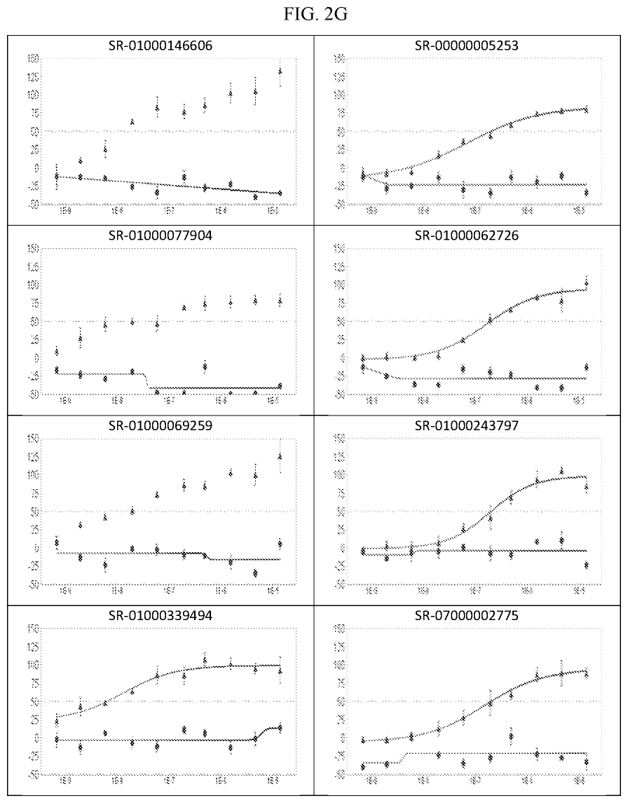

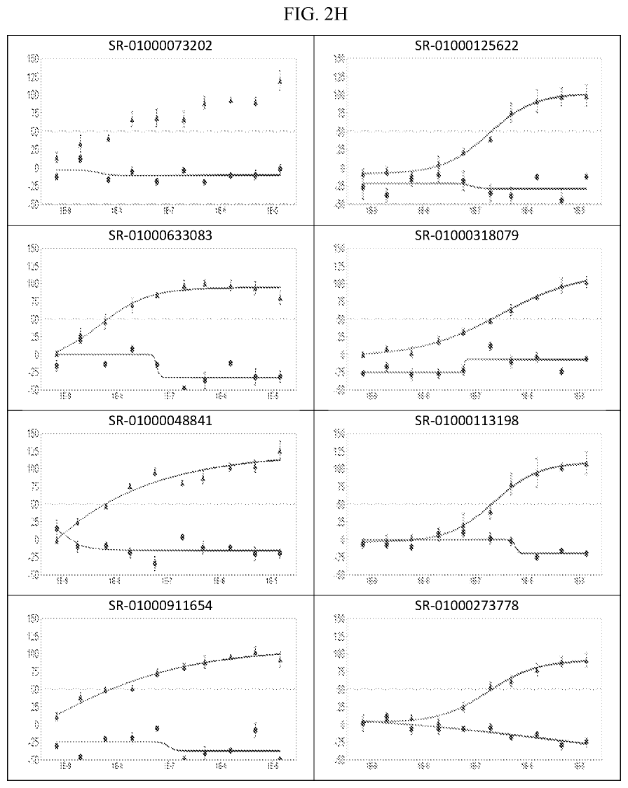

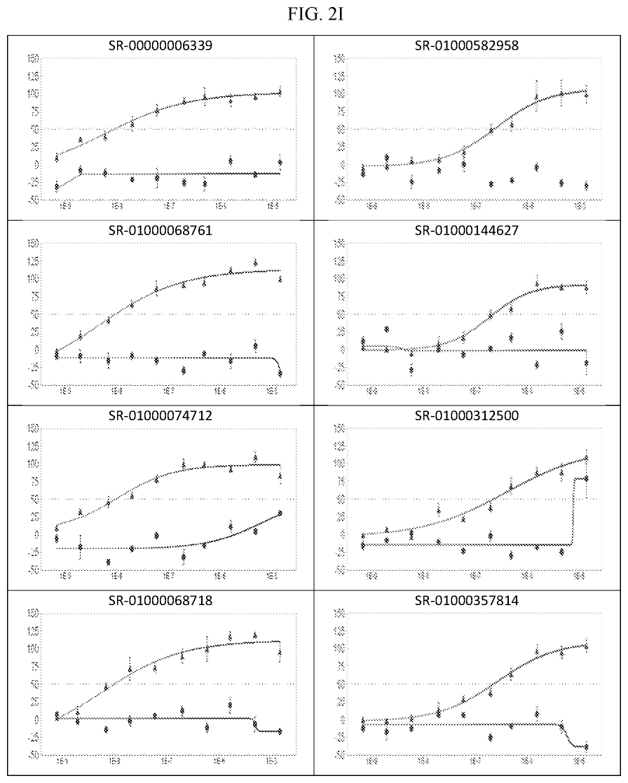

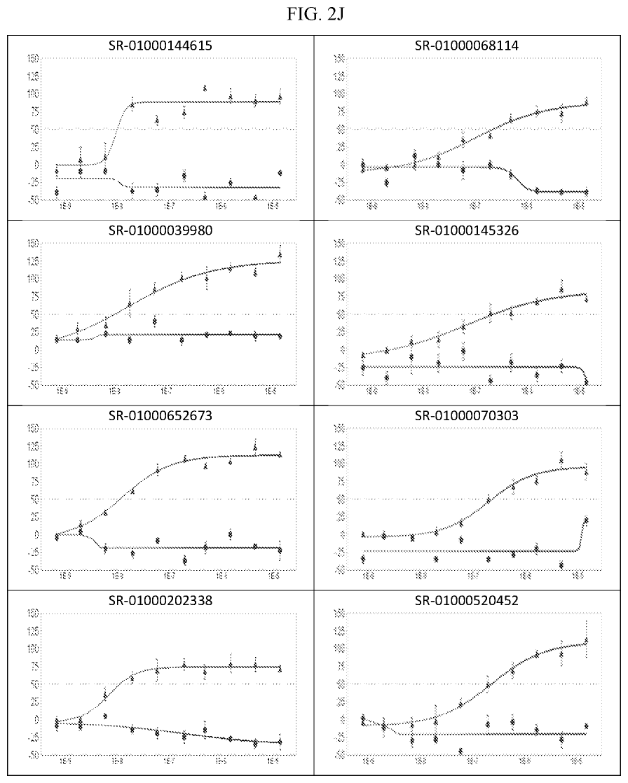

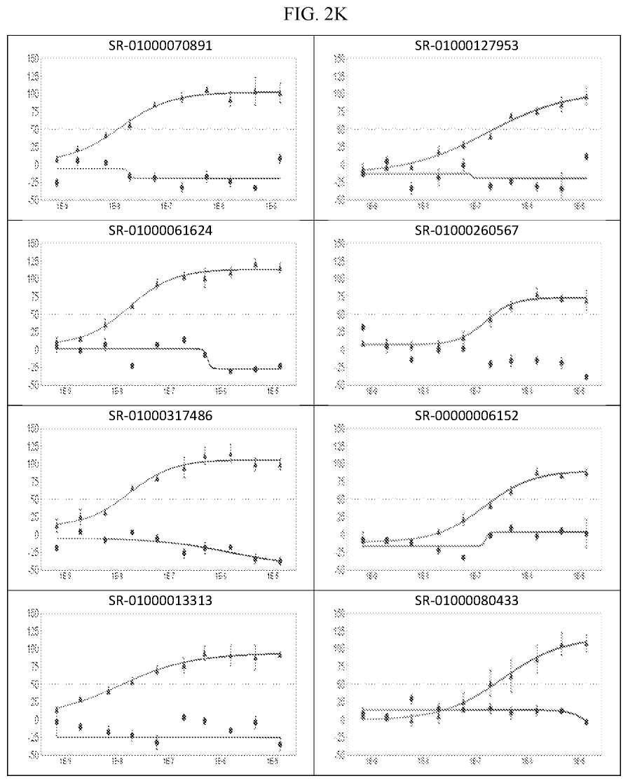

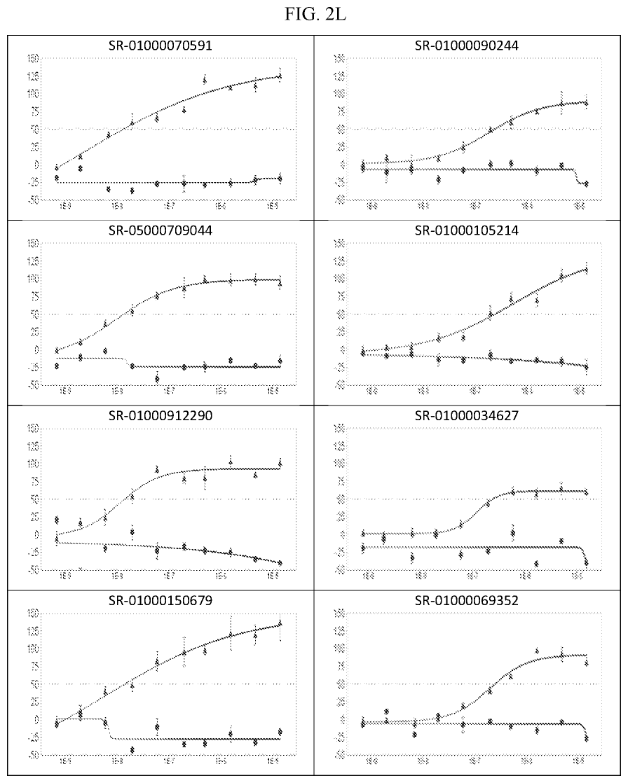

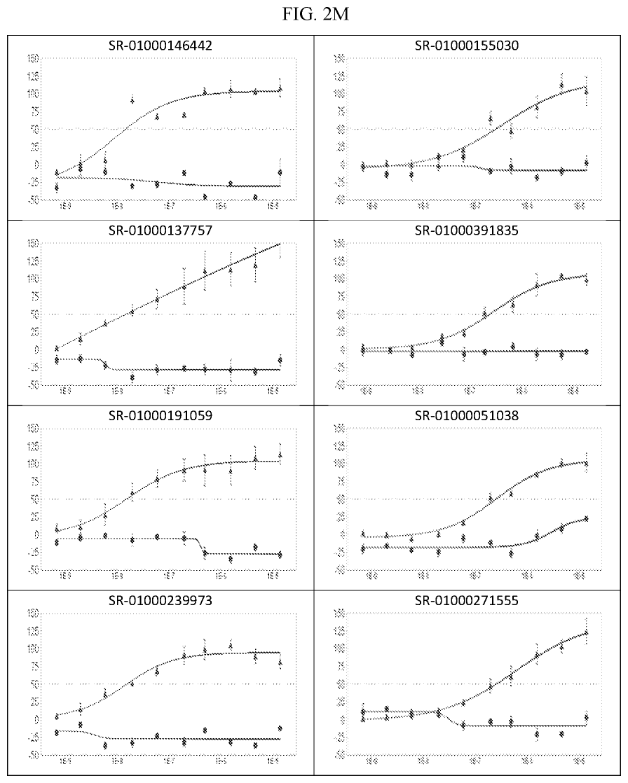

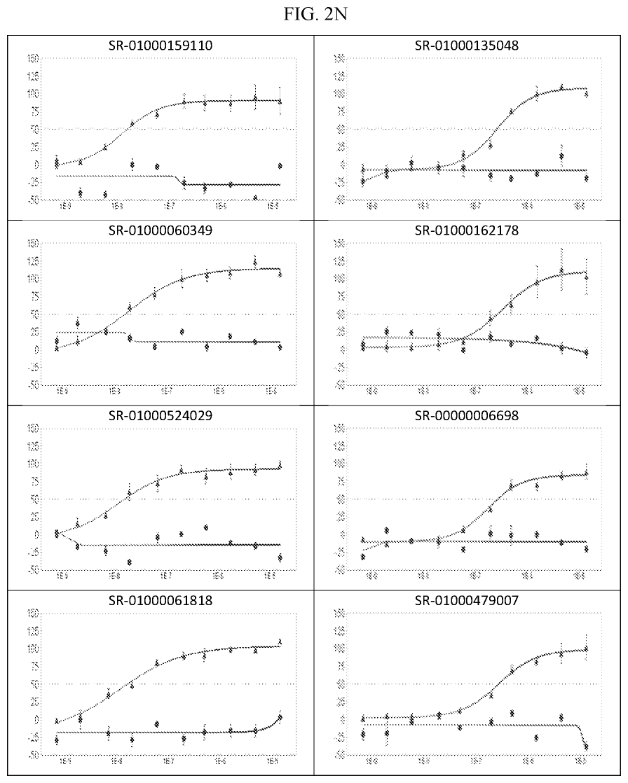

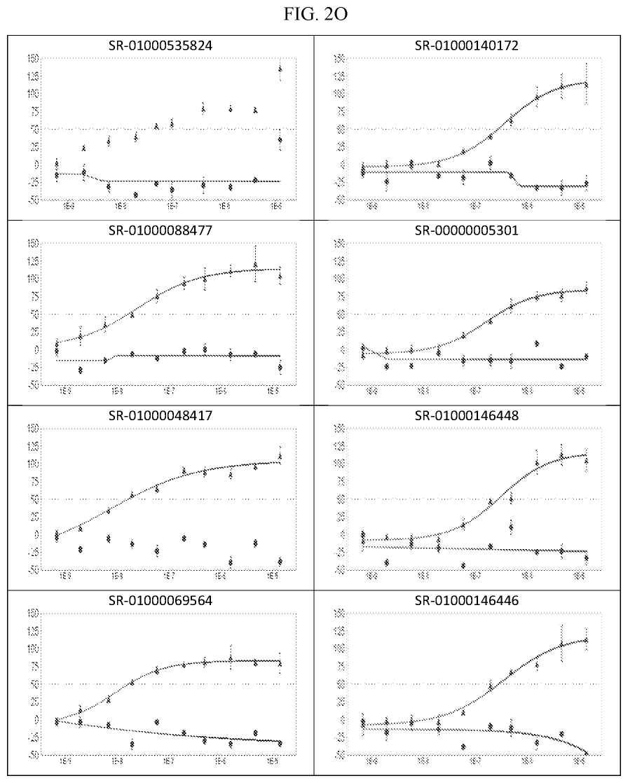

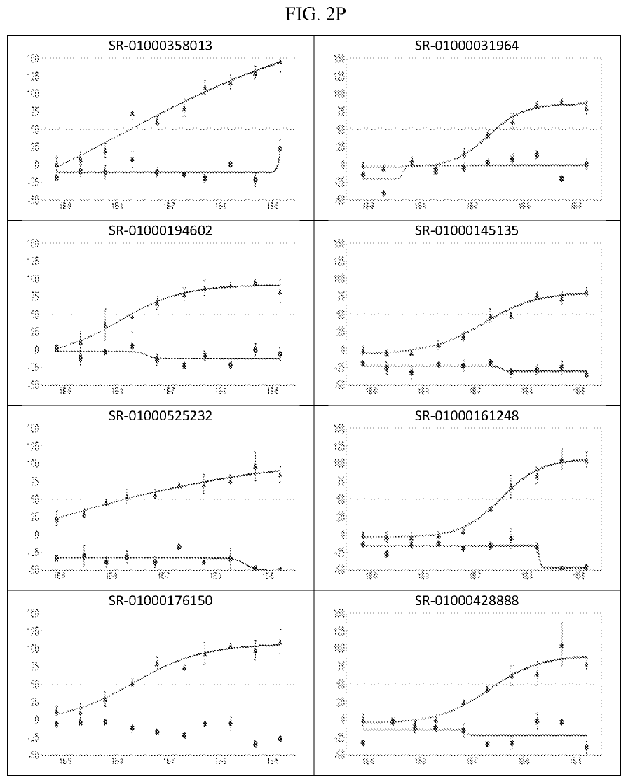

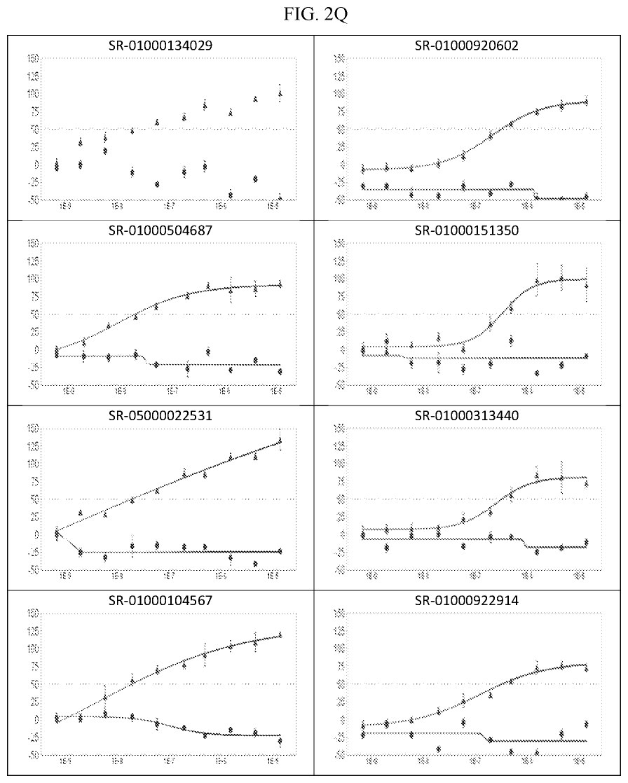

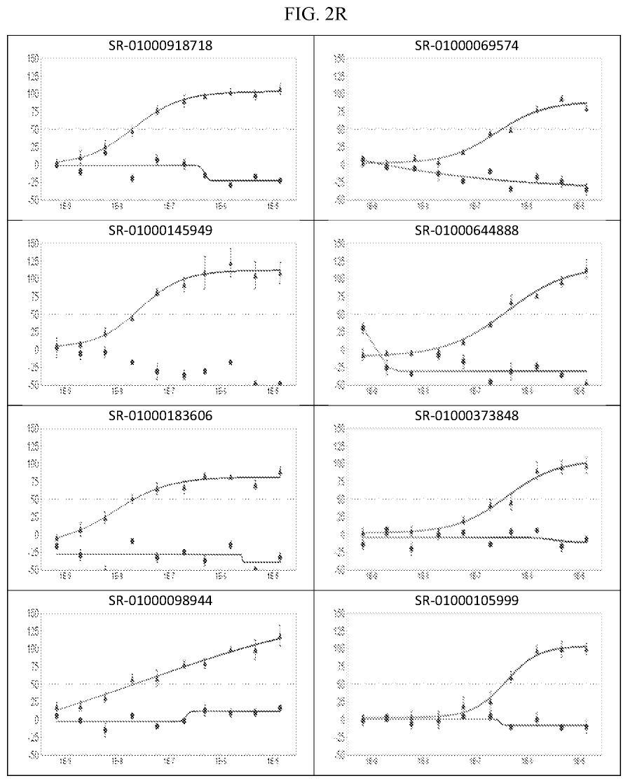

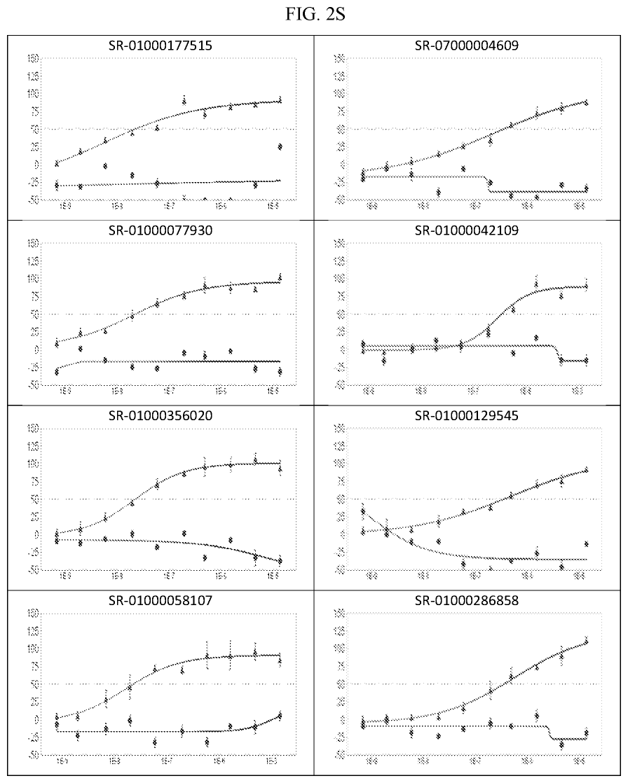

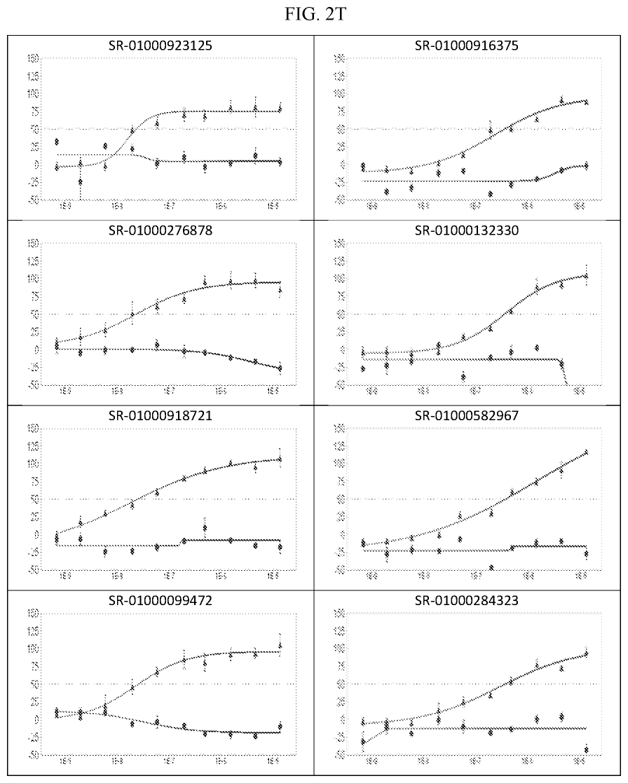

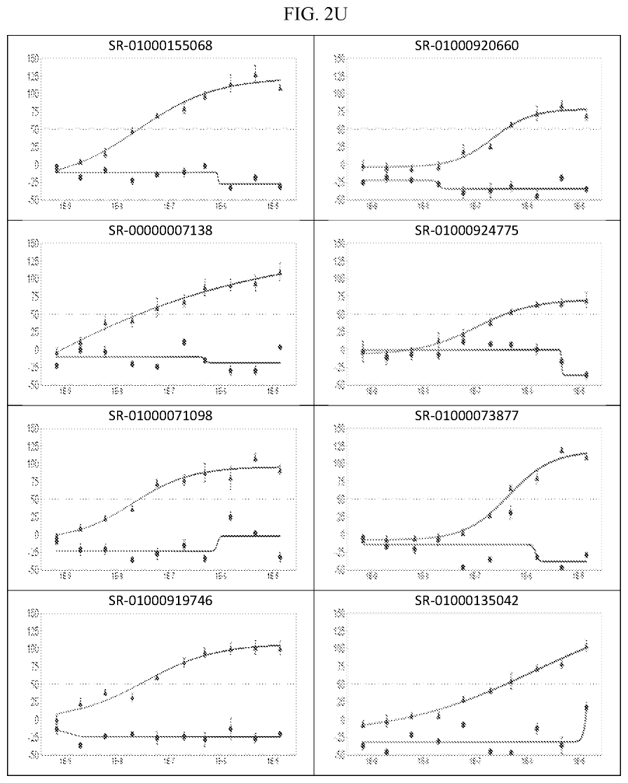

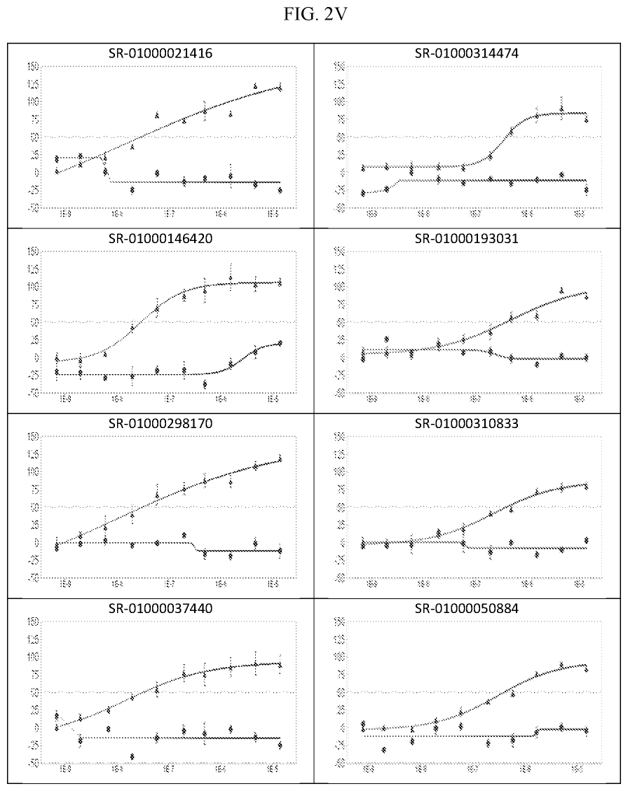

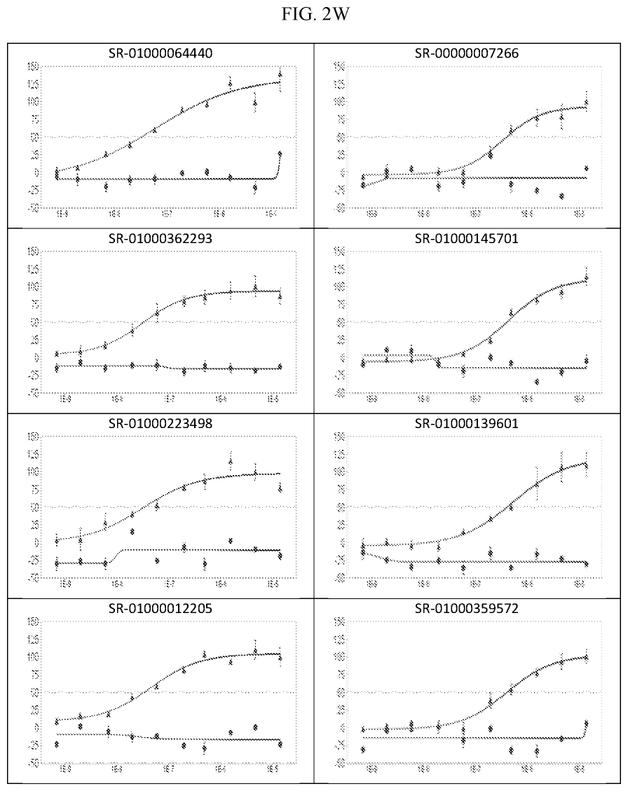

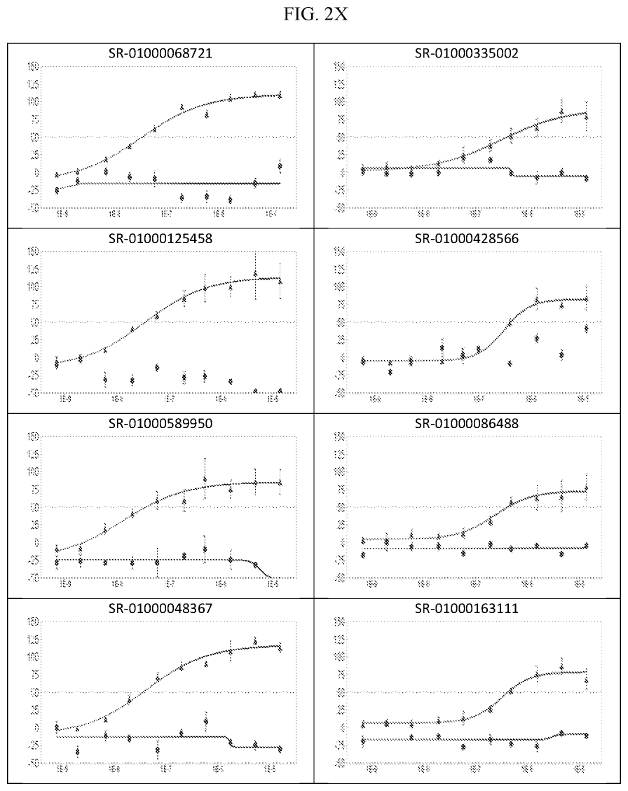

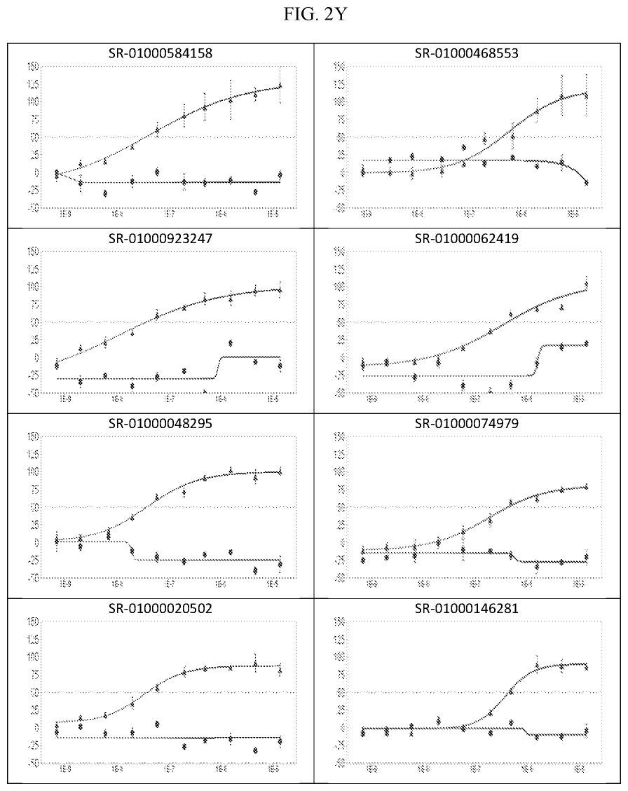

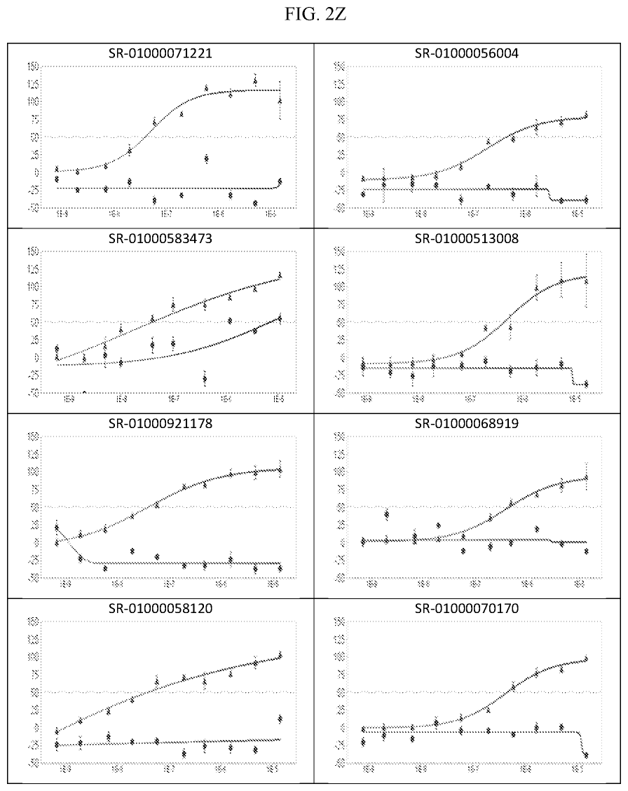

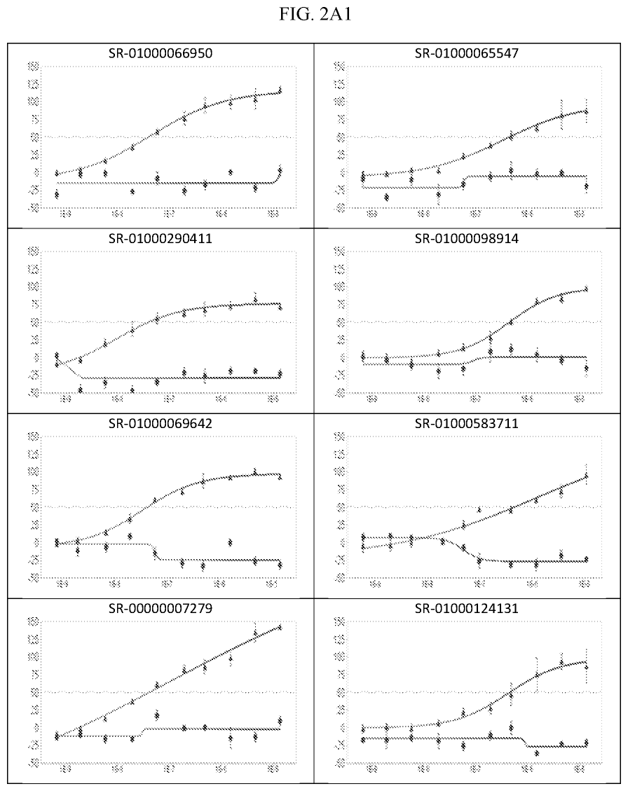

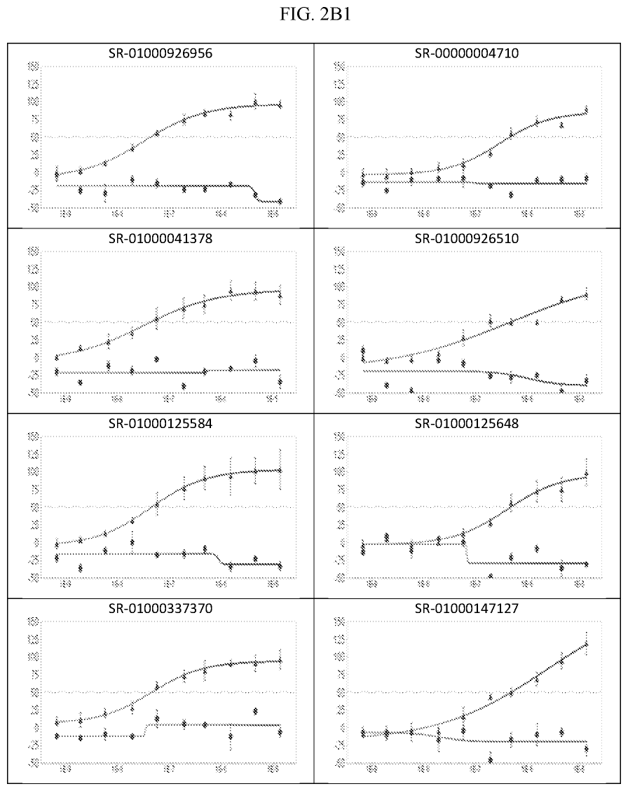

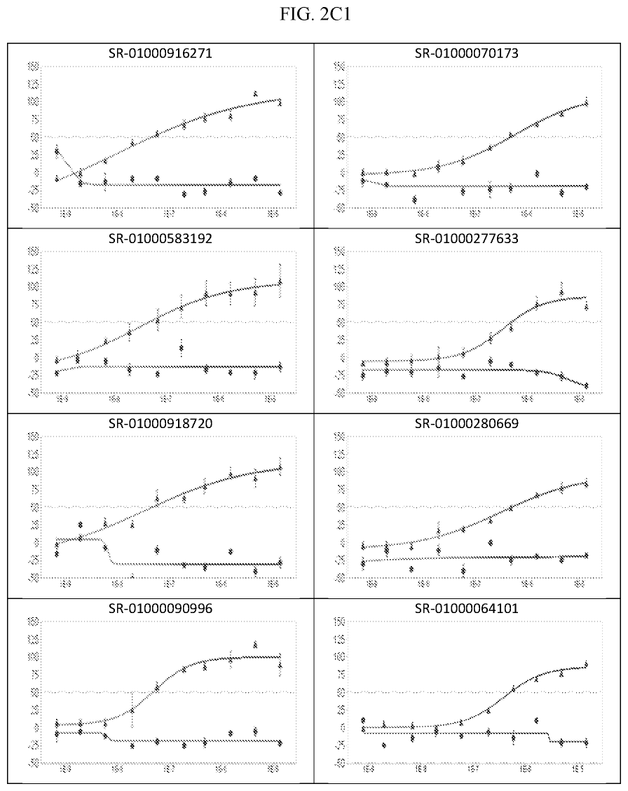

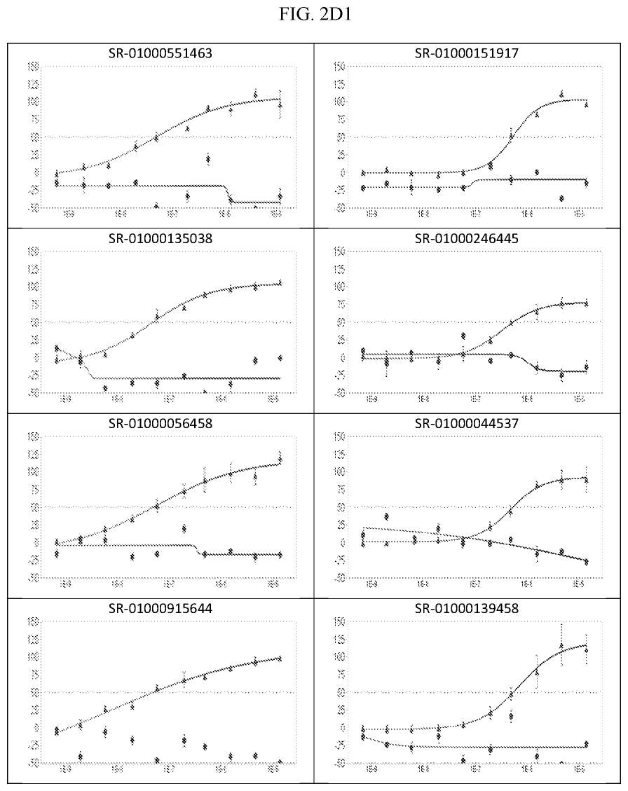

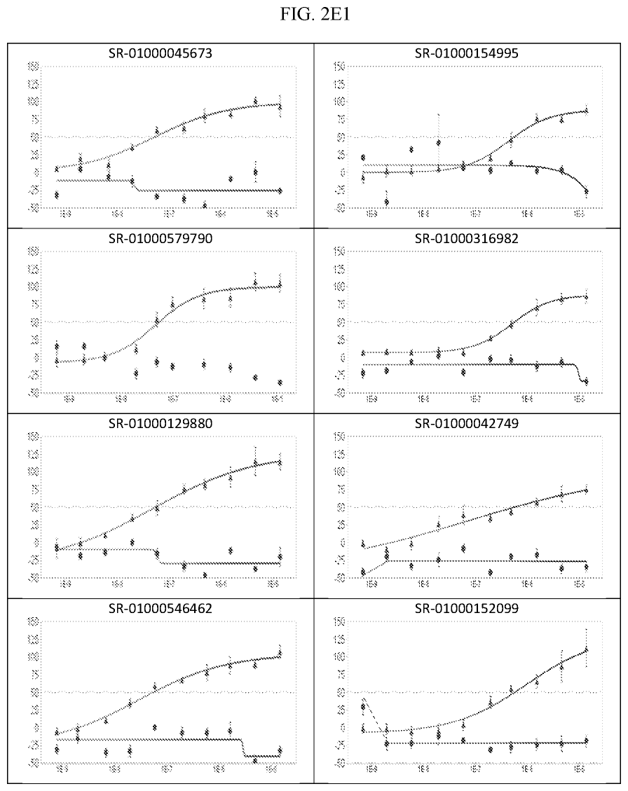

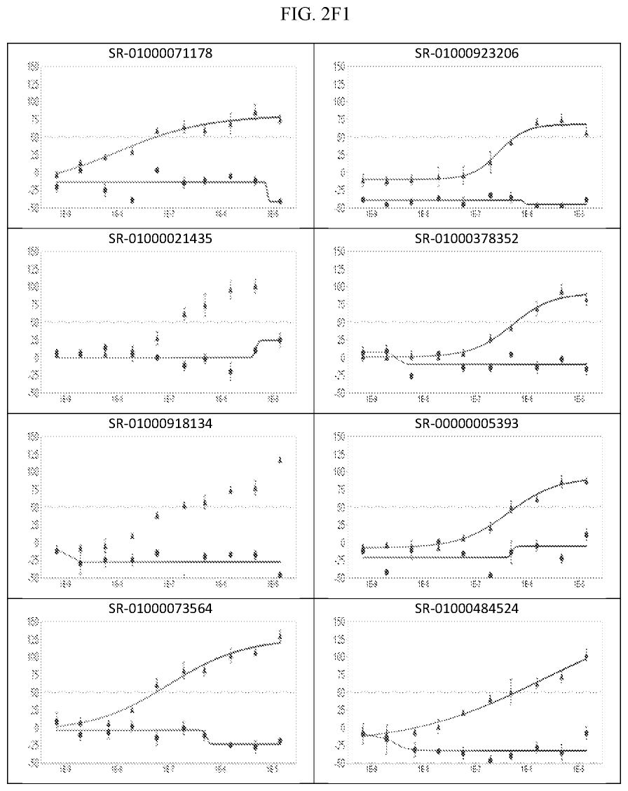

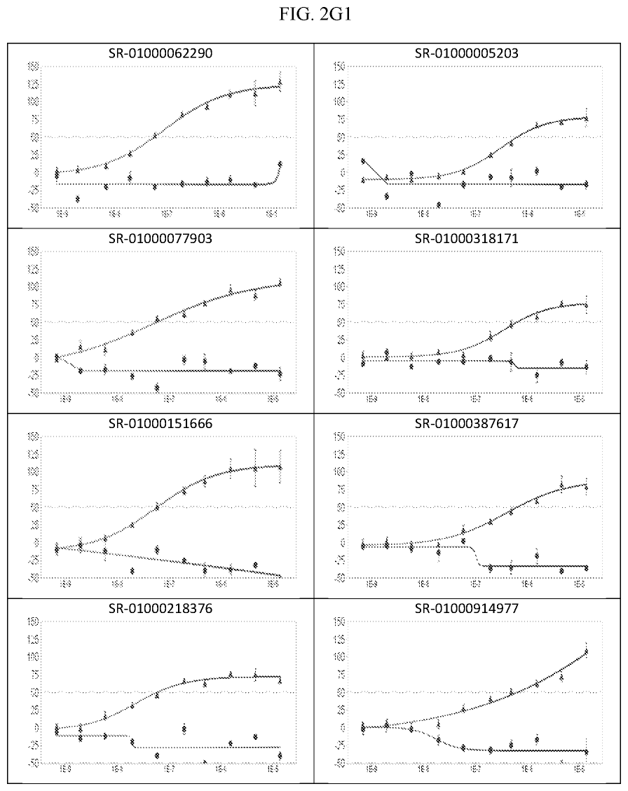

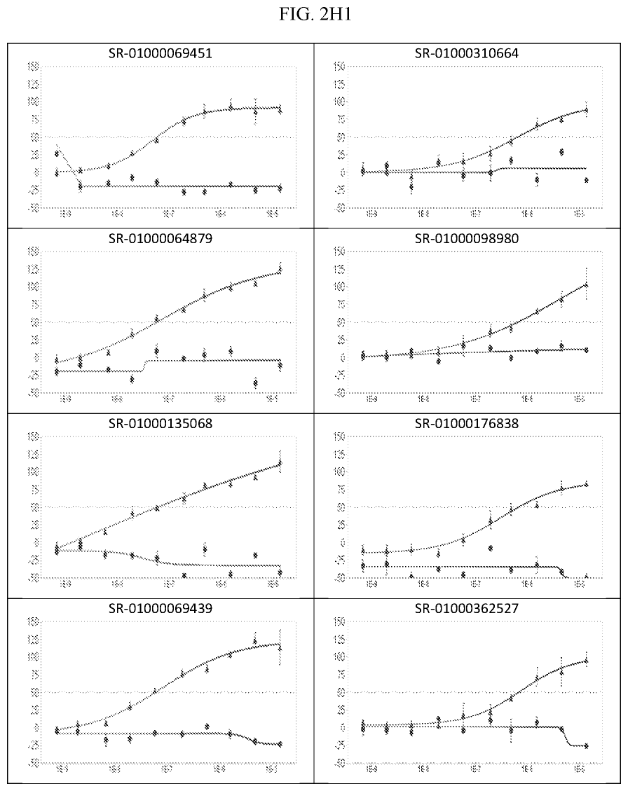

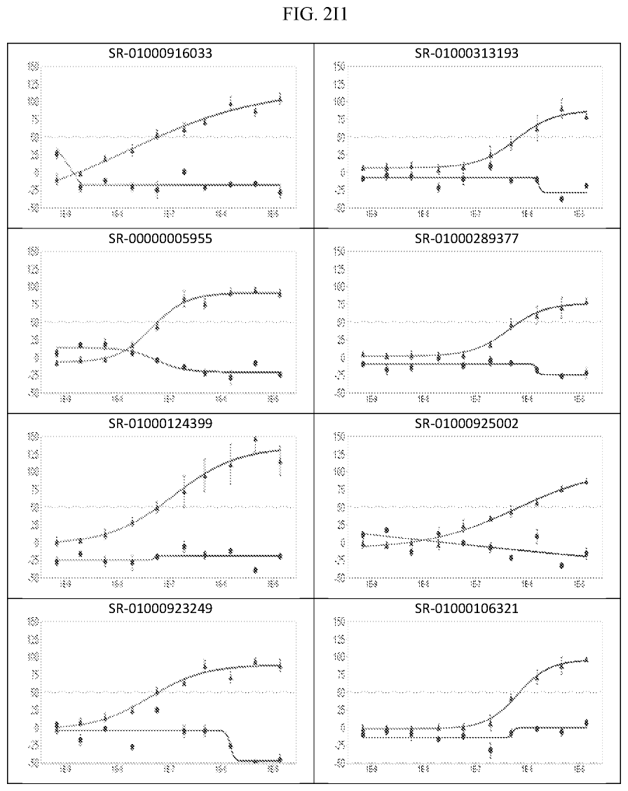

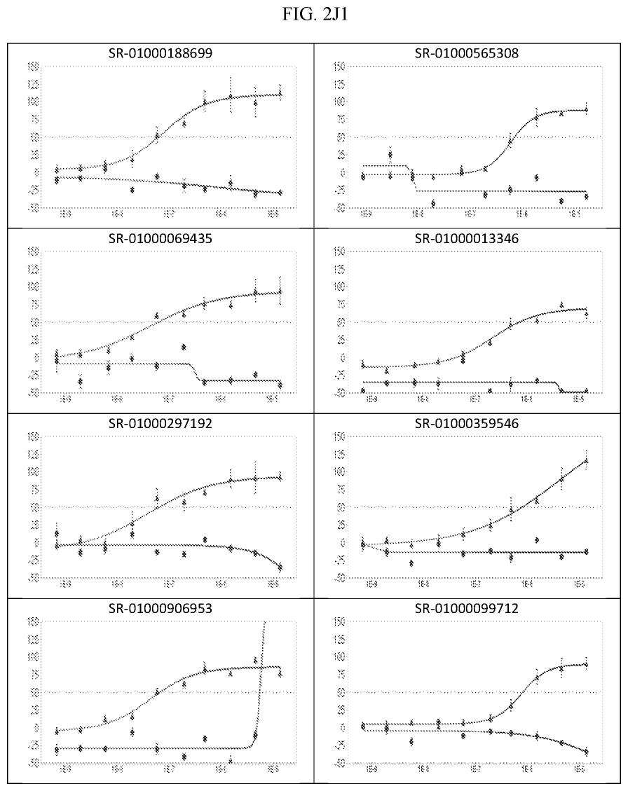

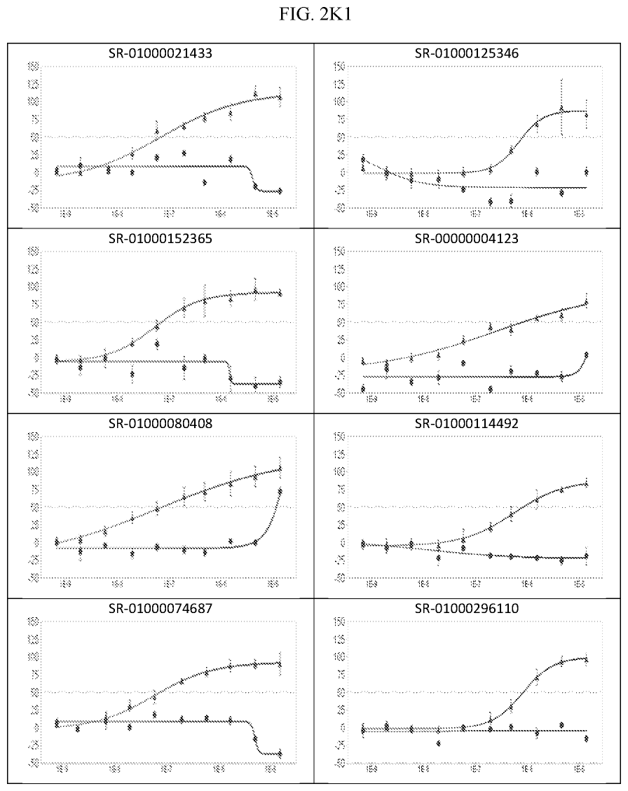

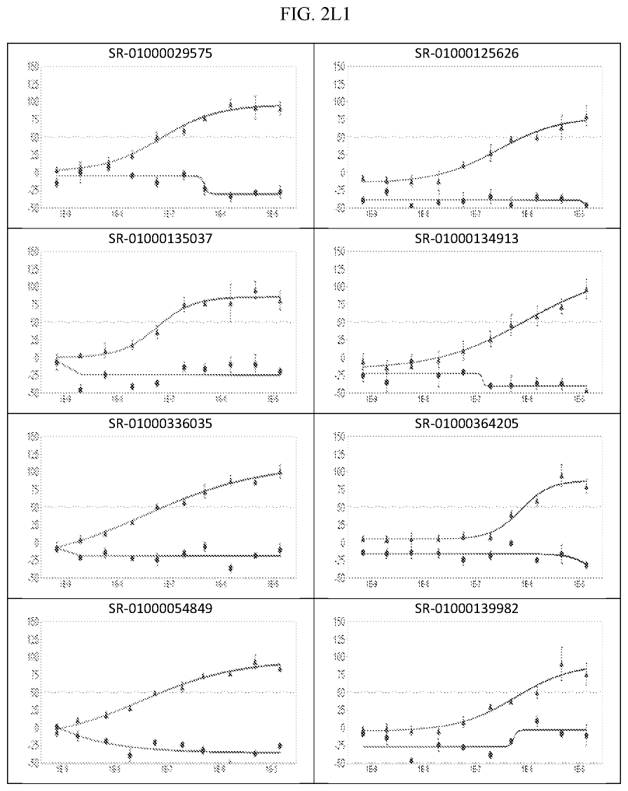

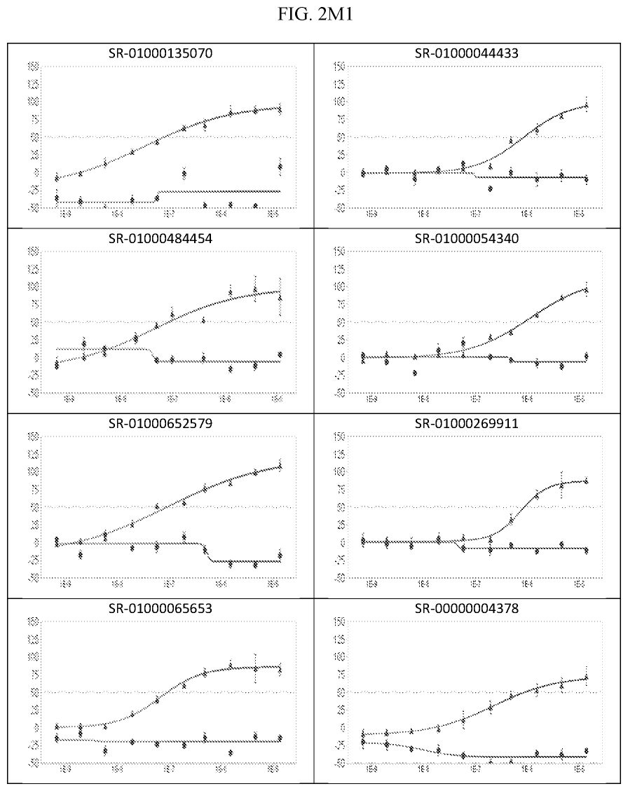

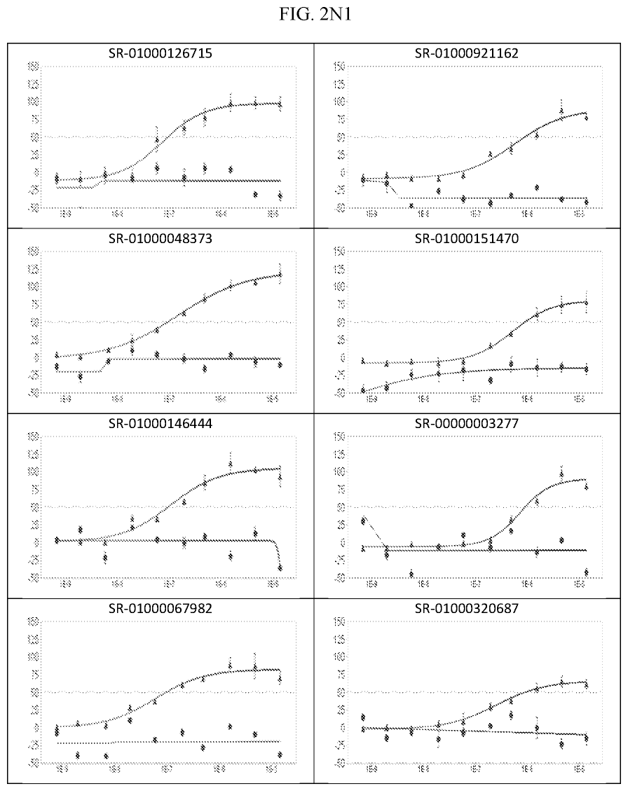

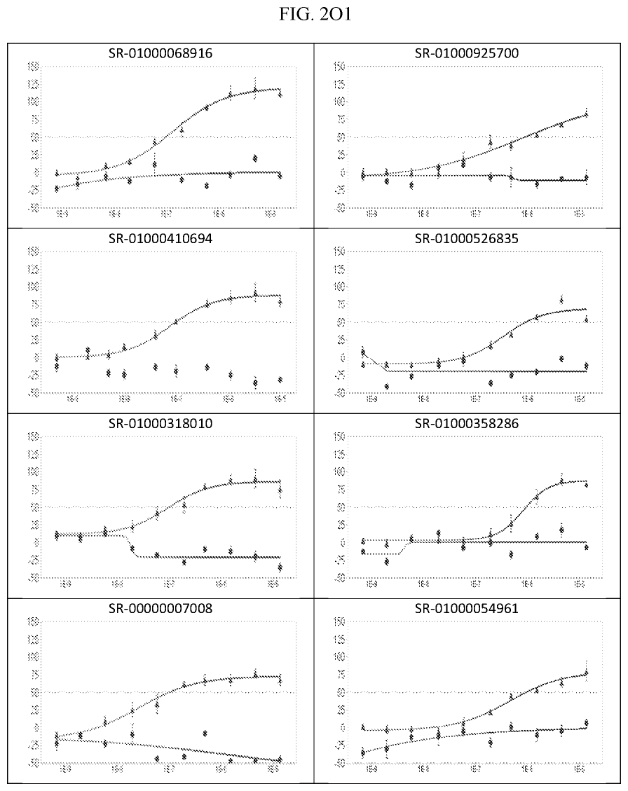

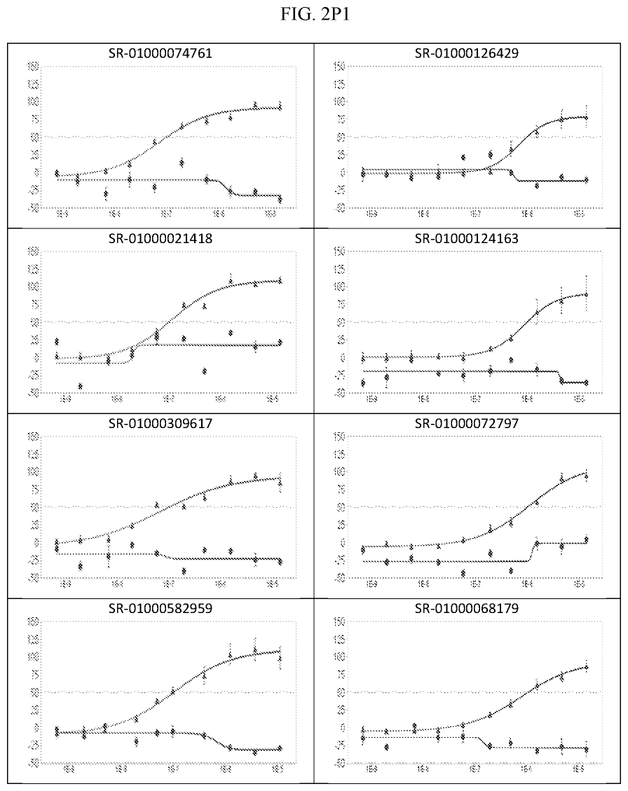

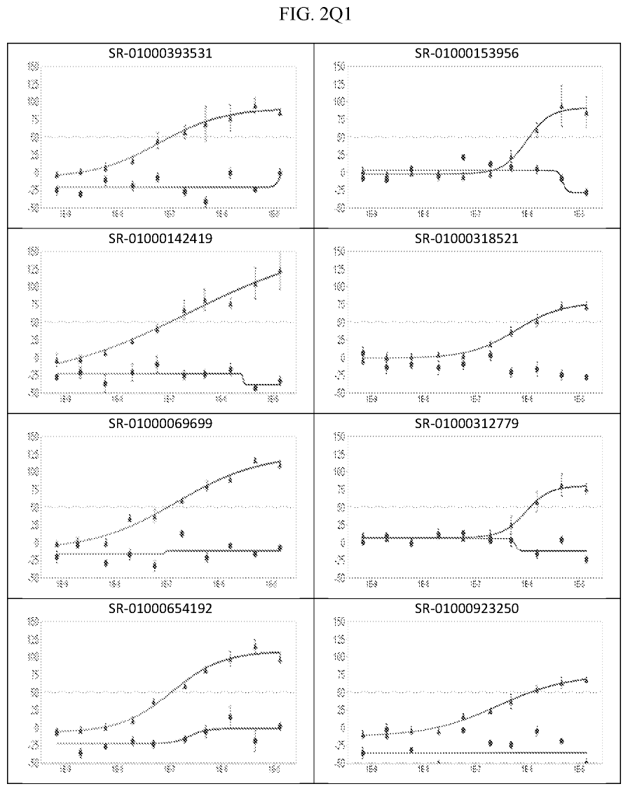

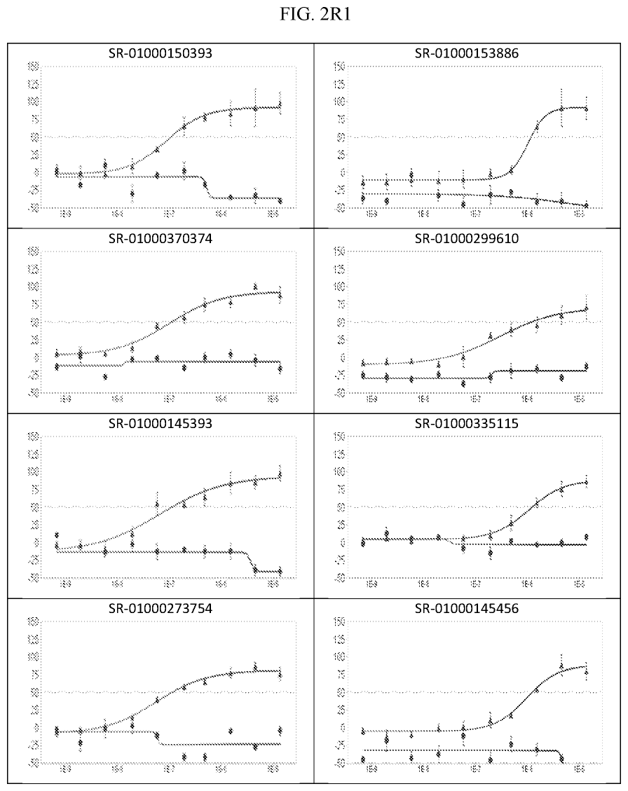

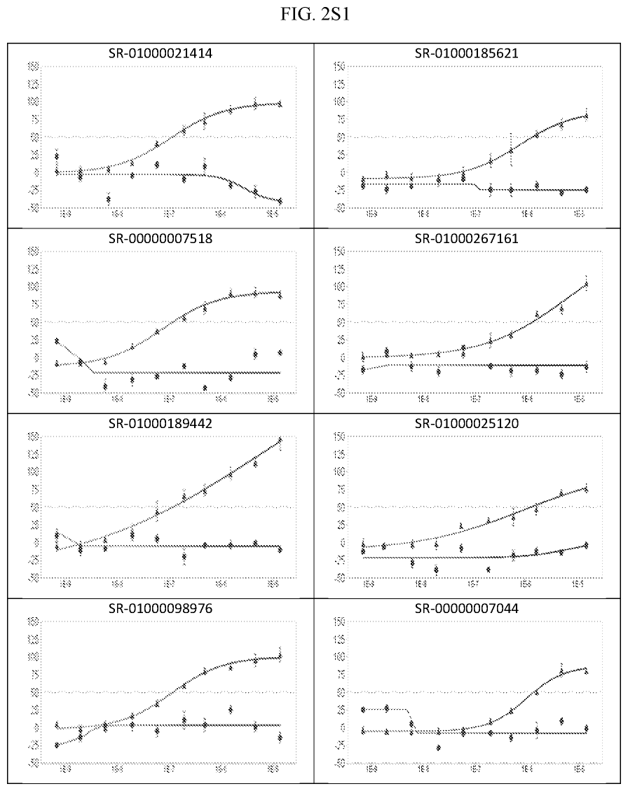

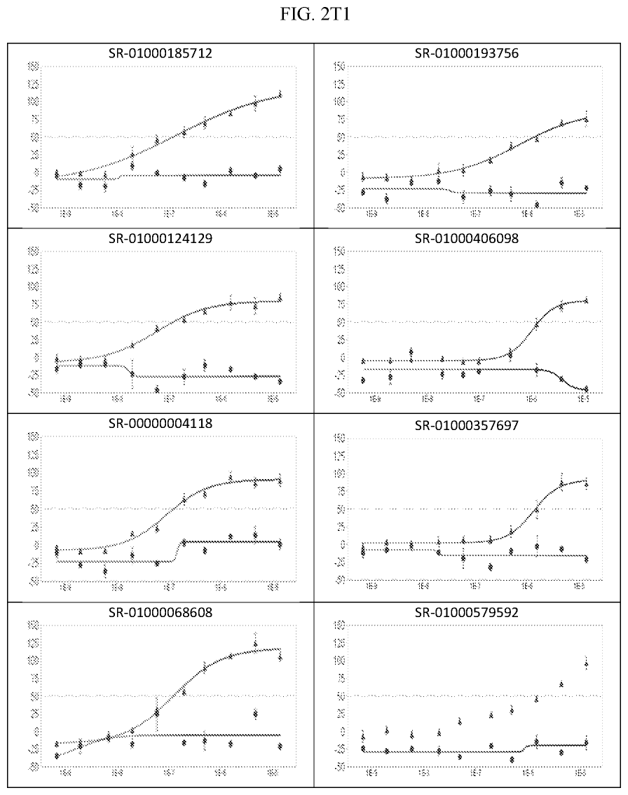

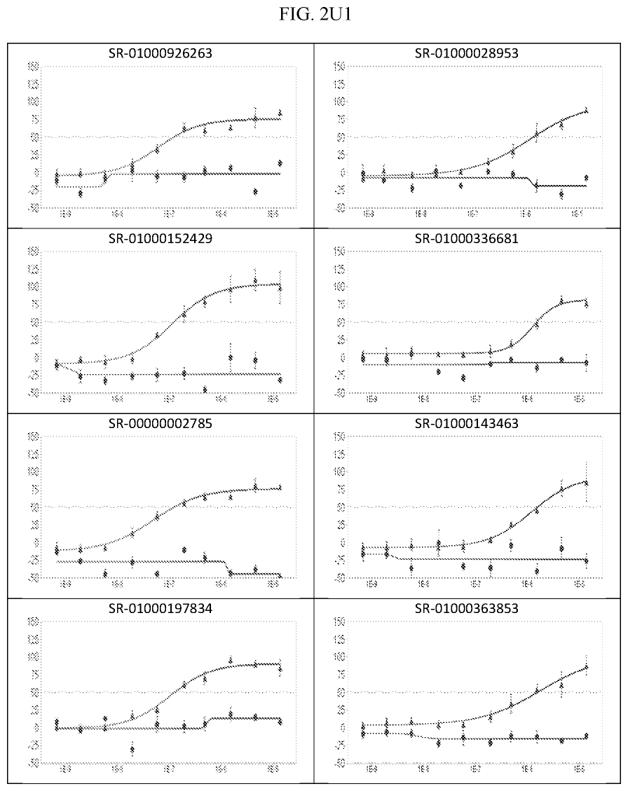

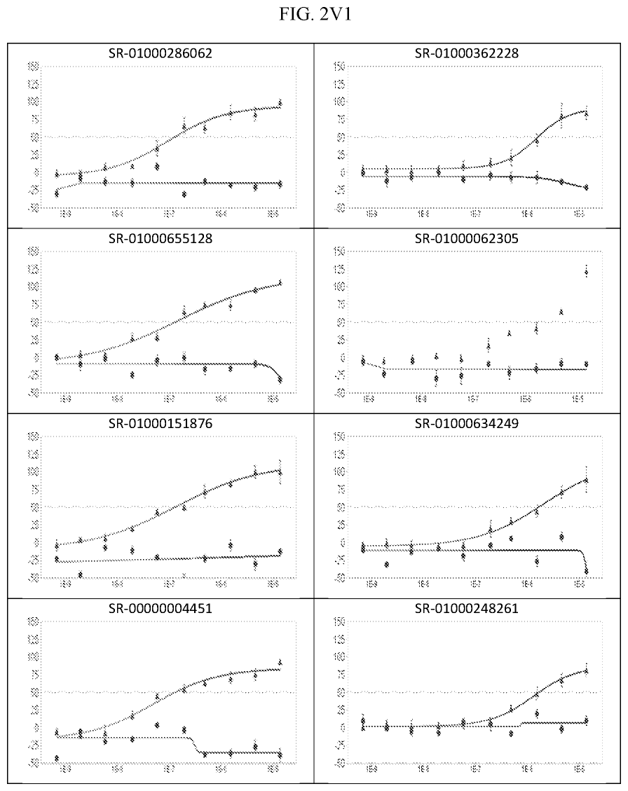

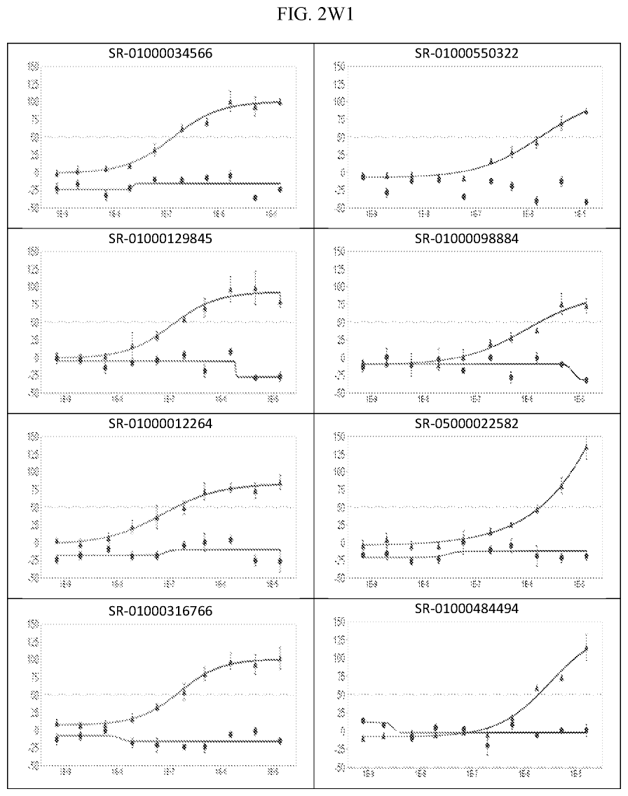

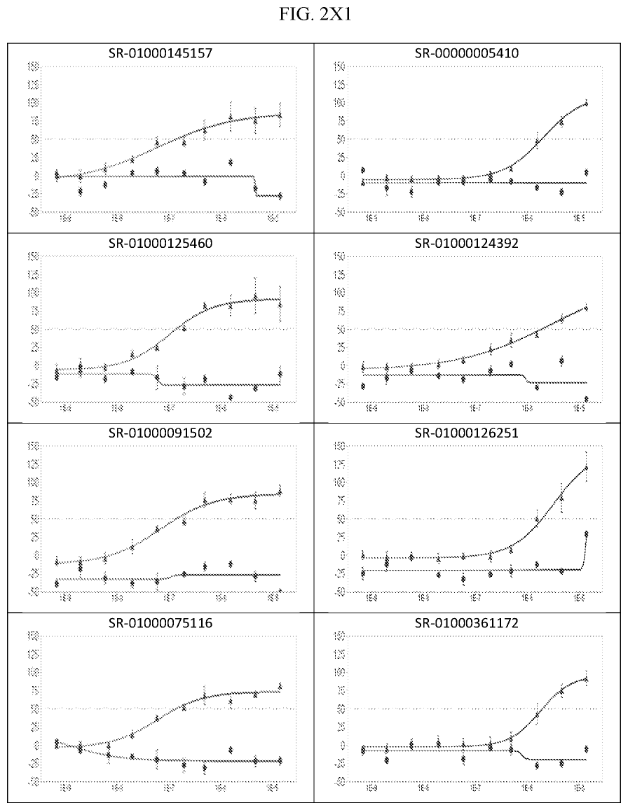

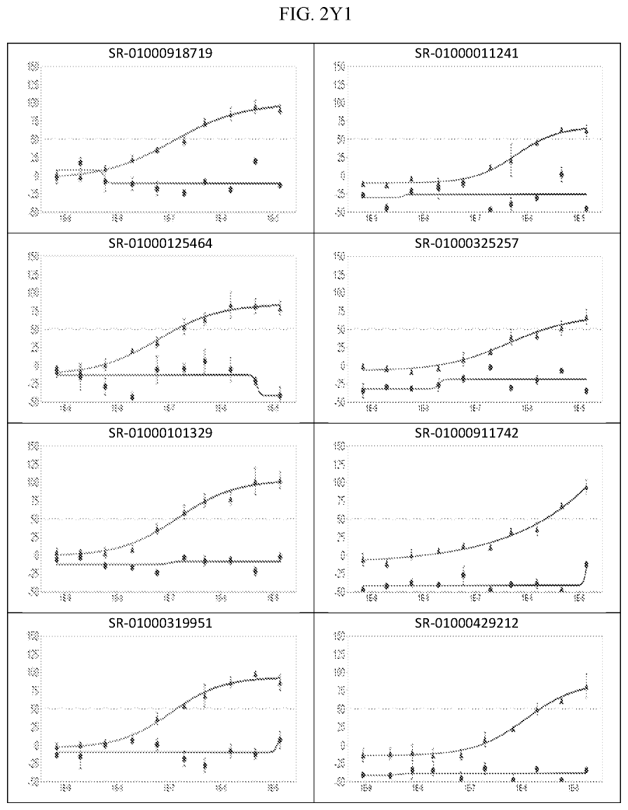

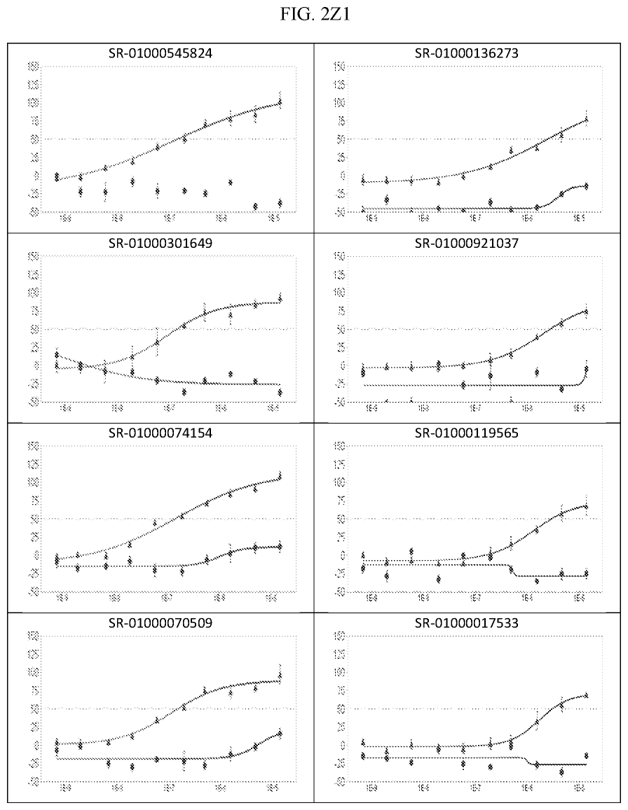

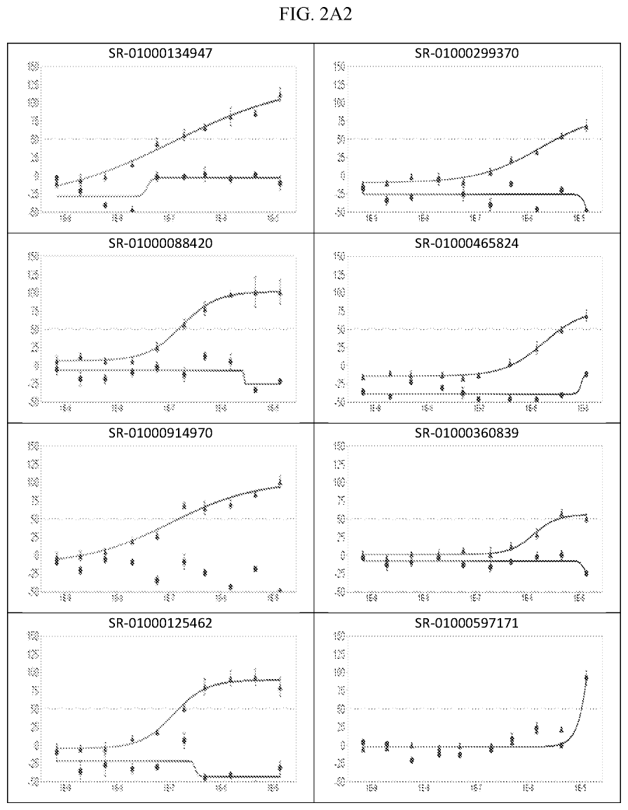

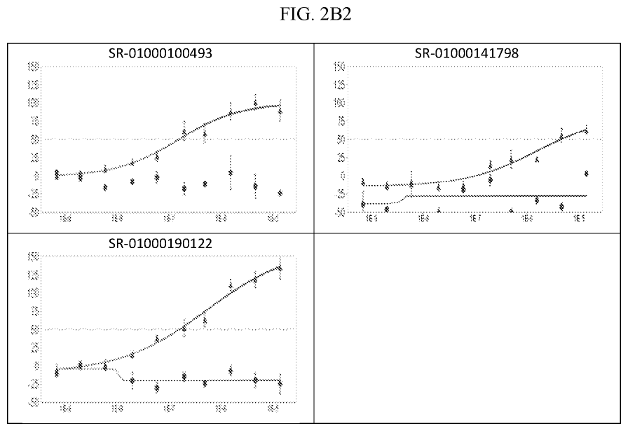

In some embodiments, methods of activating, or increasing, GPR139 activity and/or GPR139 signaling activity are provided. The methods comprise administering to a subject a therapeutically effective amount of any one or more of GPR139 agonists of FIG. 2 (A-B2). Administering a therapeutically effective amount of the GPR139 agonist includes, but is not limited to, administering to the subject a pharmaceutical composition that contains the therapeutically effective amount of the GPR139 agonist. In some embodiments, the GPR139 agonist increases GPR139 signaling or GPR139 GPCR function.

In some embodiments, methods are provided for down-regulating or decreasing MOR-mediated signaling activities. The methods comprise administering to a subject one or more GPR139 agonists of FIG. 2 (A-B2). In some embodiments, the GPR139 agonist increases GPR139 signaling or GPR139 GPCR function. Decreasing MOR-mediated signaling activities can be used to suppress or diminish reinforcing effects of opioids, or to decrease addiction to or dependence on opioid use.

In some embodiments, a GPR139 agonist is administered to a subject to reduce the reward (e.g., euphoric effects) associated with opioid use or to diminish the reinforcing effects of opioid use. Reducing the reward associated with opioid use or diminishing the reinforcing effects of opioid use can be used to treat or prevent addiction in a subject. Methods of treating or preventing opioid addiction comprise administering to the subject an effective amount of one or more GPR139 agonists of Table 2. Administering the GPR agonist to the subject can increase expression or cellular activity of GPR139 or an ortholog thereof and/or increase GPR139 signaling activity. The subject can be, but is not limited to, a human.

TABLE 2
GPR139 Agonists.
COMPOUNDS/STRUCTURES
SR-01000410744

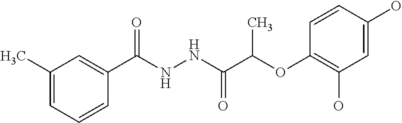 [SMILES: Cc1cccc(C(=O)NNC(=O)C(C)Oc2ccc(O)cc2O)c1]

SR-01000520454

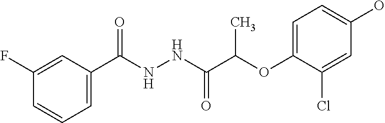 [SMILES: CC(Oc1ccc(O)cc1Cl)C(=O)NNC(=O)c1cccc(F)c1]

SR-01000907288

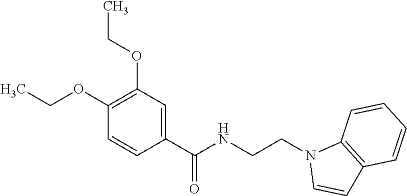 [SMILES: CCOc1ccc(C(=O)NCCn2ccc3ccccc32)cc1OCC]

SR-01000093460

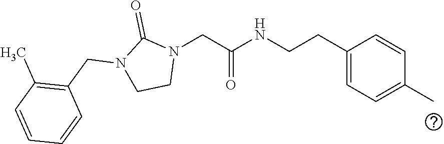 [SMILES: Cc1ccc(CCNC(=O)CN2CCN(Cc3ccccc3C)C2=O)cc1]

SR-01000095280

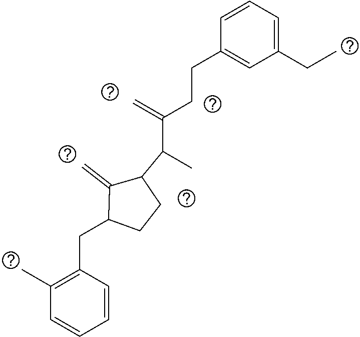 [SMILES: C=C(CCc1cccc(CC)c1)C(C)C1CCC(Cc2ccccc2C)C1=C]

SR-01000295701

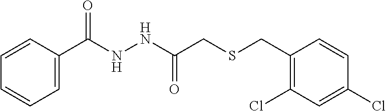 [SMILES: O=C(CSCc1ccc(Cl)cc1Cl)NNC(=O)c1ccccc1]

SR-01000918303

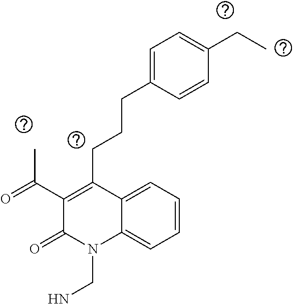 [SMILES: CCc1ccc(CCCc2c(C(C)=O)c(=O)n(CN)c3ccccc23)cc1]

SR-01000069391

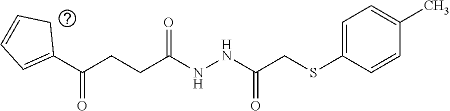 [SMILES: Cc1ccc(SCC(=O)NNC(=O)CCC(=O)C2=CC=CC2)cc1]

SR-01000131962

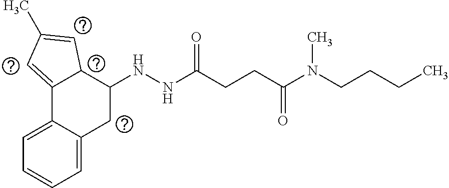 [SMILES: CCCCN(C)C(=O)CCC(=O)NNC1Cc2ccccc2C2=CC(C)=CC21]

SR-01000042100

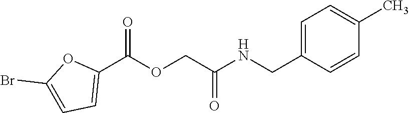 [SMILES: Cc1ccc(CNC(=O)COC(=O)c2ccc(Br)o2)cc1]

SR-01000913573

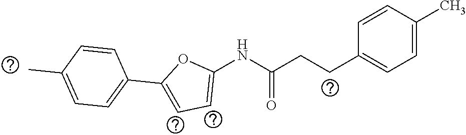

SR-01000152760

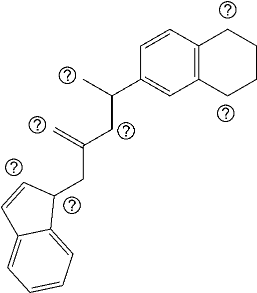 [SMILES: C=C(CC(C)c1ccc2c(c1)CCCC2)CC1C=Cc2ccccc21]

SR-01000068729

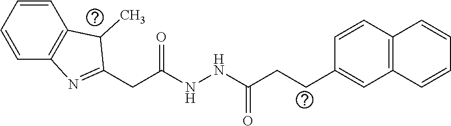 [SMILES: CC1C(CC(=O)NNC(=O)CCc2ccc3ccccc3c2)=Nc2ccccc21]

SR-01000031674

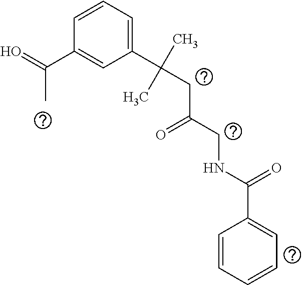 [SMILES: CC(=O)c1cccc(C(C)(C)CC(=O)CNC(=O)c2ccccc2)c1]

SR-01000140260

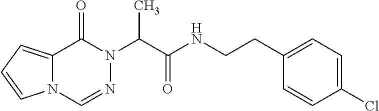 [SMILES: CC(C(=O)NCCc1ccc(Cl)cc1)n1ncn2cccc2c1=O]

SR-07000000296

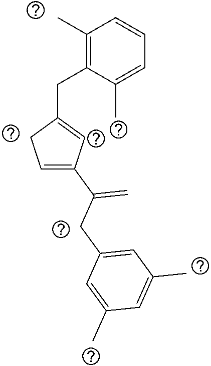 [SMILES: C=C(Cc1cc(C)cc(C)c1)C1=CCC(Cc2c(C)cccc2C)=C1]

SR-01000918716

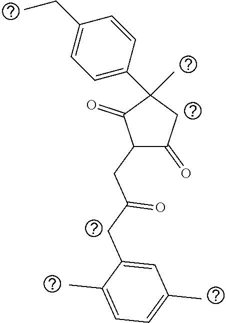 [SMILES: CCc1ccc(C2(C)CC(=O)C(CC(=O)Cc3cc(C)ccc3C)C2=O)cc1]

SR-01000138428

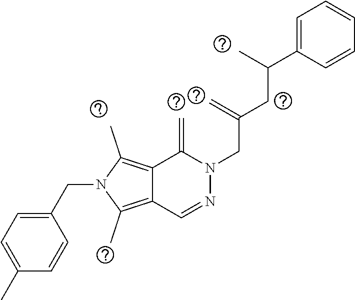 [SMILES: C=C(CC(C)c1ccccc1)CN1N=Cc2c(c(C)n(Cc3ccc(C)cc3)c2C)C1=C]

SR-01000134941

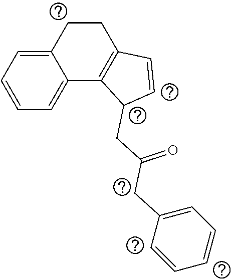 [SMILES: O=C(Cc1ccccc1)CC1C=CC2=C1c1ccccc1CC2]

SR-01000207758

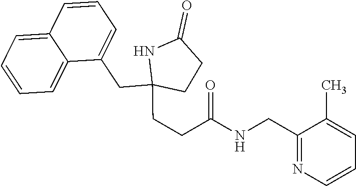 [SMILES: Cc1cccnc1CNC(=O)CCC1(Cc2cccc3ccccc23)CCC(=O)N1]

SR-01000103876

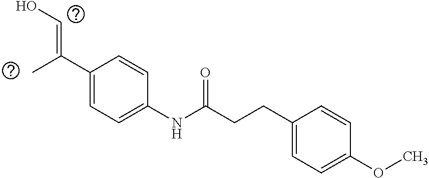 [SMILES: COc1ccc(CCC(=O)Nc2ccc(/C(C)=C/O)cc2)cc1]

SR-01000098920

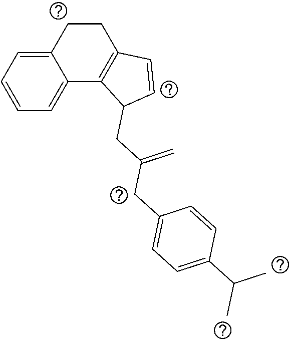 [SMILES: C=C(Cc1ccc(C(C)C)cc1)CC1C=CC2=C1c1ccccc1CC2]

SR-01000052065

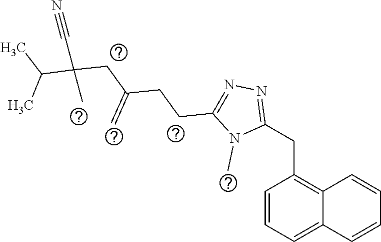 [SMILES: C=C(CCc1nnc(Cc2cccc3ccccc23)n1C)CC(C)(C#N)C(C)C]

SR-01000114500

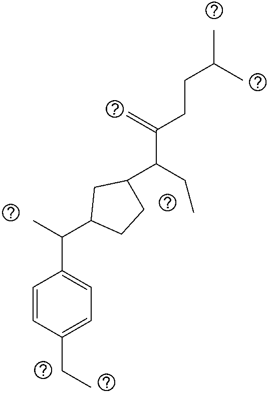 [SMILES: C=C(CCC(C)C)C(CC)C1CCC(C(C)c2ccc(CC)cc2)C1]

SR-01000205482

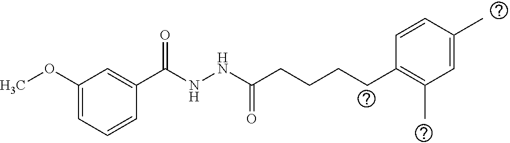

SR-01000071222

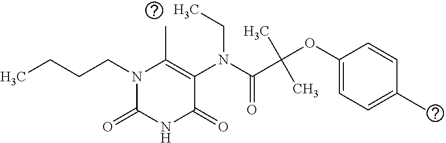 [SMILES: CCCCn1c(C)c(N(CC)C(=O)C(C)(C)Oc2ccc(C)cc2)c(=O)[nH]c1=O]

SR-01000155701

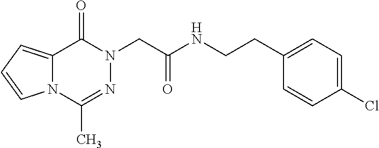 [SMILES: Cc1nn(CC(=O)NCCc2ccc(Cl)cc2)c(=O)c2cccn12]

SR-01000068229

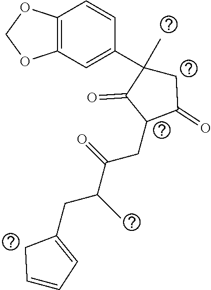 [SMILES: CC(CC1=CC=CC1)C(=O)CC1C(=O)CC(C)(c2ccc3c(c2)OCO3)C1=O]

SR-01000069345

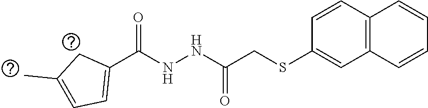 [SMILES: CC1=CC=C(C(=O)NNC(=O)CSc2ccc3ccccc3c2)C1]

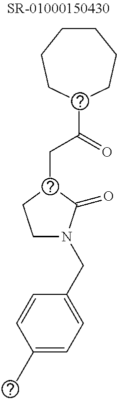 [SMILES: Cc1ccc(CN2CCC(CC(=O)C3CCCCCC3)C2=O)cc1]

SR-00000005389

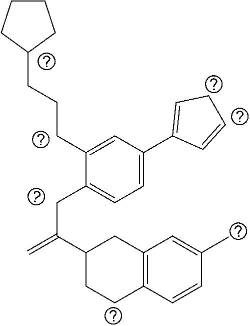 [SMILES: C=C(Cc1ccc(C2=CCC=C2)cc1CCCC1CCCC1)C1CCc2ccc(C)cc2C1]

SR-01000150389

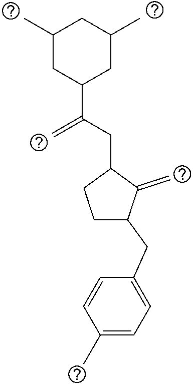

SR-00000005347

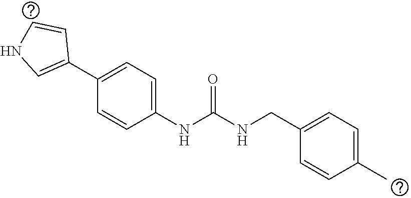 [SMILES: Cc1ccc(CNC(=O)Nc2ccc(-c3cc[nH]c3)cc2)cc1]

SR-01000654639

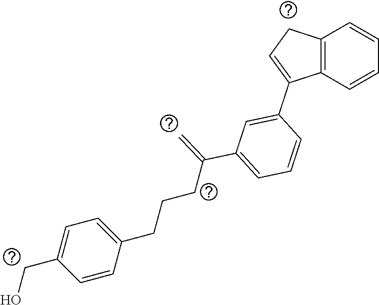 [SMILES: C=C(CCCc1ccc(CO)cc1)c1cccc(C2=CCc3ccccc32)c1]

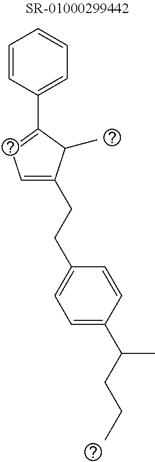 [SMILES: CCCC(C)c1ccc(CCC2=CC=C(c3ccccc3)C2C)cc1]

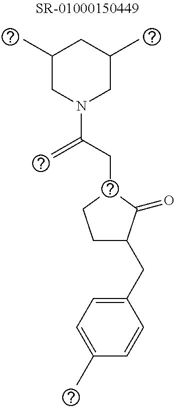 [SMILES: C=C(CC1CCC(Cc2ccc(C)cc2)C1=O)N1CC(C)CC(C)C1]

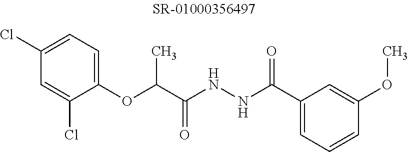 [SMILES: COc1cccc(C(=O)NNC(=O)C(C)Oc2ccc(Cl)cc2Cl)c1]

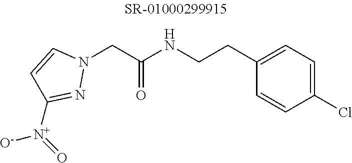 [SMILES: O=C(Cn1ccc([N+](=O)[O-])n1)NCCc1ccc(Cl)cc1]

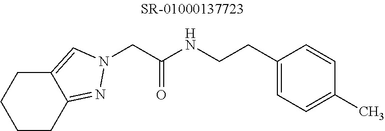 [SMILES: Cc1ccc(CCNC(=O)Cn2cc3c(n2)CCCC3)cc1]

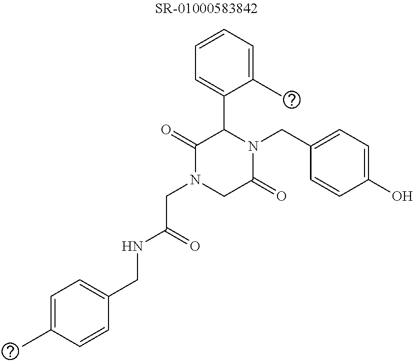 [SMILES: Cc1ccc(CNC(=O)CN2CC(=O)N(Cc3ccc(O)cc3)C(c3ccccc3C)C2=O)cc1]

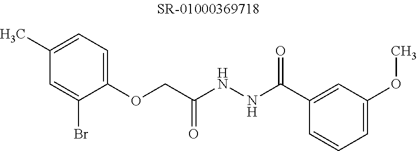

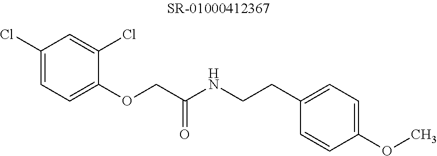 [SMILES: COc1ccc(CCNC(=O)COc2ccc(Cl)cc2Cl)cc1]

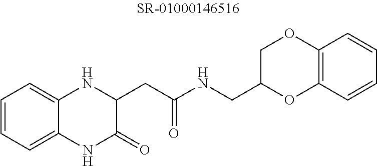 [SMILES: O=C(CC1Nc2ccccc2NC1=O)NCC1COc2ccccc2O1]

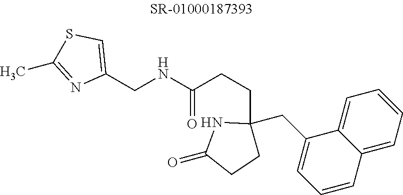 [SMILES: Cc1nc(CNC(=O)CCC2(Cc3cccc4ccccc34)CCC(=O)N2)cs1]

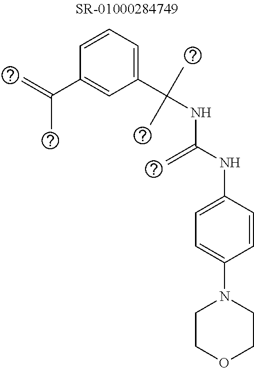 [SMILES: C=C(Nc1ccc(N2CCOCC2)cc1)NC(C)(C)c1cccc(C(=C)C)c1]

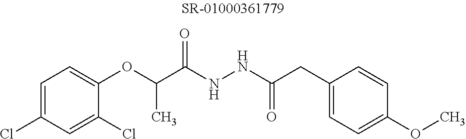 [SMILES: COc1ccc(CC(=O)NNC(=O)C(C)Oc2ccc(Cl)cc2Cl)cc1]

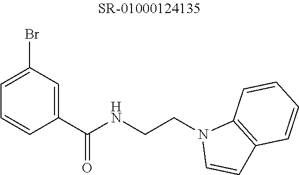 [SMILES: O=C(NCCn1ccc2ccccc21)c1cccc(Br)c1]

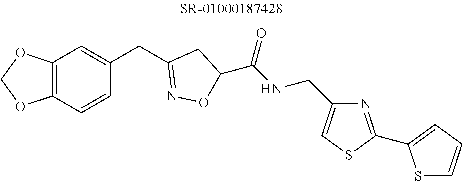 [SMILES: O=C(NCc1csc(-c2cccs2)n1)C1CC(Cc2ccc3c(c2)OCO3)=NO1]

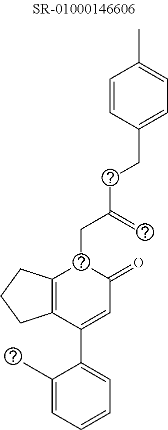 [SMILES: C=C(CCc1ccc(C)cc1)CC1C(=O)C=C(c2ccccc2C)C2=C1CCC2]

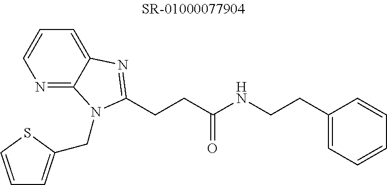 [SMILES: O=C(CCc1nc2cccnc2n1Cc1cccs1)NCCc1ccccc1]

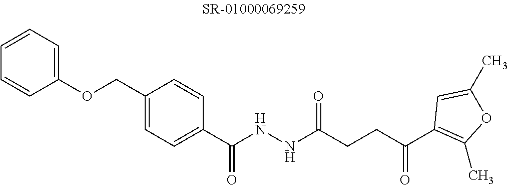 [SMILES: Cc1cc(C(=O)CCC(=O)NNC(=O)c2ccc(COc3ccccc3)cc2)c(C)o1]

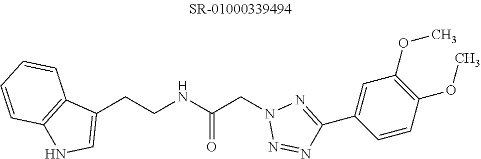 [SMILES: COc1ccc(-c2nnn(CC(=O)NCCc3c[nH]c4ccccc34)n2)cc1OC]

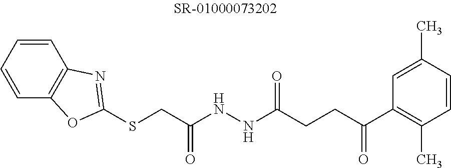 [SMILES: Cc1ccc(C)c(C(=O)CCC(=O)NNC(=O)CSc2nc3ccccc3o2)c1]

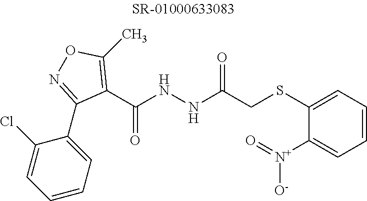 [SMILES: Cc1onc(-c2ccccc2Cl)c1C(=O)NNC(=O)CSc1ccccc1[N+](=O)[O-]]

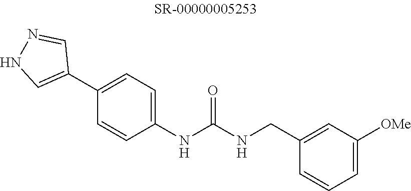 [SMILES: COc1cccc(CNC(=O)Nc2ccc(-c3cn[nH]c3)cc2)c1]

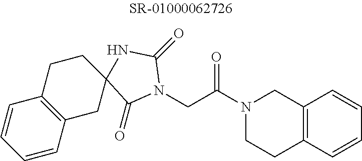 [SMILES: O=C(CN1C(=O)NC2(CCc3ccccc3C2)C1=O)N1CCc2ccccc2C1]

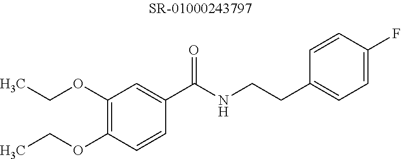 [SMILES: CCOc1ccc(C(=O)NCCc2ccc(F)cc2)cc1OCC]

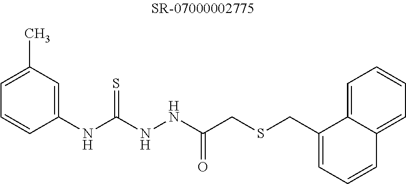 [SMILES: Cc1cccc(NC(=S)NNC(=O)CSCc2cccc3ccccc23)c1]

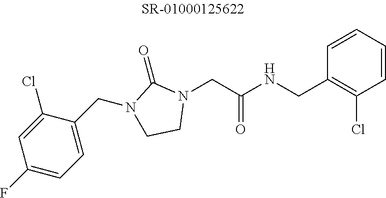 [SMILES: O=C(CN1CCN(Cc2ccc(F)cc2Cl)C1=O)NCc1ccccc1Cl]

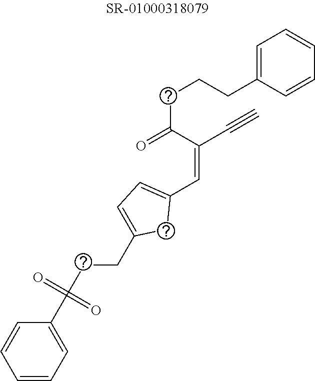

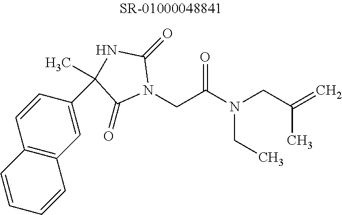 [SMILES: C=C(C)CN(CC)C(=O)CN1C(=O)NC(C)(c2ccc3ccccc3c2)C1=O]

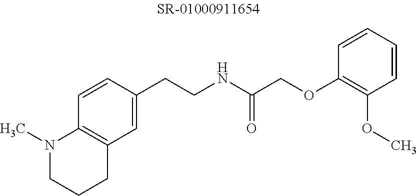

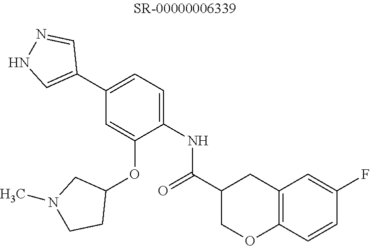 [SMILES: CN1CCC(Oc2cc(-c3cn[nH]c3)ccc2NC(=O)C2COc3ccc(F)cc3C2)C1]

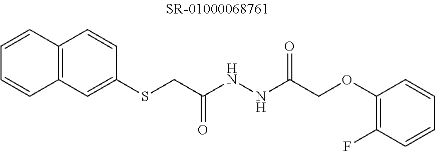 [SMILES: O=C(COc1ccccc1F)NNC(=O)CSc1ccc2ccccc2c1]

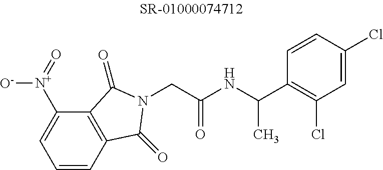 [SMILES: CC(NC(=O)CN1C(=O)c2cccc([N+](=O)[O-])c2C1=O)c1ccc(Cl)cc1Cl]

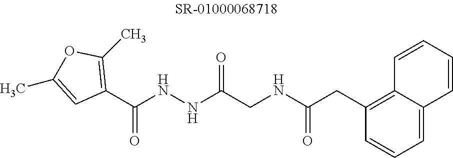 [SMILES: Cc1cc(C(=O)NNC(=O)CNC(=O)Cc2cccc3ccccc23)c(C)o1]

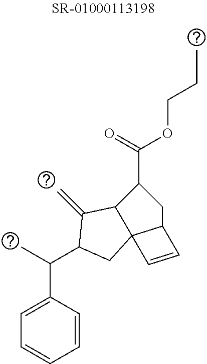 [SMILES: C=C1C(C(C)c2ccccc2)CC23C=CC2CC(C(=O)OCCC)C13]

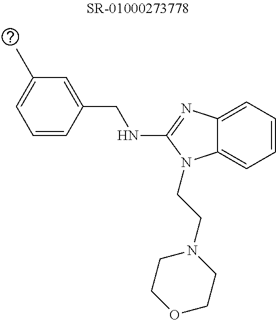 [SMILES: Cc1cccc(CNc2nc3ccccc3n2CCN2CCOCC2)c1]

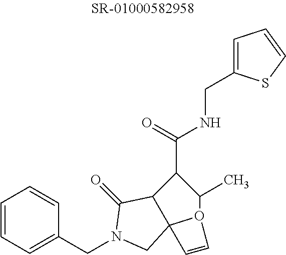

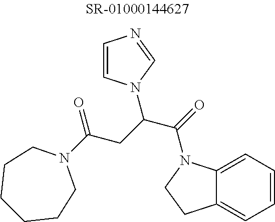 [SMILES: O=C(CC(C(=O)N1CCc2ccccc21)n1ccnc1)N1CCCCCC1]

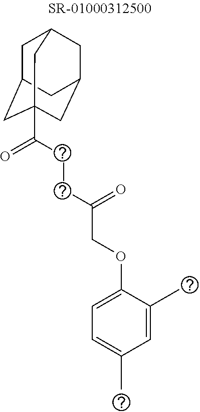 [SMILES: Cc1ccc(OCC(=O)CCC(=O)C23CC4CC(CC(C4)C2)C3)c(C)c1]

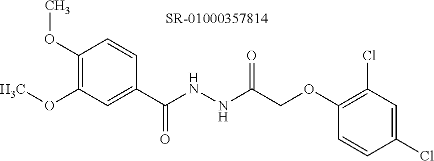 [SMILES: COc1ccc(C(=O)NNC(=O)COc2ccc(Cl)cc2Cl)cc1OC]

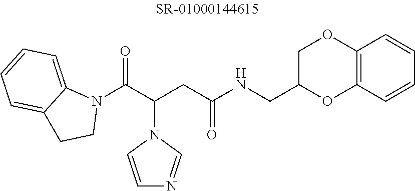 [SMILES: O=C(CC(C(=O)N1CCc2ccccc21)n1ccnc1)NCC1COc2ccccc2O1]

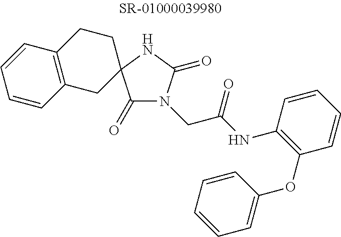 [SMILES: O=C(CN1C(=O)NC2(CCc3ccccc3C2)C1=O)Nc1ccccc1Oc1ccccc1]

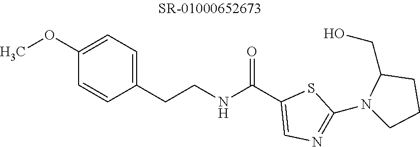 [SMILES: COc1ccc(CCNC(=O)c2cnc(N3CCCC3CO)s2)cc1]

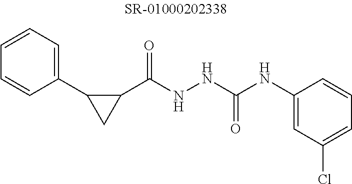 [SMILES: O=C(NNC(=O)C1CC1c1ccccc1)Nc1cccc(Cl)c1]

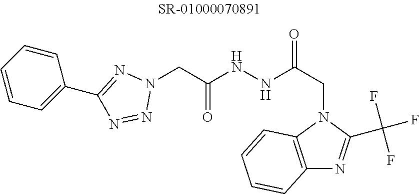 [SMILES: O=C(Cn1nnc(-c2ccccc2)n1)NNC(=O)Cn1c(C(F)(F)F)nc2ccccc21]

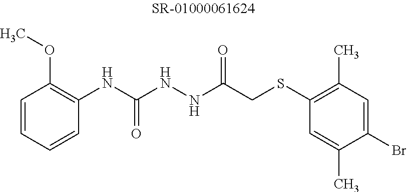 [SMILES: COc1ccccc1NC(=O)NNC(=O)CSc1cc(C)c(Br)cc1C]

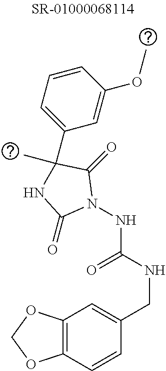 [SMILES: COc1cccc(C2(C)NC(=O)N(NC(=O)NCc3ccc4c(c3)OCO4)C2=O)c1]

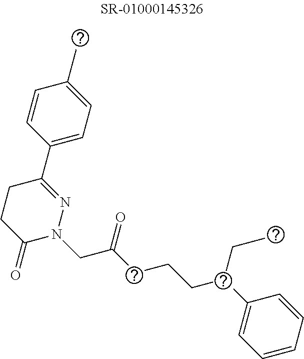 [SMILES: CCC(CCCC(=O)CN1N=C(c2ccc(C)cc2)CCC1=O)c1ccccc1]

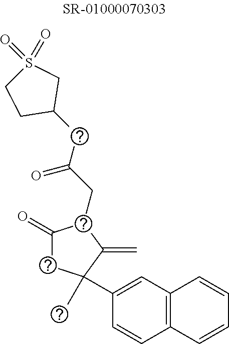 [SMILES: C=C1C(CC(=O)CC2CCS(=O)(=O)C2)C(=O)CC1(C)c1ccc2ccccc2c1]

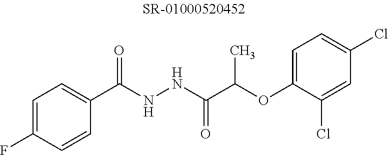 [SMILES: CC(Oc1ccc(Cl)cc1Cl)C(=O)NNC(=O)c1ccc(F)cc1]

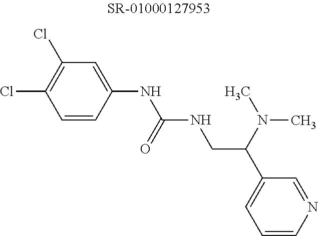 [SMILES: CN(C)C(CNC(=O)Nc1ccc(Cl)c(Cl)c1)c1cccnc1]

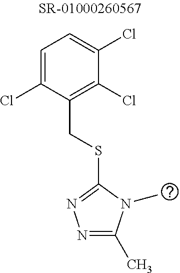 [SMILES: Cc1nnc(SCc2c(Cl)ccc(Cl)c2Cl)n1C]

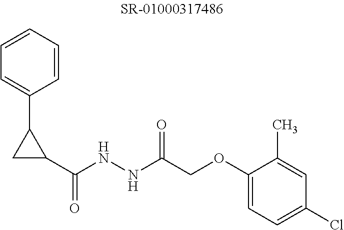 [SMILES: Cc1cc(Cl)ccc1OCC(=O)NNC(=O)C1CC1c1ccccc1]

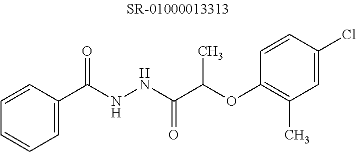 [SMILES: Cc1cc(Cl)ccc1OC(C)C(=O)NNC(=O)c1ccccc1]

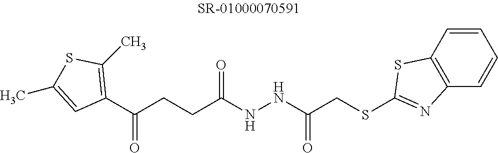 [SMILES: Cc1cc(C(=O)CCC(=O)NNC(=O)CSc2nc3ccccc3s2)c(C)s1]

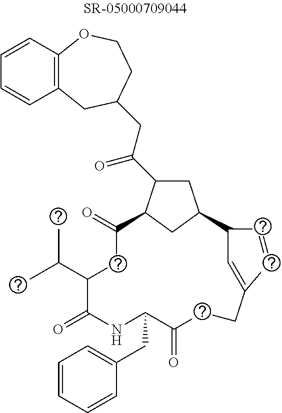 [SMILES: CC(C)C1CC(=O)[C@@H]2C[C@@H](CC2C(=O)CC2CCOc3ccccc3C2)C2C=CC(=C2)CCC(=O)[C@@H](Cc2ccccc2)NC1=O]

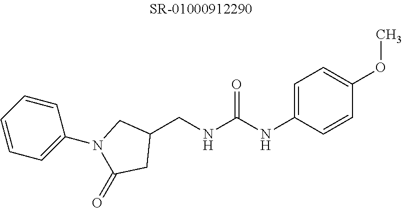 [SMILES: COc1ccc(NC(=O)NCC2CC(=O)N(c3ccccc3)C2)cc1]

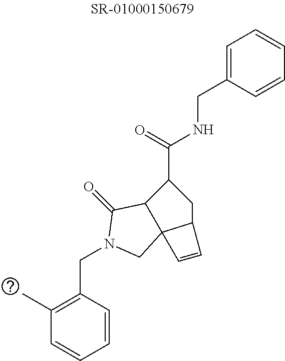 [SMILES: Cc1ccccc1CN1CC23C=CC2CC(C(=O)NCc2ccccc2)C3C1=O]

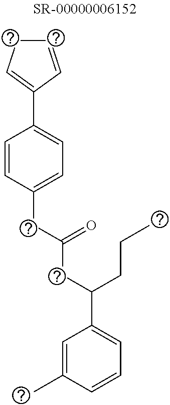 [SMILES: CCCC(CC(=O)Cc1ccc(C2=CCC=C2)cc1)c1cccc(C)c1]

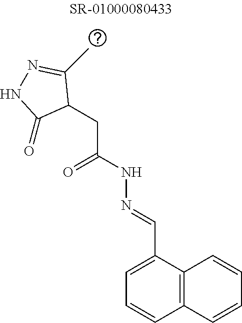 [SMILES: CC1=NNC(=O)C1CC(=O)N/N=C/c1cccc2ccccc12]

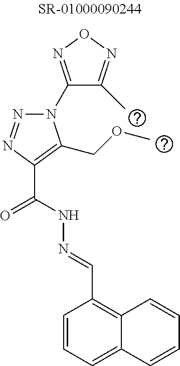 [SMILES: COCc1c(C(=O)N/N=C/c2cccc3ccccc23)nnn1-c1nonc1C]

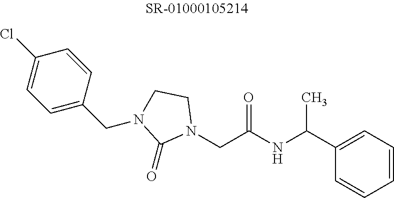 [SMILES: CC(NC(=O)CN1CCN(Cc2ccc(Cl)cc2)C1=O)c1ccccc1]

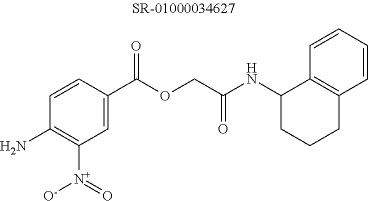 [SMILES: Nc1ccc(C(=O)OCC(=O)NC2CCCc3ccccc32)cc1[N+](=O)[O-]]

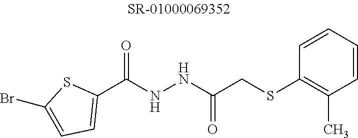 [SMILES: Cc1ccccc1SCC(=O)NNC(=O)c1ccc(Br)s1]

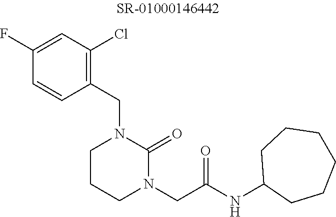 [SMILES: O=C(CN1CCCN(Cc2ccc(F)cc2Cl)C1=O)NC1CCCCCC1]

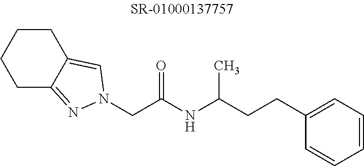 [SMILES: CC(CCc1ccccc1)NC(=O)Cn1cc2c(n1)CCCC2]

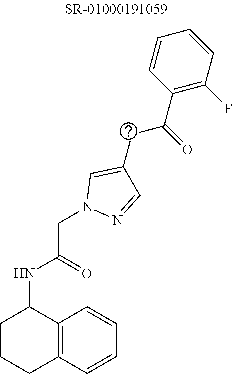 [SMILES: O=C(Cn1cc(CC(=O)c2ccccc2F)cn1)NC1CCCc2ccccc21]

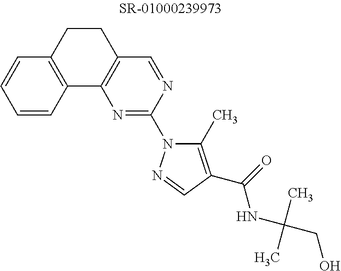 [SMILES: Cc1c(C(=O)NC(C)(C)CO)cnn1-c1ncc2c(n1)-c1ccccc1CC2]

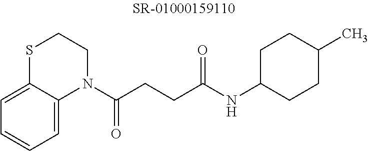 [SMILES: CC1CCC(NC(=O)CCC(=O)N2CCSc3ccccc32)CC1]

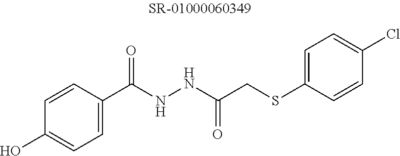 [SMILES: O=C(CSc1ccc(Cl)cc1)NNC(=O)c1ccc(O)cc1]

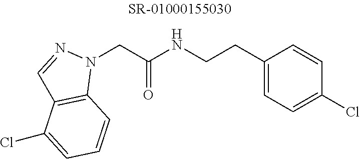 [SMILES: O=C(Cn1ncc2c(Cl)cccc21)NCCc1ccc(Cl)cc1]

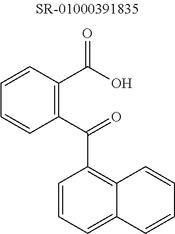 [SMILES: O=C(O)c1ccccc1C(=O)c1cccc2ccccc12]

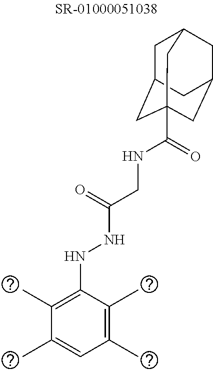 [SMILES: Cc1cc(C)c(C)c(NNC(=O)CNC(=O)C23CC4CC(CC(C4)C2)C3)c1C]

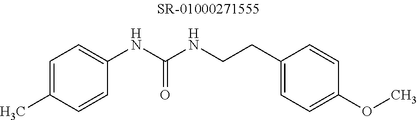

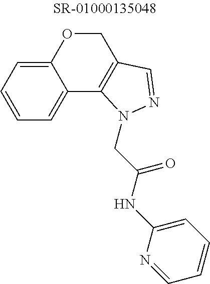 [SMILES: O=C(Cn1ncc2c1-c1ccccc1OC2)Nc1ccccn1]

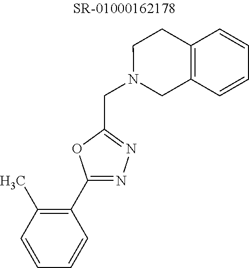 [SMILES: Cc1ccccc1-c1nnc(CN2CCc3ccccc3C2)o1]

SR-01000524029

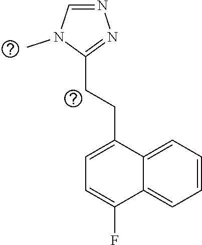 [SMILES: Cn1cnnc1CCc1ccc(F)c2ccccc12]

SR-01000061818

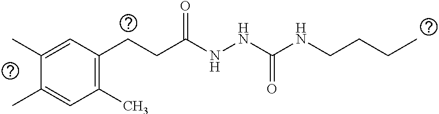 [SMILES: CCCCNC(=O)NNC(=O)CCc1cc(C)c(C)cc1C]

SR-01000535824

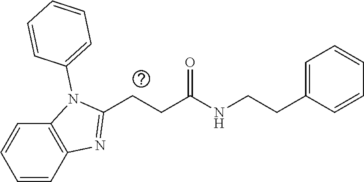 [SMILES: O=C(CCc1nc2ccccc2n1-c1ccccc1)NCCc1ccccc1]

SR-01000088477

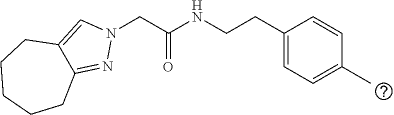 [SMILES: Cc1ccc(CCNC(=O)Cn2cc3c(n2)CCCCC3)cc1]

SR-01000048417

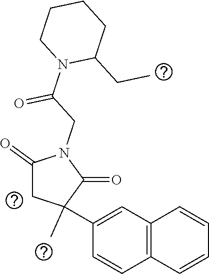 [SMILES: CCC1CCCCN1C(=O)CN1C(=O)CC(C)(c2ccc3ccccc3c2)C1=O]

SR-01000069564

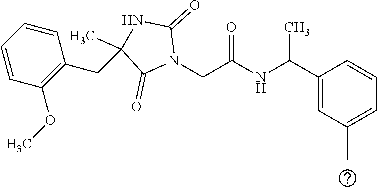 [SMILES: COc1ccccc1CC1(C)NC(=O)N(CC(=O)NC(C)c2cccc(C)c2)C1=O]

SR-00000006698

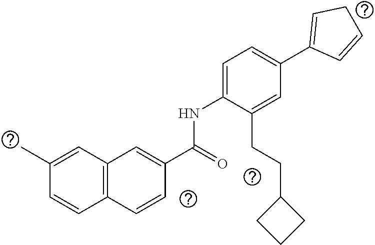 [SMILES: Cc1ccc2ccc(C(=O)Nc3ccc(C4=CCC=C4)cc3CCC3CCC3)cc2c1]

SR-01000479007

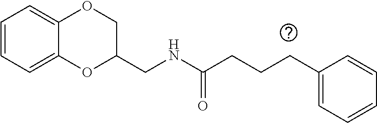 [SMILES: O=C(CCCc1ccccc1)NCC1COc2ccccc2O1]

SR-01000140172

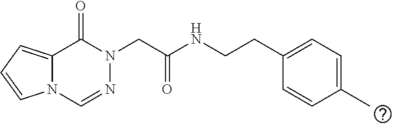 [SMILES: Cc1ccc(CCNC(=O)Cn2ncn3cccc3c2=O)cc1]

SR-00000005301

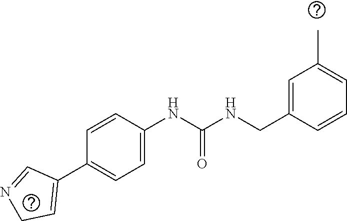 [SMILES: Cc1cccc(CNC(=O)Nc2ccc(C3=CCN=C3)cc2)c1]

SR-01000146448

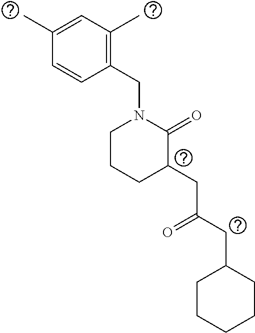 [SMILES: Cc1ccc(CN2CCCC(CC(=O)CC3CCCCC3)C2=O)c(C)c1]

SR-01000146446

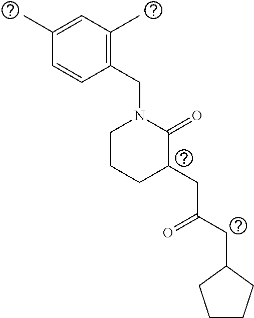 [SMILES: Cc1ccc(CN2CCCC(CC(=O)CC3CCCC3)C2=O)c(C)c1]

SR-01000358013

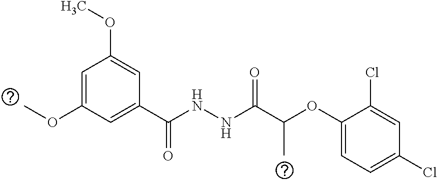 [SMILES: COc1cc(OC)cc(C(=O)NNC(=O)C(C)Oc2ccc(Cl)cc2Cl)c1]

SR-01000194602

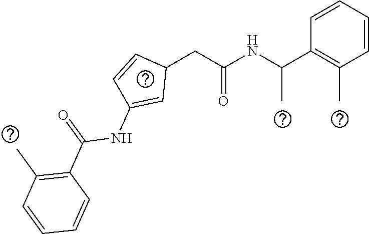 [SMILES: Cc1ccccc1C(=O)NC1=CC(CC(=O)NC(C)c2ccccc2C)C=C1]

SR-01000525232

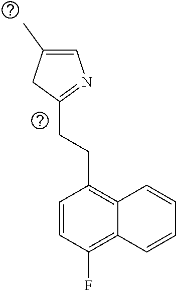 [SMILES: CC1=CN=C(CCc2ccc(F)c3ccccc23)C1]

SR-01000176150

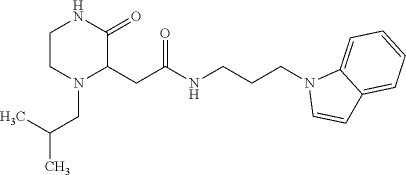 [SMILES: CC(C)CN1CCNC(=O)C1CC(=O)NCCCn1ccc2ccccc21]

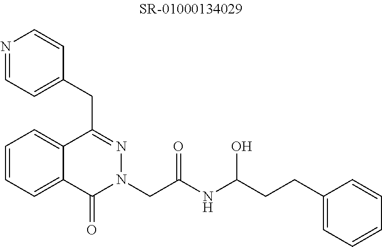 [SMILES: O=C(Cn1nc(Cc2ccncc2)c2ccccc2c1=O)NC(O)CCc1ccccc1]

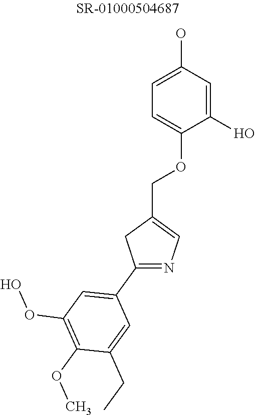 [SMILES: CCc1cc(C2=NC=C(COc3ccc(O)cc3O)C2)cc(OO)c1OC]

SR-01000031964

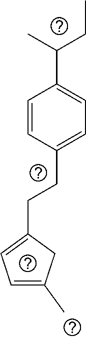 [SMILES: CCC(C)c1ccc(CCC2=CC=C(C)C2)cc1]

SR-01000145135

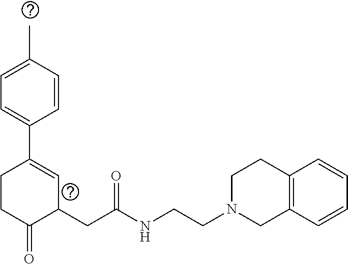 [SMILES: Cc1ccc(C2=CC(CC(=O)NCCN3CCc4ccccc4C3)C(=O)CC2)cc1]

SR-01000161248

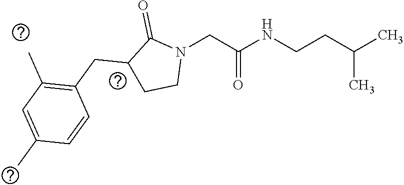 [SMILES: Cc1ccc(CC2CCN(CC(=O)NCCC(C)C)C2=O)c(C)c1]

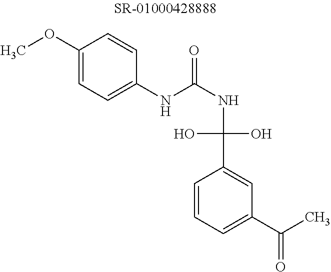 [SMILES: COc1ccc(NC(=O)NC(O)(O)c2cccc(C(C)=O)c2)cc1]

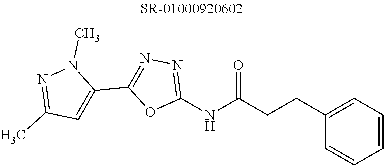 [SMILES: Cc1cc(-c2nnc(NC(=O)CCc3ccccc3)o2)n(C)n1]

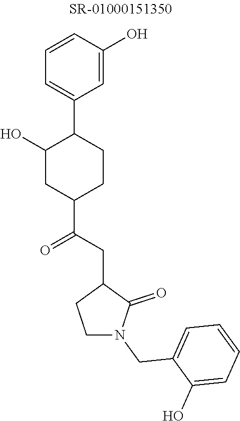 [SMILES: O=C(CC1CCN(Cc2ccccc2O)C1=O)C1CCC(c2cccc(O)c2)C(O)C1]

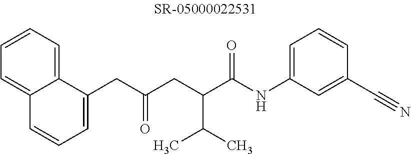 [SMILES: CC(C)C(CC(=O)Cc1cccc2ccccc12)C(=O)Nc1cccc(C#N)c1]

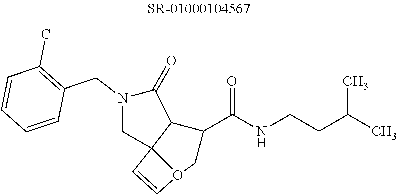

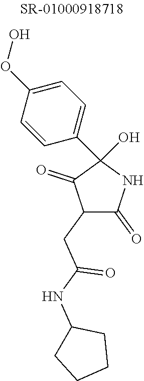 [SMILES: O=C(CC1C(=O)NC(O)(c2ccc(OO)cc2)C1=O)NC1CCCC1]

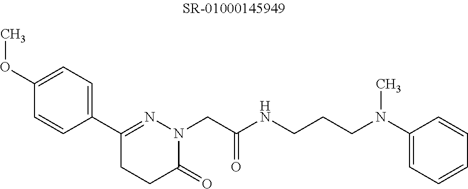 [SMILES: COc1ccc(C2=NN(CC(=O)NCCCN(C)c3ccccc3)C(=O)CC2)cc1]

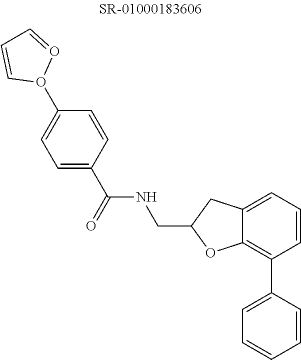

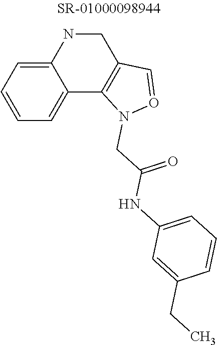

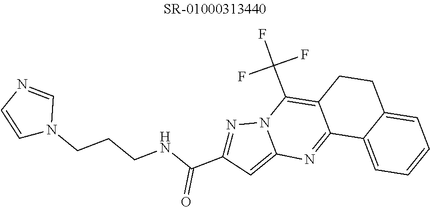 [SMILES: O=C(NCCCn1ccnc1)c1cc2nc3c(c(C(F)(F)F)n2n1)CCc1ccccc1-3]

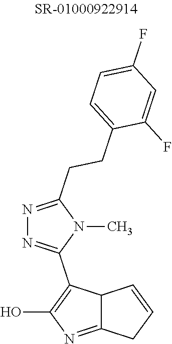 [SMILES: Cn1c(CCc2ccc(F)cc2F)nnc1C1=C(O)N=C2CC=CC21]

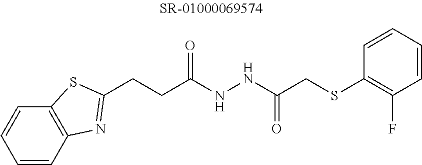 [SMILES: O=C(CCc1nc2ccccc2s1)NNC(=O)CSc1ccccc1F]

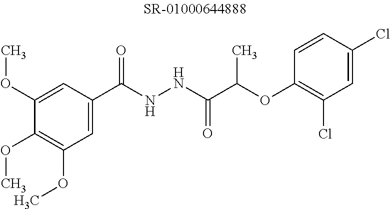 [SMILES: COc1cc(C(=O)NNC(=O)C(C)Oc2ccc(Cl)cc2Cl)cc(OC)c1OC]

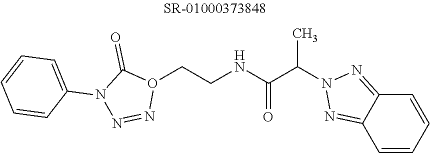

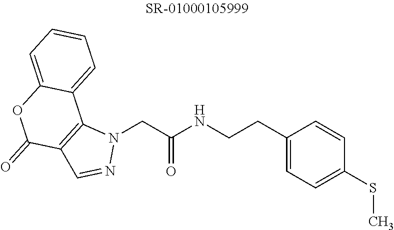 [SMILES: CSc1ccc(CCNC(=O)Cn2ncc3c(=O)oc4ccccc4c32)cc1]

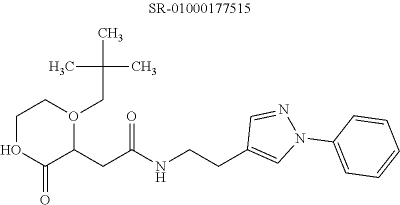

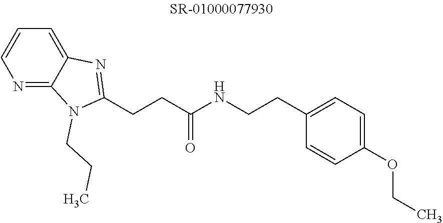 [SMILES: CCCn1c(CCC(=O)NCCc2ccc(OCC)cc2)nc2cccnc21]

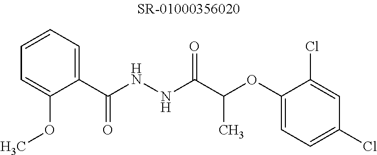 [SMILES: COc1ccccc1C(=O)NNC(=O)C(C)Oc1ccc(Cl)cc1Cl]

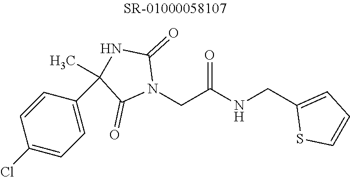 [SMILES: CC1(c2ccc(Cl)cc2)NC(=O)N(CC(=O)NCc2cccs2)C1=O]

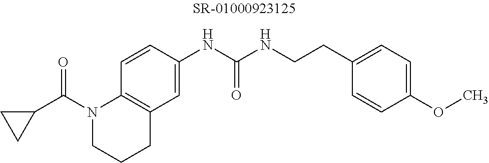 [SMILES: COc1ccc(CCNC(=O)Nc2ccc3c(c2)CCCN3C(=O)C2CC2)cc1]

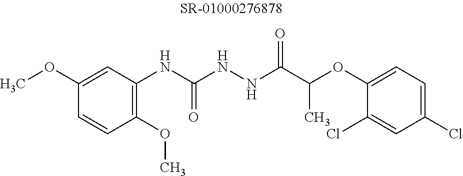 [SMILES: COc1ccc(OC)c(NC(=O)NNC(=O)C(C)Oc2ccc(Cl)cc2Cl)c1]

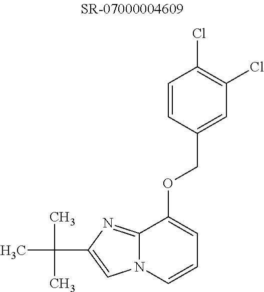 [SMILES: CC(C)(C)c1cn2cccc(OCc3ccc(Cl)c(Cl)c3)c2n1]

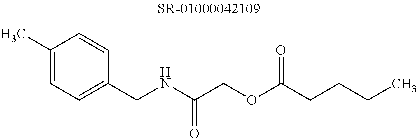 [SMILES: CCCCC(=O)OCC(=O)NCc1ccc(C)cc1]

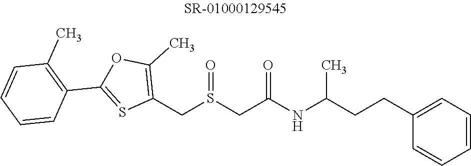

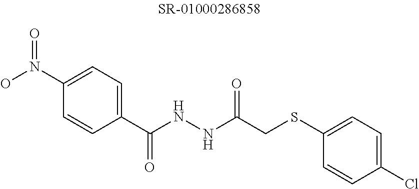

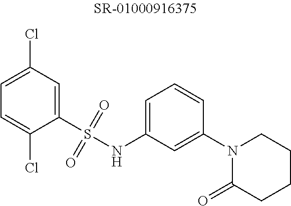 [SMILES: O=C1CCCCN1c1cccc(NS(=O)(=O)c2cc(Cl)ccc2Cl)c1]

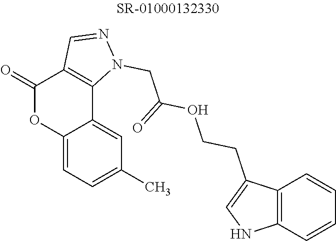 [SMILES: Cc1ccc2oc(=O)c3cnn(CC(=O)OCCc4c[nH]c5ccccc45)c3c2c1]

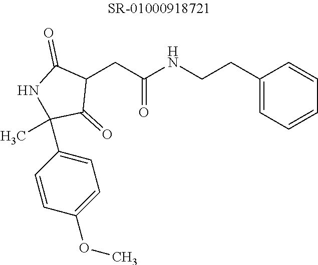 [SMILES: COc1ccc(C2(C)NC(=O)C(CC(=O)NCCc3ccccc3)C2=O)cc1]

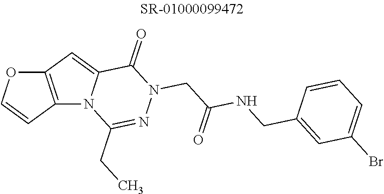 [SMILES: CCc1nn(CC(=O)NCc2cccc(Br)c2)c(=O)c2cc3occc3n12]

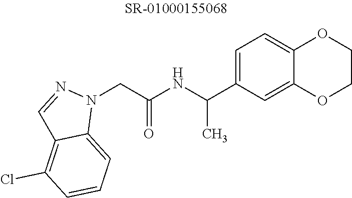 [SMILES: CC(NC(=O)Cn1ncc2c(Cl)cccc21)c1ccc2c(c1)OCCO2]

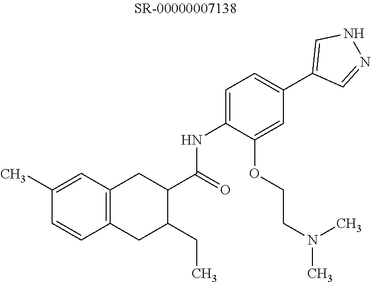 [SMILES: CCC1Cc2ccc(C)cc2CC1C(=O)Nc1ccc(-c2cn[nH]c2)cc1OCCN(C)C]

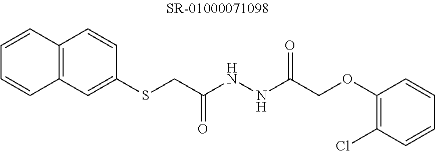 [SMILES: O=C(COc1ccccc1Cl)NNC(=O)CSc1ccc2ccccc2c1]

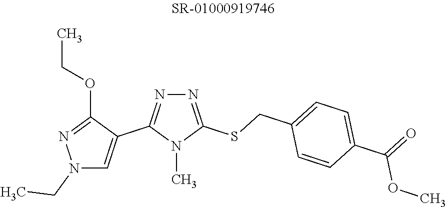 [SMILES: CCOc1nn(CC)cc1-c1nnc(SCc2ccc(C(=O)OC)cc2)n1C]

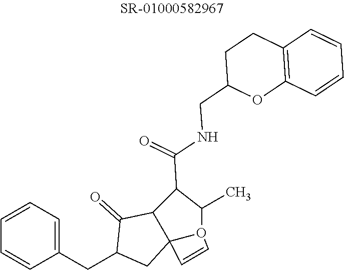

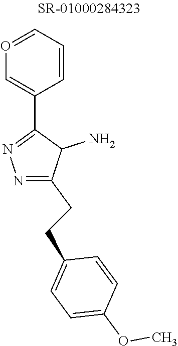

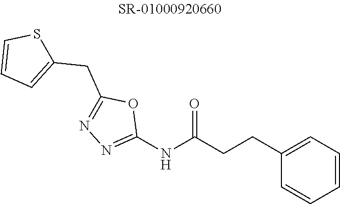 [SMILES: O=C(CCc1ccccc1)Nc1nnc(Cc2cccs2)o1]

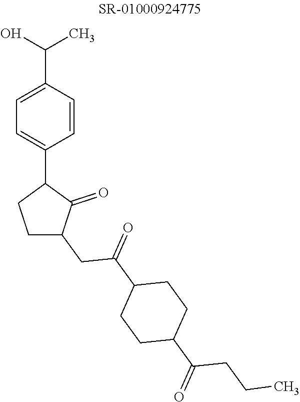 [SMILES: CCCC(=O)C1CCC(C(=O)CC2CCC(c3ccc(C(C)O)cc3)C2=O)CC1]

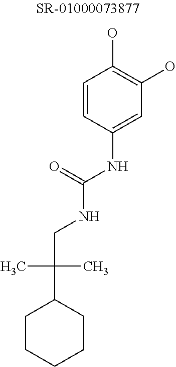 [SMILES: CC(C)(CNC(=O)Nc1ccc(O)c(O)c1)C1CCCCC1]

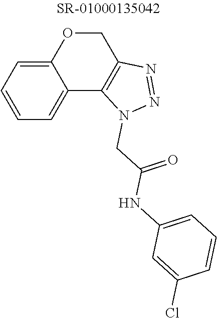 [SMILES: O=C(Cn1nnc2c1-c1ccccc1OC2)Nc1cccc(Cl)c1]

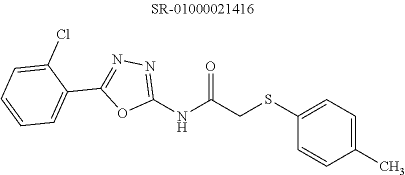 [SMILES: Cc1ccc(SCC(=O)Nc2nnc(-c3ccccc3Cl)o2)cc1]

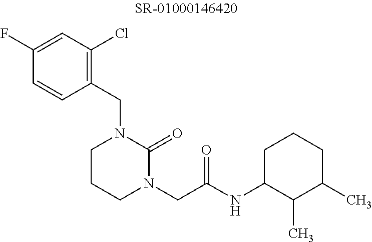 [SMILES: CC1CCCC(NC(=O)CN2CCCN(Cc3ccc(F)cc3Cl)C2=O)C1C]

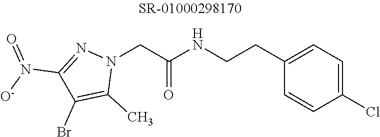

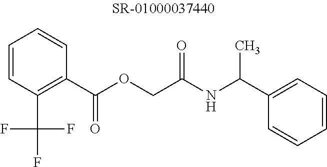 [SMILES: CC(NC(=O)COC(=O)c1ccccc1C(F)(F)F)c1ccccc1]

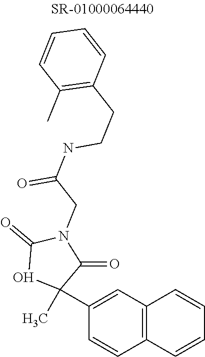 [SMILES: Cc1ccccc1CCNC(=O)CN1C(=O)OC(C)(c2ccc3ccccc3c2)C1=O]

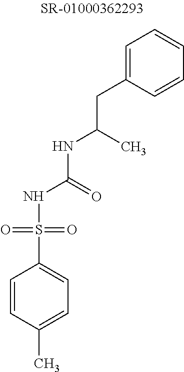 [SMILES: Cc1ccc(S(=O)(=O)NC(=O)NC(C)Cc2ccccc2)cc1]

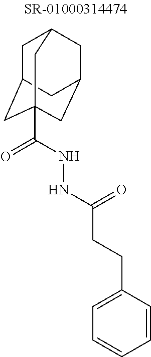 [SMILES: O=C(CCc1ccccc1)NNC(=O)C12CC3CC(CC(C3)C1)C2]

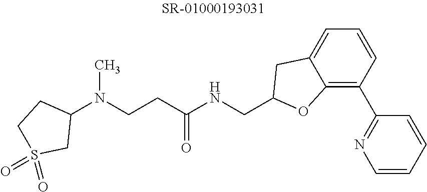 [SMILES: CN(CCC(=O)NCC1Cc2cccc(-c3ccccn3)c2O1)C1CCS(=O)(=O)C1]

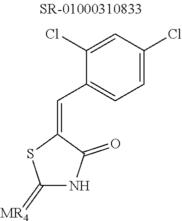

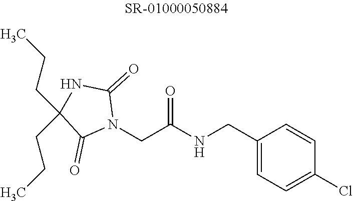 [SMILES: CCCC1(CCC)NC(=O)N(CC(=O)NCc2ccc(Cl)cc2)C1=O]

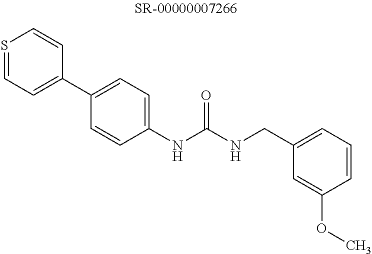

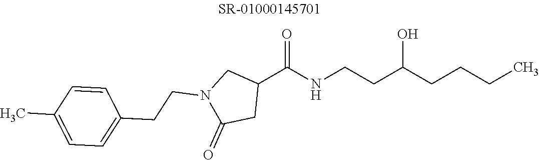 [SMILES: CCCCC(O)CCNC(=O)C1CC(=O)N(CCc2ccc(C)cc2)C1]

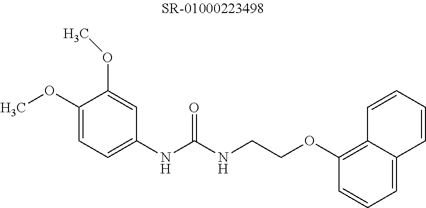 [SMILES: COc1ccc(NC(=O)NCCOc2cccc3ccccc23)cc1OC]

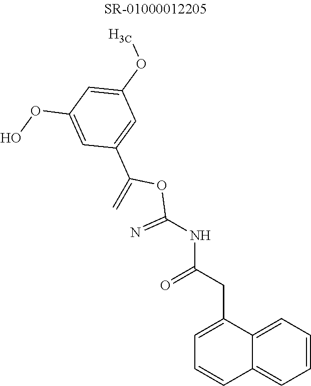 [SMILES: C=C(OC(=N)NC(=O)Cc1cccc2ccccc12)c1cc(OC)cc(OO)c1]

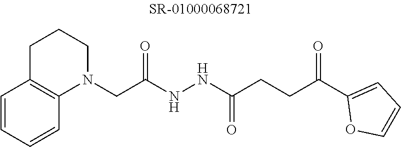 [SMILES: O=C(CCC(=O)c1ccco1)NNC(=O)CN1CCCc2ccccc21]

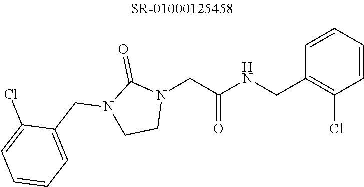 [SMILES: O=C(CN1CCN(Cc2ccccc2Cl)C1=O)NCc1ccccc1Cl]

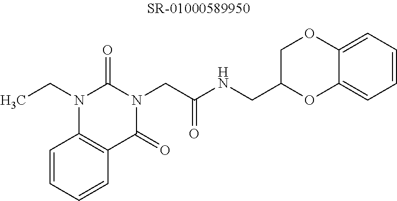 [SMILES: CCn1c(=O)n(CC(=O)NCC2COc3ccccc3O2)c(=O)c2ccccc21]

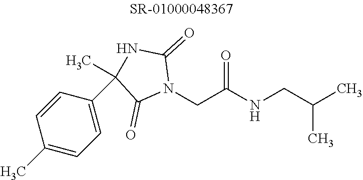 [SMILES: Cc1ccc(C2(C)NC(=O)N(CC(=O)NCC(C)C)C2=O)cc1]

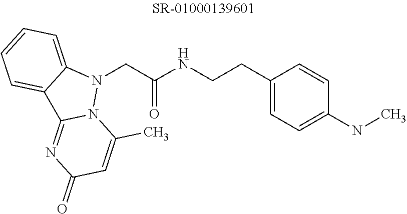 [SMILES: CNc1ccc(CCNC(=O)Cn2c3ccccc3c3nc(=O)cc(C)n32)cc1]

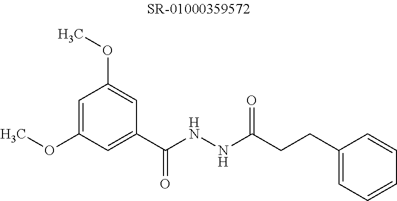 [SMILES: COc1cc(OC)cc(C(=O)NNC(=O)CCc2ccccc2)c1]

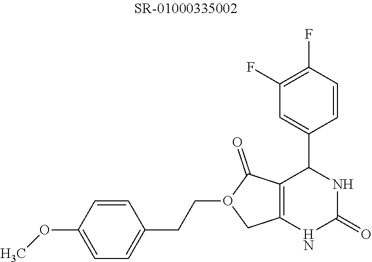

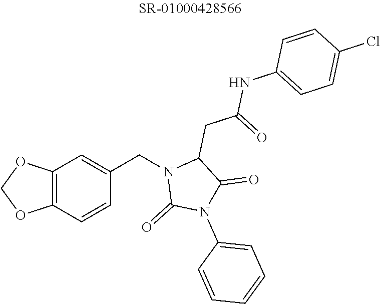 [SMILES: O=C(CC1C(=O)N(c2ccccc2)C(=O)N1Cc1ccc2c(c1)OCO2)Nc1ccc(Cl)cc1]

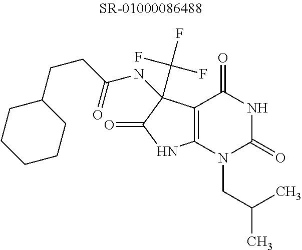 [SMILES: CC(C)Cn1c2c(c(=O)[nH]c1=O)C(NC(=O)CCC1CCCCC1)(C(F)(F)F)C(=O)N2]

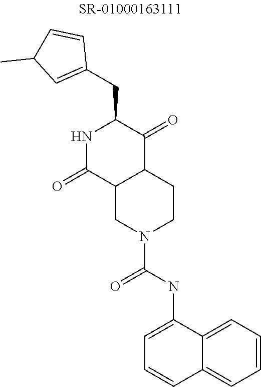 [SMILES: CC1C=CC(C[C@@H]2NC(=O)C3CN(C(=O)Nc4cccc5ccccc45)CCC3C2=O)=C1]

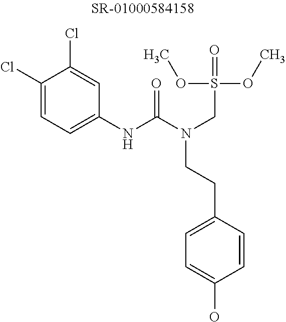

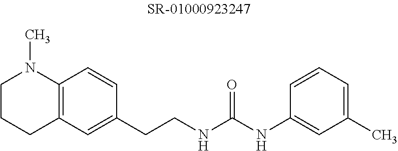 [SMILES: Cc1cccc(NC(=O)NCCc2ccc3c(c2)CCCN3C)c1]

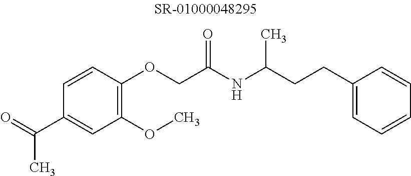 [SMILES: COc1cc(C(C)=O)ccc1OCC(=O)NC(C)CCc1ccccc1]

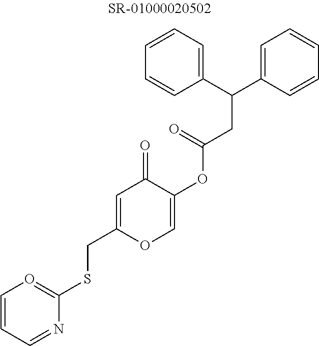

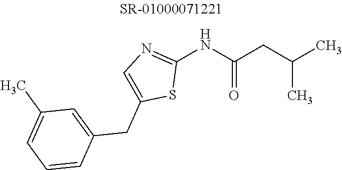 [SMILES: Cc1cccc(Cc2cnc(NC(=O)CC(C)C)s2)c1]

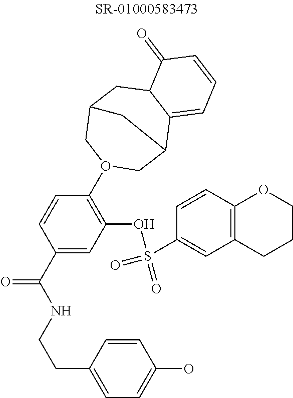

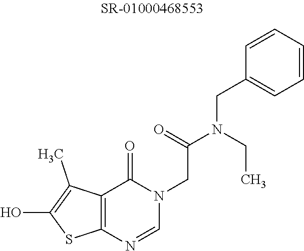 [SMILES: CCN(Cc1ccccc1)C(=O)Cn1cnc2sc(O)c(C)c2c1=O]

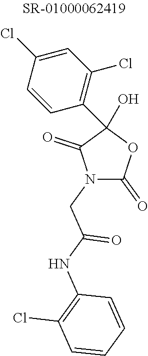 [SMILES: O=C(CN1C(=O)OC(O)(c2ccc(Cl)cc2Cl)C1=O)Nc1ccccc1Cl]

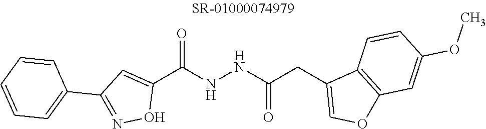 [SMILES: COc1ccc2c(CC(=O)NNC(=O)c3cc(-c4ccccc4)no3)coc2c1]

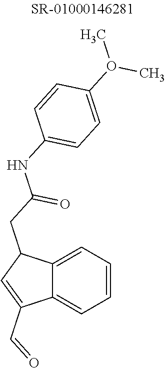

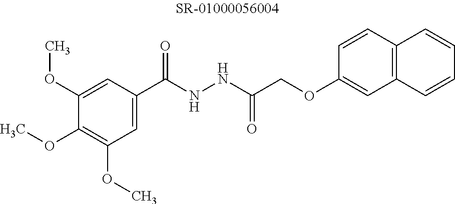 [SMILES: COc1cc(C(=O)NNC(=O)COc2ccc3ccccc3c2)cc(OC)c1OC]

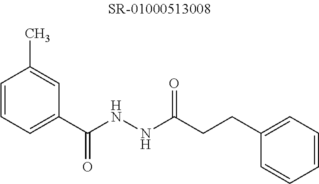 [SMILES: Cc1cccc(C(=O)NNC(=O)CCc2ccccc2)c1]

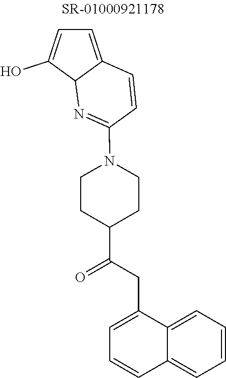 [SMILES: O=C(Cc1cccc2ccccc12)C1CCN(C2=NC3C(O)=CC=C3C=C2)CC1]

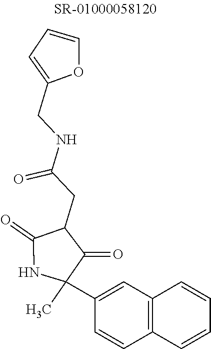 [SMILES: CC1(c2ccc3ccccc3c2)NC(=O)C(CC(=O)NCc2ccco2)C1=O]

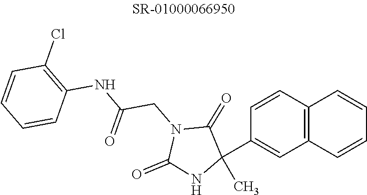 [SMILES: CC1(c2ccc3ccccc3c2)NC(=O)N(CC(=O)Nc2ccccc2Cl)C1=O]

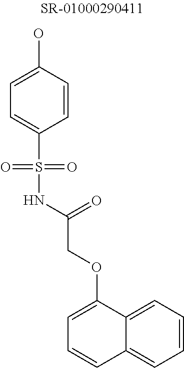 [SMILES: O=C(COc1cccc2ccccc12)NS(=O)(=O)c1ccc(O)cc1]

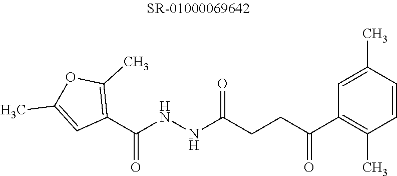 [SMILES: Cc1ccc(C)c(C(=O)CCC(=O)NNC(=O)c2cc(C)oc2C)c1]

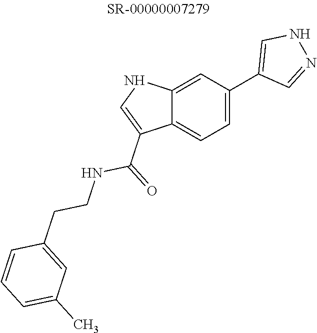 [SMILES: Cc1cccc(CCNC(=O)c2c[nH]c3cc(-c4cn[nH]c4)ccc23)c1]

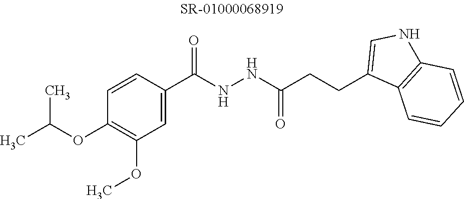 [SMILES: COc1cc(C(=O)NNC(=O)CCc2c[nH]c3ccccc23)ccc1OC(C)C]

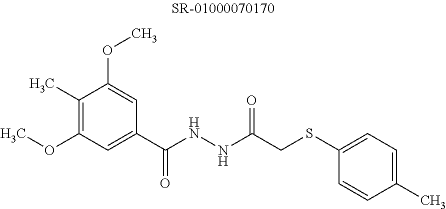

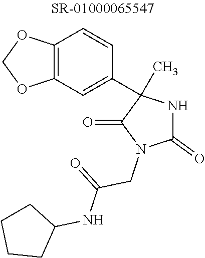 [SMILES: CC1(c2ccc3c(c2)OCO3)NC(=O)N(CC(=O)NC2CCCC2)C1=O]

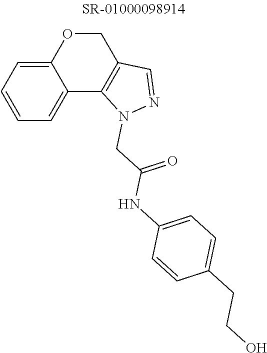 [SMILES: O=C(Cn1ncc2c1-c1ccccc1OC2)Nc1ccc(CCO)cc1]

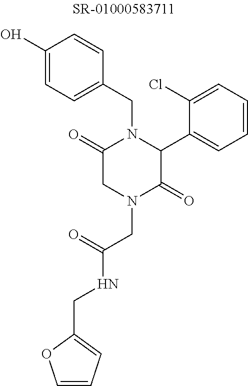 [SMILES: O=C(CN1CC(=O)N(Cc2ccc(O)cc2)C(c2ccccc2Cl)C1=O)NCc1ccco1]

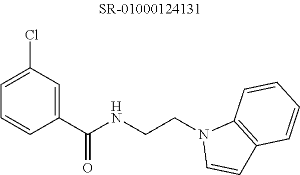 [SMILES: O=C(NCCn1ccc2ccccc21)c1cccc(Cl)c1]

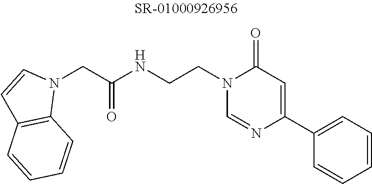 [SMILES: O=C(Cn1ccc2ccccc21)NCCn1cnc(-c2ccccc2)cc1=O]

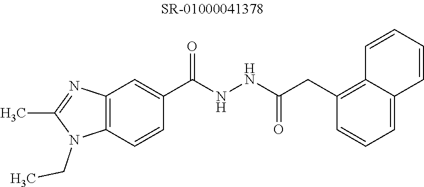 [SMILES: CCn1c(C)nc2cc(C(=O)NNC(=O)Cc3cccc4ccccc34)ccc21]

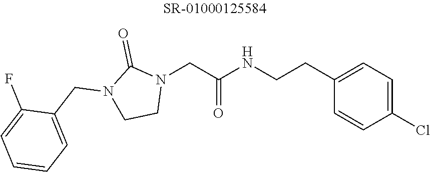 [SMILES: O=C(CN1CCN(Cc2ccccc2F)C1=O)NCCc1ccc(Cl)cc1]

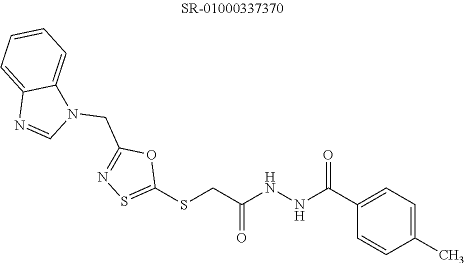

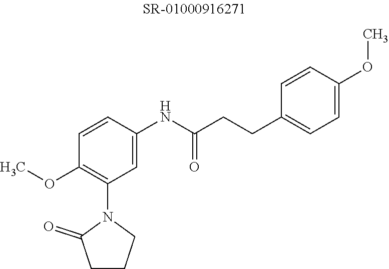

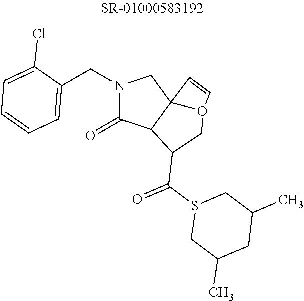

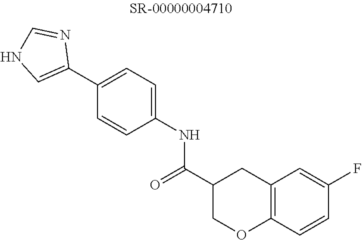 [SMILES: O=C(Nc1ccc(-c2c[nH]cn2)cc1)C1COc2ccc(F)cc2C1]

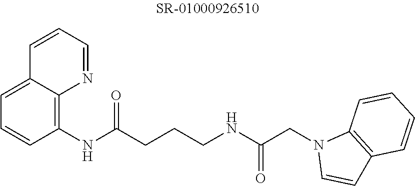 [SMILES: O=C(Cn1ccc2ccccc21)NCCCC(=O)Nc1cccc2cccnc12]

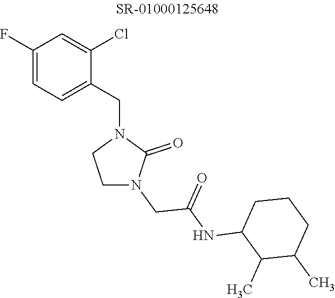 [SMILES: CC1CCCC(NC(=O)CN2CCN(Cc3ccc(F)cc3Cl)C2=O)C1C]

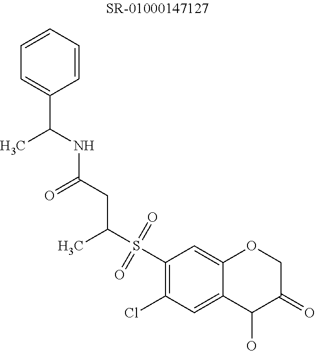 [SMILES: CC(NC(=O)CC(C)S(=O)(=O)c1cc2c(cc1Cl)C(O)C(=O)CO2)c1ccccc1]

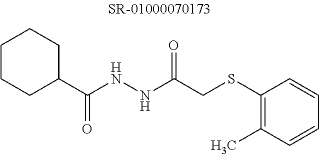 [SMILES: Cc1ccccc1SCC(=O)NNC(=O)C1CCCCC1]

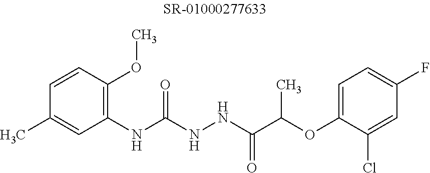 [SMILES: COc1ccc(C)cc1NC(=O)NNC(=O)C(C)Oc1ccc(F)cc1Cl]

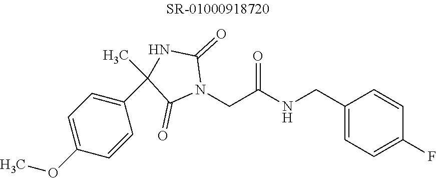 [SMILES: COc1ccc(C2(C)NC(=O)N(CC(=O)NCc3ccc(F)cc3)C2=O)cc1]

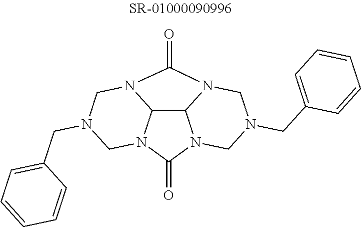 [SMILES: O=C1N2CN(Cc3ccccc3)CN3C(=O)N4CN(Cc5ccccc5)CN1C4C23]

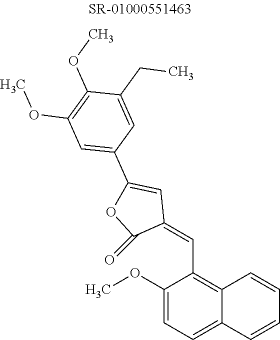 [SMILES: CCc1cc(C2=C/C(=C/c3c(OC)ccc4ccccc34)C(=O)O2)cc(OC)c1OC]

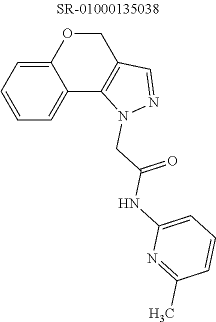 [SMILES: Cc1cccc(NC(=O)Cn2ncc3c2-c2ccccc2OC3)n1]

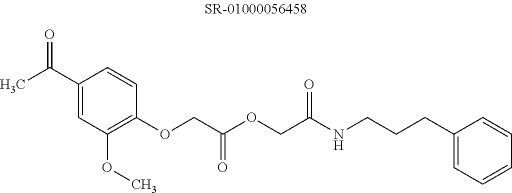 [SMILES: COc1cc(C(C)=O)ccc1OCC(=O)OCC(=O)NCCCc1ccccc1]

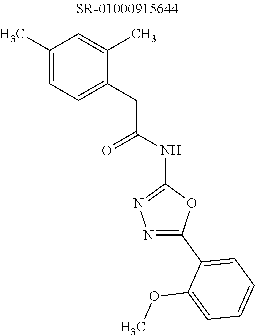 [SMILES: COc1ccccc1-c1nnc(NC(=O)Cc2ccc(C)cc2C)o1]

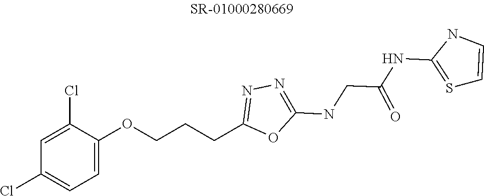

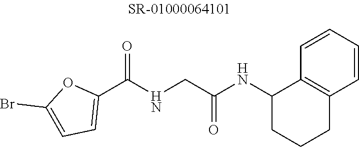 [SMILES: O=C(CNC(=O)c1ccc(Br)o1)NC1CCCc2ccccc21]

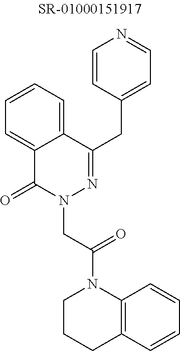 [SMILES: O=C(Cn1nc(Cc2ccncc2)c2ccccc2c1=O)N1CCCc2ccccc21]

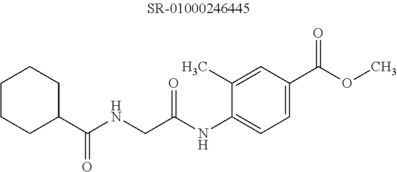 [SMILES: COC(=O)c1ccc(NC(=O)CNC(=O)C2CCCCC2)c(C)c1]

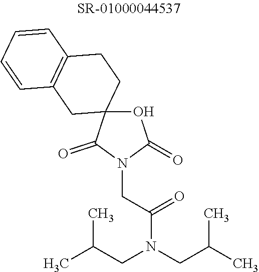 [SMILES: CC(C)CN(CC(C)C)C(=O)CN1C(=O)OC2(CCc3ccccc3C2)C1=O]

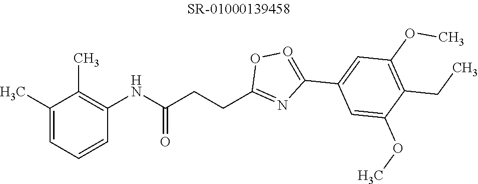

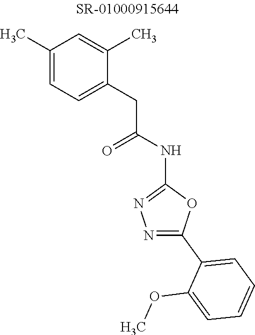 [SMILES: COc1ccccc1-c1nnc(NC(=O)Cc2ccc(C)cc2C)o1]

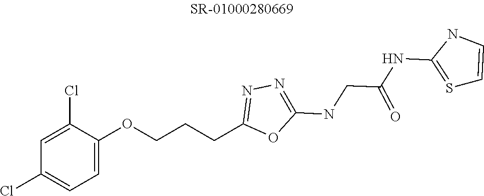

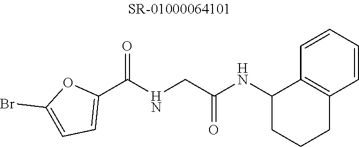 [SMILES: O=C(CNC(=O)c1ccc(Br)o1)NC1CCCc2ccccc21]

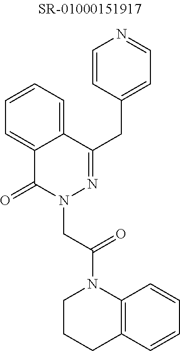 [SMILES: O=C(Cn1nc(Cc2ccncc2)c2ccccc2c1=O)N1CCCc2ccccc21]

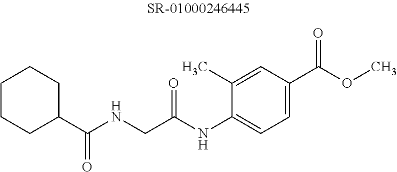 [SMILES: COC(=O)c1ccc(NC(=O)CNC(=O)C2CCCCC2)c(C)c1]

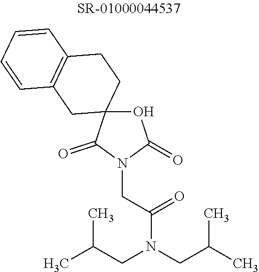 [SMILES: CC(C)CN(CC(C)C)C(=O)CN1C(=O)OC2(CCc3ccccc3C2)C1=O]

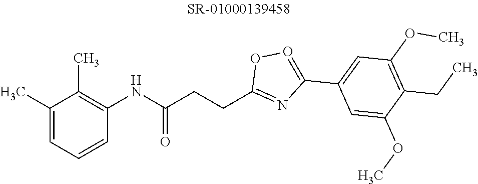

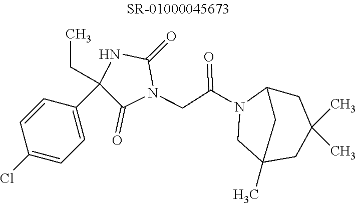 [SMILES: CCC1(c2ccc(Cl)cc2)NC(=O)N(CC(=O)N2CC3(C)CC2CC(C)(C)C3)C1=O]

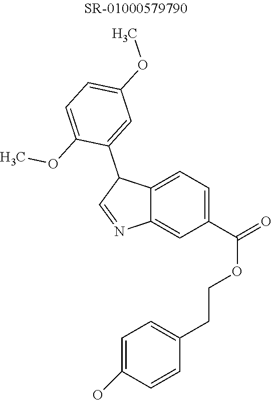 [SMILES: COc1ccc(OC)c(C2C=Nc3cc(C(=O)OCCc4ccc(O)cc4)ccc32)c1]

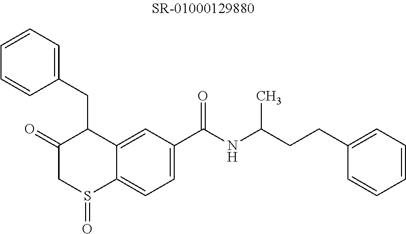 [SMILES: CC(CCc1ccccc1)NC(=O)c1ccc2c(c1)C(Cc1ccccc1)C(=O)CS2=O]

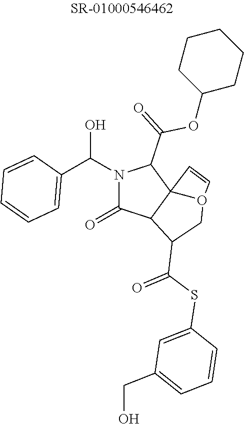

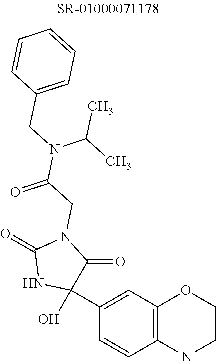 [SMILES: CC(C)N(Cc1ccccc1)C(=O)CN1C(=O)NC(O)(c2ccc3c(c2)OCCN3)C1=O]

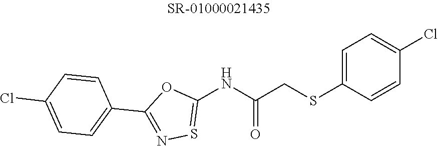

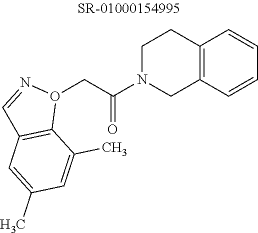

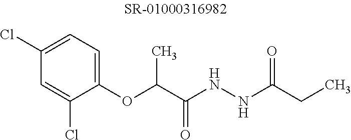 [SMILES: CCC(=O)NNC(=O)C(C)Oc1ccc(Cl)cc1Cl]

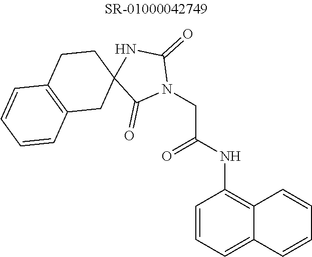 [SMILES: O=C(CN1C(=O)NC2(CCc3ccccc3C2)C1=O)Nc1cccc2ccccc12]

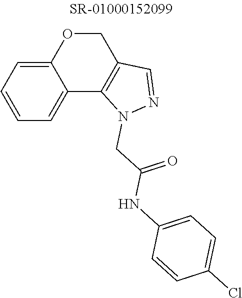 [SMILES: O=C(Cn1ncc2c1-c1ccccc1OC2)Nc1ccc(Cl)cc1]

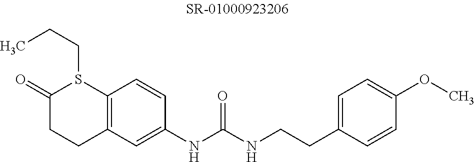

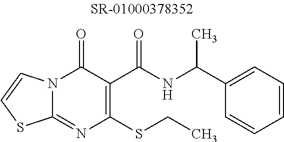 [SMILES: CCSc1nc2sccn2c(=O)c1C(=O)NC(C)c1ccccc1]

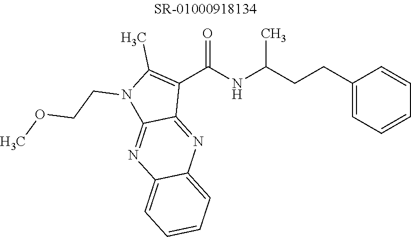 [SMILES: COCCn1c(C)c(C(=O)NC(C)CCc2ccccc2)c2nc3ccccc3nc21]

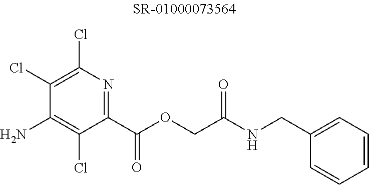 [SMILES: Nc1c(Cl)c(Cl)nc(C(=O)OCC(=O)NCc2ccccc2)c1Cl]

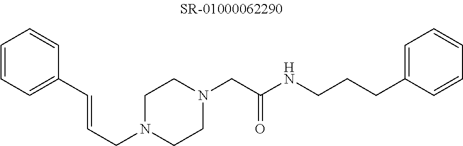 [SMILES: O=C(CN1CCN(C/C=C/c2ccccc2)CC1)NCCCc1ccccc1]

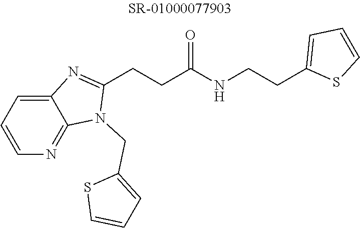 [SMILES: O=C(CCc1nc2cccnc2n1Cc1cccs1)NCCc1cccs1]

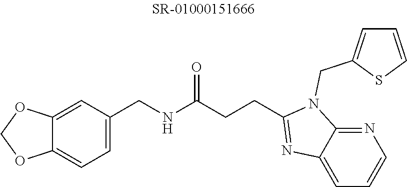 [SMILES: O=C(CCc1nc2cccnc2n1Cc1cccs1)NCc1ccc2c(c1)OCO2]

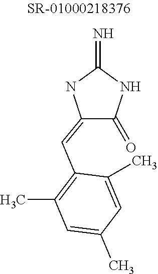

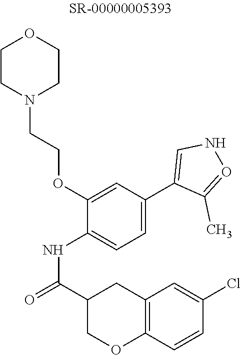

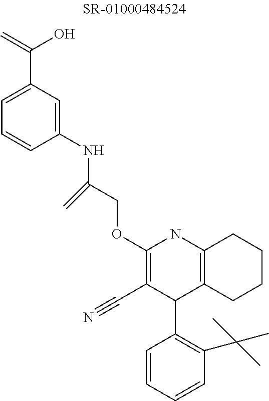 [SMILES: C=C(COC1=C(C#N)C(c2ccccc2C(C)(C)C)C2=C(CCCC2)N1)Nc1cccc(C(=C)O)c1]

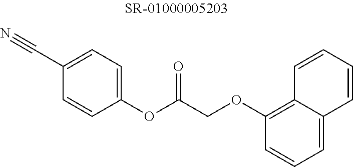 [SMILES: N#Cc1ccc(OC(=O)COc2cccc3ccccc23)cc1]

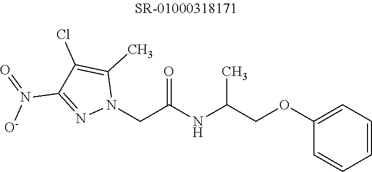

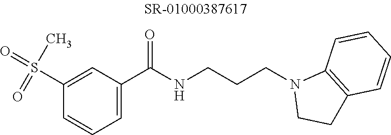 [SMILES: CS(=O)(=O)c1cccc(C(=O)NCCCN2CCc3ccccc32)c1]

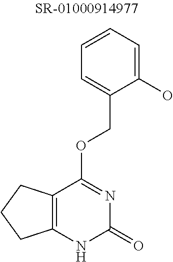 [SMILES: O=c1nc(OCc2ccccc2O)c2c([nH]1)CCC2]

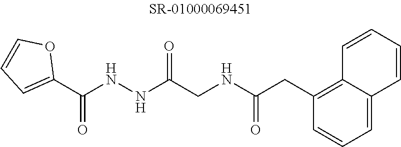 [SMILES: O=C(Cc1cccc2ccccc12)NCC(=O)NNC(=O)c1ccco1]

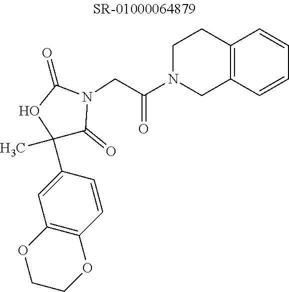 [SMILES: CC1(c2ccc3c(c2)OCCO3)OC(=O)N(CC(=O)N2CCc3ccccc3C2)C1=O]

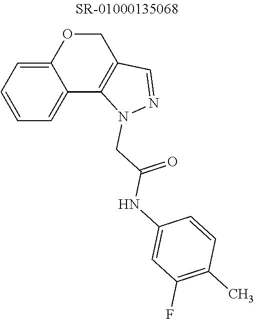 [SMILES: Cc1ccc(NC(=O)Cn2ncc3c2-c2ccccc2OC3)cc1F]

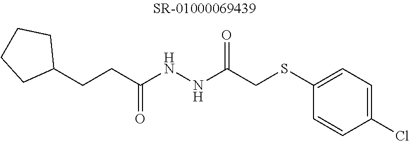 [SMILES: O=C(CCC1CCCC1)NNC(=O)CSc1ccc(Cl)cc1]

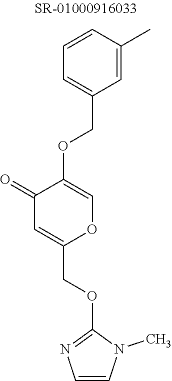 [SMILES: Cc1cccc(COc2coc(COc3nccn3C)cc2=O)c1]

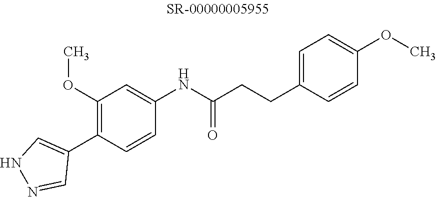 [SMILES: COc1ccc(CCC(=O)Nc2ccc(-c3cn[nH]c3)c(OC)c2)cc1]

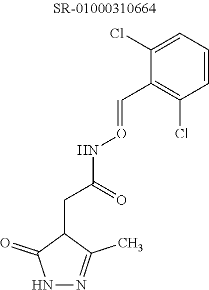

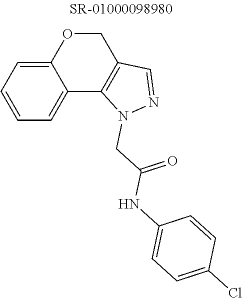 [SMILES: O=C(Cn1ncc2c1-c1ccccc1OC2)Nc1ccc(Cl)cc1]

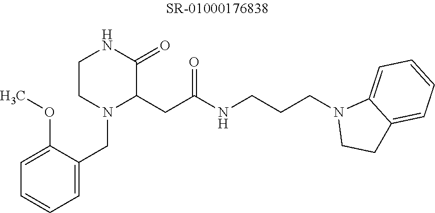 [SMILES: COc1ccccc1CN1CCNC(=O)C1CC(=O)NCCCN1CCc2ccccc21]

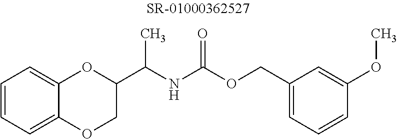 [SMILES: COc1cccc(COC(=O)NC(C)C2COc3ccccc3O2)c1]

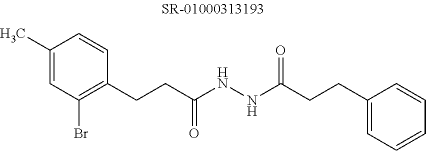 [SMILES: Cc1ccc(CCC(=O)NNC(=O)CCc2ccccc2)c(Br)c1]

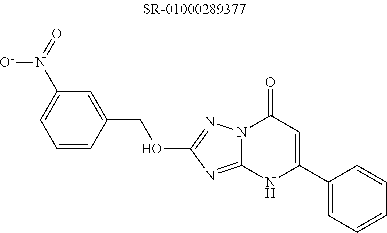

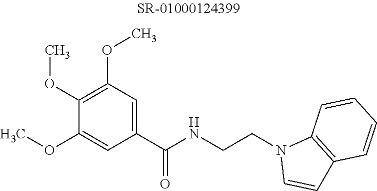 [SMILES: COc1cc(C(=O)NCCn2ccc3ccccc32)cc(OC)c1OC]

SR-01000923249

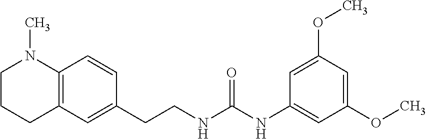 [SMILES: COc1cc(NC(=O)NCCc2ccc3c(c2)CCCN3C)cc(OC)c1]

SR-01000188699

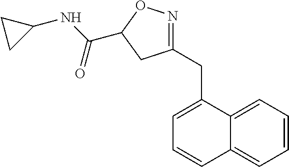 [SMILES: O=C(NC1CC1)C1CC(Cc2cccc3ccccc23)=NO1]

SR-01000069435

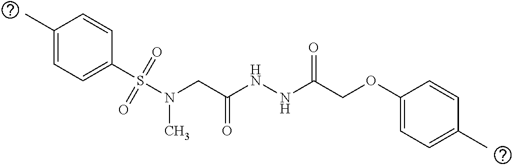

SR-01000297292

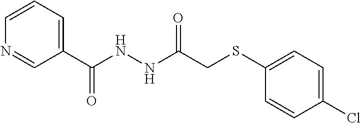 [SMILES: O=C(CSc1ccc(Cl)cc1)NNC(=O)c1cccnc1]

SR-01000906953

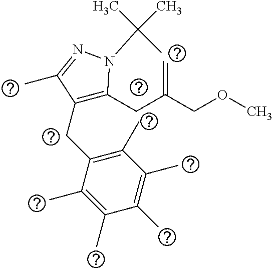

SR-01000925002

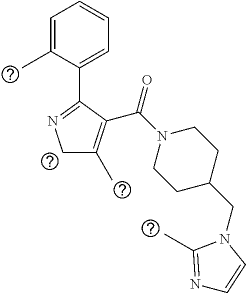 [SMILES: CC1=C(C(=O)N2CCC(Cn3ccnc3C)CC2)C(c2ccccc2C)=NC1]

SR-01000106321

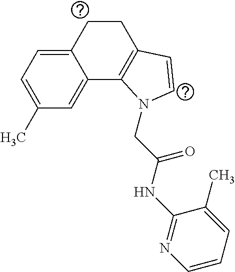 [SMILES: Cc1ccc2c(c1)-c1c(ccn1CC(=O)Nc1ncccc1C)CC2]

SR-01000565308

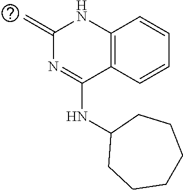 [SMILES: C=C1N=C(NC2CCCCCC2)c2ccccc2N1]

SR-01000013346

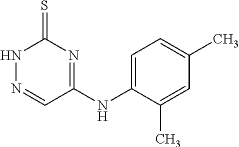 [SMILES: Cc1ccc(Nc2cn[nH]c(=S)n2)c(C)c1]

SR-01000359546

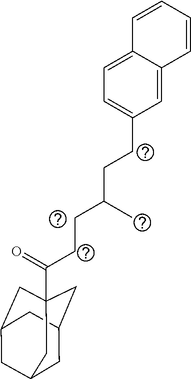 [SMILES: CC(CCC(=O)C12CC3CC(CC(C3)C1)C2)CCc1ccc2ccccc2c1]

SR-01000099712

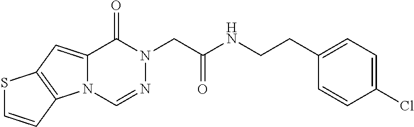 [SMILES: O=C(Cn1ncn2c(cc3sccc32)c1=O)NCCc1ccc(Cl)cc1]

SR-01000021433

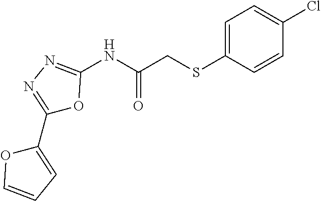 [SMILES: O=C(CSc1ccc(Cl)cc1)Nc1nnc(-c2ccco2)o1]

SR-01000152365

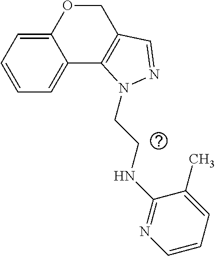 [SMILES: Cc1cccnc1NCCn1ncc2c1-c1ccccc1OC2]

SR-01000080408

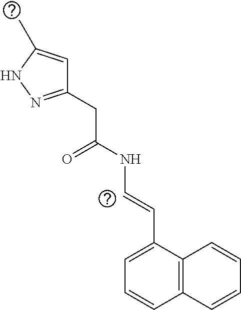 [SMILES: Cc1cc(CC(=O)N/C=C/c2cccc3ccccc23)n[nH]1]

SR-01000074687

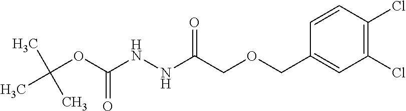 [SMILES: CC(C)(C)OC(=O)NNC(=O)COCc1ccc(Cl)c(Cl)c1]

SR-01000029575

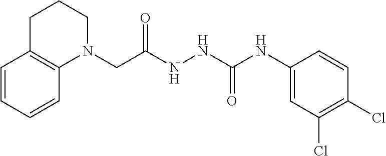 [SMILES: O=C(CN1CCCc2ccccc21)NNC(=O)Nc1ccc(Cl)c(Cl)c1]

SR-01000135037

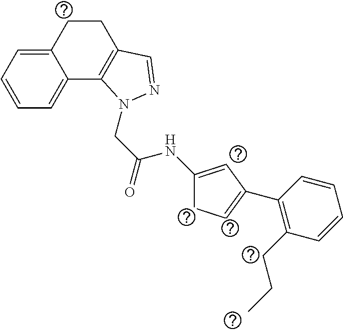 [SMILES: CCCc1ccccc1C1=CCC(NC(=O)Cn2ncc3c2-c2ccccc2CC3)=C1]

SR-01000125346

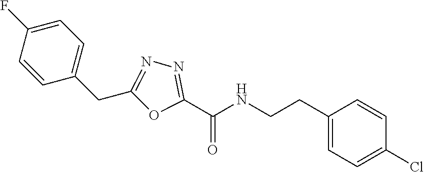 [SMILES: O=C(NCCc1ccc(Cl)cc1)c1nnc(Cc2ccc(F)cc2)o1]

SR-01000004123

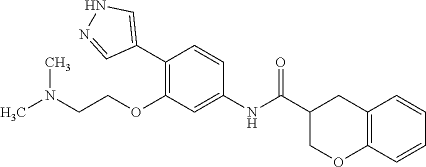 [SMILES: CN(C)CCOc1cc(NC(=O)C2COc3ccccc3C2)ccc1-c1cn[nH]c1]

SR-01000114492

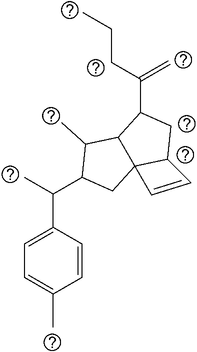 [SMILES: C=C(CCC)C1CC2C=CC23CC(C(C)c2ccc(C)cc2)C(C)C13]

SR-01000296100

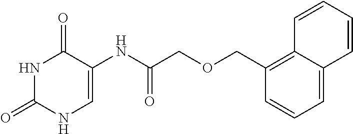 [SMILES: O=C(COCc1cccc2ccccc12)Nc1c[nH]c(=O)[nH]c1=O]

SR-01000125626

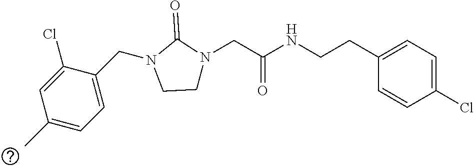 [SMILES: Cc1ccc(CN2CCN(CC(=O)NCCc3ccc(Cl)cc3)C2=O)c(Cl)c1]

SR-01000134913

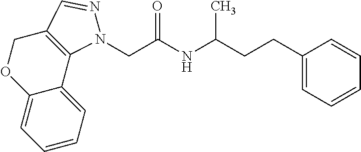 [SMILES: CC(CCc1ccccc1)NC(=O)Cn1ncc2c1-c1ccccc1OC2]

SR-01000336035

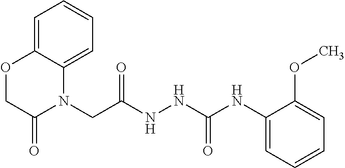 [SMILES: COc1ccccc1NC(=O)NNC(=O)CN1C(=O)COc2ccccc21]

SR-01000054849

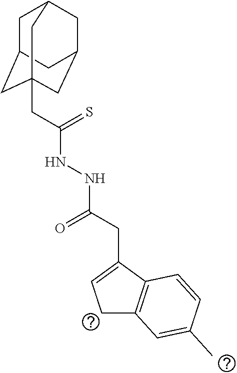 [SMILES: Cc1ccc2c(c1)CC=C2CC(=O)NNC(=S)CC12CC3CC(CC(C3)C1)C2]

SR-01000135070

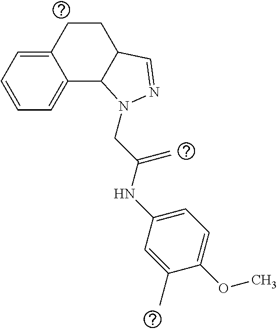 [SMILES: C=C(CN1N=CC2CCc3ccccc3C21)Nc1ccc(OC)c(C)c1]

SR-01000484454

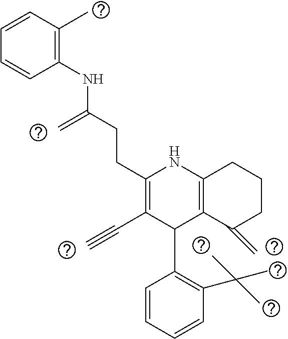 [SMILES: C#CC1=C(CCC(=C)Nc2ccccc2C)NC2=C(C(=C)CCC2)C1c1ccccc1C(C)(C)C]

SR-01000652579

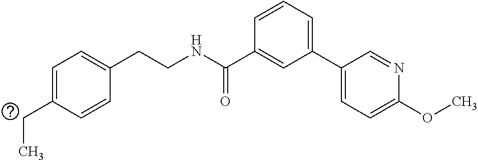 [SMILES: CCc1ccc(CCNC(=O)c2cccc(-c3ccc(OC)nc3)c2)cc1]

SR-01000065653

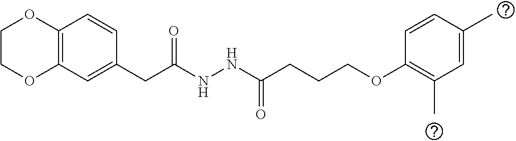 [SMILES: Cc1ccc(OCCCC(=O)NNC(=O)Cc2ccc3c(c2)OCCO3)c(C)c1]

SR-01000364205

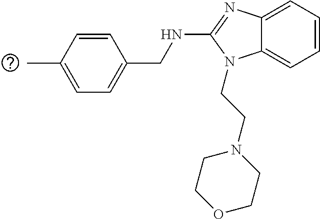 [SMILES: Cc1ccc(CNc2nc3ccccc3n2CCN2CCOCC2)cc1]

SR-01000139982

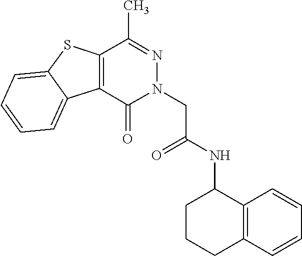 [SMILES: Cc1nn(CC(=O)NC2CCCc3ccccc32)c(=O)c2c1sc1ccccc12]

SR-01000044433

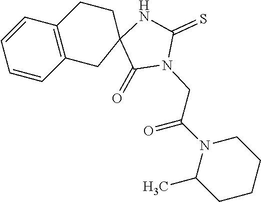 [SMILES: CC1CCCCN1C(=O)CN1C(=O)C2(CCc3ccccc3C2)NC1=S]

SR-01000054340

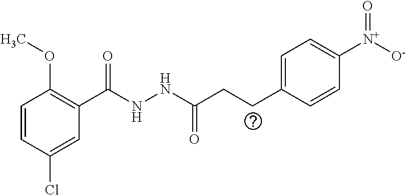 [SMILES: COc1ccc(Cl)cc1C(=O)NNC(=O)CCc1ccc([N+](=O)[O-])cc1]

SR-01000269911

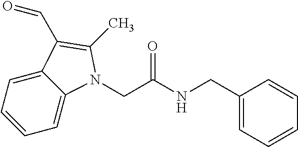 [SMILES: Cc1c(C=O)c2ccccc2n1CC(=O)NCc1ccccc1]

SR-01000004378

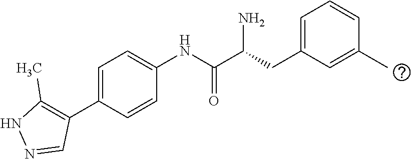 [SMILES: Cc1cccc(C[C@@H](N)C(=O)Nc2ccc(-c3cn[nH]c3C)cc2)c1]

SR-01000126715

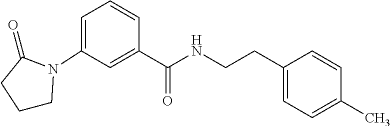 [SMILES: Cc1ccc(CCNC(=O)c2cccc(N3CCCC3=O)c2)cc1]

SR-01000048373

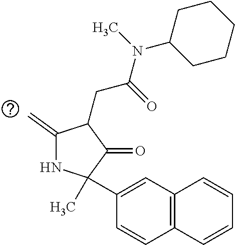 [SMILES: C=C1NC(C)(c2ccc3ccccc3c2)C(=O)C1CC(=O)N(C)C1CCCCC1]

SR-01000146444

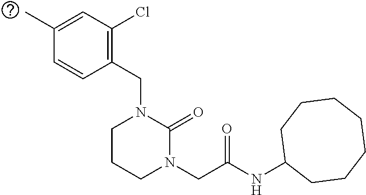 [SMILES: Cc1ccc(CN2CCCN(CC(=O)NC3CCCCCCC3)C2=O)c(Cl)c1]

SR-01000067982

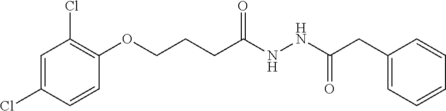 [SMILES: O=C(CCCOc1ccc(Cl)cc1Cl)NNC(=O)Cc1ccccc1]

SR-01000068916

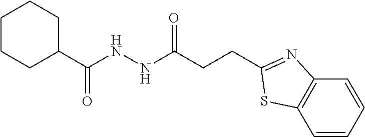 [SMILES: O=C(CCc1nc2ccccc2s1)NNC(=O)C1CCCCC1]

SR-01000410694

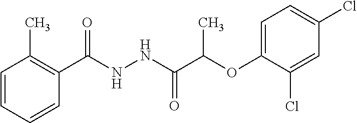 [SMILES: Cc1ccccc1C(=O)NNC(=O)C(C)Oc1ccc(Cl)cc1Cl]

SR-01000921162

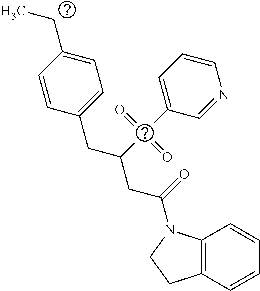

SR-01000151470

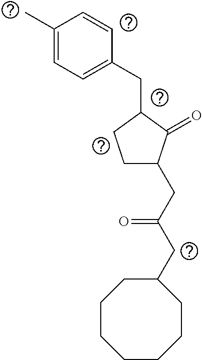 [SMILES: Cc1ccc(CC2CCC(CC(=O)CC3CCCCCCC3)C2=O)cc1]

SR-01000003277

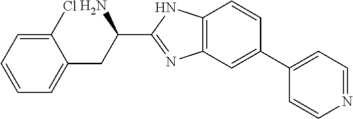 [SMILES: N[C@H](Cc1ccccc1Cl)c1nc2cc(-c3ccncc3)ccc2[nH]1]

SR-01000320687

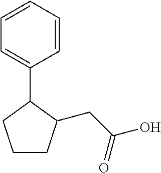 [SMILES: O=C(O)CC1CCCC1c1ccccc1]

SR-01000925700

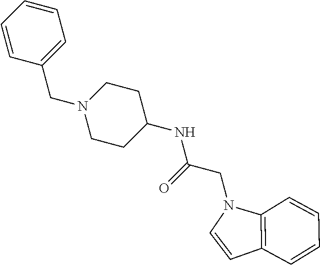 [SMILES: O=C(Cn1ccc2ccccc21)NC1CCN(Cc2ccccc2)CC1]

SR-01000526835

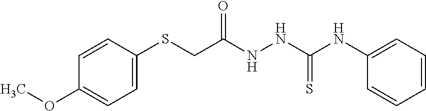 [SMILES: COc1ccc(SCC(=O)NNC(=S)Nc2ccccc2)cc1]

SR-01000318010

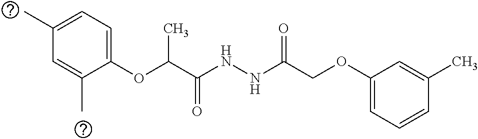

SR-01000007008

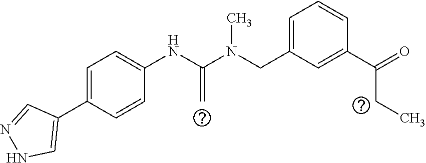 [SMILES: C=C(Nc1ccc(-c2cn[nH]c2)cc1)N(C)Cc1cccc(C(=O)CC)c1]

SR-01000074761

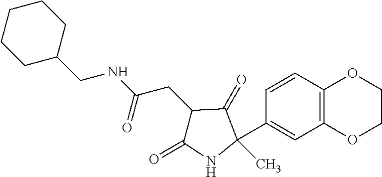 [SMILES: CC1(c2ccc3c(c2)OCCO3)NC(=O)C(CC(=O)NCC2CCCCC2)C1=O]

SR-01000021418

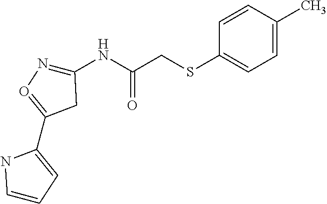

SR-01000309617

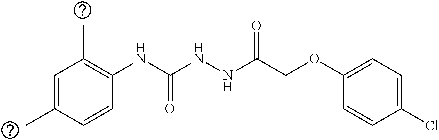 [SMILES: Cc1ccc(NC(=O)NNC(=O)COc2ccc(Cl)cc2)c(C)c1]

SR-01000582959

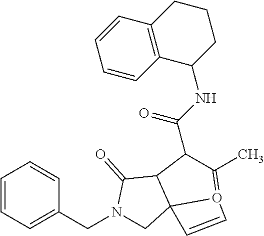

SR-01000358286

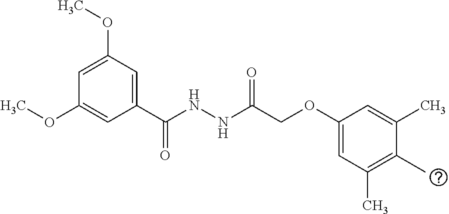

SR-01000054961

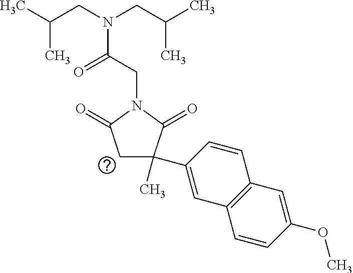 [SMILES: COc1ccc2cc(C3(C)CC(=O)N(CC(=O)N(CC(C)C)CC(C)C)C3=O)ccc2c1]

SR-01000126429

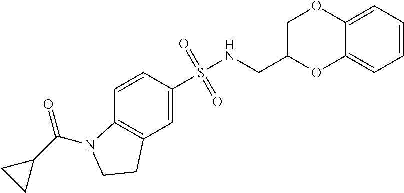 [SMILES: O=C(C1CC1)N1CCc2cc(S(=O)(=O)NCC3COc4ccccc4O3)ccc21]

SR-01000124163

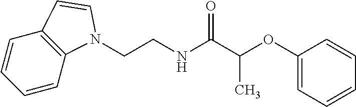 [SMILES: CC(Oc1ccccc1)C(=O)NCCn1ccc2ccccc21]

SR-01000072797

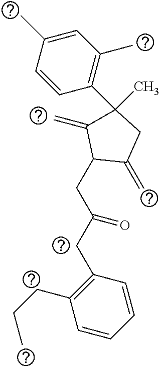 [SMILES: C=C1CC(C)(c2ccc(C)cc2C)C(=C)C1CC(=O)Cc1ccccc1CCC]

SR-01000068179

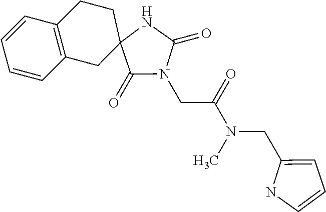 [SMILES: CN(Cc1ccc[nH]1)C(=O)CN1C(=O)NC2(CCc3ccccc3C2)C1=O]

SR-01000393531

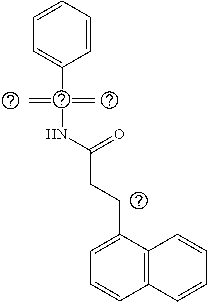

SR-01000142419

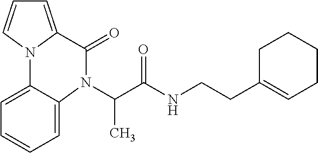 [SMILES: CC(C(=O)NCCC1=CCCCC1)n1c(=O)c2cccn2c2ccccc21]

SR-01000069699

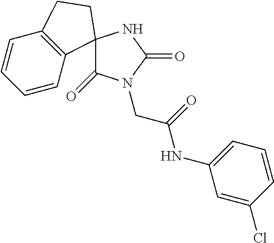 [SMILES: O=C(CN1C(=O)NC2(CCc3ccccc32)C1=O)Nc1cccc(Cl)c1]

SR-01000654192

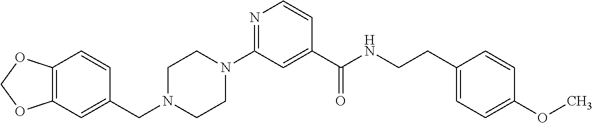 [SMILES: COc1ccc(CCNC(=O)c2ccnc(N3CCN(Cc4ccc5c(c4)OCO5)CC3)c2)cc1]

SR-01000150393

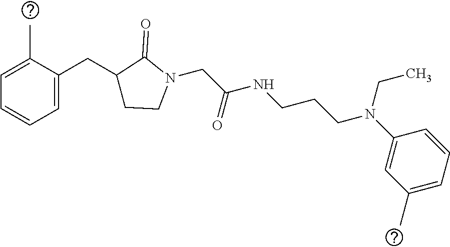 [SMILES: CCN(CCCNC(=O)CN1CCC(Cc2ccccc2C)C1=O)c1cccc(C)c1]

SR-01000370374

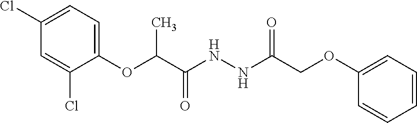 [SMILES: CC(Oc1ccc(Cl)cc1Cl)C(=O)NNC(=O)COc1ccccc1]

SR-01000153956

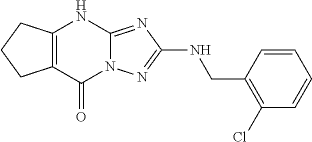 [SMILES: O=c1c2c([nH]c3nc(NCc4ccccc4Cl)nn13)CCC2]

SR-01000318521

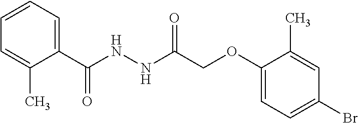 [SMILES: Cc1cc(Br)ccc1OCC(=O)NNC(=O)c1ccccc1C]

SR-01000312779

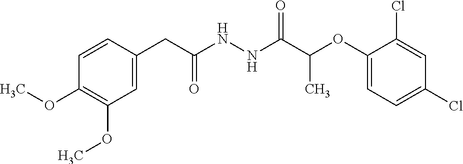 [SMILES: COc1ccc(CC(=O)NNC(=O)C(C)Oc2ccc(Cl)cc2Cl)cc1OC]

SR-01000923250

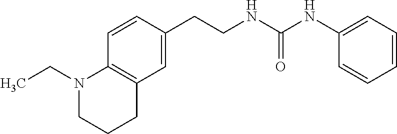 [SMILES: CCN1CCCc2cc(CCNC(=O)Nc3ccccc3)ccc21]

SR-01000153886

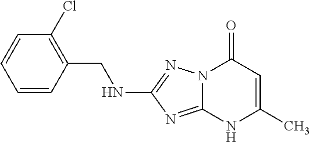 [SMILES: Cc1cc(=O)n2nc(NCc3ccccc3Cl)nc2[nH]1]

SR-01000299610

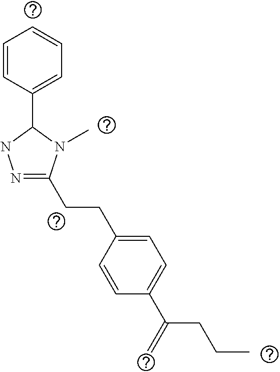 [SMILES: C=C(CCC)c1ccc(CCC2=NNC(c3ccccc3)N2C)cc1]

SR-01000145393

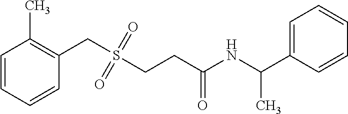 [SMILES: Cc1ccccc1CS(=O)(=O)CCC(=O)NC(C)c1ccccc1]

SR-01000273754

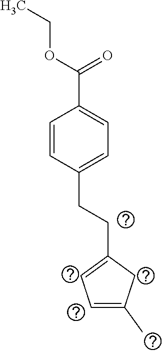 [SMILES: CCOC(=O)c1ccc(CCC2=CC=C(C)C2)cc1]

SR-01000021414

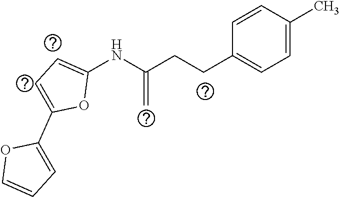 [SMILES: C=C(CCc1ccc(C)cc1)Nc1ccc(-c2ccco2)o1]

SR-01000007518

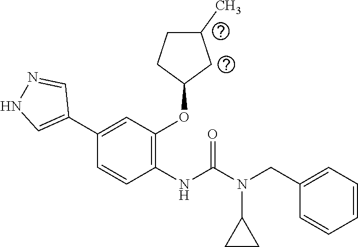 [SMILES: CC1CC[C@H](Oc2cc(-c3cn[nH]c3)ccc2NC(=O)N(Cc2ccccc2)C2CC2)C1]

SR-01000189442

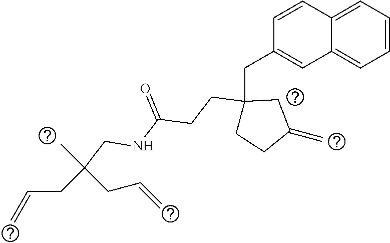 [SMILES: C=CCC(C)(CC=C)CNC(=O)CCC1(Cc2ccc3ccccc3c2)CCC(=C)C1]

SR-01000098976

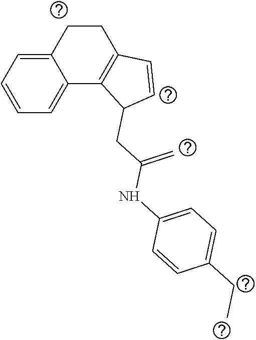 [SMILES: C=C(CC1C=CC2=C1c1ccccc1CC2)Nc1ccc(CC)cc1]

SR-01000335115

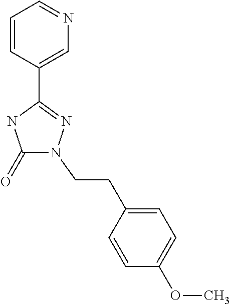 [SMILES: COc1ccc(CCn2nc(-c3cccnc3)[nH]c2=O)cc1]

SR-01000145456

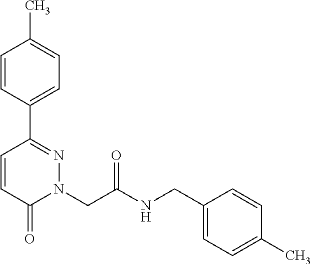 [SMILES: Cc1ccc(CNC(=O)Cn2nc(-c3ccc(C)cc3)ccc2=O)cc1]

SR-01000185621

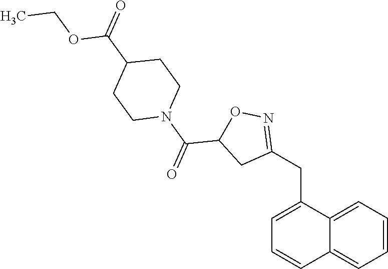 [SMILES: CCOC(=O)C1CCN(C(=O)C2CC(Cc3cccc4ccccc34)=NO2)CC1]

SR-01000167161

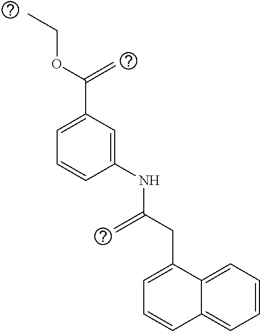 [SMILES: C=C(Cc1cccc2ccccc12)Nc1cccc(C(=C)OCC)c1]

SR-01000025120

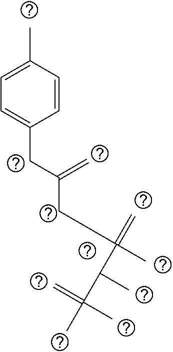

SR-01000007044

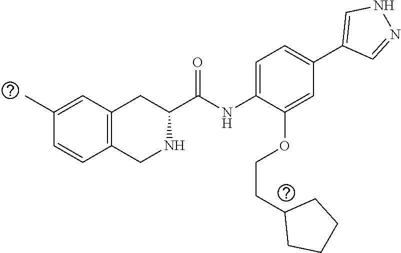 [SMILES: Cc1ccc2c(c1)C[C@H](C(=O)Nc1ccc(-c3cn[nH]c3)cc1OCCC1CCCC1)NC2]

SR-01000185712

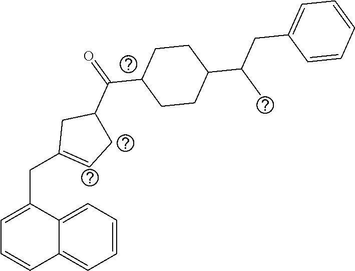 [SMILES: CC(Cc1ccccc1)C1CCC(C(=O)C2CC=C(Cc3cccc4ccccc34)C2)CC1]

SR-01000124129

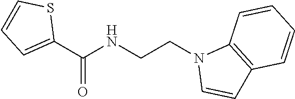 [SMILES: O=C(NCCn1ccc2ccccc21)c1cccs1]

SR-01000004118

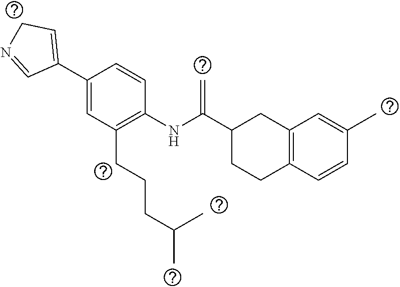 [SMILES: C=C(Nc1ccc(C2=CCN=C2)cc1CCCC(C)C)C1CCc2ccc(C)cc2C1]

SR-01000068608

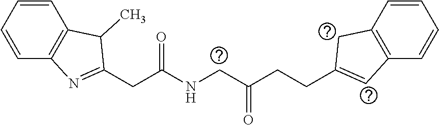 [SMILES: CC1C(CC(=O)NCC(=O)CCC2=Cc3ccccc3C2)=Nc2ccccc21]

SR-01000926263

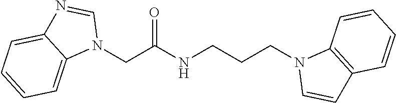 [SMILES: O=C(Cn1cnc2ccccc21)NCCCn1ccc2ccccc21]

SR-01000152429

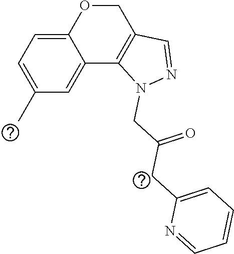 [SMILES: Cc1ccc2c(c1)-c1c(cnn1CC(=O)Cc1ccccn1)CO2]

SR-01000193756

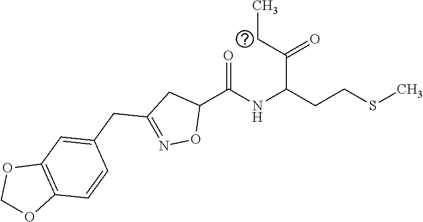 [SMILES: CCC(=O)C(CCSC)NC(=O)C1CC(Cc2ccc3c(c2)OCO3)=NO1]

SR-01000406098

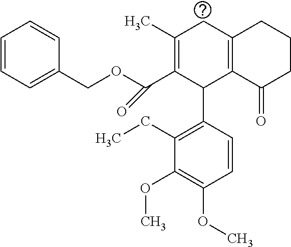 [SMILES: CCc1c(C2C(C(=O)OCc3ccccc3)=C(C)CC3=C2C(=O)CCC3)ccc(OC)c1OC]

SR-01000357697

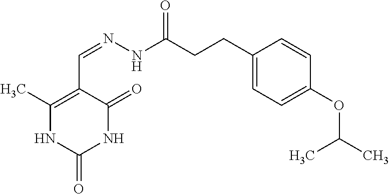 [SMILES: Cc1[nH]c(=O)[nH]c(=O)c1/C=N\NC(=O)CCc1ccc(OC(C)C)cc1]

SR-01000579592

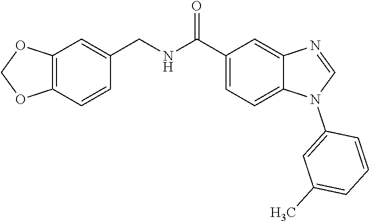 [SMILES: Cc1cccc(-n2cnc3cc(C(=O)NCc4ccc5c(c4)OCO5)ccc32)c1]

SR-01000028953

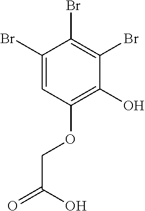 [SMILES: O=C(O)COc1cc(Br)c(Br)c(Br)c1O]

SR-01000336681

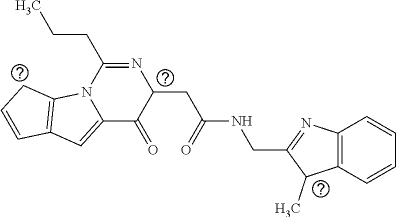 [SMILES: CCCC1=NC(CC(=O)NCC2=Nc3ccccc3C2C)C(=O)c2cc3c(n21)CC=C3]

SR-01000002785

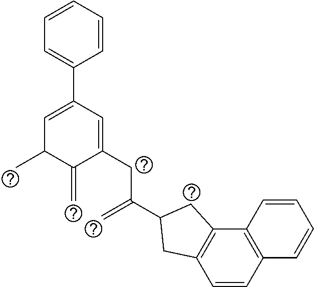 [SMILES: C=C1C(CC(=C)C2Cc3ccc4ccccc4c3C2)=CC(c2ccccc2)=CC1C]

SR-01000197834

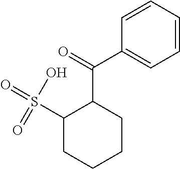 [SMILES: O=C(c1ccccc1)C1CCCCC1S(=O)(=O)O]

SR-01000286062

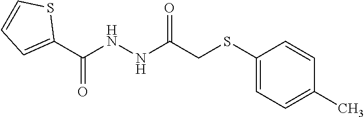 [SMILES: Cc1ccc(SCC(=O)NNC(=O)c2cccs2)cc1]

SR-01000655128

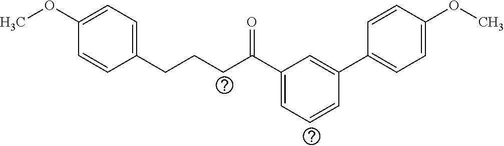

SR-01000151876

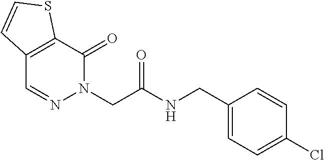 [SMILES: O=C(Cn1ncc2ccsc2c1=O)NCc1ccc(Cl)cc1]

SR-01000004451

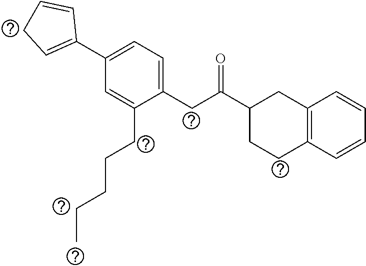 [SMILES: CCCCCc1cc(C2=CCC=C2)ccc1CC(=O)C1CCc2ccccc2C1]

SR-01000143463

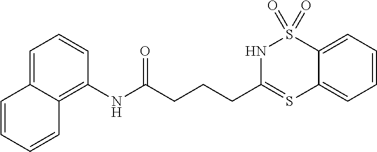

SR-01000363853

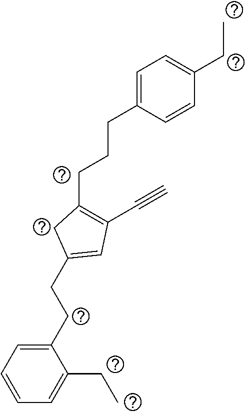 [SMILES: C#CC1=C(CCCc2ccc(CC)cc2)CC(CCc2ccccc2CC)=C1]

SR-01000362228

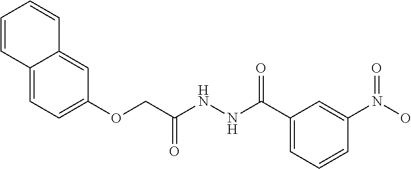

SR-01000062305

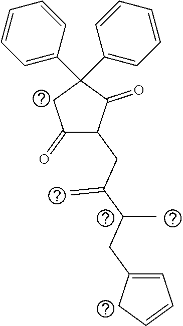 [SMILES: C=C(CC1C(=O)CC(c2ccccc2)(c2ccccc2)C1=O)C(C)CC1=CC=CC1]

SR-01000634249

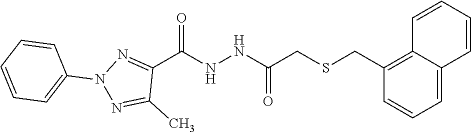 [SMILES: Cc1nn(-c2ccccc2)nc1C(=O)NNC(=O)CSCc1cccc2ccccc12]

SR-01000248261

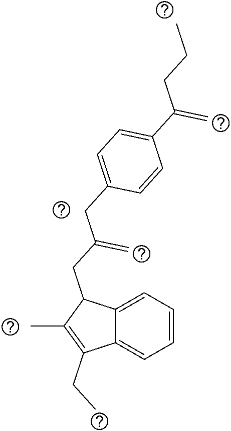 [SMILES: C=C(Cc1ccc(C(=C)CCC)cc1)CC1C(C)=C(CC)c2ccccc21]

SR-01000034566

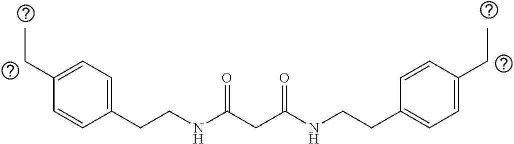 [SMILES: CCc1ccc(CCNC(=O)CC(=O)NCCc2ccc(CC)cc2)cc1]

SR-01000129845

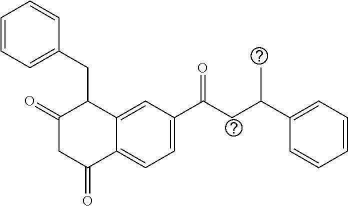 [SMILES: CC(CC(=O)c1ccc2c(c1)C(Cc1ccccc1)C(=O)CC2=O)c1ccccc1]

SR-01000012264

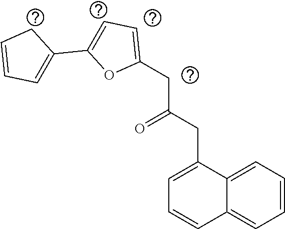 [SMILES: O=C(Cc1ccc(C2=CC=CC2)o1)Cc1cccc2ccccc12]

SR-01000316766

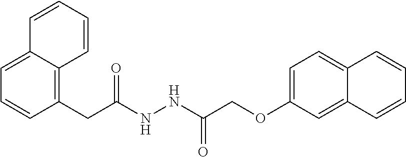 [SMILES: O=C(COc1ccc2ccccc2c1)NNC(=O)Cc1cccc2ccccc12]

SR-01000145157

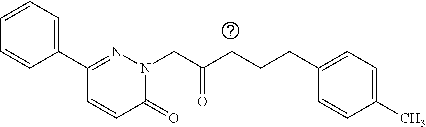 [SMILES: Cc1ccc(CCCC(=O)Cn2nc(-c3ccccc3)ccc2=O)cc1]

SR-01000125460

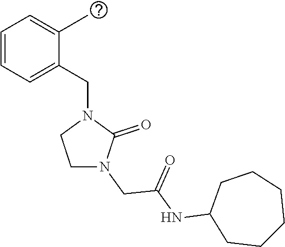 [SMILES: Cc1ccccc1CN1CCN(CC(=O)NC2CCCCCC2)C1=O]

SR-01000550322

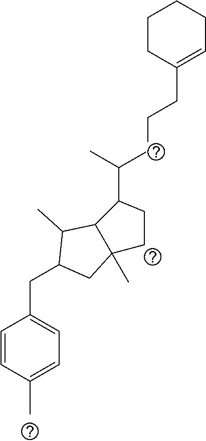 [SMILES: Cc1ccc(CC2CC3(C)CCC(C(C)CCCC4=CCCCC4)C3C2C)cc1]

SR-01000098884

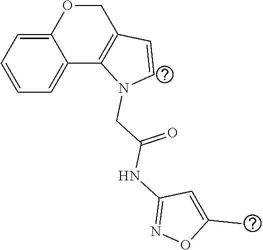 [SMILES: Cc1cc(NC(=O)Cn2ccc3c2-c2ccccc2OC3)no1]

SR-01000022582

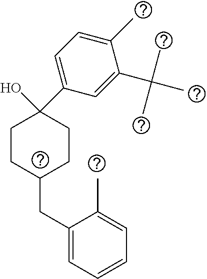 [SMILES: Cc1ccccc1CC1CCC(O)(c2ccc(C)c(C(C)(C)C)c2)CC1]

SR-01000484494

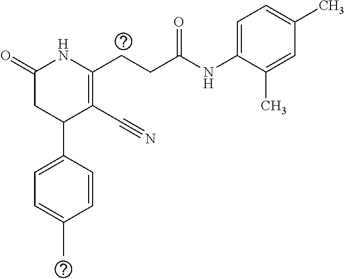 [SMILES: Cc1ccc(C2CC(=O)NC(CCC(=O)Nc3ccc(C)cc3C)=C2C#N)cc1]

SR-01000005410

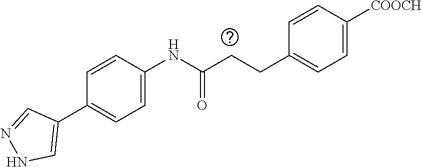

SR-01000124392

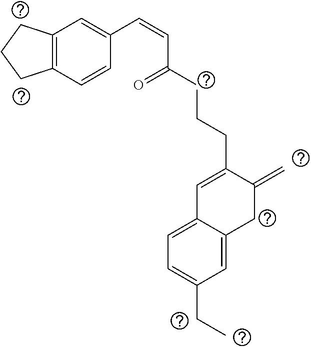 [SMILES: C=C1Cc2cc(CC)ccc2C=C1CCCC(=O)/C=C\c1ccc2c(c1)CCC2]

SR-01000091502

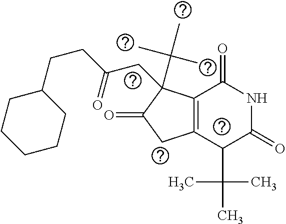 [SMILES: CC(C)(C)C1C(=O)NC(=O)C2=C1CC(=O)C2(CC(=O)CCC1CCCCC1)C(C)(C)C]

SR-01000075116

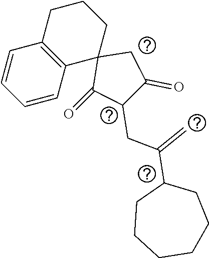 [SMILES: C=C(CC1C(=O)CC2(CCCc3ccccc32)C1=O)C1CCCCCC1]

SR-01000918719

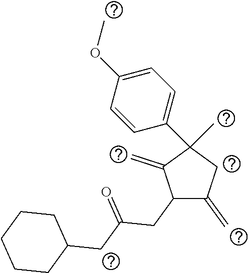 [SMILES: C=C1CC(C)(c2ccc(OC)cc2)C(=C)C1CC(=O)CC1CCCCC1]

SR-01000125464

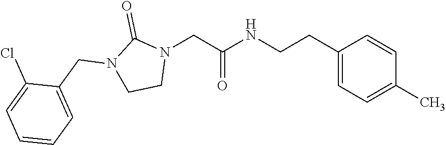 [SMILES: Cc1ccc(CCNC(=O)CN2CCN(Cc3ccccc3Cl)C2=O)cc1]

SR-01000101329

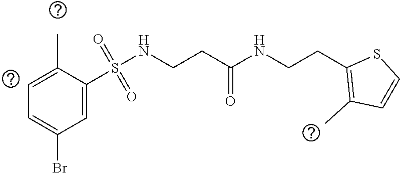 [SMILES: Cc1ccc(Br)cc1S(=O)(=O)NCCC(=O)NCCc1sccc1C]

SR-01000319951

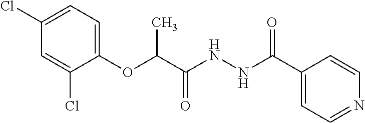 [SMILES: CC(Oc1ccc(Cl)cc1Cl)C(=O)NNC(=O)c1ccncc1]

SR-01000126251

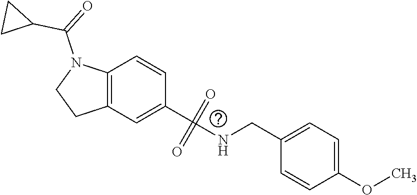

SR-01000361172

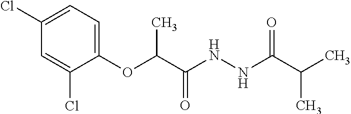 [SMILES: CC(C)C(=O)NNC(=O)C(C)Oc1ccc(Cl)cc1Cl]

SR-01000011241

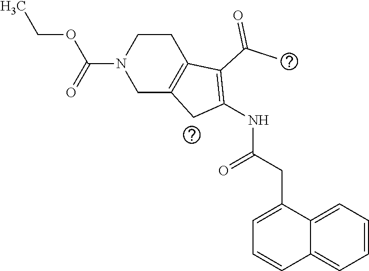 [SMILES: CCOC(=O)N1CCC2=C(CC(NC(=O)Cc3cccc4ccccc34)=C2C(C)=O)C1]

SR-01000325257

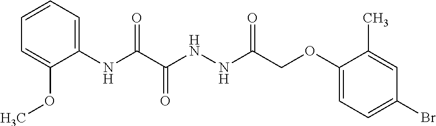 [SMILES: COc1ccccc1NC(=O)C(=O)NNC(=O)COc1ccc(Br)cc1C]

SR-01000911742

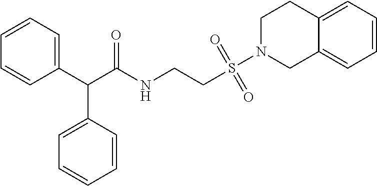 [SMILES: O=C(NCCS(=O)(=O)N1CCc2ccccc2C1)C(c1ccccc1)c1ccccc1]

SR-01000429212

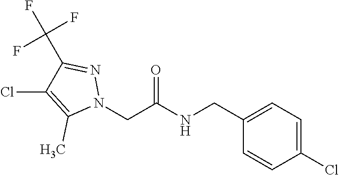 [SMILES: Cc1c(Cl)c(C(F)(F)F)nn1CC(=O)NCc1ccc(Cl)cc1]

SR-01000545824

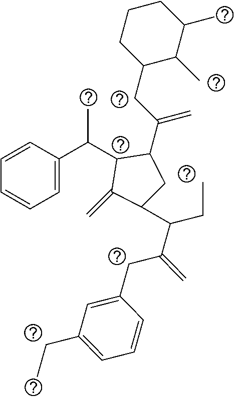 [SMILES: C=C(Cc1cccc(CC)c1)C(CC)C1CC(C(=C)CC2CCCC(C)C2C)C(C(C)c2ccccc2)C1=C]

SR-01000601649

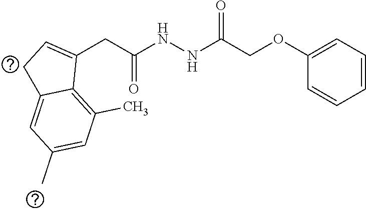 [SMILES: Cc1cc(C)c2c(c1)CC=C2CC(=O)NNC(=O)COc1ccccc1]

SR-01000074154

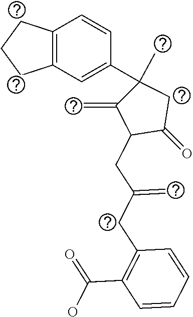 [SMILES: C=C(Cc1ccccc1C(=O)O)CC1C(=C)C(C)(c2ccc3c(c2)CCC3)CC1=O]

SR-01000070509

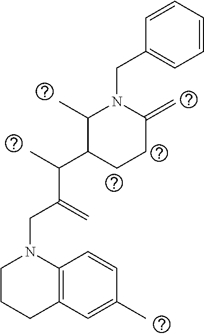 [SMILES: C=C(CN1CCCc2cc(C)ccc21)C(C)C1CCC(=C)N(Cc2ccccc2)C1C]

SR-01000134947

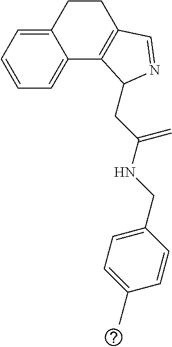 [SMILES: C=C(CC1N=CC2=C1c1ccccc1CC2)NCc1ccc(C)cc1]

SR-01000088420

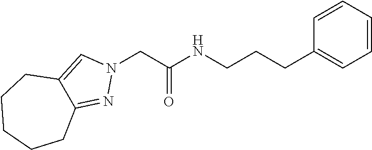 [SMILES: O=C(Cn1cc2c(n1)CCCCC2)NCCCc1ccccc1]

SR-01000136273

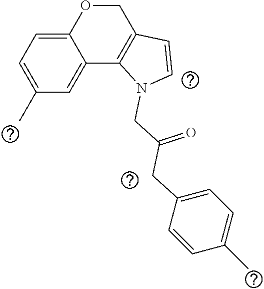 [SMILES: Cc1ccc(CC(=O)Cn2ccc3c2-c2cc(C)ccc2OC3)cc1]

SR-01000921037

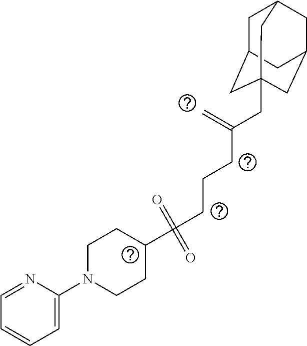

SR-01000119565

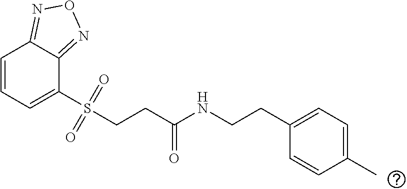 [SMILES: Cc1ccc(CCNC(=O)CCS(=O)(=O)c2cccc3nonc23)cc1]

SR-01000017533

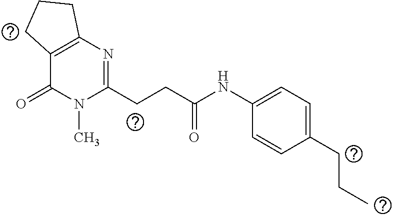 [SMILES: CCCc1ccc(NC(=O)CCc2nc3c(c(=O)n2C)CCC3)cc1]

SR-01000299370

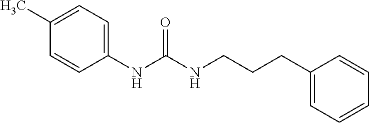 [SMILES: Cc1ccc(NC(=O)NCCCc2ccccc2)cc1]

SR-01000465824

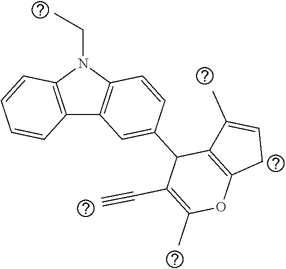 [SMILES: C#CC1=C(C)OC2=C(C(C)=CC2)C1c1ccc2c(c1)c1ccccc1n2CC]

SR-01000914970

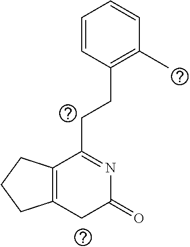 [SMILES: Cc1ccccc1CCC1=NC(=O)CC2=C1CCC2]

SR-01000125462

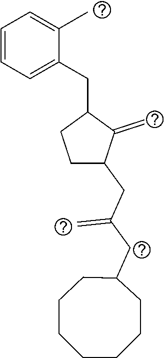 [SMILES: C=C(CC1CCCCCCC1)CC1CCC(Cc2ccccc2C)C1=C]

SR-01000100493

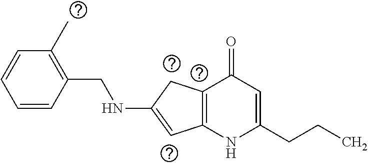 [SMILES: CCCc1cc(=O)c2c([nH]1)C=C(NCc1ccccc1C)C2]

SR-01000190122

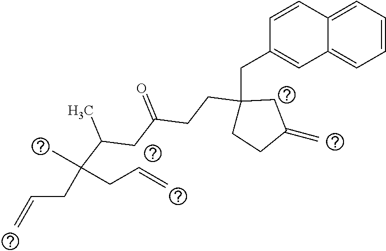 [SMILES: C=CCC(C)(CC=C)C(C)CC(=O)CCC1(Cc2ccc3ccccc3c2)CCC(=C)C1]

SR-01000360839

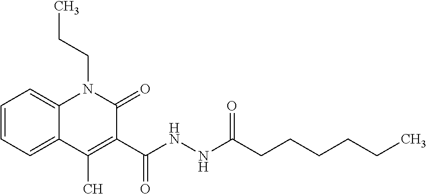 [SMILES: CCCCCCC(=O)NNC(=O)c1c(C)c2ccccc2n(CCC)c1=O]

SR-010005997171

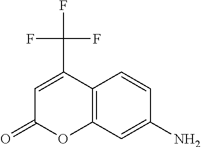 [SMILES: Nc1ccc2c(C(F)(F)F)cc(=O)oc2c1]

SR-01000141798

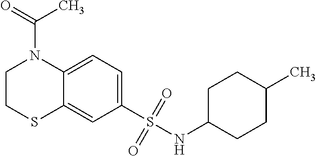 [SMILES: CC(=O)N1CCSc2cc(S(=O)(=O)NC3CCC(C)CC3)ccc21]

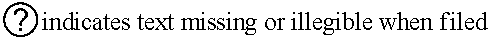

GPR139 has been shown to play a role in neuropsychiatric behavior (Dao et al. “The role of orphan receptor GPR139 in neuropsychiatric behavior” Neuropsychopharmacology 2021 (published online 21 Jan. 2021). Dao et al. found that mice lacking GPR139 were found to exhibit delayed onset hyperactivity and prominent neuropsychiatric manifestations including elevated stereotypy, increased anxiety-related traits, delayed acquisition of operant responsiveness, disruption of cued fear conditioning, and social interaction deficits. Mice lacking GPR139 also exhibited loss of pre-pulse inhibition and developed spontaneous ‘hallucinogenic’ head-twitches. The behavioral deficits were rescued by the administration of a μ-opioid antagonist (naltrexone) and a D2 dopamine receptor (D2R) antagonist (haloperidol), suggesting that loss of neuropsychiatric manifestations in mice lacking GPR139 are driven by opioidergic and dopaminergic hyper-functionality. These observations define the role of GPR139 is thought to play a role in controlling behavior.

In some embodiments, the GPR139 agonists can be used to treat neuropsychiatric disorders or conditions or psychoses. Described are methods of treating neuropsychiatric disorders or conditions or psychoses in a subject comprising administering to the subject an effective amount of one or more GPR139 agonists of Table 2. Also described are methods of treating one or more symptoms associated with a neuropsychiatric disorder or condition or psychosis in a subject comprising administering to the subject an effective amount of one or more GPR139 agonists of Table 2. Treating one or more symptoms associated with a neuropsychiatric disorder or condition or psychosis includes, but is not limited to, decreasing or suppressing the one or more symptoms, decreasing the frequency of the one or more symptoms, decreasing the severity of the one or more symptoms, or decreasing progression of the one or more symptoms. Symptoms associated with neuropsychiatric disorder, such as schizophrenia include, but are not limited to, delusions, hallucinations, confused or disorganized thinking, trouble with logical thinking, confused or disordered speech, abnormal movements, paranoia, inability to express emotion, inability to find pleasure, and exaggerated or distorted perceptions, beliefs, and behaviors. Treating the neuropsychiatric disorders or conditions or psychoses can also include improving the quality of life of the person suffering from the neuropsychiatric disorder or condition or psychosis or decreasing the dosage of one or more other therapeutics taken by the subject to treat the neuropsychiatric disorder or condition or psychosis. The neuropsychiatric disorder or condition or psychosis can be, but is not limited to, schizophrenia, a schizophrenia-related disorder, a schizotypal personality disorder, an obsessive-compulsive disorder, Huntington's disease, a deficit in social interactions, a prepulse inhibition disorder, attention deficit hyperactivity disorder (ADHD), Parkinson's, dementia, mental retardation, an autism spectrum disorder, and spontaneous or involuntary head twitching or other spontaneous or involuntary twitches. The GPR139 agonist can be administered to a subject to, for example, suppress hyperactivity, promote movement initiation, correct or improve sensorimotor integration, reduce hallucination, enhance cognitive activity, increase sociability, and/or increase social interactions. The GPR139 agonists can be used as stand alone antipsychotics or they may be combined with one or more additional antipsychotics. The subject can be, but is not limited to, a human.

In some embodiments, a described GP139 agonist or a pharmaceutical composition containing the GP139 agonist is administered with one or more of an anti-psychotic therapeutic, a mood stabilizer, and an antidepressant. In some embodiments, a pharmaceutical composition containing a GP139 agonist further comprises with an additional anti-psychotic therapeutic, a mood stabilizer, and/or an antidepressant. The antipsychotic can be, but is not limited to, chlorpromazine, fluphenazine, haloperidol, perphenazine, thioridazine, thiothixene, trifluoperazine, aripiprazole, aripiprazole lauroxil, asenapine, brexpiprazole, cariprazine, clozapine, iloperidone, lumateperonee, lurasidone, olanzapine, olanzapine/samidorphan, paliperidone, paliperidone palmitate, quetiapine, risperidone, and ziprasidone.

The compounds and pharmaceutical compositions disclosed herein can be administered to a subject once per day, more than once a day, for example, 2, 3, 4, 5, or 6 times a day, or as needed.

In some embodiments, the GPR139 modulators (GPR139 antagonists and GPR139 agonists) are formulated with one or more pharmaceutically acceptable excipients (including vehicles, carriers, diluents, and/or delivery polymers), thereby forming a pharmaceutical composition or medicament suitable for in vivo delivery to a subject, such as a human.

A pharmaceutical composition or medicament includes a pharmacologically effective amount of the active compound and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support, or enhance stability, bioavailability or patient acceptability of the API, c) assist in product identification, and/or d) enhance any other attribute of the overall safety and effectiveness of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

A carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants, such as preservatives, wetting agents, emulsifying agents, and dispersing agents. A carrier may also contain isotonic agents, such as sugars, polyalcohols, sodium chloride, and the like into the compositions.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

The phrase “pharmaceutically acceptable” indicates that the substance or composition must be compatible chemically and/or toxicologically, with the other ingredients comprising a formulation, and/or the mammal being treated therewith. Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals, and more particularly in humans.

In some embodiments, the pharmaceutical compositions further comprise one or more additional active ingredients. The additional active pharmaceutical ingredients can be, but are not limited to, an opiate, an analgesic, an NSAID, acetaminophen, an antipsychotic, a mood stabilizer, and an antidepressant.

A pharmaceutical composition containing a GPR139 modulating agent (e.g., an antagonist or agonist compound) and other therapeutic agents described herein (e.g., an opioid drug) can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations or compositions to administer such compositions to subjects. Any appropriate route of administration may be employed, for example, but not limited to, oral administration, intravenous, parenteral, transcutaneous, subcutaneous, and intramuscular administration.

Any of the described GPR modulators or pharmaceutical compositions identified herein can be formulated as a liquid formulation, as a solid formulation (including a powder or lyophilized formulation), or for aerosol administration. The compounds and pharmaceutical compositions can be formulated as a capsule or tablet, a time-release capsule or tablet, a powder, granules, a solution, a suspension in an aqueous liquid or non-aqueous liquid, an oil-in-water emulsion, or as a water-in-oil liquid emulsion. The compounds and pharmaceutical compositions can be formulated for oral administration, aerosol or inhalation administration, nasal administration, injection, infusion, topical administration, rectal administration, transmucosal administration, transdermal administration, intravenous administration, intradermal administration, subcutaneous administration, intramuscular administration, or intraperitoneal administration. In some embodiments, the pharmaceutical composition is administered parenterally.

Any of the compounds or pharmaceutical compositions identified herein can be formulated or packaged in single-dose or multi-dose format. In some embodiments, any of the compounds or pharmaceutical compositions identified herein can be formulated for repeat dosing.

The GPR139 modulating agent (GPR139 antagonist or GPR139 agonist) for use in the described methods is administered to a subject in an amount that is sufficient to achieve the desired therapeutic effect in the subject. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject.

The selected dosage level depends upon a variety of pharmacokinetic factors, including the activity of the particular GPR139 modulating compound employed, or the ester, salt or amide thereof, the route of administration, the time of administration, or the rate of excretion of the particular compound being employed. Dosage can also depend on the duration of the treatment, or other drugs, compounds, and/or materials used in combination with the employed GPR139 modulating compound. Age, gender, weight, condition, general health, and prior medical history of the subject being treated can also affect dosage. Methods for determining optimal dosages are described in the art, e.g., Remington: The Science and Practice of Pharmacy, Mack Publishing Co., 20th ed., 2000.

In some embodiments, kits containing one or more of the described GPR139 modulators or a pharmaceutical composition containing one or more GPR139 modulators are described. In some embodiments, the kits comprise GPR139 modulators or a pharmaceutical composition containing one or more GPR139 modulators further comprise instructions for use. Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

In some embodiments, a kit further comprises two or more components, including at least one active pharmaceutical ingredient and one or more inactive ingredients, excipient, diluents, and the like, and optionally instructions for preparation of the dosage form by the patient or person administering the drug to the patient. In some embodiments, a kit may further comprise optional components that aid in the administration of the unit dose to a subject, including but not limited to: vials for reconstituting powder forms, syringes for injection, customized IV delivery systems, inhalers, etc. Additionally, a kit can contain instructions for preparation and administration of the compositions. The kit can be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

In some embodiments, a kit further includes an additional therapeutic agent. The additional therapeutic agent can be, but is not limited to, an opioid or an antipsychotic therapeutic.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1. Identification of GPR139 Agonists and Antagonists

Agonists and antagonists of GPR139 were identified using HEK Aequorin cells transfected with GPR139 protein (HEK-GPR139 cells) using the Maxcyte transient transfection system and high-throughput screening (HTS). Cheminformatics was used to identify compounds that demonstrated authentic agonist or antagonist pharmacology and non-promiscuous activity profiles across other primary screens run against the SDDL. 666120 compounds were analyzed for agonist and antagonist activity at completion of the project. An initial screen was performed using 5.63 μM compound. Positive from the first screen were tested in a second round comparing activity in HEK-GPR139 cells with activity in parental HEK cells. Titration assays were then performed on positives from the second round. 476 compounds demonstrated selectivity with nominal potency (EC₅₀<1 μM) in the GPR139 Agonist assay and, 81 compounds demonstrated nominal potency (IC₅₀<5 μM) in the GPR139 Antagonist assay. None of these were active in the HEK parental counterscreens. All compounds selected for titration were also subjected to LC-MS analysis to confirm mass and sample purity.

GPR activity was assessed using a calcium detection assay. GPR139 agonists stimulate an increase in intracellular calcium in HEK-GPR139 cells. Agonists increase the release of intracellular calcium, while antagonists decrease the release of intracellular calcium, as compared to an EC80 stimulation of the know agonist S-JNJ-63533054. The positive control for the agonist mode was cells treated with EC100 of known agonist S-JNJ-63533054. Negative control samples were treated with DMSO. The antagonist high control in cells was EC80 of S-JNJ-63533054+IC100 S-JNJ-3792165, while cells were treated with EC80 of S-JNJ-63533054+DMSO for the low control. Intracellular calcium was measured using Calcium 5.

HEK cells were transfected the DNA encoding GPR139 and incubated for 24 hours. 3000 cells/well in 3 μL HEPES and 1% DMSO were seeded into 1536-well plates. 3 μL Calcium 5 was then added to each well and cells were incubated for 60 min. at 37° C. in 5% CO₂ and 95% relative humidity. Cells were pretreated with 50 nL DMSO (5 min. at room temperature in the dark) to stabilize response and desensitize cells to DMSO. A 5 second basal read was recorded and compounds in 30 nL were added to the cells, followed by a 150 second read. Readings were taken at 470 nm/535 nM.

The first step of the HTS was primary screening library containing 666,120 compounds. In this primary screen, compounds were tested at a single concentration in triplicate at a final nominal concentration 5.63 μM. Raw assay data was analyzed using Symyx software. Activity of each compound was calculated on a per-plate basis using the following equation:

$$Percent Response of compound = 100 \times ( \frac{Test Well - Median Data Wells}{Median High Control - Median Data Wells} )$$

A. Agonist Mode: The “High Control” represented wells containing cells with GPR139+EC100 agonist. “Low Control” and sample field represented wells containing cells with GPR139 with DMSO or compounds. The Z′ and S:B for this assay were calculated using the High Control and Low Control wells.

An average Z′ of 0.50±0.10 and an average signal-to-background ratio (S:B) of 4.65±0.55 (n=536 plates) was observed. A mathematical algorithm was used to determine active compounds. Three values were calculated:
(1) the average value for data wells which have activity values between avg minus 3 st. dev. of the low control and avg plus 3sd of high control wells;
(2) the st. dev. value for the same set of data wells in expression 1; and
(3) The sum of these two values was used as a cutoff parameter, i.e. any compound that exhibited greater percent activation than the cutoff parameter was declared active.
   Using this “Interval Based Cutoff” criteria of 45.97% the primary assay yielded 20,600 active compounds (“hits”).

B. Antagonist Mode: The “High Control” represented wells containing cells with GPR139+Antagonist IC100+Agonist EC80. “Low Control” represented wells containing cells with GPR139 with EC100 Agonist. “0% control” and sample field represented wells containing cells with GPR139 with EC80 Agonist with DMSO or compounds. The Z′ and S:B for this assay were calculated using the High Control and Low Control wells.

An average Z′ of 0.70±0.06 and an average signal-to-background ratio (S:B) of 28.57±5.37 (n=536 plates) was observed. The average plus 3 st. dev. of all compounds tested was used as the hit cutoff. Using this “Standard Cutoff” criteria of 42.48% the primary assay yielded 10,074 active compounds (“hits”).

The active compounds from both the agonist and antagonist screens were compared using Venn diagrams to compile a complete list to pick for confirmation assays.

For all active compounds, 3167 were antagonists only, 13,693 were agonists only, and 6907 were agonists that triggered an antagonist response.

For active compounds in which PAINS and compounds with promiscuity greater than 5 were removed, 2004 compounds were antagonist only, 10,338 compounds were agonist only, and 4000 compounds were agonists that triggered an antagonist response.

For agonist compounds, 2535 were OX1R agonist only, 4027 were OX2R agonist only, 13802 were GPR139 agonist only, 211 were OX1R and GPR139 agonist, and 325 were OX2R and GPR139 agonist.

For final compound selection the top 6,000 agonists were selected by cluster ranking. The 14,338 primary actives were clustered based on structure and the top compounds from each cluster were selected until 6,000 compounds were picked.

C. Secondary Assays. The GPR139 Agonist confirmation assays employed the same reagents, protocols, and detection systems as the primary assays, but tested each of the 5,998 agonist compounds as single point 5.6 μM concentration in triplicate. The HEK parental counterscreen utilized the same cells but not transfected with the plasmid. The agonist and antagonist modes were run the same way as the primary screen but acetylcholine was used in place of GPR139 agonist as the control compound.

GPR139 Agonist Confirmation Assay: An average Z′ of 0.54±0.03 and a S:B of 6.84±1.52 for N=21 plates (6,089 compounds) was observed.

HEK Agonist Counterscreen Assay: An average Z′ of 0.37±0.14 and a S:B of 4.23±1.10 for N=21 plates (6,089 compounds) was observed. The high control for the agonist counterscreen was Forskolin not acetylcholine.

The GPR139 Antagonist confirmation assays employed the same reagents, protocols, and detection systems as the primary assays, but tested each of the 2,001 antagonist compounds as single point 5.6 uM concentration in triplicate. The HEK parental counterscreen utilized the same cells but not transfected with the plasmid. The antagonist modes assays utilized the cells being plated in the 1536 well plate overnight in growth media prior to dye loading in PDL coated plates and acetylcholine was used in place of GPR139 agonist as the control compound.

GPR139 Antagonist Confirmation Assay: An average Z′ of 0.72±0.05 and a S:B of 35.10±4.26 for N=9 plates (2,001 compounds) was observed.

HEK Antagonist Counterscreen Assay: An average Z′ of 0.62±0.04 and a S:B of 5.25±0.43 for N=9 plates (2,001 compounds) was observed. The high control for the antagonist counterscreen was acetylcholine.

1292 compounds were identified and selective for GPR139.

D. Titration Assays. The GPR139 Agonist titration assays employed the same reagents, protocols, and detection systems as the primary assays, but tested each of the 500 agonist compounds as 10 point dose response starting at 14.1 μM concentration in triplicate with 1:3. The HEK parental counterscreen utilized the same cells but not transfected with the plasmid. Acetylcholine was used in place of GPR139 agonist as the control compound. The cells were plated overnight, similar to the HEK antagonist counterscreen protocol.

GPR139 Agonist Titration Assay: An average Z′ of 0.37±0.10 and a S:B of 7.43±2.25 for N=18 plates (500 compounds) was observed.

HEK Agonist Counterscreen Assay: An average Z′ of 0.25±0.13 and a S:B of 2.97±0.29 for N=18 plates (500 compounds) was observed.

The GPR139 Antagonist titration assays employed the same reagents, protocols, and detection systems as the primary assays, but tested each of the 798 agonist compounds as 10 point dose response starting at 14.1 uM concentration in triplicate with 1:3 dilutions. The HEK parental counterscreen utilized the same cells but not transfected with the plasmid. Acetylcholine was used in place of GPR139 agonist as the control compound. The cells were plated overnight, similar to the HEK antagonist counterscreen protocol. The GPR139 Agonist assay was also run against these 798 compounds to determine authentic antagonist selectivity and not agonist activity eliciting the antagonist response. Any compound that had agonist activity was not considered to be a GPR139 antagonist.

GPR139 Antagonist Titration Assay: An average Z′ of 0.71±0.06 and a S:B of 37.62±5.71 for N=27 plates (798 compounds) was observed.

GPR139 Agonist Counterscreen Assay: An average Z′ of 0.35±0.18 and a S:B of 7.48±2.33 for N=27 plates (798 compounds) was observed.

HEK Antagonist Counterscreen Assay: An average Z′ of 0.34±0.08 and a S:B of 2.11±0.08 for N=27 plates (798 compounds) was observed.

The GPR139 Agonist and Antagonist titration assays employed the same reagents, protocols, and detection systems as the primary assays, but tested each as 10 point dose response starting at 676 nM concentration in triplicate with 1:3 dilutions. The HEK parental counterscreens utilized the same cells but not transfected with the plasmid. Acetylcholine was used in place of GPR139 agonist as the control compound. The cells were plated overnight, similar to the HEK antagonist counterscreen protocol.

GPR139 Agonist Titration Assay: An average Z′ of 0.43±0.02 and a S:B of 7.88±3.32 for N=3 plates was observed.

GPR139 Antagonist Titration Assay: An average Z′ of 0.72±0.03 and a S:B of 38.89±11.81 for N=3 plates was observed.

HEK Agonist Counterscreen Assay: An average Z′ of 0.00±0.13 and a S:B of 3.47±0.56 for N=3 plates was observed.

HEK Antagonist Counterscreen Assay: An average Z′ of 0.28±0.07 and a S:B of 2.25±0.06 for N=3 plates was observed.

For each test compound, percent activation was plotted against compound concentration. A four parameter equation describing a sigmoidal dose-response curve was then fitted with adjustable baseline using Assay Explorer software (Symyx Technologies Inc.). The reported EC50 or IC50 values were generated from fitted curves by solving for the X-intercept value at the 50% activation level of the Y-intercept value. The following rule was used to declare a compound as “active” or “inactive”: Compounds with an EC50 or IC50 greater than 5 micromolar for antagonist mode and 1 micromolar for agonist were considered inactive. Compounds with an EC50 or IC50 equal to or less than 5 micromolar for antagonist mode and 1 micromolar for agonist mode were considered active. Of those, 476 compounds were selective for GPR139 agonists and 81 compounds were selective for GPR139 antagonists.

All compounds analyzed by titration assay were analyzed by LC-MS to confirm purity and identify mass.

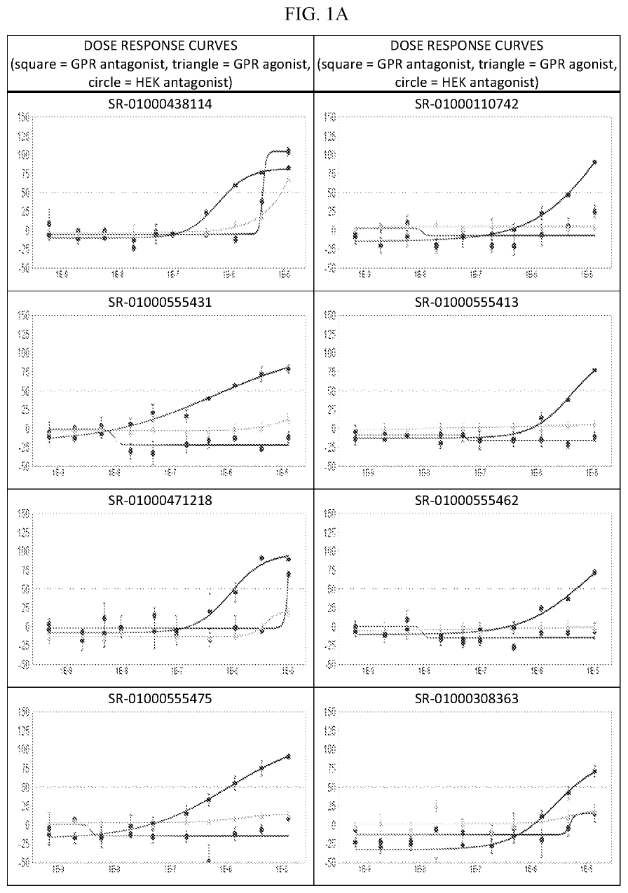

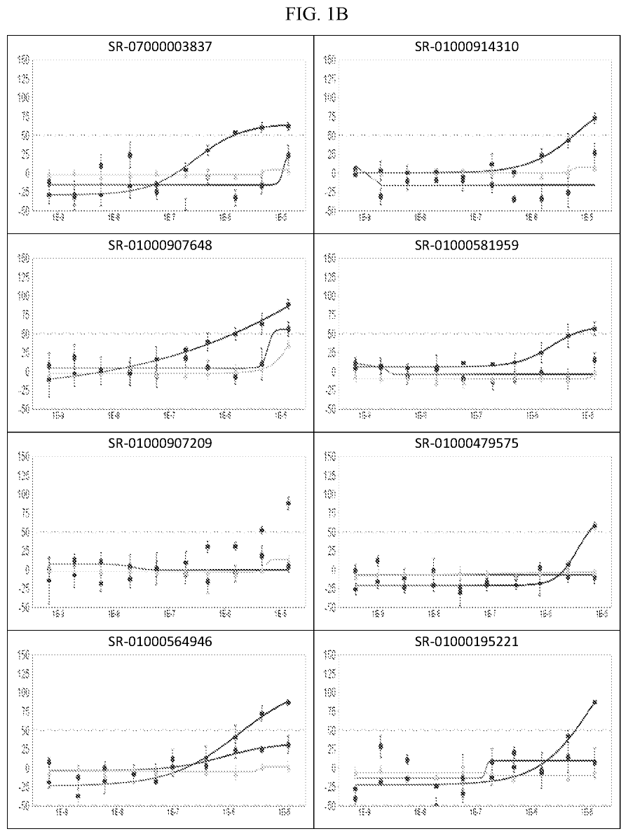

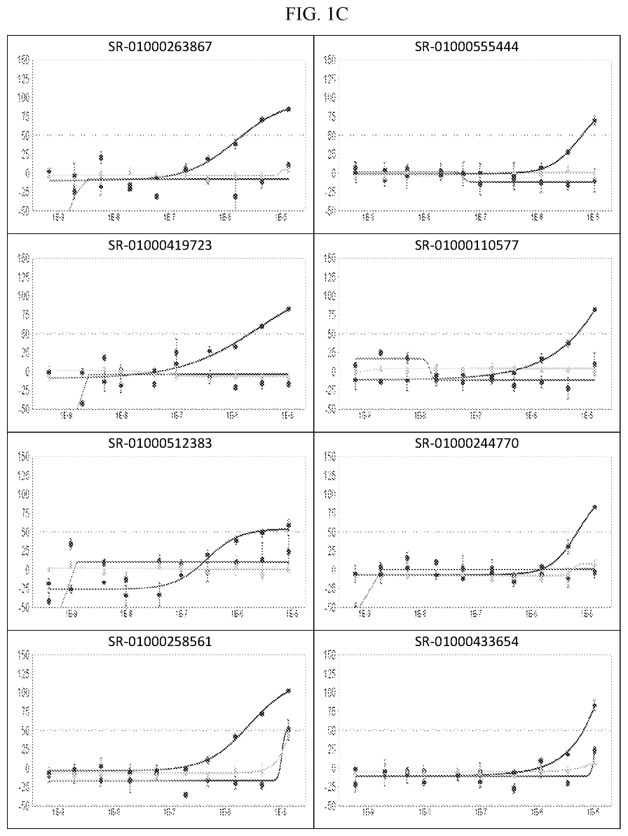

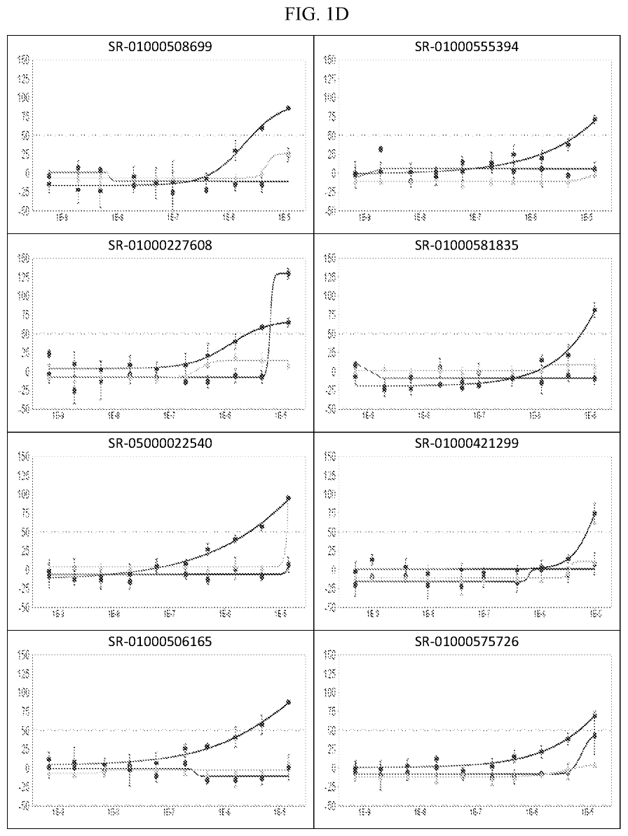

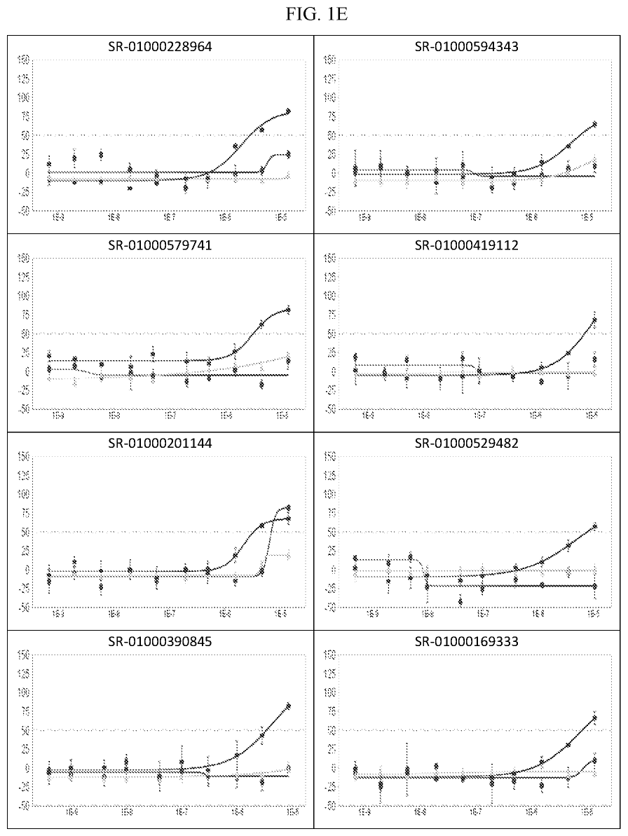

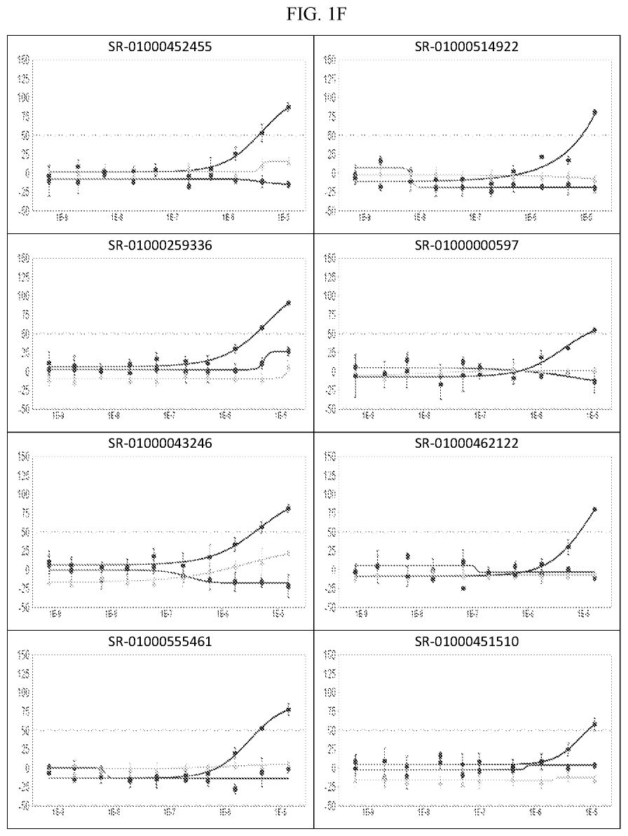

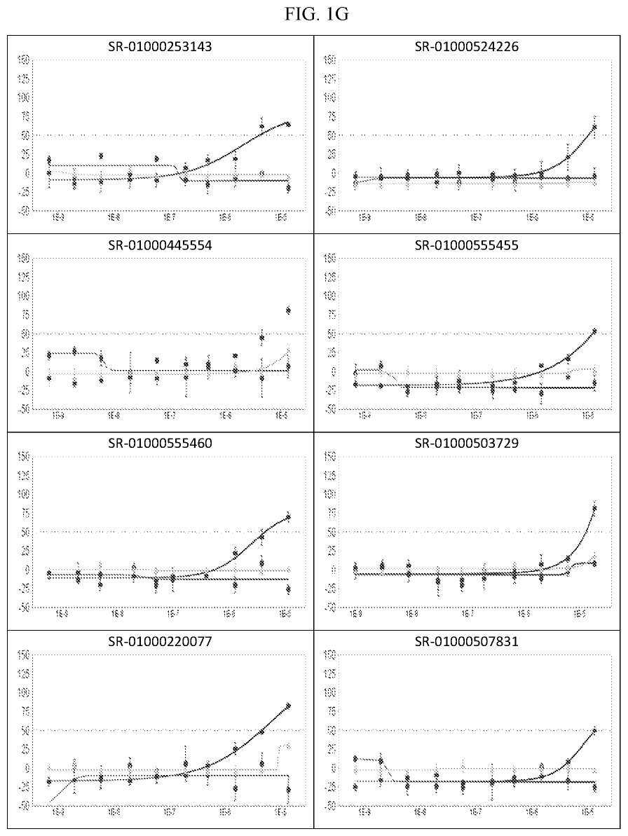

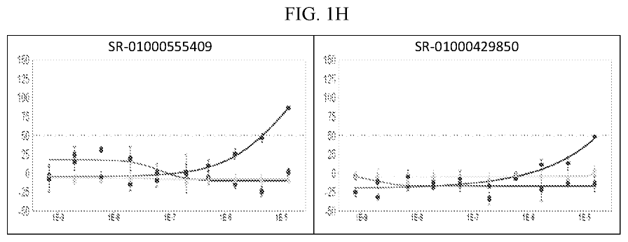

Results for antagonists are shown in FIG. 1(A-H) and Table 3. Results for agonists are shown in FIG. 2 (A-B2) and Table 4. Averaged Max % Response is the activity relative to known control. Hill Slope, or Hill coefficient, provides a way to quantify the degree of interaction between ligand binding sites. A slope greater than one indicates positively cooperative binding between the receptor and the ligand, while a slope less than one indicates negatively cooperative binding. The Hill Slope is a measure of sensitivity.

TABLE 3
Binding and modulation activities of the GPR139 antagonists.

| | GPR139 Antagonist Activity | | | | | | |
|---|---|---|---|---|---|---|---|
| | | Averaged | | | | | GRP139 |
| | | Max % | | | Lipophilic | Lipophilic | Agonist |
| | | Response | Hill | Ligand | Efficiency | Efficiency | activity |
| compound | EC₅₀ | (±St. Dev.) | Slope | Efficiency | (logP) | (logD) | EC₅₀ |
| SR-01000438114 | 980.3E−9 | 82.02 ± 2.67 | 1.59 | 0.37 | 1.57 | 2.55 | 9.1E−6 |
| SR-01000555431 | 992.E−9 | 78.69 ± 5.10 | 0.44 | 0.26 | −548.6E−3 | −548.5E−3 | >13.2E−6 |
| SR-01000471218 | 1.1E−6 | 90.79 ± 3.84 | 1.41 | 0.24 | −130.5E−3 | −130.5E−3 | >10.5E−6 |
| SR-01000555475 | 1.1E−6 | 90.42 ± 3.10 | 0.57 | 0.28 | −768.8E−3 | −768.6E−3 | >13.2E−6 |
| SR-07000003837 | 1.5E−6 | 61.98 ± 5.15 | 1.01 | 0.28 | 1.77 | 1.78 | >14.1E−6 |
| SR-01000907648 | 1.5E−6 | 88.60 ± 6.25 | 0.23 | 0.25 | 1.51 | 1.51 | >14.1E−6 |
| SR-01000907209 | >1.6E−6 | 87.64 ± 8.11 | | 0.33 | 0.62 | 0.62 | >14.1E−6 |
| SR-01000564946 | 1.9E−6 | 86.39 ± 3.49 | 0.71 | 0.24 | 0.81 | 0.81 | >12.1E−6 |
| SR-01000263867 | 2.1E−6 | 84.30 ± 2.68 | 0.99 | 0.31 | 1.99 | 1.99 | >14.1E−6 |
| SR-01000419723 | 2.1E−6 | 82.63 ± 2.69 | 0.63 | 0.23 | 1.36 | 1.36 | >10.6E−6 |
| SR-01000512383 | 2.2E−6 | 58.56 ± 7.31 | 1.46 | 0.27 | −1.6E+0 | −1.6E+0 | >8.9E−6 |
| SR-01000258561 | 2.3E−6 | 102.3 ± 0.93 | 1.14 | 0.34 | 1.45 | 1.45 | >14.1E−6 |
| SR-01000508699 | 2.7E−6 | 85.61 ± 2.70 | 1.30 | 0.39 | 0.57 | 0.57 | >12.E−6 |
| SR-01000227608 | 2.7E−6 | 64.68 ± 5.80 | 1.24 | 0.35 | 2.71 | 3.07 | >14.1E−6 |
| SR-05000022540 | 2.8E−6 | 94.71 ± 3.14 | 0.39 | 0.18 | −780.8E−3 | −546.E−3 | 13.2E−6 |
| SR-01000506165 | 2.8E−6 | 86.87 ± 2.82 | 0.52 | 0.30 | 1.14 | 1.14 | >14.6E−6 |
| SR-01000228964 | 2.9E−6 | 82.24 ± 3.16 | 1.53 | 0.30 | 2.79 | 2.79 | >14.1E−6 |
| SR-01000579741 | 3.1E−6 | 81.29 ± 5.44 | 2.25 | 0.28 | −63.1E−3 | −62.7E−3 | >13.E−6 |
| SR-01000201144 | 3.3E−6 | 67.66 ± 7.67 | 2.60 | 0.27 | 0.66 | 4.02 | >14.1E−6 |
| SR-01000390845 | 3.4E−6 | 82.13 ± 4.52 | 1.16 | 0.26 | 0.28 | 0.28 | >8.6E−6 |
| SR-01000452455 | 3.6E−6 | 87.35 ± 5.76 | 1.28 | 0.32 | 0.49 | 0.49 | >12.5E−6 |
| SR-01000259336 | 3.7E−6 | 90.45 ± 2.75 | 1.05 | 0.33 | 1.62 | 1.62 | >14.1E−6 |
| SR-01000043246 | 3.9E−6 | 80.83 ± 4.99 | 1.01 | 0.47 | 2.61 | 2.61 | >15.3E−6 |
| SR-01000555461 | 4.1E−6 | 77.29 ± 7.71 | 1.40 | 0.25 | −954.4E−3 | −954.3E−3 | >14.E−6 |
| SR-01000253143 | 4.3E−6 | 63.84 ± 2.29 | 0.87 | 0.42 | 0.78 | 0.78 | >14.1E−6 |
| SR-01000445554 | >4.5E−6 | 80.54 ± 5.09 | | 0.29 | 0.71 | 0.71 | >13.6E−6 |
| SR-01000555460 | 4.6E−6 | 69.58 ± 6.90 | 1.30 | 0.24 | −1.9E+0 | −1.9E+0 | >11.9E−6 |
| SR-01000220077 | 4.7E−6 | 82.15 ± 3.63 | 0.74 | 0.32 | 1.20 | 1.23 | >14.1E−6 |
| SR-01000555409 | 4.7E−6 | 86.26 ± 1.14 | 0.79 | 0.24 | −708.9E−3 | −708.8E−3 | >13.6E−6 |
| SR-01000110742 | 4.9E−6 | 90.37 ± 1.31 | 0.68 | 0.28 | 1.94 | 1.95 | >14.1E−6 |
| SR-01000555413 | 5.E−6 | 77.15 ± 0.74 | 1.24 | 0.26 | −521.7E−3 | −521.6E−3 | >11.3E−6 |
| SR-01000555462 | 5.2E−6 | 71.75 ± 3.35 | 0.81 | 0.25 | −1.4E+0 | −1.4E+0 | >11.E−6 |
| SR-01000308363 | 5.9E−6 | 70.81 ± 7.08 | 1.05 | 0.39 | 2.11 | 2.11 | >14.3E−6 |
| SR-01000914310 | 6.E−6 | 72.33 ± 6.63 | 1.00 | 0.29 | 1.56 | 1.70 | >14.1E−6 |
| SR-01000581959 | 6.E−6 | 56.13 ± 9.03 | 1.46 | 0.22 | 2.24 | 2.24 | >13.4E−6 |
| SR-01000479575 | 6.E−6 | 58.09 ± 4.56 | 2.37 | 0.24 | 0.55 | 0.55 | >7.6E−6 |
| SR-01000195221 | 6.3E−6 | 86.87 ± 1.46 | 0.88 | 0.33 | 1.71 | 1.71 | >14.1E−6 |
| SR-01000555444 | 6.6E−6 | 69.32 ± 5.67 | 1.62 | 0.24 | −1.5E+0 | −1.5E+0 | >11.E−6 |
| SR-01000110577 | 6.9E−6 | 81.68 ± 2.49 | 0.62 | 0.28 | 2.23 | 2.48 | >14.1E−6 |
| SR-01000244770 | 6.9E−6 | 82.77 ± 1.38 | 1.77 | 0.31 | 1.07 | 1.07 | >14.1E−6 |
| SR-01000433654 | 7.E−6 | 82.91 ± 7.41 | 0.97 | 0.25 | −712.7E−3 | −712.7E−3 | >10.9E−6 |
| SR-01000555394 | 7.E−6 | 71.06 ± 5.55 | 0.45 | 0.25 | −672.4E−3 | −672.3E−3 | >14.5E−6 |
| SR-01000581835 | 7.E−6 | 81.12 ± 9.72 | 0.67 | 0.23 | −850.1E−3 | −850.1E−3 | >12.1E−6 |
| SR-01000421299 | 7.2E−6 | 74.1 ± 13.55 | 1.98 | 0.26 | 0.17 | 0.17 | >9.8E−6 |
| SR-01000575726 | 7.3E−6 | 68.96 ± 6.17 | 0.63 | 0.18 | 1.85 | 2.05 | >13.9E−6 |
| SR-01000594343 | 7.7E−6 | 64.21 ± 3.85 | −1.40 | 0.26 | 0.36 | 0.51 | >14.1E−6 |
| SR-01000419112 | 7.8E−6 | 67.9 ± 10.41 | 1.28 | 0.23 | 0.44 | 0.44 | >11.7E−6 |
| SR-01000529482 | 7.9E−6 | 56.80 ± 4.90 | 0.94 | 0.22 | −1.1E+0 | −1.1E+0 | >10.8E−6 |
| SR-01000169333 | 8.4E−6 | 66.19 ± 8.60 | 1.11 | 0.22 | 1.37 | 1.64 | >14.1E−6 |
| SR-01000514922 | 8.6E−6 | 80.63 ± 3.57 | 0.70 | 0.28 | 0.55 | 1.63 | >14.7E−6 |
| SR-01000000597 | 8.6E−6 | 54.78 ± 3.56 | 1.21 | 0.24 | −52.4E−3 | −52.3E−3 | >11.2E−6 |
| SR-01000462122 | 8.7E−6 | 79.69 ± 2.61 | 1.19 | 0.32 | 1.04 | 1.04 | >16.2E−6 |
| SR-01000451510 | 8.8E−6 | 57.55 ± 8.35 | 1.89 | 0.25 | −866.9E−3 | −866.9E−3 | >11.7E−6 |
| SR-01000524226 | 10.2E−6 | 60.57 ± 14.37 | 1.32 | 0.30 | 1.28 | 1.28 | >13.3E−6 |
| SR-01000555455 | 12.5E−6 | 52.97 ± 3.58 | 0.71 | 0.24 | −1.3E+0 | −1.3E+0 | >13.3E−6 |
| SR-01000503729 | 13.3E−6 | 81.28 ± 9.79 | 1.28 | 0.30 | 1.36 | 1.36 | >18.9E−6 |
| SR-01000507831 | >13.9E−6 | 49.37 ± 5.06 | | 0.24 | −233.9E−3 | −233.9E−3 | >13.9E−6 |
| SR-01000429850 | >15.5E−6 | 48.35 ± 1.75 | | 0.31 | 1.00 | 1.00 | >15.5E−6 |

| | GRP139 Agonist activity | | HEK antagonist activity | | |
|---|---|---|---|---|---|
| | Averaged | | | Averaged | |
| | Max % | | | Max % | |
| | Response | Hill | | Response | Hill |
| compound | (±St. Dev.) | Slope | EC₅₀ | (±St. Dev.) | Slope |
| SR-01000438114 | 68.19 ± 17.63 | 1.01 | 4.2E−6 | 104.21 ± 5.67 | 13.82 |
| SR-01000555431 | 12.88 ± 6.35 | | >13.2E−6 | 3.15 ± 12.32 | |
| SR-01000471218 | 19.55 ± 6.15 | | 10.1E−6 | 69.47 ± 1.93 | 10.22 |
| SR-01000555475 | 13.90 ± 4.27 | | >13.2E−6 | 9.07 ± 0.74 | |
| SR-07000003837 | 4.29 ± 6.99 | | >14.1E−6 | 23.73 ± 16.97 | |
| SR-01000907648 | 35.82 ± 4.87 | | 7.6E−6 | 55.77 ± 9.26 | 8.81 |
| SR-01000907209 | 13.37 ± 4.41 | | >14.1E−6 | 18.39 ± 12.65 | |
| SR-01000564946 | 1.62 ± 7.06 | | >12.1E−6 | 30.88 ± 11.77 | |
| SR-01000263867 | 4.67 ± 4.93 | | >14.1E−6 | 20.24 ± 7.73 | |
| SR-01000419723 | 3.98 ± 7.81 | | >10.6E−6 | 25.06 ± 17.50 | |
| SR-01000512383 | 6.60 ± 4.93 | | >8.9E−6 | 33.99 ± 7.44 | |
| SR-01000258561 | 45.23 ± 2.15 | | 13.8E−6 | 50.40 ± 13.43 | 14.89 |
| SR-01000508699 | 25.84 ± 3.55 | | >12.E−6 | 23.85 ± 9.06 | |
| SR-01000227608 | 19.40 ± 2.31 | | 6.5E−6 | 129.40 ± 6.90 | 16.87 |
| SR-05000022540 | 96.44 ± 1.50 | 10.54 | >14.1E−6 | 6.21 ± 10.10 | |
| SR-01000506165 | 7.66 ± 5.35 | | >14.6E−6 | 5.72 ± 9.39 | |
| SR-01000228964 | −2.6E+0 ± 5.18 | | >14.1E−6 | 24.51 ± 4.86 | |
| SR-01000579741 | 21.81 ± 2.59 | | >13.E−6 | 13.52 ± 10.95 | |
| SR-01000201144 | 19.36 ± 5.93 | | 6.9E−6 | 81.74 ± 3.36 | 8.01 |
| SR-01000390845 | −1.7E+0 ± 9.13 | | >8.6E−6 | 7.94 ± 9.35 | |
| SR-01000452455 | 15.11 ± 5.37 | | >12.5E−6 | 0.92 ± 5.75 | |
| SR-01000259336 | 6.87 ± 7.17 | | >14.1E−6 | 26.21 ± 5.54 | |
| SR-01000043246 | 21.98 ± 2.69 | | >15.3E−6 | 6.06 ± 11.65 | |
| SR-01000555461 | 5.29 ± 2.24 | | >14.E−6 | 0.37 ± 8.79 | |
| SR-01000253143 | 4.38 ± 3.28 | | >14.1E−6 | 22.82 ± 1.84 | |
| SR-01000445554 | 28.29 ± 7.43 | | >13.6E−6 | 26.51 ± 5.44 | |
| SR-01000555460 | 7.97 ± 3.98 | | >11.9E−6 | 8.34 ± 10.04 | |
| SR-01000220077 | 29.53 ± 3.67 | | >14.1E−6 | 5.29 ± 23.62 | |
| SR-01000555409 | −4.8E+0 ± 5.84 | | >13.6E−6 | 31.02 ± 1.30 | |
| SR-01000110742 | 8.07 ± 1.27 | | >14.1E−6 | 24.28 ± 7.95 | |
| SR-01000555413 | 5.85 ± 3.71 | | >11.3E−6 | −6.1E+0 ± 7.39 | |
| SR-01000555462 | 1.48 ± 6.53 | | >11.E−6 | 9.21 ± 12.02 | |
| SR-01000308363 | 25.24 ± 7.47 | | >14.3E−6 | 14.98 ± 11.31 | |
| SR-01000914310 | 7.64 ± 5.47 | | >14.1E−6 | 26.24 ± 12.88 | |
| SR-01000581959 | −987E−3 ± 9.16 | | >13.4E−6 | 14.54 ± 9.66 | |
| SR-01000479575 | −2.2E+0 ± 2.25 | | >7.6E−6 | 11.60 ± 7.12 | |
| SR-01000195221 | 3.53 ± 6.53 | | >14.1E−6 | 28.17 ± 14.51 | |
| SR-01000555444 | 4.27 ± 6.30 | | >11.E−6 | 6.69 ± 8.37 | |
| SR-01000110577 | 5.61 ± 4.33 | | >14.1E−6 | 24.57 ± 3.75 | |
| SR-01000244770 | 7.91 ± 4.93 | | >14.1E−6 | 15.65 ± 7.33 | |
| SR-01000433654 | 7.71 ± 3.46 | | >10.9E−6 | 23.11 ± 4.71 | |
| SR-01000555394 | −854E−3 ± 6.10 | | >14.5E−6 | 31.37 ± 1.24 | |
| SR-01000581835 | 9.47 ± 9.31 | | >12.1E−6 | 8.20 ± 4.35 | |
| SR-01000421299 | 10.58 ± 2.83 | | >9.8E−6 | 7.76 ± 14.27 | |
| SR-01000575726 | 4.26 ± 2.46 | | >13.9E−6 | 42.61 ± 24.54 | |
| SR-01000594343 | 16.98 ± 6.81 | | >14.1E−6 | 10.38 ± 17.91 | |
| SR-01000419112 | 0.45 ± 5.31 | | >11.7E−6 | 18.74 ± 4.66 | |
| SR-01000529482 | 3.89 ± 5.42 | | >10.8E−6 | 16.45 ± 6.46 | |
| SR-01000169333 | 2.42 ± 7.61 | | >14.1E−6 | 9.54 ± 10.14 | |
| SR-01000514922 | 1.17 ± 6.37 | | >14.7E−6 | 16.24 ± 5.67 | |
| SR-01000000597 | 3.20 ± 6.96 | | >11.2E−6 | 14.40 ± 10.68 | |
| SR-01000462122 | −2.6E+0 ± 5.95 | | >16.2E−6 | 17.80 ± 3.32 | |
| SR-01000451510 | −9.5E+0 ± 9.17 | | >11.7E−6 | 16.06 ± 4.81 | |
| SR-01000524226 | −11.2 ± 4.96 | | >13.3E−6 | −2.2E+0 ± 7.28 | |
| SR-01000555455 | 2.28 ± 5.10 | | >13.3E−6 | 6.61 ± 6.46 | |
| SR-01000503729 | 19.07 ± 3.26 | | >18.9E−6 | 8.15 ± 3.75 | |
| SR-01000507831 | 2.74 ± 8.83 | | >13.9E−6 | 11.97 ± 1.54 | |
| SR-01000429850 | 4.88 ± 6.79 | | >15.5E−6 | −3.2E+0 ± 5.20 | |

TABLE 4
Binding and modulation activities of the GPR139 agonists.

| | GPR139 Agonist Activity | | | | | |
|---|---|---|---|---|---|---|
| | | Averaged | | | | |
| | | Max % | | | Lipophilic | Lipophilic |
| | | Response | Hill | Efficiency | Efficiency | Efficiency |
| compound | EC₅₀ | (±St. Dev.) | Slope | Ligand | (logP) | (logD) |
| SR-01000410744 | <517.4E−12 | 114.68 ± 11.76 | | 0.54 | 5.24 | 5.27 |
| SR-01000520454 | >703.1E−12 | 113.77 ± 7.21 | | 0.53 | 5.48 | 5.60 |
| SR-01000907288 | >715.7E−12 | 110.51 ± 12.41 | | 0.49 | 5.58 | 5.58 |
| SR-01000295701 | <715.7E−12 | 126.44 ± 4.55 | | 0.56 | 5.47 | 5.48 |
| SR-01000093460 | >715.7E−12 | 112.00 ± 16.24 | | 0.47 | 6.04 | 6.04 |
| SR-01000095280 | <715.7E−12 | 122.65 ± 11.25 | | 0.41 | 6.68 | 6.68 |
| SR-01000068729 | >778.5E−12 | 126.00 ± 7.94 | | 0.44 | 6.03 | 6.03 |
| SR-01000031674 | 1.2E−9 | 113.33 ± 7.65 | 0.44 | 0.50 | 6.57 | 6.57 |
| SR-01000140260 | 1.3E−9 | 114.94 ± 7.97 | 0.54 | 0.52 | 6.86 | 6.86 |
| SR-07000000296 | 1.5E−9 | 115.54 ± 13.78 | 0.89 | 0.49 | 1.98 | 1.98 |
| SR-01000138428 | >2.1E−9 | 81.78 ± 16.61 | | 0.38 | 4.85 | 4.85 |
| SR-01000918716 | >2.1E−9 | 111.04 ± 7.77 | | 0.43 | 5.74 | 5.74 |
| SR-01000205482 | >2.1E−9 | 120.13 ± 6.96 | | 0.47 | 5.34 | 5.35 |
| SR-01000071222 | >2.2E−9 | 126.32 ± 18.07 | | 0.42 | 5.82 | 5.83 |
| SR-01000155701 | 2.6E−9 | 117.99 ± 3.71 | 0.43 | 0.50 | 7.10 | 7.10 |
| SR-01000068229 | 2.9E−9 | 112.57 ± 13.42 | 0.64 | 0.43 | 7.01 | 7.01 |
| SR-01000069345 | 3.7E−9 | 114.67 ± 3.82 | 1.18 | 0.49 | 4.37 | 4.37 |
| SR-01000150430 | 4.1E−9 | 128.13 ± 17.85 | 0.30 | 0.49 | 6.28 | 6.28 |
| SR-01000356497 | 4.9E−9 | 126.50 ± 10.38 | 0.32 | 0.47 | 4.94 | 4.97 |
| SR-01000299915 | 5.E−9 | 99.72 ± 18.10 | 1.13 | 0.55 | 5.86 | 5.86 |
| SR-01000137723 | 5.7E−9 | 136.99 ± 21.58 | 0.44 | 0.52 | 5.09 | 5.09 |
| SR-01000583842 | >5.8E−9 | 160.56 ± 29.47 | | 0.33 | 4.92 | 4.92 |
| SR-01000369718 | 5.9E−9 | 111.46 ± 12.99 | 0.33 | 0.48 | 5.36 | 5.36 |
| SR-01000412367 | 6.2E−9 | 112.23 ± 25.44 | 0.32 | 0.50 | 4.43 | 4.43 |
| SR-01000077904 | >6.4E−9 | 79.43 ± 7.55 | | 0.41 | 4.54 | 4.54 |
| SR-01000146606 | >6.4E−9 | 133.44 ± 24.52 | | 0.40 | 4.56 | 4.56 |
| SR-01000069259 | >6.5E−9 | 126.19 ± 25.88 | | 0.37 | 3.74 | 3.74 |
| SR-01000339494 | 6.5E−9 | 107.79 ± 9.70 | 0.87 | 0.38 | 5.30 | 5.30 |
| SR-01000073202 | >6.6E−9 | 119.67 ± 15.04 | | 0.39 | 4.38 | 4.38 |
| SR-01000633083 | 6.7E−9 | 100.43 ± 4.95 | 0.91 | 0.38 | 4.81 | 4.82 |
| SR-01000048841 | 6.7E−9 | 125.61 ± 14.18 | 0.27 | 0.41 | 5.59 | 5.59 |
| SR-01000911654 | 7.9E−9 | 102.69 ± 7.41 | 0.31 | 0.44 | 4.94 | 4.95 |
| SR-00000006339 | 9.E−9 | 103.42 ± 7.86 | 0.54 | 0.35 | 5.03 | 6.10 |
| SR-01000068761 | 9.7E−9 | 123.40 ± 6.48 | 0.55 | 0.42 | 4.99 | 5.00 |
| SR-01000074712 | 9.7E−9 | 109.67 ± 8.84 | 0.80 | 0.40 | 4.91 | 4.91 |
| SR-01000068718 | 10.1E−9 | 119.00 ± 0.36 | 0.58 | 0.40 | 6.50 | 6.50 |
| SR-01000144615 | 10.3E−9 | 108.00 ± 3.42 | 4.63 | 0.35 | 6.38 | 6.43 |
| SR-01000039980 | 10.4E−9 | 134.67 ± 13.58 | 0.55 | 0.34 | 4.01 | 4.01 |
| SR-01000652673 | 11.5E−9 | 123.26 ± 12.43 | 0.93 | 0.44 | 5.68 | 5.68 |
| SR-01000202338 | 11.6E−9 | 79.48 ± 15.63 | 1.63 | 0.48 | 4.84 | 4.84 |
| SR-01000070891 | 11.9E−9 | 105.87 ± 4.30 | 0.88 | 0.35 | 4.93 | 4.97 |
| SR-01000061624 | 12.E−9 | 120.81 ± 8.57 | 1.00 | 0.43 | 4.00 | 4.01 |
| SR-01000317486 | 12.3E−9 | 114.27 ± 15.21 | 1.01 | 0.44 | 4.70 | 4.70 |
| SR-01000013313 | 13.3E−9 | 93.40 ± 11.14 | 0.63 | 0.48 | 4.44 | 4.45 |
| SR-01000070591 | 13.6E−9 | 126.06 ± 10.32 | 0.31 | 0.39 | 4.02 | 4.02 |
| SR-05000709044 | 13.8E−9 | 99.08 ± 8.57 | 0.79 | 0.24 | 6.95 | 6.97 |
| SR-01000912290 | 14.3E−9 | 103.55 ± 8.13 | 1.13 | 0.44 | 6.11 | 6.11 |
| SR-01000150679 | 14.5E−9 | 137.16 ± 28.97 | 0.34 | 0.38 | 6.35 | 6.35 |
| SR-01000146442 | 14.9E−9 | 108.38 ± 13.78 | 0.79 | 0.41 | 4.80 | 4.80 |
| SR-01000137757 | 15.3E−9 | 160.07 ± 33.89 | 0.09 | 0.48 | 4.32 | 4.32 |
| SR-01000191059 | 15.4E−9 | 113.14 ± 16.27 | 0.80 | 0.38 | 4.57 | 4.57 |
| SR-01000239973 | 15.7E−9 | 105.15 ± 8.65 | 0.92 | 0.39 | 5.11 | 5.11 |
| SR-01000159110 | 16.1E−9 | 95.57 ± 19.71 | 1.18 | 0.45 | 5.19 | 5.19 |
| SR-01000060349 | 16.2E−9 | 123.74 ± 9.56 | 0.81 | 0.50 | 5.26 | 5.31 |
| SR-01000524029 | 16.2E−9 | 98.34 ± 6.18 | 0.92 | 0.57 | 4.61 | 4.61 |
| SR-01000061818 | 16.3E−9 | 110.53 ± 3.36 | 0.71 | 0.50 | 4.41 | 4.41 |
| SR-01000535824 | >16.6E−9 | 136.02 ± 19.68 | | 0.39 | 2.73 | 2.73 |
| SR-01000088477 | 16.7E−9 | 120.85 ± 28.06 | 0.72 | 0.47 | 4.18 | 4.18 |
| SR-01000048417 | 16.9E−9 | 111.02 ± 13.88 | 0.52 | 0.38 | 4.73 | 4.73 |
| SR-01000069564 | 17.2E−9 | 87.90 ± 18.60 | 0.93 | 0.36 | 4.85 | 4.85 |
| SR-01000358013 | 18.7E−9 | 146.26 ± 17.42 | 0.21 | 0.40 | 4.52 | 4.56 |
| SR-01000194602 | 19.1E−9 | 94.95 ± 1.55 | 0.71 | 0.39 | 4.44 | 4.44 |
| SR-01000525232 | 19.1E−9 | 96.70 ± 23.19 | 0.19 | 0.57 | 4.05 | 4.05 |
| SR-01000176150 | 19.2E−9 | 110.06 ± 19.04 | 0.64 | 0.40 | 6.03 | 6.35 |
| SR-01000134029 | >19.3E−9 | 100.82 ± 13.50 | | 0.34 | 4.27 | 4.27 |
| SR-01000504687 | 19.4E−9 | 92.66 ± 5.93 | 0.62 | 0.40 | 4.45 | 4.45 |
| SR-05000022531 | 19.6E−9 | 133.92 ± 16.41 | 0.09 | 0.37 | 3.47 | 3.47 |
| SR-01000104567 | 19.8E−9 | 119.91 ± 2.22 | 0.32 | 0.40 | 5.19 | 5.19 |
| SR-01000918718 | 19.9E−9 | 106.93 ± 8.46 | 0.93 | 0.43 | 6.47 | 6.47 |
| SR-01000145949 | 21.5E−9 | 122.18 ± 22.07 | 1.04 | 0.36 | 5.73 | 5.74 |
| SR-01000183606 | 22.E−9 | 89.43 ± 6.88 | 0.82 | 0.36 | 3.96 | 3.97 |
| SR-01000098944 | 22.4E−9 | 118.40 ± 16.09 | 0.20 | 0.43 | 4.22 | 4.22 |
| SR-01000177515 | 22.6E−9 | 91.26 ± 4.77 | 0.48 | 0.37 | 5.67 | 5.92 |
| SR-01000077930 | 22.8E−9 | 102.55 ± 5.37 | 0.67 | 0.38 | 4.55 | 4.55 |
| SR-01000356020 | 23.E−9 | 106.05 ± 9.09 | 0.91 | 0.43 | 4.27 | 4.30 |
| SR-01000058107 | 23.3E−9 | 95.61 ± 13.32 | 0.85 | 0.43 | 5.35 | 5.35 |
| SR-01000923125 | 23.7E−9 | 81.24 ± 9.74 | 2.01 | 0.37 | 4.30 | 4.30 |
| SR-01000276878 | 23.9E−9 | 97.36 ± 11.63 | 0.75 | 0.38 | 4.36 | 4.40 |
| SR-01000918721 | 25.6E−9 | 107.86 ± 14.78 | 0.48 | 0.38 | 5.70 | 5.70 |
| SR-01000099472 | 26.3E−9 | 105.06 ± 17.21 | 0.88 | 0.39 | 5.32 | 5.32 |
| SR-01000155068 | 26.7E−9 | 127.51 ± 13.72 | 0.58 | 0.41 | 5.00 | 5.00 |
| SR-00000007138 | 27.1E−9 | 109.71 ± 13.62 | 0.17 | 0.31 | 4.48 | 5.84 |
| SR-01000071098 | 27.4E−9 | 108.04 ± 6.43 | 0.77 | 0.39 | 4.08 | 4.08 |
| SR-01000919746 | 28.1E−9 | 101.90 ± 10.65 | 0.63 | 0.38 | 3.89 | 3.89 |
| SR-01000021416 | 29.1E−9 | 122.23 ± 2.65 | 0.24 | 0.44 | 3.53 | 3.53 |
| SR-01000146420 | 29.6E−9 | 113.46 ± 20.46 | 1.05 | 0.38 | 4.29 | 4.29 |
| SR-01000298170 | 31.6E−9 | 119.00 ± 5.31 | 0.27 | 0.46 | 4.08 | 4.08 |
| SR-01000037440 | 32.9E−9 | 90.68 ± 18.21 | 0.54 | 0.42 | 3.59 | 3.59 |
| SR-01000064440 | 33.3E−9 | 139.74 ± 28.19 | 0.54 | 0.34 | 4.46 | 4.46 |
| SR-01000362293 | 33.5E−9 | 100.19 ± 16.30 | 1.02 | 0.46 | 4.07 | 5.01 |
| SR-01000223498 | 33.9E−9 | 115.59 ± 14.29 | 0.81 | 0.39 | 4.05 | 4.05 |
| SR-01000012205 | 34.4E−9 | 109.87 ± 14.69 | 0.93 | 0.36 | 4.05 | 4.06 |
| SR-01000068721 | 34.7E−9 | 109.68 ± 5.18 | 0.71 | 0.40 | 5.32 | 5.32 |
| SR-01000125458 | 35.8E−9 | 119.40 ± 41.91 | 0.73 | 0.40 | 4.54 | 4.54 |
| SR-01000589950 | 36.4E−9 | 90.30 ± 31.36 | 0.72 | 0.36 | 5.98 | 5.98 |
| SR-01000048367 | 36.5E−9 | 122.38 ± 5.77 | 0.74 | 0.45 | 5.64 | 5.64 |
| SR-01000584158 | 36.6E−9 | 123.61 ± 28.77 | 0.47 | 0.37 | 2.25 | 2.25 |
| SR-01000923247 | 37.1E−9 | 95.66 ± 12.25 | 0.45 | 0.43 | 3.21 | 3.21 |
| SR-01000048295 | 38.3E−9 | 102.89 ± 3.61 | 0.88 | 0.40 | 4.43 | 4.43 |
| SR-01000020502 | 38.7E−9 | 91.42 ± 14.52 | 1.13 | 0.32 | 2.69 | 2.69 |
| SR-01000071221 | 39.6E−9 | 129.99 ± 9.49 | 1.13 | 0.52 | 2.68 | 2.68 |
| SR-01000583473 | 40.1E−9 | 116.62 ± 3.81 | 0.26 | 0.23 | 3.71 | 3.89 |
| SR-01000921178 | 40.4E−9 | 103.82 ± 12.81 | 0.61 | 0.36 | 4.98 | 4.98 |
| SR-01000058120 | 40.5E−9 | 103.66 ± 2.94 | 0.17 | 0.37 | 4.73 | 4.73 |
| SR-01000066950 | 40.5E−9 | 117.24 ± 4.32 | 0.61 | 0.36 | 3.74 | 3.74 |
| SR-01000290411 | 41.3E−9 | 82.58 ± 9.37 | 0.72 | 0.41 | 3.71 | 4.65 |
| SR-01000069642 | 43.1E−9 | 100.05 ± 4.70 | 0.80 | 0.41 | 4.99 | 4.99 |
| SR-00000007279 | 43.1E−9 | 142.11 ± 3.85 | 0.18 | 0.38 | 4.80 | 4.81 |
| SR-01000926956 | 45.1E−9 | 100.27 ± 11.90 | 0.74 | 0.37 | 5.36 | 5.36 |
| SR-01000041378 | 46.1E−9 | 94.48 ± 15.59 | 0.61 | 0.35 | 4.29 | 4.29 |
| SR-01000125584 | 47.1E−9 | 103.06 ± 30.95 | 0.81 | 0.38 | 4.59 | 4.59 |
| SR-01000337370 | 47.5E−9 | 96.26 ± 15.22 | 0.87 | 0.34 | 5.61 | 5.62 |
| SR-01000916271 | 47.9E−9 | 111.55 ± 2.57 | 0.34 | 0.38 | 4.82 | 4.82 |
| SR-01000583192 | 48.1E−9 | 108.22 ± 25.63 | 0.51 | 0.35 | 4.61 | 4.61 |
| SR-01000918720 | 48.9E−9 | 107.14 ± 13.14 | 0.42 | 0.37 | 5.56 | 5.56 |
| SR-01000090996 | 49.3E−9 | 117.44 ± 4.30 | 1.21 | 0.34 | 4.52 | 4.52 |
| SR-01000551463 | 49.6E−9 | 110.88 ± 6.83 | 0.64 | 0.33 | 3.11 | 3.11 |
| SR-01000135038 | 50.5E−9 | 105.92 ± 4.10 | 0.77 | 0.43 | 5.31 | 5.32 |
| SR-01000056458 | 50.9E−9 | 119.47 ± 9.11 | 0.52 | 0.35 | 5.10 | 5.10 |
| SR-01000915644 | 51.E−9 | 98.09 ± 3.30 | 0.31 | 0.41 | 3.69 | 3.69 |
| SR-01000045673 | 54.3E−9 | 101.61 ± 5.25 | 0.62 | 0.34 | 3.67 | 3.67 |
| SR-01000579790 | 55.E−9 | 106.58 ± 14.13 | 1.04 | 0.33 | 2.74 | 2.74 |
| SR-01000129880 | 56.1E−9 | 114.68 ± 22.09 | 0.44 | 0.32 | 3.61 | 3.62 |
| SR-01000546462 | 56.5E−9 | 107.13 ± 9.94 | 0.51 | 0.26 | 2.98 | 2.98 |
| SR-01000071178 | 56.8E−9 | 85.52 ± 11.85 | 0.50 | 0.32 | 4.97 | 4.97 |
| SR-01000021435 | >57.4E−9 | 162.60 ± 13.73 | | 0.42 | 3.60 | 3.60 |
| SR-01000918134 | >58.0E−9 | 116.95 ± 4.02 | | 0.33 | 3.05 | 3.05 |
| SR-01000073564 | 58.6E−9 | 129.62 ± 8.33 | 0.59 | 0.42 | 4.46 | 4.46 |
| SR-01000062290 | 58.8E−9 | 128.31 ± 15.25 | 0.74 | 0.36 | 3.51 | 3.75 |
| SR-01000077903 | 60.2E−9 | 105.59 ± 5.95 | 0.45 | 0.37 | 3.66 | 3.66 |
| SR-01000151666 | 60.3E−9 | 107.14 ± 25.50 | 0.74 | 0.34 | 4.24 | 4.24 |
| SR-01000218376 | 60.5E−9 | 76.44 ± 0.80 | 0.92 | 0.59 | 3.82 | 3.82 |
| SR-01000069451 | 60.5E−9 | 95.18 ± 9.10 | 1.06 | 0.39 | 6.20 | 6.21 |
| SR-01000064879 | 61.5E−9 | 125.68 ± 8.94 | 0.45 | 0.33 | 5.67 | 5.67 |
| SR-01000135068 | 61.5E−9 | 114.56 ± 16.65 | 0.14 | 0.40 | 4.08 | 4.08 |
| SR-01000069439 | 61.7E−9 | 123.43 ± 12.00 | 0.62 | 0.46 | 3.93 | 3.93 |
| SR-01000916033 | 61.9E−9 | 104.22 ± 8.56 | 0.34 | 0.42 | 5.04 | 5.04 |
| SR-00000005955 | 62.E−9 | 95.13 ± 3.75 | 1.31 | 0.39 | 4.08 | 4.08 |
| SR-01000124399 | 62.9E−9 | 147.35 ± 21.79 | 0.70 | 0.39 | 4.50 | 4.50 |
| SR-01000923249 | 62.9E−9 | 93.59 ± 4.95 | 0.82 | 0.37 | 3.81 | 3.81 |
| SR-01000188699 | 63.8E−9 | 112.57 ± 11.99 | 1.05 | 0.46 | 4.37 | 4.37 |
| SR-01000069435 | 64.6E−9 | 94.34 ± 21.26 | 0.66 | 0.36 | 5.43 | 5.44 |
| SR-01000297192 | 65.7E−9 | 93.06 ± 7.15 | 0.70 | 0.48 | 5.57 | 5.58 |
| SR-01000906953 | 68.1E−9 | 95.58 ± 3.98 | 1.02 | 0.36 | 3.16 | 3.16 |
| SR-01000021433 | 71.8E−9 | 111.88 ± 11.27 | 0.56 | 0.45 | 4.59 | 4.59 |
| SR-01000152365 | 73.E−9 | 96.01 ± 17.34 | 0.99 | 0.42 | 4.77 | 4.77 |
| SR-01000080408 | 75.E−9 | 105.88 ± 16.21 | 0.34 | 0.45 | 4.72 | 6.30 |
| SR-01000074687 | 77.1E−9 | 90.02 ± 17.44 | 0.77 | 0.45 | 3.62 | 3.63 |
| SR-01000029575 | 78.1E−9 | 96.54 ± 7.56 | 0.79 | 0.38 | 3.34 | 3.35 |
| SR-01000135037 | 78.9E−9 | 95.23 ± 13.49 | 1.23 | 0.32 | 3.94 | 3.95 |
| SR-01000336035 | 79.7E−9 | 100.35 ± 10.43 | 0.39 | 0.37 | 6.65 | 6.65 |
| SR-01000054849 | 81.6E−9 | 93.14 ± 10.62 | 0.47 | 0.35 | 3.67 | 3.67 |
| SR-01000135070 | 82.9E−9 | 89.37 ± 8.48 | 0.51 | 0.38 | 4.16 | 4.16 |
| SR-01000484454 | 85.3E−9 | 96.91 ± 19.53 | 0.52 | 0.28 | 1.98 | 1.98 |
| SR-01000652579 | 93.2E−9 | 108.92 ± 9.07 | 0.44 | 0.36 | 3.26 | 3.26 |
| SR-01000065653 | 94.4E−9 | 89.15 ± 7.00 | 1.10 | 0.34 | 4.05 | 4.06 |
| SR-01000126715 | 96.3E−9 | 98.66 ± 13.78 | 1.08 | 0.41 | 4.11 | 4.11 |
| SR-01000048373 | 96.7E−9 | 118.34 ± 15.06 | 0.66 | 0.34 | 3.96 | 3.96 |
| SR-01000146444 | 97.E−9 | 112.24 ± 16.06 | 0.93 | 0.35 | 3.54 | 3.54 |
| SR-01000067982 | 98.6E−9 | 88.62 ± 11.28 | 0.94 | 0.39 | 3.54 | 3.55 |
| SR-01000068916 | 98.9E−9 | 118.50 ± 16.12 | 0.84 | 0.43 | 4.23 | 4.23 |
| SR-01000410694 | 100.E−9 | 91.99 ± 13.68 | 1.03 | 0.41 | 2.96 | 2.98 |
| SR-01000318010 | 102.6E−9 | 90.09 ± 14.61 | 1.08 | 0.38 | 3.28 | 3.32 |
| SR-00000007008 | 103.E−9 | 75.98 ± 6.44 | 0.79 | 0.36 | 3.98 | 3.98 |
| SR-01000074761 | 103.2E−9 | 95.96 ± 3.33 | 0.92 | 0.34 | 5.32 | 5.32 |
| SR-01000021418 | 103.4E−9 | 109.05 ± 4.97 | 0.97 | 0.44 | 3.80 | 3.80 |
| SR-01000309617 | 103.4E−9 | 94.36 ± 3.49 | 0.63 | 0.41 | 3.82 | 3.88 |
| SR-01000582959 | 104.1E−9 | 110.54 ± 17.48 | 0.78 | 0.31 | 3.69 | 3.69 |
| SR-01000393531 | 105.7E−9 | 94.39 ± 11.70 | 0.70 | 0.41 | 3.90 | 4.84 |
| SR-01000142419 | 106.3E−9 | 123.62 ± 30.65 | 0.32 | 0.36 | 4.22 | 4.22 |
| SR-01000069699 | 108.1E−9 | 116.05 ± 3.28 | 0.54 | 0.38 | 4.18 | 4.18 |
| SR-01000654192 | 109.5E−9 | 115.41 ± 10.16 | 0.89 | 0.28 | 3.38 | 3.51 |
| SR-01000150393 | 110.1E−9 | 97.57 ± 16.88 | 1.18 | 0.31 | 3.54 | 3.55 |
| SR-01000370374 | 114.2E−9 | 100.37 ± 4.50 | 0.87 | 0.39 | 3.75 | 3.80 |
| SR-01000145393 | 115.6E−9 | 97.82 ± 12.25 | 0.70 | 0.40 | 4.42 | 4.42 |
| SR-01000273754 | 117.7E−9 | 86.25 ± 6.17 | 0.89 | 0.51 | 4.03 | 4.03 |
| SR-01000021414 | 118.7E−9 | 97.10 ± 8.84 | 0.83 | 0.44 | 4.46 | 4.46 |
| SR-00000007518 | 120.2E−9 | 91.59 ± 7.88 | 0.81 | 0.30 | 3.64 | 4.71 |
| SR-01000189442 | 120.7E−9 | 147.06 ± 19.10 | 0.25 | 0.31 | 3.76 | 3.76 |
| SR-01000098976 | 120.7E−9 | 103.10 ± 11.12 | 0.87 | 0.39 | 4.60 | 4.60 |
| SR-01000185712 | 122.1E−9 | 110.27 ± 5.24 | 0.46 | 0.29 | 2.48 | 2.48 |
| SR-01000124129 | 122.9E−9 | 84.79 ± 6.25 | 0.88 | 0.51 | 3.83 | 3.83 |
| SR-00000004118 | 125.8E−9 | 94.30 ± 8.24 | 1.08 | 0.31 | 3.83 | 5.16 |
| SR-01000068608 | 125.9E−9 | 124.32 ± 15.50 | 0.93 | 0.34 | 4.51 | 4.52 |
| SR-01000926263 | 126.7E−9 | 84.49 ± 4.10 | 1.10 | 0.39 | 4.34 | 4.34 |
| SR-01000152429 | 127.5E−9 | 109.51 ± 16.91 | 1.07 | 0.40 | 4.53 | 4.53 |
| SR-00000002785 | 128.E−9 | 80.59 ± 9.85 | 0.95 | 0.32 | 4.66 | 4.66 |
| SR-01000197834 | 131.2E−9 | 95.96 ± 5.70 | 1.09 | 0.54 | 4.61 | 6.98 |
| SR-01000286062 | 131.7E−9 | 99.20 ± 4.08 | 0.78 | 0.48 | 4.23 | 4.23 |
| SR-01000655128 | 133.9E−9 | 106.36 ± 3.74 | 0.50 | 0.36 | 3.70 | 3.70 |
| SR-01000151876 | 134.E−9 | 99.93 ± 9.60 | 0.53 | 0.44 | 4.52 | 4.52 |
| SR-00000004451 | 134.4E−9 | 92.93 ± 0.53 | 0.70 | 0.33 | 4.33 | 6.38 |
| SR-01000034566 | 136.8E−9 | 100.73 ± 15.83 | 0.96 | 0.36 | 4.65 | 4.65 |
| SR-01000129845 | 137.6E−9 | 98.04 ± 26.06 | 1.01 | 0.32 | 3.96 | 3.96 |
| SR-01000012264 | 138.2E−9 | 85.12 ± 11.10 | 0.80 | 0.40 | 3.36 | 3.36 |
| SR-01000316766 | 138.3E−9 | 101.45 ± 17.20 | 1.07 | 0.33 | 3.15 | 3.15 |
| SR-01000145157 | 140.3E−9 | 83.58 ± 17.45 | 0.58 | 0.37 | 3.85 | 3.85 |
| SR-01000125460 | 140.6E−9 | 95.59 ± 27.48 | 1.03 | 0.38 | 4.03 | 4.03 |
| SR-01000091502 | 141.3E−9 | 88.58 ± 8.42 | 0.85 | 0.31 | 4.59 | 4.63 |
| SR-01000075116 | 141.9E−9 | 81.52 ± 3.66 | 0.99 | 0.37 | 4.72 | 4.72 |
| SR-01000918719 | 142.2E−9 | 94.25 ± 9.34 | 0.65 | 0.37 | 5.17 | 5.17 |
| SR-01000125464 | 142.9E−9 | 83.25 ± 20.00 | 0.73 | 0.35 | 3.74 | 3.74 |
| SR-01000101329 | 143.1E−9 | 102.96 ± 12.58 | 0.79 | 0.38 | 5.15 | 5.16 |
| SR-01000319951 | 143.1E−9 | 97.49 ± 3.70 | 0.88 | 0.42 | 4.53 | 4.59 |
| SR-01000545824 | 145.1E−9 | 103.05 ± 12.22 | 0.41 | 0.23 | 1.92 | 1.92 |
| SR-01000301649 | 145.4E−9 | 93.20 ± 6.78 | 0.91 | 0.37 | 3.92 | 3.92 |
| SR-01000074154 | 146.7E−9 | 109.38 ± 5.55 | 0.54 | 0.32 | 5.22 | 5.22 |
| SR-01000070509 | 152.6E−9 | 96.94 ± 14.18 | 0.89 | 0.30 | 4.29 | 4.29 |
| SR-01000134947 | 156.E−9 | 111.20 ± 10.15 | 0.36 | 0.38 | 4.02 | 4.02 |
| SR-01000088420 | 156.5E−9 | 100.76 ± 18.47 | 1.29 | 0.41 | 3.28 | 3.28 |
| SR-01000914970 | 157.9E−9 | 101.00 ± 9.27 | 0.53 | 0.50 | 3.97 | 3.98 |
| SR-01000125462 | 159.1E−9 | 92.90 ± 12.57 | 1.38 | 0.37 | 3.53 | 3.53 |
| SR-01000100493 | 159.5E−9 | 100.68 ± 11.72 | 0.73 | 0.43 | 3.60 | 3.60 |
| SR-01000190122 | 161.E−9 | 134.89 ± 17.50 | 0.52 | 0.30 | 3.22 | 3.22 |
| SR-01000918303 | 166.2E−9 | 84.68 ± 8.82 | 0.73 | 0.35 | 4.29 | 4.29 |
| SR-01000069391 | 170.9E−9 | 99.00 ± 11.26 | 0.48 | 0.39 | 4.39 | 4.39 |
| SR-01000131962 | 173.3E−9 | 113.53 ± 12.66 | 1.14 | 0.34 | 4.28 | 4.28 |
| SR-01000042100 | 173.9E−9 | 100.32 ± 4.53 | 0.79 | 0.45 | 4.12 | 4.12 |
| SR-01000913573 | >173.9E−9 | 186.79 ± 37.86 | | 0.39 | 2.75 | 2.75 |
| SR-01000152760 | 174.4E−9 | 85.23 ± 10.03 | 0.63 | 0.38 | 4.79 | 4.79 |
| SR-01000134941 | 175.6E−9 | 109.36 ± 34.06 | 0.39 | 0.39 | 4.30 | 4.30 |
| SR-01000207758 | 178.3E−9 | 124.78 ± 10.06 | 0.77 | 0.31 | 3.82 | 3.82 |
| SR-01000103876 | 179.3E−9 | 114.46 ± 17.21 | 0.82 | 0.41 | 4.50 | 4.50 |
| SR-01000098920 | 181.2E−9 | 139.09 ± 25.80 | 0.40 | 0.36 | 3.02 | 3.02 |
| SR-01000052065 | 182.1E−9 | 98.20 ± 7.98 | 0.53 | 0.33 | 4.54 | 4.63 |
| SR-01000114500 | 184.2E−9 | 93.61 ± 11.95 | 1.06 | 0.34 | 4.21 | 4.21 |
| SR-00000005389 | 185.6E−9 | 116.35 ± 10.47 | 0.77 | 0.29 | 3.26 | 4.67 |
| SR-01000150389 | 188.9E−9 | 112.97 ± 21.84 | 0.95 | 0.38 | 4.43 | 4.43 |
| SR-00000005347 | 189.4E−9 | 110.92 ± 4.82 | 0.58 | 0.39 | 4.10 | 4.10 |
| SR-01000654639 | 190.6E−9 | 99.13 ± 2.27 | 1.05 | 0.34 | 2.51 | 2.51 |
| SR-01000299442 | 192.2E−9 | 96.21 ± 12.55 | 0.83 | 0.38 | 3.47 | 3.47 |
| SR-01000150449 | 196.E−9 | 107.14 ± 14.60 | 0.60 | 0.38 | 4.32 | 4.32 |
| SR-01000146516 | 196.E−9 | 88.18 ± 13.52 | 0.77 | 0.36 | 5.60 | 5.60 |
| SR-01000187393 | 196.5E−9 | 99.15 ± 6.04 | 0.44 | 0.32 | 4.29 | 4.29 |
| SR-01000284749 | 196.7E−9 | 121.73 ± 17.10 | 1.20 | 0.34 | 2.26 | 2.26 |
| SR-01000361779 | 197.4E−9 | 89.14 ± 2.93 | 0.85 | 0.36 | 3.35 | 3.38 |
| SR-01000124135 | 197.5E−9 | 71.94 ± 4.54 | 0.57 | 0.45 | 2.76 | 2.76 |
| SR-01000187428 | 200.6E−9 | 99.03 ± 14.80 | 1.55 | 0.32 | 3.43 | 3.43 |
| SR-00000005253 | 201.8E−9 | 80.15 ± 4.78 | 0.64 | 0.39 | 4.08 | 4.08 |
| SR-01000062726 | 204.6E−9 | 103.63 ± 9.70 | 0.89 | 0.32 | 4.25 | 4.25 |
| SR-01000243797 | 204.8E−9 | 105.74 ± 4.40 | 1.05 | 0.39 | 3.09 | 3.09 |
| SR-07000002775 | 204.9E−9 | 88.97 ± 19.06 | 0.76 | 0.35 | 1.77 | 1.77 |
| SR-01000125622 | 208.5E−9 | 99.14 ± 16.79 | 0.99 | 0.35 | 3.63 | 3.63 |
| SR-01000318079 | 210.1E−9 | 102.46 ± 8.64 | 0.56 | 0.30 | 3.77 | 3.78 |
| SR-01000113198 | 211.3E−9 | 107.83 ± 17.88 | 1.00 | 0.37 | 4.35 | 4.35 |
| SR-01000273778 | 214.9E−9 | 90.77 ± 11.24 | 0.93 | 0.36 | 2.73 | 2.94 |
| SR-01000582958 | 228.1E−9 | 101.07 ± 20.22 | 0.87 | 0.33 | 5.28 | 5.28 |
| SR-01000144627 | 229.4E−9 | 93.44 ± 12.43 | 1.08 | 0.34 | 4.88 | 4.94 |
| SR-01000312500 | 229.7E−9 | 109.77 ± 10.41 | 0.54 | 0.36 | 3.02 | 3.02 |
| SR-01000357814 | 230.9E−9 | 103.91 ± 10.30 | 0.77 | 0.36 | 3.99 | 4.04 |
| SR-01000068114 | 230.9E−9 | 88.78 ± 5.98 | 0.64 | 0.31 | 4.34 | 4.34 |
| SR-01000145326 | 233.8E−9 | 85.13 ± 14.28 | 0.54 | 0.32 | 3.73 | 3.73 |
| SR-01000070303 | 234.7E−9 | 105.64 ± 11.39 | 1.02 | 0.32 | 6.82 | 6.82 |
| SR-01000520452 | 235.E−9 | 113.17 ± 28.03 | 0.85 | 0.39 | 2.96 | 3.08 |
| SR-01000127953 | 236.2E−9 | 96.92 ± 13.57 | 0.56 | 0.40 | 3.73 | 4.13 |
| SR-01000260567 | 238.5E−9 | 78.32 ± 9.32 | 1.65 | 0.52 | 3.83 | 3.83 |
| SR-00000006152 | 240.8E−9 | 88.32 ± 5.94 | 0.93 | 0.36 | 3.81 | 3.81 |
| SR-01000080433 | 242.5E−9 | 107.91 ± 12.91 | 0.75 | 0.40 | 4.98 | 4.98 |
| SR-01000090244 | 243.E−9 | 88.22 ± 10.77 | 0.86 | 0.32 | 4.83 | 4.83 |
| SR-01000105214 | 244.8E−9 | 114.03 ± 9.34 | 0.51 | 0.36 | 3.89 | 3.89 |
| SR-01000034627 | 244.8E−9 | 64.98 ± 9.75 | 2.03 | 0.34 | 3.27 | 3.27 |
| SR-01000069352 | 247.7E−9 | 97.44 ± 1.25 | 1.19 | 0.44 | 3.02 | 3.03 |
| SR-01000155030 | 251.9E−9 | 113.63 ± 16.89 | 0.65 | 0.40 | 3.06 | 3.06 |
| SR-01000391835 | 253.5E−9 | 104.57 ± 1.81 | 0.80 | 0.44 | 2.52 | 5.91 |
| SR-01000051038 | 256.9E−9 | 101.50 ± 14.12 | 0.86 | 0.33 | 3.45 | 3.45 |
| SR-01000271555 | 260.E−9 | 124.00 ± 20.16 | 0.62 | 0.44 | 3.11 | 3.11 |
| SR-01000135048 | 265.7E−9 | 109.25 ± 4.64 | 1.29 | 0.40 | 4.72 | 4.72 |
| SR-01000162178 | 271.8E−9 | 112.85 ± 32.26 | 1.13 | 0.40 | 3.29 | 3.44 |
| SR-00000006698 | 274.3E−9 | 88.34 ± 12.18 | 1.33 | 0.29 | 4.82 | 7.81 |
| SR-01000479007 | 277.1E−9 | 101.79 ± 19.12 | 1.21 | 0.40 | 4.24 | 4.24 |
| SR-01000140172 | 277.7E−9 | 113.63 ± 31.77 | 0.89 | 0.40 | 5.11 | 5.11 |
| SR-00000005301 | 281.2E−9 | 86.84 ± 8.43 | 0.94 | 0.40 | 3.26 | 3.26 |
| SR-01000146448 | 281.2E−9 | 112.56 ± 16.12 | 0.92 | 0.35 | 3.97 | 3.97 |
| SR-01000146446 | 288.7E−9 | 113.08 ± 17.13 | 0.77 | 0.37 | 4.40 | 4.40 |
| SR-01000031964 | 289.8E−9 | 89.11 ± 2.96 | 1.27 | 0.51 | 4.00 | 4.00 |
| SR-01000145135 | 300.6E−9 | 81.84 ± 7.77 | 0.84 | 0.30 | 4.01 | 4.49 |
| SR-01000161248 | 309.4E−9 | 106.33 ± 15.91 | 1.12 | 0.38 | 4.17 | 4.17 |
| SR-01000428888 | 313.5E−9 | 105.90 ± 33.97 | 0.88 | 0.38 | 2.10 | 2.10 |
| SR-01000920602 | 324.6E−9 | 90.23 ± 7.66 | 0.91 | 0.39 | 4.83 | 4.83 |
| SR-01000151350 | 328.E−9 | 101.74 ± 19.26 | 1.51 | 0.29 | 3.10 | 3.10 |
| SR-01000313440 | 331.5E−9 | 84.05 ± 13.31 | 1.29 | 0.28 | 3.01 | 3.07 |
| SR-01000922914 | 335.1E−9 | 76.15 ± 6.21 | 0.68 | 0.36 | 2.32 | 2.32 |
| SR-01000069574 | 338.7E−9 | 93.05 ± 4.55 | 0.94 | 0.35 | 3.67 | 3.68 |
| SR-01000644888 | 352.4E−9 | 113.87 ± 14.45 | 0.74 | 0.31 | 3.40 | 3.44 |
| SR-01000373848 | 353.8E−9 | 97.42 ± 13.41 | 0.90 | 0.32 | 3.07 | 3.07 |
| SR-01000105999 | 355.3E−9 | 100.11 ± 8.55 | 1.37 | 0.32 | 3.64 | 3.64 |
| SR-07000004609 | 358.1E−9 | 87.84 ± 4.08 | 0.44 | 0.39 | 0.98 | 0.98 |
| SR-01000042109 | 363.2E−9 | 92.84 ± 12.11 | 1.40 | 0.47 | 3.80 | 3.80 |
| SR-01000129545 | 365.4E−9 | 92.13 ± 1.72 | 0.47 | 0.30 | 2.68 | 2.69 |
| SR-01000286858 | 366.4E−9 | 111.41 ± 5.44 | 0.60 | 0.38 | 3.67 | 3.68 |
| SR-01000916375 | 372.7E−9 | 91.29 ± 4.98 | 0.68 | 0.36 | 2.99 | 3.43 |
| SR-01000132330 | 375.E−9 | 105.13 ± 15.28 | 0.90 | 0.30 | 3.63 | 3.63 |
| SR-01000582967 | 375.3E−9 | 117.27 ± 2.34 | 0.38 | 0.27 | 4.47 | 4.47 |
| SR-01000284323 | 386.5E−9 | 94.97 ± 7.83 | 0.57 | 0.41 | 4.90 | 4.90 |
| SR-01000920660 | 386.6E−9 | 83.52 ± 6.71 | 1.17 | 0.41 | 3.39 | 3.39 |
| SR-01000924775 | 387.2E−9 | 69.93 ± 11.99 | 0.79 | 0.31 | 4.98 | 4.98 |
| SR-01000073877 | 394.7E−9 | 119.18 ± 2.86 | 1.03 | 0.41 | 3.17 | 3.26 |
| SR-01000135042 | 401.1E−9 | 104.44 ± 8.42 | 0.33 | 0.37 | 3.32 | 3.32 |
| SR-01000314474 | 401.2E−9 | 90.74 ± 17.54 | 2.04 | 0.37 | 3.13 | 3.13 |
| SR-01000193031 | 406.5E−9 | 95.01 ± 4.20 | 0.62 | 0.30 | 5.73 | 5.90 |
| SR-01000310833 | 407.2E−9 | 80.08 ± 5.83 | 0.71 | 0.56 | 3.32 | 3.32 |
| SR-01000050884 | 407.6E−9 | 89.45 ± 3.28 | 0.73 | 0.36 | 3.46 | 3.46 |
| SR-00000007266 | 408.5E−9 | 100.59 ± 15.12 | 1.16 | 0.36 | 3.29 | 3.29 |
| SR-01000145701 | 413.8E−9 | 114.02 ± 14.51 | 0.99 | 0.34 | 4.07 | 5.67 |
| SR-01000139601 | 416.5E−9 | 109.30 ± 18.80 | 0.85 | 0.31 | 3.57 | 3.57 |
| SR-01000359572 | 418.1E−9 | 100.90 ± 10.01 | 0.98 | 0.37 | 4.18 | 4.18 |
| SR-01000335002 | 422.1E−9 | 86.79 ± 17.90 | 0.68 | 0.31 | 4.62 | 4.62 |
| SR-01000428566 | 423.6E−9 | 84.07 ± 18.43 | 1.72 | 0.26 | 1.52 | 1.52 |
| SR-01000086488 | 430.1E−9 | 78.41 ± 20.14 | 1.22 | 0.29 | 3.92 | 3.95 |
| SR-01000163111 | 430.7E−9 | 86.57 ± 12.28 | 1.57 | 0.29 | 5.66 | 5.71 |
| SR-01000468553 | 437.1E−9 | 109.12 ± 32.80 | 0.84 | 0.36 | 3.11 | 3.11 |
| SR-01000062419 | 438.2E−9 | 105.27 ± 9.38 | 0.63 | 0.33 | 2.49 | 2.50 |
| SR-01000074979 | 441.5E−9 | 79.45 ± 3.43 | 0.80 | 0.31 | 4.03 | 4.03 |
| SR-01000146281 | 448.9E−9 | 89.20 ± 13.55 | 1.64 | 0.37 | 3.32 | 3.32 |
| SR-01000056004 | 450.E−9 | 81.95 ± 3.88 | 0.89 | 0.30 | 4.08 | 4.08 |
| SR-01000513008 | 453.8E−9 | 109.30 ± 27.76 | 0.93 | 0.42 | 3.32 | 3.32 |
| SR-01000068919 | 460.E−9 | 93.45 ± 21.44 | 0.88 | 0.31 | 3.27 | 3.27 |
| SR-01000070170 | 462.4E−9 | 98.34 ± 1.36 | 0.96 | 0.34 | 3.40 | 3.40 |
| SR-01000065547 | 465.4E−9 | 87.19 ± 17.95 | 0.59 | 0.34 | 5.32 | 5.32 |
| SR-01000098914 | 467.7E−9 | 97.42 ± 2.76 | 0.93 | 0.34 | 3.66 | 3.66 |
| SR-01000583711 | 469.2E−9 | 95.83 ± 14.92 | 0.32 | 0.27 | 3.24 | 3.24 |
| SR-01000124131 | 472.E−9 | 93.55 ± 12.47 | 0.85 | 0.42 | 2.55 | 2.55 |
| SR-00000004710 | 476.6E−9 | 89.55 ± 3.66 | 0.90 | 0.35 | 3.11 | 3.12 |
| SR-01000926510 | 489.8E−9 | 89.57 ± 9.18 | 0.38 | 0.30 | 3.52 | 3.52 |
| SR-01000125648 | 490.4E−9 | 98.80 ± 20.98 | 0.84 | 0.33 | 3.13 | 3.13 |
| SR-01000147127 | 503.7E−9 | 119.36 ± 17.64 | 0.42 | 0.30 | 4.85 | 4.85 |
| SR-01000070173 | 507.3E−9 | 99.23 ± 7.18 | 0.62 | 0.42 | 3.30 | 3.30 |
| SR-01000277633 | 508.4E−9 | 93.18 ± 13.55 | 1.11 | 0.33 | 2.82 | 2.85 |
| SR-01000280669 | 515.6E−9 | 83.43 ± 6.82 | 0.56 | 0.33 | 2.70 | 2.70 |
| SR-01000064101 | 527.E−9 | 90.33 ± 2.09 | 1.15 | 0.38 | 4.09 | 4.09 |
| SR-01000151917 | 536.6E−9 | 110.81 ± 5.15 | 1.73 | 0.28 | 3.33 | 3.34 |
| SR-01000246445 | 553.9E−9 | 77.11 ± 5.52 | 1.22 | 0.36 | 3.52 | 3.52 |
| SR-01000044537 | 554.8E−9 | 89.04 ± 19.24 | 1.39 | 0.31 | 3.09 | 3.09 |
| SR-01000139458 | 556.2E−9 | 116.90 ± 31.97 | 1.12 | 0.29 | 2.20 | 2.20 |
| SR-01000154995 | 571.7E−9 | 88.51 ± 8.87 | 1.08 | 0.36 | 2.91 | 2.91 |
| SR-01000316982 | 579.9E−9 | 86.53 ± 11.45 | 1.19 | 0.46 | 3.86 | 3.90 |
| SR-01000042749 | 584.5E−9 | 74.83 ± 7.16 | 0.27 | 0.29 | 2.77 | 2.77 |
| SR-01000152099 | 589.3E−9 | 112.22 ± 29.96 | 0.62 | 0.36 | 3.61 | 3.61 |
| SR-01000923206 | 603.5E−9 | 74.00 ± 9.00 | 1.67 | 0.31 | 2.98 | 2.98 |
| SR-01000378352 | 615.7E−9 | 93.30 ± 10.83 | 1.14 | 0.36 | 2.89 | 2.89 |
| SR-00000005393 | 629.2E−9 | 87.23 ± 6.46 | 0.94 | 0.25 | 3.22 | 3.27 |
| SR-01000484524 | 629.6E−9 | 102.54 ± 9.28 | 0.34 | 0.23 | 2.07 | 2.07 |
| SR-01000005203 | 650.8E−9 | 77.18 ± 14.00 | 1.01 | 0.38 | 2.24 | 2.24 |
| SR-01000318171 | 656.3E−9 | 76.40 ± 3.97 | 1.01 | 0.36 | 3.49 | 3.49 |
| SR-01000387617 | 660.9E−9 | 81.90 ± 12.88 | 0.75 | 0.35 | 4.28 | 4.28 |
| SR-01000914977 | 671.5E−9 | 109.58 ± 11.58 | 0.25 | 0.45 | 2.88 | 2.89 |
| SR-01000310664 | 672.1E−9 | 89.42 ± 11.78 | 0.70 | 0.41 | 4.32 | 4.32 |
| SR-01000098980 | 679.6E−9 | 103.90 ± 24.19 | 0.43 | 0.36 | 3.09 | 3.09 |
| SR-01000176838 | 697.E−9 | 83.30 ± 1.40 | 0.77 | 0.27 | 4.32 | 4.34 |
| SR-01000362527 | 700.9E−9 | 96.08 ± 11.27 | 0.88 | 0.34 | 2.98 | 2.98 |
| SR-01000313193 | 718.6E−9 | 90.41 ± 15.26 | 1.19 | 0.36 | 2.69 | 2.69 |
| SR-01000289377 | 726.7E−9 | 79.17 ± 5.74 | 1.25 | 0.32 | 3.01 | 3.01 |
| SR-01000925002 | 726.8E−9 | 86.62 ± 3.28 | 0.51 | 0.31 | 3.17 | 3.41 |
| SR-01000106321 | 731.7E−9 | 96.96 ± 1.13 | 1.43 | 0.34 | 3.26 | 3.26 |
| SR-01000565308 | 738.2E−9 | 90.10 ± 8.93 | 1.89 | 0.45 | 2.36 | 2.40 |
| SR-01000013346 | 742.6E−9 | 74.67 ± 0.55 | 0.99 | 0.54 | 3.24 | 3.66 |
| SR-01000359546 | 817.7E−9 | 116.44 ± 14.37 | 0.53 | 0.30 | 2.60 | 2.60 |
| SR-01000099712 | 838.6E−9 | 90.08 ± 9.76 | 1.73 | 0.33 | 3.73 | 3.73 |
| SR-01000125346 | 840.1E−9 | 91.83 ± 43.39 | 1.86 | 0.34 | 2.95 | 2.95 |
| SR-00000004123 | 847.E−9 | 79.85 ± 11.38 | 0.37 | 0.28 | 3.15 | 4.48 |
| SR-01000114492 | 852.7E−9 | 84.13 ± 7.28 | 0.90 | 0.34 | 3.50 | 3.50 |
| SR-01000296110 | 857.2E−9 | 96.78 ± 9.37 | 1.48 | 0.35 | 4.76 | 4.77 |
| SR-01000125626 | 861.4E−9 | 79.66 ± 15.90 | 0.73 | 0.30 | 2.72 | 2.72 |
| SR-01000134913 | 863.E−9 | 96.78 ± 14.90 | 0.51 | 0.31 | 2.73 | 2.73 |
| SR-01000364205 | 866.5E−9 | 94.68 ± 16.83 | 1.55 | 0.33 | 2.29 | 2.50 |
| SR-01000139982 | 890.E−9 | 89.89 ± 26.17 | 0.79 | 0.29 | 2.38 | 2.38 |
| SR-01000044433 | 896.1E−9 | 96.03 ± 12.11 | 0.97 | 0.33 | 4.10 | 4.10 |
| SR-01000054340 | 897.9E−9 | 96.13 ± 10.69 | 0.74 | 0.33 | 3.43 | 3.45 |
| SR-01000269911 | 898.2E−9 | 88.11 ± 5.39 | 1.69 | 0.37 | 3.22 | 3.22 |
| SR-00000004378 | 914.1E−9 | 72.99 ± 14.39 | 0.72 | 0.34 | 4.17 | 4.84 |
| SR-01000921162 | 918.9E−9 | 88.33 ± 16.11 | 0.90 | 0.27 | 3.84 | 3.84 |
| SR-01000151470 | 924.6E−9 | 78.27 ± 16.46 | 1.14 | 0.31 | 2.62 | 2.62 |
| SR-00000003277 | 928.5E−9 | 97.54 ± 11.30 | 1.48 | 0.34 | 2.32 | 3.23 |
| SR-01000320687 | 929.5E−9 | 65.23 ± 8.63 | 0.93 | 0.56 | 2.98 | 5.53 |
| SR-01000925700 | 931.4E−9 | 83.76 ± 7.63 | 0.44 | 0.32 | 3.09 | 4.08 |
| SR-01000526835 | 955.4E−9 | 81.65 ± 6.38 | 1.15 | 0.37 | 3.01 | 3.01 |
| SR-01000358286 | 971.3E−9 | 88.73 ± 9.49 | 1.77 | 0.31 | 2.95 | 2.95 |
| SR-01000054961 | 979.7E−9 | 79.59 ± 15.93 | 0.85 | 0.26 | 2.43 | 2.43 |
| SR-01000126429 | 1.E−6 | 78.64 ± 16.53 | 1.58 | 0.29 | 3.90 | 3.90 |
| SR-01000124163 | 1.E−6 | 90.02 ± 28.06 | 1.37 | 0.36 | 2.59 | 2.59 |
| SR-01000072797 | 1.E−6 | 94.94 ± 8.96 | 0.87 | 0.29 | 2.52 | 2.52 |
| SR-01000068179 | 1.1E−6 | 87.03 ± 9.26 | 0.84 | 0.31 | 3.64 | 3.64 |
| SR-01000153956 | 1.1E−6 | 93.85 ± 32.13 | 1.89 | 0.38 | 2.80 | 2.80 |
| SR-01000318521 | 1.2E−6 | 72.38 ± 5.96 | 0.97 | 0.36 | 2.39 | 2.40 |
| SR-01000312779 | 1.2E−6 | 81.17 ± 17.61 | 1.91 | 0.30 | 2.74 | 2.77 |
| SR-01000923250 | 1.2E−6 | 67.60 ± 3.63 | 0.64 | 0.35 | 1.86 | 1.87 |
| SR-01000153886 | 1.2E−6 | 90.96 ± 29.33 | 2.51 | 0.41 | 3.22 | 3.22 |
| SR-01000299610 | 1.2E−6 | 71.34 ± 19.37 | 0.79 | 0.33 | 3.88 | 3.88 |
| SR-01000335115 | 1.3E−6 | 86.17 ± 9.84 | 1.29 | 0.38 | 3.37 | 3.37 |
| SR-01000145456 | 1.3E−6 | 88.21 ± 16.68 | 1.42 | 0.32 | 2.66 | 2.66 |
| SR-01000185621 | 1.3E−6 | 81.20 ± 9.68 | 0.84 | 0.28 | 2.73 | 2.73 |
| SR-01000267161 | 1.3E−6 | 105.03 ± 11.34 | 0.55 | 0.33 | 1.49 | 1.49 |
| SR-01000025120 | 1.3E−6 | 76.46 ± 7.01 | 0.43 | 0.36 | 5.56 | 10.50 |
| SR-00000007044 | 1.4E−6 | 81.04 ± 10.01 | 1.19 | 0.24 | 3.11 | 5.00 |
| SR-01000193756 | 1.4E−6 | 75.38 ± 12.05 | 0.69 | 0.30 | 4.01 | 4.01 |
| SR-01000406098 | 1.4E−6 | 80.99 ± 4.70 | 1.97 | 0.24 | 2.46 | 2.46 |
| SR-01000357697 | 1.5E−6 | 87.67 ± 13.61 | 1.75 | 0.31 | 4.74 | 4.74 |
| SR-01000579592 | >1.5E−6 | 96.78 ± 10.47 | | 0.28 | 1.75 | 1.75 |
| SR-01000028953 | 1.5E−6 | 88.05 ± 4.24 | 0.80 | 0.54 | 2.53 | 6.30 |
| SR-01000336681 | 1.6E−6 | 81.02 ± 6.69 | 1.92 | 0.26 | 3.33 | 3.33 |
| SR-01000143463 | 1.6E−6 | 85.31 ± 30.99 | 1.05 | 0.29 | 2.57 | 2.84 |
| SR-01000363853 | 1.6E−6 | 87.50 ± 15.37 | 0.77 | 0.29 | 2.73 | 2.73 |
| SR-01000362228 | 1.6E−6 | 84.08 ± 9.93 | 1.38 | 0.30 | 3.10 | 3.11 |
| SR-01000062305 | >1.6E−6 | 121.63 ± 9.56 | | 0.27 | 2.66 | 2.66 |
| SR-01000634249 | 1.7E−6 | 89.30 ± 20.10 | 0.69 | 0.26 | 2.57 | 2.57 |
| SR-01000248261 | 1.7E−6 | 81.40 ± 9.66 | 1.06 | 0.30 | 2.28 | 2.28 |
| SR-01000550322 | 1.8E−6 | 86.57 ± 2.15 | 0.73 | 0.27 | 2.87 | 2.87 |
| SR-01000098884 | 1.8E−6 | 75.62 ± 16.43 | 0.72 | 0.35 | 4.12 | 4.12 |
| SR-05000022582 | 1.8E−6 | 135.48 ± 20.60 | 0.53 | 0.31 | 0.87 | 2.32 |
| SR-01000484494 | 1.9E−6 | 114.96 ± 19.78 | 0.89 | 0.28 | 1.82 | 1.82 |
| SR-00000005410 | 1.9E−6 | 99.92 ± 5.04 | 1.14 | 0.31 | 2.93 | 2.93 |
| SR-01000124392 | 2.E−6 | 80.01 ± 6.26 | 0.49 | 0.28 | 3.17 | 3.17 |
| SR-01000126251 | 2.E−6 | 121.39 ± 22.69 | 1.11 | 0.30 | 3.41 | 3.41 |
| SR-01000361172 | 2.E−6 | 91.50 ± 11.81 | 1.48 | 0.40 | 2.78 | 2.81 |
| SR-01000011241 | 2.E−6 | 63.10 ± 2.62 | 1.07 | 0.26 | 1.89 | 1.89 |
| SR-01000325257 | 2.3E−6 | 66.69 ± 11.44 | 0.69 | 0.29 | 3.22 | 3.27 |
| SR-01000911742 | 2.3E−6 | 93.88 ± 11.00 | 0.32 | 0.25 | 2.28 | 2.28 |
| SR-01000429212 | 2.3E−6 | 80.96 ± 18.51 | 0.88 | 0.34 | 1.94 | 1.94 |
| SR-01000136273 | 2.5E−6 | 78.44 ± 11.98 | 0.56 | 0.31 | 2.47 | 2.47 |
| SR-01000921037 | 2.9E−6 | 75.54 ± 11.00 | 0.88 | 0.25 | 3.81 | 3.85 |
| SR-01000119565 | 3.E−6 | 68.12 ± 14.87 | 1.07 | 0.30 | 3.46 | 3.46 |
| SR-01000017533 | 3.2E−6 | 69.12 ± 3.13 | 1.55 | 0.31 | 3.33 | 3.33 |
| SR-01000299370 | 3.7E−6 | 68.10 ± 8.73 | 0.74 | 0.38 | 1.36 | 1.36 |
| SR-01000465824 | 3.8E−6 | 68.48 ± 7.96 | 1.13 | 0.27 | 2.16 | 2.21 |
| SR-01000360839 | 4.1E−6 | 57.17 ± 6.17 | 1.91 | 0.28 | 3.13 | 4.95 |
| SR-01000597171 | >4.4E−6 | 177.62 ± 11.26 | | 0.47 | 3.74 | 3.74 |
| SR-01000141798 | 4.6E−6 | 62.31 ± 7.50 | 0.65 | 0.31 | 3.15 | 3.15 |

| | HEK Agonist Activity | | | | | |
|---|---|---|---|---|---|---|
| | | Averaged | | | | |
| | | Max % | | | Lipophilic | Lipophilic |
| | | Response | Hill | Efficiency | Efficiency | Efficiency |
| compound | EC₅₀ | (±St. Dev.) | Slope | Ligand | (logP) | (logD) |
| SR-01000410744 | >10.2E−6 | 17.10 ± 14.93 | | 0.29 | 0.95 | 0.98 |
| SR-01000520454 | >13.8E−6 | 15.83 ± 2.96 | | 0.28 | 1.19 | 1.31 |
| SR-01000907288 | >14.1E−6 | 14.88 ± 7.51 | | 0.26 | 1.28 | 1.28 |
| SR-01000295701 | >14.1E−6 | −4.01 ± 2.60 | | 0.30 | 1.17 | 1.19 |
| SR-01000093460 | 11.E−6 | 109.44 ± 22.66 | 15.09 | 0.26 | 1.85 | 1.85 |
| SR-01000095280 | >14.1E−6 | 25.01 ± 8.12 | | 0.22 | 2.38 | 2.38 |
| SR-01000068729 | >15.3E−6 | −3.41 ± 1.08 | | 0.23 | 1.73 | 1.73 |
| SR-01000031674 | >15.E−6 | 35.54 ± 2.15 | | 0.27 | 2.46 | 2.46 |
| SR-01000140260 | >14.1E−6 | 19.72 ± 7.61 | | 0.28 | 2.84 | 2.84 |
| SR-07000000296 | >14.1E−6 | 16.33 ± 1.82 | | 0.27 | −2.00 | −2.00 |
| SR-01000138428 | >14.1E−6 | −0.31 ± 7.41 | | 0.21 | 1.03 | 1.03 |
| SR-01000918716 | 5.5E−6 | 92.04 ± 16.23 | 0.85 | 0.26 | 2.34 | 2.34 |
| SR-01000205482 | >14.1E−6 | −5.45 ± 10.43 | | 0.26 | 1.52 | 1.53 |
| SR-01000071222 | >14.7E−6 | 4.62 ± 5.04 | | 0.23 | 2.01 | 2.01 |
| SR-01000155701 | >14.1E−6 | 40.29 ± 5.74 | | 0.28 | 3.36 | 3.36 |
| SR-01000068229 | >15.4E−6 | −2.42 ± 7.08 | | 0.24 | 3.29 | 3.29 |
| SR-01000069345 | >14.7E−6 | 15.34 ± 2.20 | | 0.28 | 0.77 | 0.77 |
| SR-01000150430 | >14.1E−6 | 30.94 ± 8.86 | | 0.28 | 2.75 | 2.75 |
| SR-01000356497 | >14.1E−6 | 2.54 ± 3.30 | | 0.27 | 1.48 | 1.52 |
| SR-01000299915 | >14.1E−6 | 2.39 ± 2.18 | | 0.32 | 2.40 | 2.40 |
| SR-01000137723 | >14.1E−6 | −4.03 ± 1.74 | | 0.31 | 1.70 | 1.70 |
| SR-01000583842 | >12.6E−6 | 1.44 ± 5.47 | | 0.20 | 1.58 | 1.58 |
| SR-01000369718 | >14.1E−6 | 7.35 ± 13.00 | | 0.28 | 1.98 | 1.98 |
| SR-01000412367 | >13.E−6 | 5.28 ± 6.57 | | 0.30 | 1.11 | 1.11 |
| SR-01000077904 | >14.1E−6 | −12.58 ± 9.87 | | 0.24 | 1.20 | 1.20 |
| SR-01000146606 | >14.1E−6 | −12.06 ± 19.17 | | 0.23 | 1.22 | 1.22 |
| SR-01000069259 | >14.2E−6 | 7.00 ± 9.99 | | 0.22 | 0.40 | 0.40 |
| SR-01000339494 | >14.1E−6 | 13.92 ± 7.54 | | 0.23 | 1.97 | 1.97 |
| SR-01000073202 | >14.5E−6 | 12.21 ± 5.38 | | 0.23 | 1.04 | 1.04 |
| SR-01000633083 | >14.1E−6 | 21.09 ± 6.01 | | 0.23 | 1.48 | 1.49 |
| SR-01000048841 | >14.8E−6 | 15.49 ± 13.53 | | 0.24 | 2.25 | 2.25 |
| SR-01000911654 | >14.1E−6 | −5.46 ± 4.05 | | 0.26 | 1.69 | 1.69 |
| SR-00000006339 | >14.1E−6 | 5.16 ± 8.02 | | 0.21 | 1.83 | 2.91 |
| SR-01000068761 | >14.7E−6 | 4.98 ± 10.48 | | 0.25 | 1.81 | 1.82 |
| SR-01000074712 | >14.7E−6 | 30.27 ± 2.23 | | 0.24 | 1.73 | 1.73 |
| SR-01000068718 | >14.9E−6 | 20.24 ± 11.96 | | 0.24 | 3.34 | 3.34 |
| SR-01000144615 | >14.1E−6 | −9.40 ± 4.48 | | 0.21 | 3.24 | 3.29 |
| SR-01000039980 | >15.3E−6 | 39.53 ± 8.33 | | 0.20 | 0.84 | 0.84 |
| SR-01000652673 | >13.4E−6 | 3.95 ± 2.97 | | 0.27 | 2.61 | 2.61 |
| SR-01000202338 | >14.1E−6 | 4.74 ± 3.91 | | 0.30 | 1.76 | 1.76 |
| SR-01000070891 | >15.2E−6 | 8.31 ± 6.92 | | 0.21 | 1.83 | 1.86 |
| SR-01000061624 | >14.1E−6 | 13.43 ± 5.07 | | 0.26 | 0.93 | 0.94 |
| SR-01000317486 | >14.1E−6 | 3.86 ± 3.87 | | 0.27 | 1.64 | 1.64 |
| SR-01000013313 | >14.4E−6 | 2.95 ± 4.76 | | 0.29 | 1.40 | 1.41 |
| SR-01000070591 | >14.3E−6 | −5.74 ± 0.47 | | 0.24 | 1.00 | 1.00 |
| SR-05000709044 | >14.1E−6 | −2.44 ± 4.17 | | 0.15 | 3.94 | 3.96 |
| SR-01000912290 | >14.1E−6 | 20.29 ± 5.75 | | 0.27 | 3.12 | 3.12 |
| SR-01000150679 | >14.1E−6 | 8.79 ± 12.00 | | 0.23 | 3.36 | 3.36 |
| SR-01000146442 | >14.1E−6 | −6.48 ± 10.66 | | 0.25 | 1.82 | 1.82 |
| SR-01000137757 | >14.1E−6 | −12.95 ± 5.94 | | 0.30 | 1.35 | 1.35 |
| SR-01000191059 | >14.1E−6 | −1.76 ± 2.36 | | 0.23 | 1.61 | 1.61 |
| SR-01000239973 | >14.1E−6 | −7.52 ± 3.99 | | 0.24 | 2.16 | 2.16 |
| SR-01000159110 | >14.1E−6 | 3.44 ± 10.60 | | 0.28 | 2.25 | 2.25 |
| SR-01000060349 | >15.1E−6 | 36.27 ± 9.28 | | 0.31 | 2.30 | 2.34 |
| SR-01000524029 | >16.E−6 | 9.45 ± 2.06 | | 0.35 | 1.61 | 1.61 |
| SR-01000061818 | >14.5E−6 | 3.00 ± 9.38 | | 0.31 | 1.46 | 1.46 |
| SR-01000535824 | >12.1E−6 | 34.71 ± 16.19 | | 0.25 | −0.133 | −0.133 |
| SR-01000088477 | >14.1E−6 | −0.30 ± 8.57 | | 0.30 | 1.26 | 1.26 |
| SR-01000048417 | >14.3E−6 | −4.34 ± 6.43 | | 0.23 | 1.80 | 1.80 |
| SR-01000069564 | >14.4E−6 | −3.54 ± 8.88 | | 0.23 | 1.93 | 1.93 |
| SR-01000358013 | >14.1E−6 | 22.28 ± 14.14 | | 0.25 | 1.64 | 1.68 |
| SR-01000194602 | >14.1E−6 | 4.93 ± 4.93 | | 0.24 | 1.58 | 1.58 |
| SR-01000525232 | >17.4E−6 | −17.88 ± 3.50 | | 0.35 | 1.09 | 1.09 |
| SR-01000176150 | >14.1E−6 | −3.65 ± 2.66 | | 0.25 | 3.17 | 3.48 |
| SR-01000134029 | >14.1E−6 | 19.31 ± 4.61 | | 0.21 | 1.41 | 1.41 |
| SR-01000504687 | >12.2E−6 | −3.10 ± 6.59 | | 0.25 | 1.65 | 1.65 |
| SR-05000022531 | >14.1E−6 | 1.20 ± 11.04 | | 0.23 | 0.61 | 0.61 |
| SR-01000104567 | >14.1E−6 | 8.02 ± 5.87 | | 0.25 | 2.34 | 2.34 |
| SR-01000918718 | >14.1E−6 | 16.28 ± 3.44 | | 0.27 | 3.62 | 3.62 |
| SR-01000145949 | >14.1E−6 | 2.57 ± 15.21 | | 0.23 | 2.92 | 2.92 |
| SR-01000183606 | >14.1E−6 | −9.57 ± 3.95 | | 0.23 | 1.16 | 1.16 |
| SR-01000098944 | >14.1E−6 | 16.33 ± 3.15 | | 0.27 | 1.42 | 1.42 |
| SR-01000177515 | >14.1E−6 | 24.98 ± 4.71 | | 0.23 | 2.87 | 3.12 |
| SR-01000077930 | >14.1E−6 | 1.00 ± 2.79 | | 0.24 | 1.76 | 1.76 |
| SR-01000356020 | >14.1E−6 | 1.37 ± 3.30 | | 0.27 | 1.48 | 1.52 |
| SR-01000058107 | >14.9E−6 | 4.84 ± 7.51 | | 0.27 | 2.55 | 2.55 |
| SR-01000923125 | >14.1E−6 | 32.19 ± 4.93 | | 0.23 | 1.53 | 1.53 |
| SR-01000276878 | >14.1E−6 | 6.40 ± 8.50 | | 0.24 | 1.58 | 1.62 |
| SR-01000918721 | >14.1E−6 | 9.27 ± 15.15 | | 0.24 | 2.96 | 2.96 |
| SR-01000099472 | >14.1E−6 | 10.79 ± 6.86 | | 0.25 | 2.60 | 2.60 |
| SR-01000155068 | >14.1E−6 | −2.13 ± 1.88 | | 0.26 | 2.28 | 2.28 |
| SR-00000007138 | >14.1E−6 | 10.53 ± 4.93 | | 0.20 | 1.76 | 3.12 |
| SR-01000071098 | >14.1E−6 | 24.54 ± 7.58 | | 0.25 | 1.37 | 1.38 |
| SR-01000919746 | >14.1E−6 | −13.54 ± 17.63 | | 0.24 | 1.19 | 1.19 |
| SR-01000021416 | >14.1E−6 | 23.13 ± 4.18 | | 0.28 | 0.84 | 0.84 |
| SR-01000146420 | >14.1E−6 | 20.27 ± 3.59 | | 0.24 | 1.61 | 1.61 |
| SR-01000298170 | >14.1E−6 | 10.66 ± 2.50 | | 0.30 | 1.44 | 1.44 |
| SR-01000037440 | >14.4E−6 | 16.47 ± 8.15 | | 0.27 | 0.95 | 0.95 |
| SR-01000064440 | >15.0E−6 | 26.24 ± 0.88 | | 0.22 | 1.81 | 1.81 |
| SR-01000362293 | >14.1E−6 | −7.08 ± 3.92 | | 0.30 | 1.45 | 2.39 |
| SR-01000223498 | >14.1E−6 | 15.10 ± 2.28 | | 0.25 | 1.43 | 1.43 |
| SR-01000012205 | >14.5E−6 | 1.53 ± 2.99 | | 0.23 | 1.43 | 1.43 |
| SR-01000068721 | >15.2E−6 | 8.82 ± 10.34 | | 0.26 | 2.68 | 2.68 |
| SR-01000125458 | >14.1E−6 | −1.02 ± 2.16 | | 0.26 | 1.94 | 1.94 |
| SR-01000589950 | >14.1E−6 | −9.62 ± 21.31 | | 0.23 | 3.39 | 3.39 |
| SR-01000048367 | >16.0E−6 | 8.71 ± 14.83 | | 0.29 | 3.00 | 3.00 |
| SR-01000584158 | >14.1E−6 | 0.77 ± 6.52 | | 0.24 | −0.34 | −0.34 |
| SR-01000923247 | >14.1E−6 | 20.62 ± 4.90 | | 0.28 | 0.63 | 0.63 |
| SR-01000048295 | >14.3E−6 | 7.53 ± 4.49 | | 0.26 | 1.86 | 1.86 |
| SR-01000020502 | >13.9E−6 | 4.84 ± 4.90 | | 0.21 | 0.13 | 0.13 |
| SR-01000071221 | >15.6E−6 | 19.19 ± 7.12 | | 0.34 | 0.08 | 0.08 |
| SR-01000583473 | 7.2E−6 | 54.64 ± 8.92 | 0.45 | 0.16 | 1.46 | 1.64 |
| SR-01000921178 | >14.1E−6 | 20.98 ± 11.45 | | 0.23 | 2.44 | 2.44 |
| SR-01000058120 | >14.3E−6 | 11.91 ± 6.77 | | 0.24 | 2.18 | 2.18 |
| SR-01000066950 | >13.8E−6 | 2.95 ± 8.11 | | 0.23 | 1.21 | 1.21 |
| SR-01000290411 | >14.1E−6 | 2.70 ± 2.28 | | 0.27 | 1.17 | 2.11 |
| SR-01000069642 | >14.8E−6 | 8.55 ± 4.45 | | 0.27 | 2.45 | 2.45 |
| SR-00000007279 | >14.1E−6 | 17.20 ± 7.85 | | 0.25 | 2.29 | 2.30 |
| SR-01000926956 | >14.1E−6 | −3.24 ± 9.86 | | 0.24 | 2.87 | 2.87 |
| SR-01000041378 | >14.6E−6 | −2.48 ± 2.57 | | 0.23 | 1.79 | 1.79 |
| SR-01000125584 | >14.1E−6 | 0.37 ± 17.31 | | 0.25 | 2.11 | 2.11 |
| SR-01000337370 | >14.1E−6 | 23.00 ± 5.22 | | 0.23 | 3.14 | 3.15 |
| SR-01000916271 | >14.1E−6 | 29.38 ± 9.84 | | 0.25 | 2.35 | 2.35 |
| SR-01000583192 | >15.9E−6 | 13.45 ± 13.24 | | 0.23 | 2.09 | 2.09 |
| SR-01000918720 | >14.1E−6 | 24.77 ± 3.77 | | 0.24 | 3.10 | 3.10 |
| SR-01000090996 | >14.1E−6 | −5.40 ± 6.26 | | 0.23 | 2.07 | 2.07 |
| SR-01000551463 | >12.2E−6 | 18.60 ± 9.91 | | 0.22 | 0.71 | 0.71 |
| SR-01000135038 | >14.1E−6 | 13.01 ± 4.86 | | 0.28 | 2.87 | 2.87 |
| SR-01000056458 | >14.1E−6 | 19.25 ± 6.92 | | 0.23 | 2.66 | 2.66 |
| SR-01000915644 | >14.1E−6 | −2.94 ± 0.56 | | 0.27 | 1.25 | 1.25 |
| SR-01000045673 | >14.3E−6 | 4.34 ± 4.74 | | 0.23 | 1.24 | 1.24 |
| SR-01000579790 | >11.6E−6 | 15.88 ± 4.74 | | 0.22 | 0.42 | 0.42 |
| SR-01000129880 | >14.1E−6 | 0.02 ± 5.29 | | 0.21 | 1.21 | 1.22 |
| SR-01000546462 | >15.1E−6 | 0.58 ± 3.78 | | 0.17 | 0.56 | 0.56 |
| SR-01000071178 | >14.2E−6 | 2.89 ± 1.24 | | 0.21 | 2.58 | 2.58 |
| SR-01000021435 | >13.9E−6 | 24.48 ± 11.03 | | 0.28 | 1.22 | 1.22 |
| SR-01000918134 | >14.1E−6 | −11.92 ± 3.45 | | 0.22 | 0.66 | 0.66 |
| SR-01000073564 | >14.5E−6 | 8.96 ± 14.03 | | 0.28 | 2.07 | 2.07 |
| SR-01000062290 | >14.9E−6 | 11.38 ± 4.42 | | 0.24 | 1.10 | 1.34 |
| SR-01000077903 | >14.1E−6 | 1.27 ± 2.74 | | 0.25 | 1.29 | 1.29 |
| SR-01000151666 | >14.1E−6 | −3.95 ± 11.76 | | 0.23 | 1.87 | 1.87 |
| SR-01000218376 | >14.1E−6 | 1.93 ± 7.79 | | 0.40 | 1.45 | 1.45 |
| SR-01000069451 | >14.4E−6 | 25.95 ± 1.00 | | 0.26 | 3.83 | 3.83 |
| SR-01000064879 | >14.7E−6 | 9.06 ± 10.70 | | 0.22 | 3.29 | 3.29 |
| SR-01000135068 | >14.1E−6 | −6.35 ± 1.52 | | 0.27 | 1.72 | 1.72 |
| SR-01000069439 | >14.9E−6 | 1.51 ± 2.29 | | 0.31 | 1.55 | 1.55 |
| SR-01000916033 | >14.1E−6 | 25.93 ± 6.51 | | 0.28 | 2.68 | 2.68 |
| SR-00000005955 | >14.1E−6 | 18.59 ± 8.23 | | 0.26 | 1.73 | 1.73 |
| SR-01000124399 | >14.1E−6 | −5.54 ± 8.42 | | 0.26 | 2.15 | 2.15 |
| SR-01000923249 | >14.1E−6 | 25.04 ± 3.96 | | 0.25 | 1.46 | 1.46 |
| SR-01000188699 | >14.1E−6 | 4.63 ± 6.30 | | 0.31 | 2.02 | 2.02 |
| SR-01000069435 | >14.6E−6 | 14.76 ± 3.78 | | 0.24 | 3.08 | 3.09 |
| SR-01000297192 | >14.1E−6 | 12.75 ± 16.65 | | 0.32 | 3.24 | 3.25 |
| SR-01000906953 | 5.7E−6 | 192.43 ± 25.16 | 9.35 | 0.26 | 1.23 | 1.23 |
| SR-01000021433 | >14.3E−6 | 27.04 ± 4.04 | | 0.31 | 2.29 | 2.29 |
| SR-01000152365 | >14.1E−6 | 18.44 ± 8.07 | | 0.28 | 2.49 | 2.49 |
| SR-01000080408 | 11.9E−6 | 71.97 ± 7.16 | 1.95 | 0.31 | 2.51 | 4.10 |
| SR-01000074687 | >15.4E−6 | 17.98 ± 4.87 | | 0.31 | 1.32 | 1.33 |
| SR-01000029575 | >14.3E−6 | 7.49 ± 5.72 | | 0.26 | 1.08 | 1.09 |
| SR-01000135037 | >14.1E−6 | −6.83 ± 12.52 | | 0.22 | 1.69 | 1.70 |
| SR-01000336035 | >14.1E−6 | −5.99 ± 6.97 | | 0.25 | 4.40 | 4.40 |
| SR-01000054849 | >14.8E−6 | 1.19 ± 3.75 | | 0.24 | 1.41 | 1.41 |
| SR-01000135070 | >14.1E−6 | 7.87 ± 13.27 | | 0.26 | 1.93 | 1.93 |
| SR-01000484454 | >11.9E−6 | 27.15 ± 8.69 | | 0.20 | −0.16 | −0.16 |
| SR-01000652579 | >14.2E−6 | 11.17 ± 6.82 | | 0.25 | 1.08 | 1.08 |
| SR-01000065653 | >14.1E−6 | −8.07 ± 6.14 | | 0.23 | 1.87 | 1.88 |
| SR-01000126715 | >14.1E−6 | 5.89 ± 8.45 | | 0.28 | 1.95 | 1.95 |
| SR-01000048373 | >14.3E−6 | 9.79 ± 7.30 | | 0.23 | 1.79 | 1.79 |
| SR-01000146444 | >14.1E−6 | 20.98 ± 2.11 | | 0.24 | 1.38 | 1.38 |
| SR-01000067982 | >14.8E−6 | 10.05 ± 3.22 | | 0.27 | 1.37 | 1.37 |
| SR-01000068916 | >15.3E−6 | 19.72 ± 5.30 | | 0.29 | 2.04 | 2.04 |
| SR-01000410694 | >10.2E−6 | 10.08 ± 3.68 | | 0.29 | 0.95 | 0.98 |
| SR-01000318010 | >14.1E−6 | 12.20 ± 3.74 | | 0.26 | 1.15 | 1.19 |
| SR-00000007008 | >14.1E−6 | −8.12 ± 2.24 | | 0.25 | 1.85 | 1.85 |
| SR-01000074761 | >15.4E−6 | 13.40 ± 6.99 | | 0.23 | 3.15 | 3.15 |
| SR-01000021418 | >14.5E−6 | 34.36 ± 2.33 | | 0.31 | 1.66 | 1.66 |
| SR-01000309617 | >14.4E−6 | −3.26 ± 4.40 | | 0.28 | 1.67 | 1.74 |
| SR-01000582959 | >11.3E−6 | 1.95 ± 4.15 | | 0.22 | 1.66 | 1.66 |
| SR-01000393531 | >13.4E−6 | −0.87 ± 7.51 | | 0.28 | 1.80 | 2.74 |
| SR-01000142419 | >14.1E−6 | −9.79 ± 12.86 | | 0.25 | 2.10 | 2.10 |
| SR-01000069699 | >15.2E−6 | 12.32 ± 5.16 | | 0.26 | 2.03 | 2.03 |
| SR-01000654192 | >13.3E−6 | 14.64 ± 17.11 | | 0.19 | 1.29 | 1.42 |
| SR-01000150393 | >14.1E−6 | 10.10 ± 9.60 | | 0.22 | 1.43 | 1.45 |
| SR-01000370374 | >14.1E−6 | 4.76 ± 6.67 | | 0.27 | 1.66 | 1.71 |
| SR-01000145393 | >14.1E−6 | 10.41 ± 2.89 | | 0.28 | 2.34 | 2.34 |
| SR-01000273754 | >14.1E−6 | 2.38 ± 11.60 | | 0.36 | 1.95 | 1.95 |
| SR-01000021414 | >14.3E−6 | 22.31 ± 11.03 | | 0.31 | 2.38 | 2.38 |
| SR-00000007518 | >14.1E−6 | 23.13 ± 2.30 | | 0.21 | 1.58 | 2.64 |
| SR-01000189442 | >14.1E−6 | 9.63 ± 8.04 | | 0.22 | 1.69 | 1.69 |
| SR-01000098976 | >14.1E−6 | 25.21 ± 6.04 | | 0.27 | 2.53 | 2.53 |
| SR-01000185712 | >14.1E−6 | 9.02 ± 5.86 | | 0.21 | 0.41 | 0.41 |
| SR-01000124129 | >14.1E−6 | −9.08 ± 5.27 | | 0.36 | 1.77 | 1.77 |
| SR-00000004118 | >14.1E−6 | 13.55 ± 13.03 | | 0.22 | 1.78 | 3.11 |
| SR-01000068608 | >14.3E−6 | 24.17 ± 25.98 | | 0.24 | 2.46 | 2.46 |
| SR-01000926263 | >14.1E−6 | 12.91 ± 4.81 | | 0.27 | 2.29 | 2.29 |
| SR-01000152429 | >14.1E−6 | −0.18 ± 21.55 | | 0.28 | 2.49 | 2.49 |
| SR-00000002785 | >14.1E−6 | −10.50 ± 3.98 | | 0.23 | 2.62 | 2.62 |
| SR-01000197834 | >14.1E−6 | 18.65 ± 10.85 | | 0.38 | 2.58 | 4.95 |
| SR-01000286062 | >14.1E−6 | 8.31 ± 5.73 | | 0.34 | 2.20 | 2.20 |
| SR-01000655128 | >14.1E−6 | 0.74 ± 3.27 | | 0.25 | 1.68 | 1.68 |
| SR-01000151876 | >14.1E−6 | −3.95 ± 8.40 | | 0.31 | 2.50 | 2.50 |
| SR-00000004451 | >14.1E−6 | 3.04 ± 3.40 | | 0.23 | 2.31 | 4.36 |
| SR-01000034566 | >15.2E−6 | −4.69 ± 7.66 | | 0.25 | 2.60 | 2.60 |
| SR-01000129845 | >14.1E−6 | 8.19 ± 5.50 | | 0.23 | 1.95 | 1.95 |
| SR-01000012264 | >14.3E−6 | 3.88 ± 6.42 | | 0.28 | 1.34 | 1.35 |
| SR-01000316766 | >14.1E−6 | −0.94 ± 3.21 | | 0.23 | 1.14 | 1.14 |
| SR-01000145157 | >14.1E−6 | 17.61 ± 4.99 | | 0.26 | 1.85 | 1.85 |
| SR-01000125460 | >14.1E−6 | −2.02 ± 12.81 | | 0.27 | 2.02 | 2.02 |
| SR-01000091502 | >14.1E−6 | −12.00 ± 4.68 | | 0.22 | 2.59 | 2.63 |
| SR-01000075116 | >14.3E−6 | 4.31 ± 1.95 | | 0.26 | 2.72 | 2.72 |
| SR-01000918719 | >14.1E−6 | 19.77 ± 4.52 | | 0.26 | 3.17 | 3.17 |
| SR-01000125464 | >14.1E−6 | 5.57 ± 17.96 | | 0.25 | 1.74 | 1.74 |
| SR-01000101329 | >14.1E−6 | −2.50 ± 6.06 | | 0.27 | 3.16 | 3.17 |
| SR-01000319951 | >14.1E−6 | 7.14 ± 13.31 | | 0.30 | 2.54 | 2.60 |
| SR-01000545824 | >13.6E−6 | −0.19 ± 3.69 | | 0.17 | −0.052 | −0.052 |
| SR-01000301649 | >14.1E−6 | 14.38 ± 10.96 | | 0.26 | 1.93 | 1.94 |
| SR-01000074154 | >15.E−6 | 11.86 ± 8.71 | | 0.23 | 3.20 | 3.21 |
| SR-01000070509 | >14.3E−6 | 15.75 ± 7.96 | | 0.21 | 2.31 | 2.32 |
| SR-01000134947 | >14.1E−6 | 1.42 ± 10.91 | | 0.27 | 2.07 | 2.07 |
| SR-01000088420 | >14.1E−6 | 12.56 ± 5.54 | | 0.30 | 1.33 | 1.33 |
| SR-01000914970 | >14.1E−6 | −5.66 ± 4.95 | | 0.36 | 2.02 | 2.03 |
| SR-01000125462 | >14.1E−6 | 6.02 ± 12.03 | | 0.26 | 1.58 | 1.58 |
| SR-01000100493 | >14.1E−6 | 4.54 ± 3.12 | | 0.31 | 1.65 | 1.65 |
| SR-01000190122 | >14.1E−6 | 0.66 ± 6.28 | | 0.21 | 1.27 | 1.27 |
| SR-01000918303 | >14.1E−6 | −6.52 ± 9.86 | | 0.25 | 2.37 | 2.37 |
| SR-01000069391 | >15.5E−6 | 12.32 ± 6.09 | | 0.28 | 2.43 | 2.43 |
| SR-01000131962 | >14.1E−6 | −5.39 ± 5.10 | | 0.24 | 2.37 | 2.37 |
| SR-01000042100 | >14.4E−6 | 17.67 ± 3.11 | | 0.32 | 2.20 | 2.20 |
| SR-01000913573 | >14.1E−6 | 8.58 ± 4.44 | | 0.28 | 0.84 | 0.84 |
| SR-01000152760 | >14.1E−6 | 0.48 ± 8.40 | | 0.27 | 2.88 | 2.88 |
| SR-01000134941 | >14.1E−6 | 10.65 ± 6.79 | | 0.28 | 2.40 | 2.40 |
| SR-01000207758 | >14.1E−6 | 2.94 ± 8.83 | | 0.23 | 1.92 | 1.92 |
| SR-01000103876 | >14.1E−6 | 1.17 ± 3.96 | | 0.30 | 2.60 | 2.61 |
| SR-01000098920 | >14.1E−6 | 10.87 ± 9.19 | | 0.26 | 1.13 | 1.13 |
| SR-01000052065 | >15.2E−6 | 8.92 ± 9.62 | | 0.23 | 2.62 | 2.71 |
| SR-01000114500 | >14.1E−6 | 4.75 ± 6.36 | | 0.24 | 2.32 | 2.32 |
| SR-00000005389 | >14.1E−6 | 31.02 ± 4.14 | | 0.21 | 1.38 | 2.79 |
| SR-01000150389 | >14.1E−6 | 0.25 ± 10.83 | | 0.27 | 2.56 | 2.56 |
| SR-00000005347 | >14.1E−6 | 11.55 ± 3.19 | | 0.28 | 2.23 | 2.23 |
| SR-01000654639 | >14.E−6 | 1.22 ± 5.69 | | 0.24 | 0.64 | 0.64 |
| SR-01000299442 | >14.1E−6 | −11.97 ± 11.27 | | 0.27 | 1.60 | 1.60 |
| SR-01000150449 | >14.1E−6 | −1.70 ± 7.28 | | 0.27 | 2.46 | 2.46 |
| SR-01000146516 | >14.1E−6 | −2.96 ± 7.51 | | 0.26 | 3.75 | 3.75 |
| SR-01000187393 | >14.1E−6 | 6.35 ± 6.79 | | 0.23 | 2.44 | 2.44 |
| SR-01000284749 | >14.1E−6 | 9.50 ± 11.03 | | 0.24 | 0.40 | 0.40 |
| SR-01000361779 | >14.1E−6 | 9.57 ± 4.06 | | 0.26 | 1.50 | 1.53 |
| SR-01000124135 | >14.1E−6 | 7.54 ± 4.78 | | 0.32 | 0.91 | 0.91 |
| SR-01000187428 | >14.1E−6 | 6.44 ± 5.26 | | 0.23 | 1.59 | 1.59 |
| SR-00000005253 | >14.1E−6 | −10.69 ± 5.73 | | 0.28 | 2.23 | 2.23 |
| SR-01000062726 | >14.5E−6 | −12.60 ± 9.55 | | 0.23 | 2.40 | 2.40 |
| SR-01000243797 | >14.1E−6 | 10.10 ± 12.81 | | 0.28 | 1.25 | 1.25 |
| SR-07000002775 | >14.1E−6 | 2.05 ± 12.71 | | 0.25 | −0.064 | −0.064 |
| SR-01000125622 | >14.1E−6 | −10.20 ± 10.64 | | 0.25 | 1.80 | 1.80 |
| SR-01000318079 | >14.1E−6 | 11.69 ± 6.13 | | 0.22 | 1.94 | 1.96 |
| SR-01000113198 | >14.1E−6 | 10.00 ± 6.18 | | 0.27 | 2.53 | 2.53 |
| SR-01000273778 | >14.1E−6 | 11.17 ± 5.52 | | 0.26 | 0.92 | 1.13 |
| SR-01000582958 | >13.4E−6 | 10.03 ± 5.09 | | 0.24 | 3.51 | 3.51 |
| SR-01000144627 | >14.1E−6 | 28.51 ± 2.67 | | 0.25 | 3.09 | 3.15 |
| SR-01000312500 | 8.E−6 | 78.67 ± 30.35 | 62.76 | 0.27 | 1.48 | 1.49 |
| SR-01000357814 | >14.1E−6 | 7.60 ± 11.41 | | 0.26 | 2.21 | 2.25 |
| SR-01000068114 | >14.5E−6 | 12.12 ± 9.00 | | 0.23 | 2.54 | 2.54 |
| SR-01000145326 | >14.1E−6 | −2.11 ± 13.73 | | 0.23 | 1.95 | 1.95 |
| SR-01000070303 | >14.9E−6 | 19.47 ± 7.58 | | 0.23 | 5.02 | 5.02 |
| SR-01000520452 | >15.7E−6 | 1.29 ± 6.31 | | 0.28 | 1.13 | 1.26 |
| SR-01000127953 | >14.1E−6 | 10.89 ± 5.56 | | 0.30 | 1.96 | 2.35 |
| SR-01000260567 | >14.1E−6 | 31.40 ± 4.86 | | 0.38 | 2.05 | 2.05 |
| SR-00000006152 | >14.1E−6 | 8.91 ± 5.31 | | 0.26 | 2.04 | 2.04 |
| SR-01000080433 | >14.1E−6 | 29.03 ± 7.47 | | 0.30 | 3.22 | 3.22 |
| SR-01000090244 | >14.1E−6 | 1.54 ± 5.40 | | 0.23 | 3.06 | 3.06 |
| SR-01000105214 | >14.1E−6 | −5.28 ± 2.10 | | 0.26 | 2.13 | 2.13 |
| SR-01000034627 | >15.3E−6 | 2.09 ± 12.58 | | 0.25 | 1.47 | 1.47 |
| SR-01000069352 | >14.6E−6 | 11.25 ± 2.29 | | 0.32 | 1.25 | 1.25 |
| SR-01000155030 | >14.1E−6 | 10.75 ± 8.11 | | 0.30 | 1.32 | 1.32 |
| SR-01000391835 | >18.3E−6 | 10.10 ± 5.45 | | 0.32 | 0.66 | 4.05 |
| SR-01000051038 | >14.1E−6 | 22.35 ± 1.79 | | 0.24 | 1.71 | 1.71 |
| SR-01000271555 | >14.1E−6 | 14.71 ± 4.12 | | 0.32 | 1.38 | 1.38 |
| SR-01000135048 | >14.1E−6 | 12.06 ± 16.35 | | 0.30 | 3.00 | 3.00 |
| SR-01000162178 | >14.1E−6 | 25.44 ± 6.84 | | 0.30 | 1.58 | 1.73 |
| SR-00000006698 | >14.1E−6 | 5.81 ± 4.77 | | 0.21 | 3.11 | 6.10 |
| SR-01000479007 | >13.2E−6 | 8.46 ± 4.77 | | 0.30 | 2.56 | 2.56 |
| SR-01000140172 | >14.1E−6 | 2.22 ± 10.07 | | 0.30 | 3.41 | 3.41 |
| SR-00000005301 | >14.1E−6 | 8.29 ± 4.70 | | 0.30 | 1.56 | 1.56 |
| SR-01000146448 | >14.1E−6 | 10.11 ± 11.28 | | 0.26 | 2.27 | 2.27 |
| SR-01000146446 | >14.1E−6 | −2.89 ± 4.65 | | 0.27 | 2.71 | 2.71 |
| SR-01000031964 | >16.E−6 | 13.73 ± 5.82 | | 0.37 | 2.26 | 2.26 |
| SR-01000145135 | >14.1E−6 | −17.80 ± 4.24 | | 0.23 | 2.34 | 2.82 |
| SR-01000161248 | >14.1E−6 | −6.23 ± 14.84 | | 0.28 | 2.51 | 2.51 |
| SR-01000428888 | >16.4E−6 | −2.45 ± 12.32 | | 0.28 | 0.38 | 0.38 |
| SR-01000920602 | >14.1E−6 | −28.12 ± 1.91 | | 0.30 | 3.19 | 3.19 |
| SR-01000151350 | >14.1E−6 | 12.46 ± 7.98 | | 0.22 | 1.47 | 1.47 |
| SR-01000313440 | >14.1E−6 | 0.31 ± 4.15 | | 0.21 | 1.38 | 1.45 |
| SR-01000922914 | >14.1E−6 | −4.64 ± 6.81 | | 0.27 | 0.70 | 0.70 |
| SR-01000069574 | >14.5E−6 | 7.10 ± 4.83 | | 0.26 | 2.04 | 2.04 |
| SR-01000644888 | >14.1E−6 | 30.23 ± 7.95 | | 0.23 | 1.80 | 1.84 |
| SR-01000373848 | >14.1E−6 | 7.32 ± 0.96 | | 0.24 | 1.47 | 1.47 |
| SR-01000105999 | >14.1E−6 | 4.15 ± 6.01 | | 0.24 | 2.04 | 2.04 |
| SR-07000004609 | >14.1E−6 | −5.59 ± 4.81 | | 0.30 | −0.614 | −0.610 |
| SR-01000042109 | >15.E−6 | 21.12 ± 3.06 | | 0.36 | 2.18 | 2.18 |
| SR-01000129545 | >14.1E−6 | 32.45 ± 12.40 | | 0.23 | 1.09 | 1.11 |
| SR-01000286858 | >14.1E−6 | 4.98 ± 9.07 | | 0.28 | 2.08 | 2.09 |
| SR-01000916375 | >14.1E−6 | −1.52 ± 2.20 | | 0.27 | 1.41 | 1.86 |
| SR-01000132330 | >14.1E−6 | 5.54 ± 5.06 | | 0.23 | 2.06 | 2.06 |
| SR-01000582967 | >13.1E−6 | −6.63 ± 2.99 | | 0.21 | 2.93 | 2.93 |
| SR-01000284323 | >14.1E−6 | 3.28 ± 6.64 | | 0.31 | 3.34 | 3.34 |
| SR-01000920660 | >14.1E−6 | −18.29 ± 7.41 | | 0.31 | 1.83 | 1.83 |
| SR-01000924775 | >14.1E−6 | 10.92 ± 3.68 | | 0.23 | 3.42 | 3.42 |
| SR-01000073877 | >14.7E−6 | 30.72 ± 10.06 | | 0.31 | 1.60 | 1.69 |
| SR-01000135042 | >14.1E−6 | 16.89 ± 7.08 | | 0.28 | 1.77 | 1.77 |
| SR-01000314474 | >14.1E−6 | −0.07 ± 5.01 | | 0.28 | 1.59 | 1.59 |
| SR-01000193031 | >14.1E−6 | 25.60 ± 3.67 | | 0.23 | 4.19 | 4.36 |
| SR-01000310833 | >14.1E−6 | 13.95 ± 0.93 | | 0.42 | 1.78 | 1.78 |
| SR-01000050884 | >15.4E−6 | 5.22 ± 1.83 | | 0.27 | 1.88 | 1.88 |
| SR-00000007266 | >14.1E−6 | 23.72 ± 3.44 | | 0.27 | 1.75 | 1.76 |
| SR-01000145701 | >14.1E−6 | 10.54 ± 1.83 | | 0.26 | 2.54 | 4.13 |
| SR-01000139601 | >14.1E−6 | −14.37 ± 9.70 | | 0.23 | 2.04 | 2.04 |
| SR-01000359572 | >14.1E−6 | 6.12 ± 4.61 | | 0.28 | 2.65 | 2.65 |
| SR-01000335002 | >14.1E−6 | 20.28 ± 3.20 | | 0.23 | 3.09 | 3.09 |
| SR-01000428566 | >11.9E−6 | 40.80 ± 6.00 | | 0.20 | 0.07 | 0.07 |
| SR-01000086488 | >14.1E−6 | 1.08 ± 3.12 | | 0.22 | 2.40 | 2.44 |
| SR-01000163111 | >14.1E−6 | 5.61 ± 3.11 | | 0.22 | 4.15 | 4.20 |
| SR-01000468553 | >18.5E−6 | 35.00 ± 2.11 | | 0.27 | 1.48 | 1.48 |
| SR-01000062419 | >14.5E−6 | 19.92 ± 2.49 | | 0.25 | 0.97 | 0.98 |
| SR-01000074979 | >14.4E−6 | −0.33 ± 8.51 | | 0.23 | 2.51 | 2.52 |
| SR-01000146281 | >14.1E−6 | 8.26 ± 4.28 | | 0.28 | 1.82 | 1.82 |
| SR-01000056004 | >15.1E−6 | −16.39 ± 11.45 | | 0.22 | 2.55 | 2.56 |
| SR-01000513008 | >17.1E−6 | −5.44 ± 7.74 | | 0.32 | 1.74 | 1.74 |
| SR-01000068919 | >14.2E−6 | 39.50 ± 8.66 | | 0.23 | 1.78 | 1.78 |
| SR-01000070170 | >15.E−6 | 6.82 ± 9.60 | | 0.26 | 1.89 | 1.89 |
| SR-01000065547 | >14.1E−6 | 2.31 ± 13.81 | | 0.26 | 3.84 | 3.84 |
| SR-01000098914 | >14.1E−6 | 11.28 ± 8.03 | | 0.26 | 2.18 | 2.18 |
| SR-01000583711 | >11.1E−6 | 9.29 ± 2.62 | | 0.21 | 1.87 | 1.87 |
| SR-01000124131 | >14.1E−6 | −0.13 ± 10.83 | | 0.32 | 1.08 | 1.08 |
| SR-00000004710 | >14.1E−6 | −8.04 ± 14.30 | | 0.27 | 1.64 | 1.65 |
| SR-01000926510 | >14.1E−6 | 9.24 ± 7.87 | | 0.23 | 2.07 | 2.07 |
| SR-01000125648 | >14.1E−6 | 8.17 ± 5.14 | | 0.25 | 1.67 | 1.67 |
| SR-01000147127 | >14.1E−6 | −4.60 ± 15.16 | | 0.23 | 3.40 | 3.40 |
| SR-01000070173 | >14.7E−6 | 7.45 ± 6.67 | | 0.32 | 1.83 | 1.83 |
| SR-01000277633 | >14.1E−6 | −5.71 ± 6.76 | | 0.25 | 1.38 | 1.40 |
| SR-01000280669 | >14.1E−6 | −0.76 ± 4.25 | | 0.25 | 1.27 | 1.27 |
| SR-01000064101 | >14.9E−6 | 10.45 ± 2.67 | | 0.29 | 2.64 | 2.64 |
| SR-01000151917 | >14.1E−6 | 10.70 ± 4.71 | | 0.22 | 1.92 | 1.92 |
| SR-01000246445 | >14.1E−6 | 30.43 ± 4.82 | | 0.28 | 2.12 | 2.12 |
| SR-01000044537 | >14.6E−6 | 36.55 ± 5.27 | | 0.24 | 1.67 | 1.67 |
| SR-01000139458 | >14.1E−6 | 16.10 ± 9.95 | | 0.23 | 0.80 | 0.80 |
| SR-01000154995 | >14.1E−6 | 41.69 ± 44.54 | | 0.28 | 1.52 | 1.52 |
| SR-01000316982 | >14.1E−6 | 2.44 ± 4.96 | | 0.36 | 2.48 | 2.52 |
| SR-01000042749 | >14.1E−6 | −8.38 ± 6.55 | | 0.23 | 1.39 | 1.39 |
| SR-01000152099 | >14.1E−6 | 29.06 ± 12.18 | | 0.28 | 2.23 | 2.23 |
| SR-01000923206 | >14.1E−6 | −32.47 ± 4.02 | | 0.24 | 1.61 | 1.61 |
| SR-01000378352 | >14.1E−6 | 8.83 ± 9.35 | | 0.28 | 1.53 | 1.53 |
| SR-00000005393 | >14.1E−6 | 10.82 ± 8.56 | | 0.19 | 1.87 | 1.92 |
| SR-01000484524 | >14.3E−6 | −7.79 ± 9.91 | | 0.18 | 0.71 | 0.71 |
| SR-01000005203 | >13.9E−6 | 16.23 ± 3.90 | | 0.30 | 0.91 | 0.91 |
| SR-01000318171 | >14.1E−6 | 6.88 ± 5.68 | | 0.28 | 2.16 | 2.16 |
| SR-01000387617 | >14.1E−6 | 2.04 ± 4.49 | | 0.27 | 2.95 | 2.95 |
| SR-01000914977 | >14.1E−6 | 3.03 ± 10.02 | | 0.36 | 1.56 | 1.57 |
| SR-01000310664 | >14.1E−6 | 28.78 ± 5.42 | | 0.32 | 3.00 | 3.00 |
| SR-01000098980 | >14.1E−6 | 16.39 ± 16.07 | | 0.28 | 1.77 | 1.77 |
| SR-01000176838 | >14.1E−6 | −8.12 ± 1.57 | | 0.21 | 3.02 | 3.04 |
| SR-01000362527 | >14.1E−6 | 12.25 ± 2.76 | | 0.27 | 1.68 | 1.68 |
| SR-01000313193 | >14.1E−6 | 8.61 ± 6.03 | | 0.28 | 1.39 | 1.40 |
| SR-01000289377 | >14.1E−6 | −0.70 ± 7.45 | | 0.25 | 1.72 | 1.72 |
| SR-01000925002 | >14.1E−6 | 17.46 ± 3.54 | | 0.24 | 1.88 | 2.12 |
| SR-01000106321 | >14.1E−6 | 6.49 ± 5.13 | | 0.27 | 1.97 | 1.97 |
| SR-01000565308 | >17.5E−6 | 25.00 ± 13.25 | | 0.35 | 0.98 | 1.03 |
| SR-01000013346 | >14.6E−6 | −4.67 ± 3.34 | | 0.42 | 1.95 | 2.37 |
| SR-01000359546 | >14.1E−6 | 3.46 ± 3.80 | | 0.24 | 1.36 | 1.36 |
| SR-01000099712 | >14.1E−6 | 7.86 ± 5.01 | | 0.26 | 2.50 | 2.50 |
| SR-01000125346 | >14.1E−6 | 18.06 ± 8.45 | | 0.27 | 1.73 | 1.73 |
| SR-00000004123 | >14.1E−6 | 3.68 ± 4.29 | | 0.23 | 1.93 | 3.26 |
| SR-01000114492 | >14.1E−6 | −1.32 ± 3.21 | | 0.27 | 2.28 | 2.28 |
| SR-01000296110 | >14.1E−6 | 3.41 ± 3.54 | | 0.28 | 3.55 | 3.56 |
| SR-01000125626 | >14.1E−6 | −27.02 ± 6.51 | | 0.24 | 1.51 | 1.51 |
| SR-01000134913 | >14.1E−6 | −5.82 ± 11.13 | | 0.25 | 1.52 | 1.52 |
| SR-01000364205 | >14.1E−6 | −1.19 ± 1.27 | | 0.26 | 1.08 | 1.29 |
| SR-01000139982 | >14.1E−6 | 9.26 ± 7.81 | | 0.23 | 1.18 | 1.18 |
| SR-01000044433 | >14.3E−6 | 12.49 ± 3.39 | | 0.26 | 2.90 | 2.90 |
| SR-01000054340 | >14.2E−6 | 20.66 ± 8.49 | | 0.26 | 2.23 | 2.25 |
| SR-01000269911 | >14.1E−6 | 5.27 ± 9.17 | | 0.30 | 2.02 | 2.02 |
| SR-00000004378 | >14.1E−6 | −19.97 ± 10.80 | | 0.27 | 2.98 | 3.65 |
| SR-01000921162 | >14.1E−6 | −10.76 ± 9.17 | | 0.22 | 2.66 | 2.66 |
| SR-01000151470 | >14.1E−6 | −9.17 ± 11.99 | | 0.25 | 1.44 | 1.44 |
| SR-00000003277 | >14.1E−6 | 29.67 ± 5.05 | | 0.27 | 1.13 | 2.04 |
| SR-01000320687 | >14.1E−6 | 17.26 ± 6.30 | | 0.45 | 1.80 | 4.35 |
| SR-01000925700 | >14.1E−6 | 10.33 ± 2.07 | | 0.26 | 1.91 | 2.90 |
| SR-01000526835 | >13.9E−6 | 7.64 ± 8.84 | | 0.30 | 1.85 | 1.85 |
| SR-01000358286 | >14.1E−6 | 17.27 ± 10.49 | | 0.25 | 1.78 | 1.79 |
| SR-01000054961 | >14.1E−6 | 6.24 ± 6.42 | | 0.21 | 1.27 | 1.27 |
| SR-01000126429 | >14.1E−6 | 25.18 ± 6.22 | | 0.23 | 2.75 | 2.75 |
| SR-01000124163 | >14.1E−6 | −3.45 ± 3.02 | | 0.30 | 1.45 | 1.45 |
| SR-01000072797 | >14.2E−6 | 4.41 ± 6.50 | | 0.23 | 1.39 | 1.39 |
| SR-01000068179 | >14.7E−6 | 2.62 ± 3.03 | | 0.25 | 2.52 | 2.52 |
| SR-01000153956 | >14.1E−6 | 21.15 ± 4.99 | | 0.31 | 1.70 | 1.70 |
| SR-01000318521 | >14.1E−6 | 6.11 ± 9.12 | | 0.30 | 1.30 | 1.31 |
| SR-01000312779 | >14.1E−6 | 13.34 ± 1.65 | | 0.24 | 1.66 | 1.69 |
| SR-01000923250 | >14.1E−6 | −2.96 ± 8.90 | | 0.28 | 0.79 | 0.79 |
| SR-01000153886 | >14.1E−6 | −3.87 ± 9.73 | | 0.34 | 2.16 | 2.16 |
| SR-01000299610 | >14.1E−6 | −12.89 ± 6.99 | | 0.27 | 2.82 | 2.82 |
| SR-01000335115 | >14.1E−6 | 12.86 ± 9.78 | | 0.31 | 2.33 | 2.33 |
| SR-01000145456 | >14.1E−6 | −11.57 ± 15.53 | | 0.26 | 1.62 | 1.62 |
| SR-01000185621 | >14.1E−6 | −9.62 ± 7.28 | | 0.23 | 1.69 | 1.69 |
| SR-01000267161 | >14.1E−6 | 13.91 ± 0.63 | | 0.27 | 0.46 | 0.46 |
| SR-01000025120 | >14.9E−6 | −3.91 ± 5.24 | | 0.29 | 4.51 | 9.44 |
| SR-00000007044 | >14.1E−6 | 26.95 ± 5.80 | | 0.20 | 2.09 | 3.98 |
| SR-01000193756 | >14.1E−6 | −12.55 ± 2.76 | | 0.25 | 3.01 | 3.01 |
| SR-01000406098 | >11.5E−6 | 7.56 ± 6.15 | | 0.20 | 1.55 | 1.55 |
| SR-01000357697 | >14.1E−6 | −2.06 ± 7.71 | | 0.26 | 3.76 | 3.76 |
| SR-01000579592 | >13.3E−6 | −14.67 ± 10.75 | | 0.24 | 0.80 | 0.80 |
| SR-01000028953 | >15.3E−6 | 1.36 ± 9.86 | | 0.45 | 1.52 | 5.29 |
| SR-01000336681 | >14.1E−6 | 4.99 ± 7.59 | | 0.22 | 2.37 | 2.37 |
| SR-01000143463 | >14.1E−6 | −0.76 ± 20.69 | | 0.24 | 1.63 | 1.90 |
| SR-01000363853 | >14.1E−6 | −6.20 ± 7.21 | | 0.24 | 1.79 | 1.79 |
| SR-01000362228 | >14.1E−6 | −0.15 ± 6.76 | | 0.25 | 2.17 | 2.17 |
| SR-01000062305 | >14.8E−6 | −4.71 ± 5.40 | | 0.23 | 1.70 | 1.70 |
| SR-01000634249 | >14.1E−6 | 7.70 ± 7.23 | | 0.22 | 1.65 | 1.65 |
| SR-01000248261 | >14.1E−6 | 19.46 ± 6.76 | | 0.25 | 1.37 | 1.37 |
| SR-01000550322 | >13.4E−6 | −6.25 ± 1.61 | | 0.23 | 1.99 | 1.99 |
| SR-01000098884 | >14.1E−6 | 0.57 ± 13.32 | | 0.30 | 3.22 | 3.22 |
| SR-05000022582 | >14.1E−6 | 0.58 ± 18.59 | | 0.26 | −0.025 | 1.43 |
| SR-01000484494 | >16.E−6 | 14.09 ± 3.35 | | 0.23 | 0.89 | 0.89 |
| SR-00000005410 | >14.1E−6 | 7.35 ± 2.40 | | 0.26 | 2.07 | 2.07 |
| SR-01000124392 | >14.1E−6 | 5.97 ± 7.64 | | 0.23 | 2.31 | 2.31 |
| SR-01000126251 | >14.1E−6 | 29.14 ± 5.09 | | 0.25 | 2.56 | 2.56 |
| SR-01000361172 | >14.1E−6 | 2.50 ± 11.74 | | 0.34 | 1.93 | 1.97 |
| SR-01000011241 | >14.2E−6 | 1.28 ± 10.93 | | 0.22 | 1.05 | 1.05 |
| SR-01000325257 | >14.1E−6 | −2.80 ± 2.96 | | 0.25 | 2.44 | 2.48 |
| SR-01000911742 | >14.1E−6 | −12.80 ± 5.17 | | 0.22 | 1.49 | 1.49 |
| SR-01000429212 | >18.E−6 | −31.95 ± 8.26 | | 0.29 | 1.05 | 1.05 |
| SR-01000136273 | >14.1E−6 | −14.31 ± 5.07 | | 0.27 | 1.72 | 1.72 |
| SR-01000921037 | >14.1E−6 | 2.69 ± 3.11 | | 0.22 | 3.11 | 3.16 |
| SR-01000119565 | >14.1E−6 | 5.54 ± 5.16 | | 0.26 | 2.79 | 2.79 |
| SR-01000017533 | >14.2E−6 | −1.95 ± 4.81 | | 0.27 | 2.68 | 2.68 |
| SR-01000299370 | >14.1E−6 | −5.05 ± 8.64 | | 0.34 | 0.78 | 0.78 |
| SR-01000465824 | >11.8E−6 | −11.98 ± 3.62 | | 0.25 | 1.67 | 1.72 |
| SR-01000360839 | >14.1E−6 | 0.18 ± 6.80 | | 0.25 | 2.59 | 4.41 |
| SR-01000597171 | 10.7E−6 | 92.06 ± 10.88 | 2.90 | 0.43 | 3.35 | 3.35 |
| SR-01000141798 | >14.1E−6 | 2.60 ± 1.15 | | 0.28 | 2.66 | 2.66 |

### Example 2. Selective Binding of Bitopic Ligands to GPR139

Eighteen compounds were selected for additional study. The compounds were re-synthesized and tested in radioligand binding competition assays for their ability to displace [³H]-labeled reference agonist JNJ-63533054 on GPR139. The GPR139 construct used (pCDNA3.1-HASP-HA-GPR139) contained an N-terminal HA signal peptide (HASP), followed by an HA tag and human GPR139 (NM_001002911.4). HEK293T/17 cells were maintained in Dulbecco's modified Eagle's medium with 10% (v/v) fetal bovine serum, nonessential amino acids, and 1 mM sodium pyruvate. Transfections were performed as described in Masuho I et al. “Monitoring G Protein Activation in Cells with BRET.” Methods Mol Biol 2015 1335:107-113.

Competition binding assay: Radioligand binding assay used human GPR139 expressed in HEK293T/17 cells. HEK293T/17 cells were transfected pCDNA3.1-HASP-HA-GPR139. 48 h after, cells were detached from plates by pipetting with PBS, centrifuged at 500×g for 5 min, and then the fluid was aspirated away. The cell pellets were frozen with liquid nitrogen and stored at −80° C. Pellets were homogenized in TE buffer (50 mM Tris-HCl, pH 7.4, 5 mM EDTA) and centrifuged at 1000×g at 4° C. for 5 min. Supernatant was collected and recentrifuged at 30,000×g at 4° C. for 30 min. The pellet was rehomogenized in TE buffer and incubated for 60 minutes at room temperature with eight concentrations of [³H]JNJ-63533054 (specific activity 24.7 Ci/mmol) ranging from 0.024 nM to 400 nM. The binding reaction was terminated by filtration through GF/C filter (Waterman) followed by washes with cold TE buffer. Filters were dried in a hood overnight followed by the addition of scintillation fluid. Bound radioactivity was counted on a Hidex 600 liquid scintillation counter. For inhibition of [³H]JNJ-63533054 (10 mM) radioligand binding, 6-7 concentration of each of the eighteen compounds were added to the cell membranes. Nonspecific binding was determined with 10 μM JNJ-63533054. The Kd values of the radioligands and IC₅₀ of the antagonist were calculated by Graphpad Prism. Ki values were calculated as Ki=IC₅₀/(1+C/Kd), where C is concentration of the radioligand.

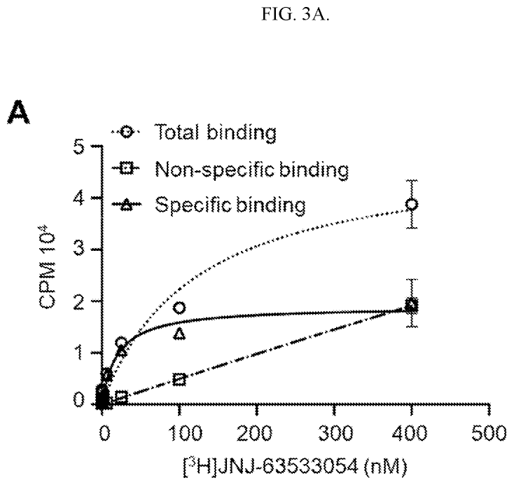

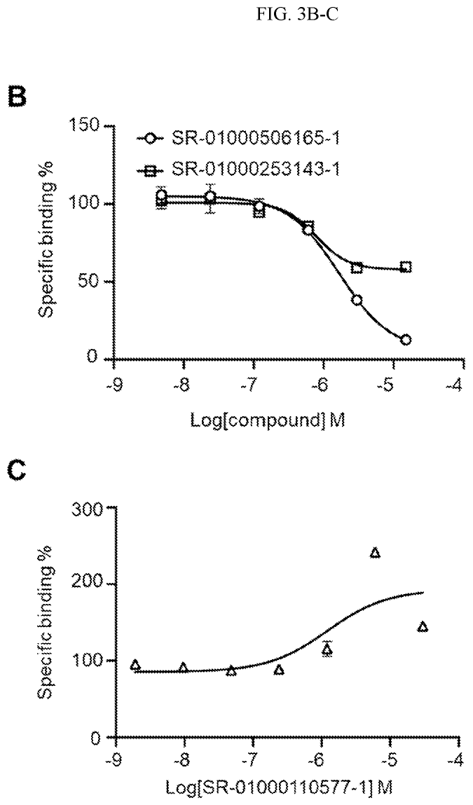

Results: Specific binding of JNJ-63533054 to GPR139 is shown in FIG. 3A. Various concentrations of selected ligands were then added in the presence of 10 nM [³H]-JNJ-63533054 and their ability to displace [³H]labeling on cellular membranes was assessed. FIGS. 3B and 3C present representative data obtained in these experiments. 17 compounds tested exhibited clear displacement of JNJ-63533054 while 1 compound (FIG. 3C) potentiated it suggesting that this could be a bitopic ligand for GPR139. The results of these experiments are presented in the Table 3 and confirm selective binding the compounds to GPR139 thereby validating their interaction with the intended target.

TABLE 5
Inhibition equilibrium constants (Ki) and % inhibition of (10
μM) JNJ-63533054 binding to membranes of HEK293T/17 cells
expressing human GPR139 by 10 μM of the indicated compound.

| | Ki ± SEM | % inhibition of |
|---|---|---|
| Sample ID | (μM) | JNJ-63533054 |
| SR-07000003837-2 analog 1 | 9.45 ± 0.21 | 88.67 ± 21.12 |
| SR-07000003837-2 analog 2 | 1.29 ± 0.03 | 51.87 ± 5.47 |
| SR-01000907648-1 analog | 6.07 ± 0.14 | 65.55 ± 0.72 |
| SR-01000263867-1 | 4.91 ± 1.10 | 59.31 ± 7.51 |
| SR-01000258561-1 | 7.14 ± 0.16 | 35.56 ± 21.16 |
| SR-01000506165-1 | 1.10 ± 0.02 | 103.18 ± 1.95 |
| SR-01000452455-1 analog | 1.68 ± 0.04 | 107.02 ± 1.95 |
| SR-01000253143-1 | 0.13 ± 0.01 | 54.27 ± 5.98 |
| SR-01000220077-1 | 7.31 ± 0.16 | 77.75 ± 5.00 |
| SR-01000914310-1 | 1.06 ± 0.02 | 72.20 ± 2.78 |
| SR-01000195221-1 | 9.98 ± 0.23 | 77.69 ± 1.19 |
| SR-01000110577-1 | 8.03 ± 0.18 | −117.18 ± 2.89 |
| SR-01000244770-1 | 146 ± 0.003 | 56.28 ± 7.80 |
| SR-01000421299-1 | 1.81 ± 0.04 | 64.98 ± 3.44 |
| SR-01000594343-1 | 3.05 ± 0.07 | 87.17 ± 2.37 |
| SR-01000419112-1 | 0.98 ± 0.02 | 102.49 ± 4.88 |
| SR-01000169333-1 | 4.09 ± 0.09 | 64.47 ± 1.23 |
| SR-01000514922-1 | 0.92 ± 0.02 | 21.83 ± 14.12 |
| SR-01000462122-1 | 13.32 ± 0.30 | 22.01 ± 2.59 |

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims

All publications, databases, patents, and patent applications cited in this specification are herein incorporated by reference as if each was specifically and individually indicated to be incorporated by reference.

1. A method for reducing GPR139 activity and/or GPR139 signaling activity in a cell comprising contacting the cell with one or more GPR139 antagonists of Table 1.

2. A method for increasing GPR139 activity and/or GPR139 signaling activity in a cell comprising contacting the cell with one or more GPR139 agonists of Table 2.

3. The method of claim 1 or 2, wherein the cell is in a subject.

4. A method for providing or enhancing an analgesic effect mediated by a μ-opioid receptor (MOR) in a subject, comprising administering to the subject an effective amount of one or more GPR139 antagonists of Table 1.

5. The method of claim 4, wherein the subject is administered an opioid drug for pain relief.

6. The method of claim 5, wherein the subject is administered the opioid drug prior to, simultaneously with, or subsequent to administration of the one or more GPR139 antagonists.

7. The method of claim 6, wherein the opioid drug is oxycodone, hydrocodone, morphine, codeine, dihydrocodeine, fentanyl, buprenorphine, or methadone.

8. The method of any one of claims 4-7, wherein the subject is a human.

9. A method for suppressing or ameliorating one or more symptoms associated with opioid withdrawal in a subject comprising administering to the subject an effective amount of one or more GPR139 antagonists of Table 1.

10. The method of claim 9, wherein the subject is suffering from the one or more withdrawal symptoms or is at risk of suffering from the one or more withdrawal symptoms.

11. The method of claim 9 or 10, wherein suppressing or ameliorating one or more symptoms associated with opioid comprises reducing dependence on an opioid drug, assisting the subject in reducing opioid use, or treating an opioid use disorder.

12. The method of any one of claims 9-11, wherein the subject has an opioid use disorder.

13. The method of any one of claims 9-12, wherein the effective amount of the one or more GPR139 antagonists is administered to the subject after discontinuing or reducing use of the opioid drug, or prior to discontinuing or reducing use of the opioid drug.

14. The method of any one of claims 9-13, wherein the opioid drug selected from the group consisting of: oxycodone, hydrocodone, morphine, codeine, dihydrocodeine, heroin, opium, and fentanyl.

15. The method of any one of claims 9-14, wherein the subject is a human.

16. A pharmaceutical composition comprising a GPR139 antagonist of Table 1.

17. The pharmaceutical composition of claim 16, further comprising an opioid.

18. The pharmaceutical composition of claim 17, wherein the opioid is selected from the group consisting of: oxycodone, hydrocodone, morphine, codeine, dihydrocodeine, and fentanyl

19. A method of reducing a reward associated with opioid use or diminishing a reinforcing effect of opioid use in a subject comprising administering to the subject an effective amount of one or more GPR139 agonists of Table 2.

20. The method of claim 19, wherein reducing the reward associated with opioid use or diminishing the reinforcing effects of opioid use is used to treat or prevent opioid addiction or dependence in the subject.

21. The method of claim 19 or 20, wherein the subject is an acute user or a chronic user of opioids.

22. A method of treating a neuropsychiatric disorder in a subject comprising administering to the subject an effective amount of one or more GPR139 agonists of Table 2.

23. The method of claim 22, wherein treating the neuropsychiatric disorder comprises treating one or more symptoms associated with the neuropsychiatric disorder.

24. The method of claim 23, wherein treating one or more symptoms associated with the neuropsychiatric disorder comprises decreasing or suppressing severity of the one or more symptoms, decreasing the frequency of the one or more symptoms, or decreasing progression of the one or more symptoms.

25. The method of claim 24, wherein the one or more symptoms are selected from the group consisting of: delusions, hallucinations, confused or disorganized thinking, trouble with logical thinking, confused or disordered speech, abnormal movements, paranoia, inability to express emotion, inability to find pleasure, and exaggerated or distorted perceptions beliefs and behaviors.

26. The method of any one of claims 22-25, wherein the neuropsychiatric disorder is selected from the group consisting of: schizophrenia, a schizophrenia-related disorder, a schizotypal personality disorder, an obsessive-compulsive disorder, Huntington's disease, a deficit in social interactions, a prepulse inhibition disorder, and spontaneous or involuntary head twitching or other spontaneous or involuntary twitches.

27. The method of any one of claims 22-26, wherein the one or more GPR139 agonists are administered in combination with an antipsychotic, a mood stabilizer, and/or an antidepressant.

28. The method of claim 27, wherein the one or more additional antipsychotics are selected from the group consisting of: chlorpromazine, fluphenazine, haloperidol, perphenazine, thioridazine, thiothixene, trifluoperazine, aripiprazole, aripiprazole lauroxil, asenapine, brexpiprazole, cariprazine, clozapine, iloperidone, lumateperonee, lurasidone, olanzapine, olanzapine/samidorphan, paliperidone, paliperidone palmitate, quetiapine, risperidone, and ziprasidone.

29. A pharmaceutical composition comprising a GPR139 agonist of Table 2.

30. The pharmaceutical composition of claim 29, further comprising an antipsychotic, a mood stabilizer, and/or an antidepressant.

31. The pharmaceutical composition of claim 30, wherein the antipsychotic is selected from the group consisting of; chlorpromazine, fluphenazine, haloperidol, perphenazine, thioridazine, thiothixene, trifluoperazine, aripiprazole, aripiprazole lauroxil, asenapine, brexpiprazole, cariprazine, clozapine, iloperidone, lumateperonee, lurasidone, olanzapine, olanzapine/samidorphan, paliperidone, paliperidone palmitate, quetiapine, risperidone, and ziprasidone.

32. A method of modulating GPR139 activity in a subject comprising administering to the subject a pharmaceutically effective dose of one or more GPR139 antagonists of Table 1 or one or more GPR139 agonists of Table 2.

33. A method of modulating GPCR signaling function in a subject comprising administering to the subject a pharmaceutically effective dose of one or more GPR139 antagonists of Table 1 or one or more GPR139 agonists of Table 2A.
